(19) United States
(12) Patent Application Publication
(10) Pub. No.: US 2026/0256407 A1
(43) Pub. Date: Sep. 3, 2026

(54) METHODS AND SYSTEMS FOR PREDICTING TREATMENT OUTCOMES, PATIENT SELECTION AND PERSONALIZED THERAPY USING PATIENT RESPONSE PROPERTIES TO SENSORY STIMULATION

(71) Applicant: Cognito Therapeutics, Inc., Cambridge, MA (US)

(72) Inventors: Zachary John Hambrecht Malchano, Boston, MA (US); Aylin Cimenser, Cambridge, MA (US); Martin Williams, San Francisco, CA (US); Mihály Hajós, Cambridge, MA (US); Brent Vaughan, Cambridge, MA (US)

(21) Appl. No.: 19/429,539

(22) Filed: Dec. 22, 2025

Related U.S. Application Data
(63) Continuation of application No. 19/204,233, filed on May 9, 2025, now abandoned.
Application No. 19/204,233 is a continuation of application No. 18/908,398, filed on Oct. 7, 2024, now abandoned.
Application No. 18/908,398 is a continuation of application No. PCT/US2023/015570, filed on Mar. 17, 2023.
(60) Provisional application No. 63/321,301, filed on Mar. 18, 2022.

(51) Int. Cl. A61B 5/372; A61B 5/00; A61B 5/378; A61B 5/38; A61B 5/383; G16H 10/60; G16H 20/30; G16H 50/30; G16H 50/70

(52) U.S. Cl. CPC A61B 5/372; A61B 5/378; A61B 5/38; A61B 5/383; A61B 5/4836; A61B 5/7275; A61B 5/742; G16H 10/60; G16H 20/30; G16H 50/30; G16H 50/70

(57) ABSTRACT

In some aspects, the present disclosure describes a method of predicting an expected treatment outcome of a subject, comprising administering a gamma oscillation-inducing non-invasive sensory stimulus to the subject, measuring a response from the subject, and predicting, using a machine learning algorithm, the expected treatment outcome of the subject based at least partially on the measured response. In some aspects, the present disclosure also provides methods for personalizing gamma therapy treatment by adjusting parameters associated with the gamma oscillation-inducing non-invasive sensory stimulus based on a subject's response to the gamma oscillation-inducing non-invasive sensory stimulus.

### CROSS-REFERENCE

This application is a continuation of U.S. application Ser. No. 19/204,233 filed May 9, 2025, which is a continuation application of U.S. application Ser. No. 18/908,398 filed Oct. 7, 2024, now abandoned, which is a continuation of International Application No. PCT/US2023/015570 filed Mar. 17, 2023, which application claims the benefit of U.S. Provisional Application No. 63/321,301 filed Mar. 18, 2022, each of which is incorporated herein by reference in its entirety.

### INCORPORATION BY REFERENCE

Each patent, publication, and non-patent literature cited in the application is hereby incorporated by reference in its entirety as if each was incorporated by reference individually.

### BACKGROUND

Neural oscillation occurs in humans or animals and includes rhythmic or repetitive neural activity in the central nervous system. Neural tissue can generate oscillatory activity by mechanisms within individual neurons or by interactions between neurons. Oscillations can appear as either oscillations in membrane potential or as rhythmic patterns of action potentials, which can produce oscillatory activation of post-synaptic neurons. Synchronized activity of a group of neurons can give rise to macroscopic oscillations, which can be observed noninvasively by electroencephalography (“EEG”). Neural oscillations can be characterized by their frequency, amplitude, and phase. These signal properties can be studied in time and/or frequency domains and can give information about the underlying brain dynamics. Neural oscillations can be elicited externally, by stimulation or drugs, subject's EEG response properties to stimulation or drugs can also give information about the underlying brain dynamics and can have information about the subject's condition.

Neurological conditions that impact the nervous systems of humans and animals can be difficult to diagnose, evaluate, and treat, due to delayed symptoms, often overlapping or similar symptoms between diseases, lack of accurate quantitative assays based on biomarkers, or long preclinical and prodromal phases.

### SUMMARY

In some aspects, the present disclosure discloses a method of predicting a likelihood of a subject benefiting from treatment, the method comprising: administering a neural oscillation inducing non-invasive sensory stimulus to the subject; measuring a response from the subject; and predicting, using a statistical algorithm, a machine learning algorithms, or a combination thereof the expected treatment outcome of the subject, wherein the prediction is based at least partially on the measuring of the response from the subject, and wherein the expected treatment outcome comprises a reduction of neurodegeneration, a slowing of neurodegeneration, a reduction or prevention of brain atrophy due to aging or neurodegeneration, a slowing of brain atrophy due to aging or neurodegeneration, an improvement in symptoms of neurodegeneration, a slowing of cognitive and functional decline, an improvement in cognition, an improvement in function, an improvement in symptoms of neurological and psychiatric diseases, or a combination thereof.

In any embodiment of the present disclosures, the gamma oscillation inducing non-invasive sensory stimulus comprises a periodic stimulus. In some certain embodiments, the periodic stimulus comprises a frequency component from about 20 Hz to about 160 Hz. In some certain embodiments, the periodic stimulus comprises a frequency component from about 25 Hz to about 80 Hz. In some certain embodiments, the periodic stimulus comprises a frequency component from about 30 Hz to about 50 Hz. In some certain embodiments, the periodic stimulus comprises a frequency component from about 35 Hz to about 45 Hz. In some certain embodiments, the periodic stimulus comprises a frequency component of about 45 Hz. In some certain embodiments, the periodic stimulus is intermittent. In some certain embodiments, the non-invasive sensory stimulus further comprises non-periodic components.

In some embodiments, the gamma oscillation inducing non-invasive sensory stimulus is a visual stimulus, an auditory stimulus, a kinesthetic stimulus, or any combination thereof.

In some embodiments, the subject is diagnosed with or is at a risk of developing a neurodegenerative disorder associated with cognitive decline.

In some embodiments, the neurodegenerative disorder comprises supranuclear palsy (PSP).

In some embodiments, the neurodegenerative disorder is a prion disease or transmissible spongiform encephalopathy.

In some embodiments, the neurodegenerative disorder is Alzheimer's disease, Creutzfeldt-Jakob disease (CJD), variant CJD, Gerstmann-Sträussler Scheinker Syndrome, Fatal Familial Insomnia, Kuru, or any combination thereof.

In some embodiments, the subject is diagnosed with or is at a risk of developing behavioral and psychological symptoms of dementia (BPSD).

In some embodiments, the subject is diagnosed or is at risk of developing a mood disorder, apathy, agitation, depression, bipolar disorder, anxiety, addiction, neurosis, anorexia, bulimia, dementia, mild cognitive impairment, subjective cognitive decline, Lewy body dementia, Parkinson's disease, sleep fragmentation, schizophrenia, or any combination thereof.

In some embodiments, the expected treatment outcome is a reduction of a frequency, duration, or severity of a symptom associated with a psychiatric or neurological disorder. In certain embodiments, the symptom is a symptom associated with bipolar disorder or schizophrenia.

In some embodiments, the subject is a subject who is treated for or diagnosed with a neurodegenerative disorder.

In some embodiments, the response is measured using an electroencephalogram (EEG).

In some embodiments, the EEG measures at least EEG coherence.

In some embodiments, the EEG coherence is correlated with a measure of the clinical outcome of the subject.

In some embodiments, the EEG coherence is correlated with a plurality of measures of the clinical outcome of the subject.

In some embodiments, the measure comprises Mini-Mental State Examination (MMSE), Alzheimer's Disease Assessment Scale (ADAS-Cog), Clinical Dementia Rating (CDR), Alzheimer's Disease Cooperative Study-Activities of Daily Living (ADCS-ADL), Neuropsychiatric Inventory (NPI), positron emission tomography (PET), or magnetic resonance imaging (MRI) volumetric data assessments. In some cases, the measure comprises a plurality of measures. In some cases, the measure comprises composite measures. In some embodiments, the composite measures comprise weighted composite measures, unweighted composite measures, or a combination thereof. In some embodiments, the measure is mapped using a Global Statistic Tests. In some embodiments, the measure comprises composite measures, weighted composites, global statistic tests, or z-scores based on the one or more measures.

In some embodiments, the gamma oscillation inducing non-invasive sensory stimulus is continuously administered for a predetermined duration of time. In some certain embodiments, the predetermined duration of time is from 10 minutes to 2 hours.

In some embodiments, the gamma oscillation inducing non-invasive sensory stimulus is administered for a plurality of discrete times. In some certain embodiments, the plurality of discrete times occur at least once a day. In some certain embodiments, the plurality of discrete times occur at least once every two days. In some certain embodiments, the plurality of discrete times occur at least once a week. In some certain embodiments, the plurality of discrete times occur at least once every two weeks. In some certain embodiments, the plurality of discrete times occur at least once a month. In some certain embodiments, the plurality of discrete times occur at least once every other month.

Alternatively, or in addition, in some certain embodiments the plurality of discrete times span over at least two days. Alternatively, or in addition, in some certain embodiments the plurality of discrete times span over at least a week. Alternatively, or in addition, in some certain embodiments the plurality of discrete times span over at least two weeks. Alternatively, or in addition, in some certain embodiments the plurality of discrete times span over at least once a month. Alternatively, or in addition, in some certain embodiments the plurality of discrete times span over at least three months. Alternatively or in addition, in some certain embodiments the plurality of discrete times span over at least six months. Alternatively or in addition, in some certain embodiments the plurality of discrete times span over at least a year. Alternatively, or in addition, in some certain embodiments the plurality of discrete times span over at least five years.

In some embodiments, the method further comprises selecting the subject as a patient who may benefit from administration of gamma oscillation inducing non-invasive sensory stimulus based therapy at least partially on the expected treatment outcome. In some embodiments, the method further comprises selecting the subject as a patient who is unlikely to benefit from administration of gamma oscillation inducing non-invasive sensory stimulus based therapy at least partially on the expected treatment outcome.

In some embodiments, the method further comprises selecting the subject as a patient for a treatment plan based at least partially on the expected treatment outcome. In some certain embodiments, the treatment plan is a gamma oscillation inducing non-invasive sensory stimulation treatment plan.

In some embodiments, the machine learning algorithm comprises a neural network, a deep learning algorithm, an ensemble, a regularization, a rule system, a regression, a Bayesian analysis, a decision tree, a dimensionality reduction, an instance-based algorithm, or a clustering algorithm.

In some embodiments, the method further comprises using network information of the subject to predict the expected treatment outcome of the subject.

In some embodiments, the method further comprises using biometric data of the subject to predict the expected treatment outcome of the subject.

In some embodiments, the biometric data is sleep data.

In some embodiments, the expected treatment outcome is an outcome of a treatment for sleep fragmentation.

In some embodiments, the expected treatment outcome comprises a reduction in brain atrophy due to aging.

In some embodiments, the expected treatment outcome comprises a slowing in brain atrophy due to aging.

In some embodiments, the expected treatment outcome comprises a reduction of neurodegeneration. In some certain embodiments, the reduction of neurodegeneration comprises a reduction in nervous system atrophy. In some specific embodiments, the reduction in nervous system atrophy comprises a reduction in brain atrophy. In some specific embodiments, the reduction in nervous system atrophy comprises a reduction in peripheral nervous system atrophy.

In some embodiments, the expected treatment outcome comprises a slowing of neurodegeneration. In some specific embodiments, the slowing of neurodegeneration comprises a slowing of the rate of nervous system atrophy. In some even more specific embodiments, the slowing of the rate of nervous system atrophy comprises a slowing of the rate of brain atrophy. In some even more specific embodiments, the slowing of the rate of nervous system atrophy comprises a slowing of the rate of peripheral nervous system atrophy.

In some embodiments, the expected treatment outcome comprises an improvement of symptoms of neurodegeneration. In some certain embodiments, the improvement of symptoms of neurodegeneration comprises improved sleep, improved cognitive abilities, improved memory, improved muscle control, improved balance, improved breathing, improved heart function, or a combination thereof.

In some aspects, the present disclosure discloses a method of identifying a biomarker associated with a distinct clinical outcome, comprising: training a machine learning algorithm to identify a statistical relationship between (i) a first dataset comprising a plurality of response measurements for a plurality of subjects, wherein the plurality of response measurements comprises a response to a gamma oscillation inducing non-invasive sensory stimulus for each subject in the plurality of subjects, and (ii) a second dataset comprising a plurality of clinical measurements for the plurality of subjects; and identifying, using statistical or machine learning algorithm, the biomarker associated with the distinct clinical outcome.

In some embodiments, the plurality of response measurements comprises a plurality of bioelectrical measurements.

In some embodiments, the plurality of bioelectrical measurements is a plurality of electroencephalogram (EEG) measurements.

In some embodiments, the biomarker is a signal pattern in the plurality of response measurements.

In some embodiments, the one or more measurements of clinical outcomes comprise Mini-Mental State Examination (MMSE), Clinical Dementia Rating (CDR), Alzheimer's Disease Cooperative Study-Activities of Daily Living (ADCS-ADL), Neuropsychiatric Inventory (NPI), positron emission tomography (PET), or magnetic resonance imaging (MRI) volumetric data assessments. In some embodiments, the one or more measurements of clinical outcomes comprise measures of daily movement, activity levels, apathy measures, actigraphy or sleep quality measures.

In some aspects, the present disclosure discloses a computer-implemented method of predicting a response to a treatment for a subject diagnosed with or at a risk of developing a neurodegenerative disorder associated with cognitive decline, the computer-implemented method comprising: providing a visual gamma oscillation inducing stimulus to the subject; and performing an encephalogram on a brain region of the subject to measure a plurality of bioelectrical signals.

In some embodiments, the gamma oscillation inducing visual stimulus comprises a periodic stimulus. In some certain embodiments, the periodic stimulus comprises a frequency component from about 20 Hz to about 160 Hz. In some certain embodiments, the periodic stimulus comprises a frequency component from about 25 Hz to about 80 Hz. In some certain embodiments, the periodic stimulus comprises a frequency component from about 30 Hz to about 50 Hz. In some certain embodiments, the periodic stimulus comprises a frequency component from about 35 Hz to about 45 Hz. In some certain embodiments, the periodic stimulus comprises a frequency component of about 45 Hz. In some certain embodiments, the periodic stimulus is intermittent. In some certain embodiments, the visual stimulus further comprises non-periodic components.

In some embodiments, the gamma oscillation inducing visual stimulus is provided using a display device.

In some aspects, the present disclosure discloses a computer-implemented method of administering a treatment and computing an expected clinical outcome score of the treatment of a subject diagnosed with Alzheimer's Disease, the computer-implemented method comprising: administering a therapeutic dose of gamma oscillation inducing non-invasive sensory stimulus to a brain of the subject; performing an encephalogram on the brain of the subject to measure a plurality of bioelectrical signals; computing, using a statistical or machine learning algorithm, the expected clinical outcome score of the subject based on the plurality of bioelectrical signals; and adjusting the therapeutic dose based at least partially on the expected clinical outcome score.

In some embodiments, adjusting the therapeutic dose comprises adjusting a parameter of the gamma oscillation inducing non-invasive sensory stimulus. In certain embodiments, adjusting a parameter of the gamma oscillation inducing non-invasive sensory stimulus comprises adjusting a duration, frequency, wavelength, duty cycle, phase, amplitude, intensity, spectrum, envelope, interstimulus interval, harmonic structure, modulation, or waveform of the stimulus. In some specific embodiments, adjusting the therapeutic dose comprises adjusting the parameters of a square-wave stimulus. In some specific embodiments, adjusting the therapeutic dose comprises adjusting the parameters of a sinusoidal wave stimulus. In some specific embodiments, adjusting the therapeutic dose comprises adjusting the parameters of a noise stimulus. In some even more specific embodiments, adjusting the therapeutic dose comprises adjusting the parameters of a white noise stimulus. In some even more specific embodiments, adjusting the therapeutic dose comprises adjusting the parameters of a pink noise stimulus. In some specific embodiments, adjusting the therapeutic dose comprises adjusting the parameters of a chirp stimulus. In some even more specific embodiments, adjusting the therapeutic dose comprises adjusting the parameters of an O-chirp stimulus. In some specific embodiments, adjusting the therapeutic dose comprises adjusting the parameters of a click stimulus.

The present disclosure further provides a method for selecting a patient population that will respond to gamma oscillation inducing non-invasive sensory stimulation, the method comprising: (a) administering a gamma oscillation inducing non-invasive sensory stimulus to a subject of the patient population; (b) measuring a response from the subject; and (c) predicting, using a statistical, machine learning algorithm, or a combination thereof the expected treatment outcome of the patient population based at least partially on the measuring of (b). In some embodiments, the expected treatment outcome comprises a reduction of neurodegeneration, a slowing of neurodegeneration, an improvement in symptoms of neurodegeneration, or a combination thereof.

Also provided herein is a method for identifying favorable parameter settings for gamma oscillation inducing non-invasive sensory stimulation therapy, the method comprising: (a) administering a gamma oscillation inducing non-invasive sensory stimulus to a subject of the patient population; (b) measuring a response from the subject; and (c) modifying a parameter of the gamma oscillation inducing non-invasive sensory stimulus, wherein the parameter of the gamma oscillation inducing non-invasive sensory stimulus is modified based at least partially on the measuring of (b), thereby identifying favorable parameter settings for gamma oscillation inducing non-invasive sensory stimulation therapy.

Further provided is a method for selecting parameters to serve as a placebo for treatment using gamma oscillation inducing non-invasive sensory stimulus, the method comprising (a) administering a stimulus to a subject of the patient population; (b) measuring a response from the subject; (c) modifying one or more parameters of the stimulus to reduce the response from the subject; and (d) repeating (a) through (c) until the response from the subject is minimal, thereby selecting parameters to serve as a placebo for treatment using gamma oscillation inducing non-invasive sensory stimulus.

In some embodiments, the parameter of the stimulus comprises a plurality of parameters of the stimulus.

The present also disclosure provides systems and methods for selecting a brain region to target with treatment, the method comprising (a) administering a non-invasive sensory stimulus to a subject of the patient population; (b) performing an encephalogram on the brain of the subject to measure a plurality of bioelectrical signals; (c) selecting, based on the plurality of bioelectrical signals, the brain region to target with treatment. In some cases, the brain region comprises a plurality of brain regions.

In some cases, the brain region has reduced amplitude of neural oscillations relative to an alternative brain region. In some cases, the brain region has reduced coherence of neural oscillations relative to an alternative brain region.

The present disclosure further provides a method for defining a unified clinical outcome for progression of a disease or disorder, the method comprising (a) administering a gamma oscillation inducing non-invasive sensory stimulus to a plurality of individuals; (b) measuring one or more biological responses of the plurality of individuals to the gamma oscillation, wherein the one or more biological responses are associated with progression of the disease or disorder; (c) normalizing values corresponding to the one or more biological responses, thereby generating normalized data points; and (d) determining, using the normalized data points, a principal component for the data points.

In some embodiments, the biological response comprises a plurality of biological responses.

In some embodiments, the brain region comprises a plurality of brain regions.

Also provided herein is method comprising: (a) administering gamma oscillation inducing non-invasive sensory stimulus to a plurality of individuals; (b) measuring a response of the plurality of individuals to the gamma oscillation inducing non-invasive sensory stimulus using an encephalogram; (c) dividing the plurality of individuals into groups based on identified shared characteristics; and (d) preparing data sets for each group of the plurality of individuals.

In some cases, the methods further comprise (e) administering gamma oscillation inducing non-invasive sensory stimulus to a subject; (f) characterizing a response of the subject to the gamma oscillation inducing non-invasive sensory stimulus based on the data sets for each group of the plurality of individuals; and (g) diagnosing, based on the data sets, the subject.

The present disclosure further provides methods for identifying stimulus parameters that target an aspect of a disease, the method comprising: (a) administering gamma oscillation inducing non-invasive sensory stimulus to a plurality of individuals; (b) measuring a biological response of the plurality of individuals to the gamma oscillation inducing non-invasive sensory stimulus; (c) evaluating, using a machine-based algorithm, the biological response of the plurality of individuals; (d) adjusting one or more parameters of the gamma oscillation inducing non-invasive sensory stimulus; and repeating (a) through (c) to identify stimulus parameters that target an aspect of a disease.

In some embodiments, the aspect of the disease comprises a plurality of aspects.

In some embodiments, the disease is a neurological disorder associated with cognitive decline. In some specific embodiments, the neurodegenerative disorder comprises supranuclear palsy (PSP). In some specific embodiments, the neurodegenerative disorder is a prion disease or transmissible spongiform encephalopathy. In some specific embodiments, the neurodegenerative disorder is Alzheimer's disease, Creutzfeldt-Jakob disease (CJD), variant CJD, Gerstmann-Sträussler Scheinker Syndrome, Fatal Familial Insomnia, Kuru, or any combination thereof. In some specific embodiments, the subject is diagnosed with or is at a risk of developing behavioral and psychological symptoms of dementia (BPSD).

In some embodiments, the subject is diagnosed or is at risk of developing a mood disorder, apathy, agitation, depression, bipolar disorder, anxiety, addiction, neurosis, anorexia, bulimia, dementia, mild cognitive impairment, subjective cognitive decline, Lewy body dementia, Parkinson's disease, sleep fragmentation, schizophrenia, or any combination thereof.

Further provided is a non-transitory computer-readable storage media encoded with instructions executable by one or more processors, wherein the instructions implement any one of the methods or the computer-implemented methods disclosed herein. Also provided is a computer-implemented system comprising: at least one digital processing device comprising at least one processor and instructions executable by the at least one processor, wherein the instructions implement any one of the methods or the computer-implemented methods disclosed herein.

### BRIEF DESCRIPTION OF THE DRAWINGS

FIG. 1 illustrates a block diagram depicting a system to perform neural stimulation via visual stimulation in accordance with an embodiment.

FIG. 2A-2F illustrate visual stimulation signals that cause neural stimulation in accordance with some embodiments.

FIG. 3A-3C illustrate fields of vision in which visual signals can be transmitted for visual stimulus induction of gamma oscillations in the brain in accordance with some embodiments.

FIG. 4A-4C illustrate devices configured to transmit visual signals for neural stimulation in accordance with some embodiments.

FIG. 5A-5D illustrate devices configured to transmit visual signals for neural stimulation in accordance with some embodiments.

FIGS. 6A and 6B illustrate devices configured to receive feedback to facilitate neural stimulation in accordance with some embodiments.

FIGS. 7A and 7B are block diagrams depicting embodiments of computing devices useful in connection with the systems and methods described herein.

FIG. 8 is a flow diagram of a method of performing neural stimulation using visual stimulation in accordance with an embodiment.

FIG. 9 is a block diagram depicting a system for neural stimulation via auditory stimulation in accordance with an embodiment.

FIG. 10A-10I illustrate audio signals and types of modulations to audio signals used to induce neural oscillations via auditory stimulation in accordance with some embodiments.

FIG. 11A illustrates audio signals generated using binaural beats, in accordance with an embodiment.

FIG. 11B illustrates acoustic pulses having isochronic tones, in accordance with an embodiment.

FIG. 11C illustrates audio signals having a modulation technique including audio filters, in accordance with an embodiment.

FIG. 12A-12C illustrate configurations of systems for neural stimulation via auditory stimulation in accordance with some embodiments.

FIG. 13 illustrates a configuration for a system for room-based auditory stimulation for neural stimulation in accordance with an embodiment.

FIG. 14 illustrates devices configured to receive feedback to facilitate neural stimulation via auditory stimulation in accordance with some embodiments.

FIG. 15 is a flow diagram of a method of performing auditory induction of gamma oscillation in the brain in accordance with an embodiment.

FIG. 16A is a block diagram depicting a system for neural stimulation via peripheral nerve stimulation in accordance with an embodiment.

FIG. 16B is a block diagram depicting a system for neural stimulation via multiple modes of stimulation in accordance with an embodiment.

FIG. 17A is a block diagram depicting a system for neural stimulation via visual stimulation and auditory stimulation in accordance with an embodiment.

FIG. 17B is a diagram depicting waveforms used for neural stimulation via visual stimulation and auditory stimulation in accordance with an embodiment.

FIG. 18 is a flow diagram of a method for neural stimulation via visual stimulation and auditory stimulation in accordance with an embodiment.

FIG. 19 is an efficacy summary chart for the modified intent to treat (mITT) population, including p-values, difference, confidence intervals (CI), and a standardized estimate of efficacy based on the values.

FIG. 20 shows the separate means analysis, on the left, and the linear model analysis, on the right, of the Alzheimer's Disease composite score (ADCOMS) as optimized for mid and moderate Alzheimer's Disease (MADCOMS) for the sham and active treatment groups.

FIG. 21 shows the separate means analysis, on the left, and a linear model analysis, on the right, of the Alzheimer's Disease Assessment Scale-Cognitive Subscale 14 (ADAS-Cog14) values for the sham and active treatment groups.

FIG. 22 shows the separate means analysis, on the left, and a linear model analysis, on the right, of the Clinical Dementia Rating Sale Sum of Boxes (CDR-SB) values for the sham and active treatment groups.

FIG. 23 shows the separate means analysis, on the left, and a linear model analysis, on the right, of the Alzheimer's Disease Cooperative Study—Activities of Daily Living Scale (ADCS-ADL) scores for the sham and active treatment groups.

FIG. 24 shows the linear model analysis of the Mini-Mental State Examination (MMSE) score, as measured after six months of treatment (i.e., at the last time point).

FIG. 25 shows the linear model analysis of magnetic resonance imaging (MRI) results of whole brain volume value, on the left, and hippocampal volume, on the right, after six months of treatment.

FIG. 26 is a table depicting a summary of efficacy findings resulting from the human clinical trial, including p-values, treatment differences, CI values and the percentage of slowing of brain atrophy.

FIG. 27 shows graphs that demonstrate the observed improvement (panels a and b) in sleep quality as measured by a reduction in sleep fragmentation, expressed as a higher frequency longer rest durations, over a 24-week period of exemplary gamma oscillation inducing non-invasive sensory stimulation treatment for a first 12-week period of treatment (indicated by the line closest to the white arrow), and second 12-week period of treatment (indicated by the line furthest from the white arrow), in mild to moderate AD subjects. Panels c and d demonstrate the observed impact of the sham treatment on sleep quality as measured by a reduction in sleep fragmentation.

FIG. 28 demonstrates power changes responsive to (1 hr) 40 Hz LED stimulus in an exemplary embodiment showing 40 Hz steady state oscillation and enhanced alpha power during and following stimulus, in a young healthy subject. Both panels illustrate the time-frequency domain decomposition of EEG activity recorded over the occipital pole (Oz, channel-64) before, during and after gamma oscillation inducing 40 Hz stimulation. The start and stop of 40 Hz stimulation are marked with STIM ON and STIM OFF boundaries in both panels. The upper panel illustrates enhanced 40 Hz power during stimulation indicating steady-state visually evoked potential (SSVEP). The lower panel shows alpha-power dynamics during eyes-open (EYO) and eyes-closed (EYC) conditions, and the enhanced alpha power both during eyes-open 40 Hz stimulation, as well as following the one-hour gamma oscillation inducing 40 Hz stimulation.

FIG. 29 provides illustrations of the composite global cognitive summary score as a function of average sleep fragmentation (panel A), and composite expression of genes enriched in aged microglia (panel B). The dotted lines show 95% confidence intervals of estimate.

FIG. 30 provides an oscilloscope capture of the visual (upper signal) and audio (lower signal) signals of an exemplary non-invasive sensory stimulus with fs equal to 40 Hz, v_{d} equal to 50%, V_{D} equal to 50%, ft equal to 7,000 Hz, and AD equal to 0.57%.

FIG. 31 shows a schematic of some aspects and parameters characterizing stimulus audio and visual components of non-invasive stimulation as delivered respectively by Audio Stimulus Module (110; FIG. 33) and Visual Stimulus Module (120; FIG. 33) of Stimulus Delivery System (170; FIG. 33). Numbers and relative dimensions of elements in FIG. 31 are adjusted for presentation and may not represent those for actual embodiments.

FIG. 32 demonstrates an overview of enrollment, treatment, and control for an exemplary embodiment of non-invasive stimulation improving sleep quality in mild to moderate AD subjects. Treatment was delivered to two thirds of the subjects (12) using 40 Hz frequency audio, and one third of subjects (6, “control”) at an alternate frequency.

FIG. 33 provides a block diagram of an exemplary stimulus delivery system and analysis and monitoring system, said analysis and monitoring system comprising modules specific to sleep-related monitoring and/or analysis.

FIG. 34 provides actigraphy data from 24 hours of activity levels (gray bar; 1501, FIG. 37) over two days for a single example patient, centered around 12 AM (indicated by double-sided arrow) along with a median filtered curve (labeled with a dotted arrow; 1507, FIG. 37). The horizontal axis of FIG. 34 shows time of day, and the vertical axis is relative activity recorded on a wrist-worn actigraphic measuring device (arbitrary log scale). Calculated sleep periods (black horizontal lines; see 1508, FIG. 37) along with individual sample rest periods (yellow horizontal lines; see 1509, FIG. 37) are shown: with the top panel (a) showing an exemplary pattern for frequent movements and short rest periods during sleep periods, and the bottom panel (b) showing an exemplary pattern of less frequent movements and longer rest periods during sleep periods.

FIG. 35 provides exemplary patterns of actigraphy (arbitrary units, see FIG. 34) over several days showing actigraphy (gray; e.g., 1501, FIG. 37), and a smooth curve is superposed. Cutoff line (black) separates active versus rest periods (e.g., 1505, FIG. 37). Black squares represent initial estimation for the mid-night point (e.g., 1507, FIG. 37). The final assessment of the mid-night points is determined through optimization algorithm (e.g., 1508, FIG. 37).

FIG. 36 provides exemplary cumulative distribution of rest periods from a single patient (e.g., 1511, FIG. 37). Data from a first exemplary 12 weeks of treatment (solid line's points, Week 0-12) and a second exemplary 12 weeks of treatment (dashed line's points) is shown. In some embodiments the distribution is characterized by an exponential distribution (e.g., 1512, FIG. 37). In a further embodiment, an increase in the exponential decay constant represents an improvement in sleep quality (e.g., 1513, FIG. 37). In the present example, tau₂=45 min, tau₁=40 min, and tau_{diff}=5 min >0.

FIG. 37 provides a flowchart of exemplary analysis steps responsive to actigraphy data, provided in some embodiments at least in part by Actigraphy Monitoring Module 130 (FIG. 33). In some embodiments, analysis is directed at determining the cumulative distribution of rest periods for one or more subjects over a period of one or more nighttime sleep periods (1511). In some embodiments analysis is further directed at fitting an exponential distribution to the determined cumulative distribution (1512). In some embodiments, analysis is further directed at computing summary statistics or characteristic parameters for the fitted exponential distribution. In an exemplary embodiment, the exponential decay constant for the fitted exponential distribution is determined (1512; FIG. 36). In FIG. 37, terms in italics in braces refer to MATLAB (R2020a) APIs employed in the corresponding steps in an exemplary embodiment, e.g., “medfilt1” refers to 1-D median filtering. In some embodiments, alternate APIs, methods, or processes, with equivalent function are employed (e.g., Wolfram Language's “ButterworthFilterModel” may be substituted for “butter”).

FIG. 38 provides sample actigraphy recordings from a single patient, said sample actigraphy recording demonstrating the effect of gamma oscillation inducing non-invasive sensory stimulation therapy on sleep through recordings taken five consecutive nights prior to treatment, and five consecutive nights following treatment. The dark gray, horizontal bars below the X axis indicate continuous activity periods, with the continuous activity periods appearing significantly higher in the actigraphy recordings taken prior to treatment than the actigraphy recordings taken following treatment.

FIG. 39 provides a cumulative distribution of rest and active durations in nighttime based on data pooled from all participants. The black squares indicate active periods, and the gray squares indicate rest periods. Panel A of FIG. 39 shows the cumulative distribution using a log-linear scale, and Panel B of FIG. 39 shows the cumulative distribution using a log-log scale.

FIG. 40 shows graphs comparing the relative change in active durations, with the Y-axis indicating change relative to Weeks 1-12 during Weeks 13-24. FIG. 40 demonstrates a reduction in duration of active periods for the treatment group and, consequently, a reduction in sleep fragmentation leading to increased sleep quality. In contrast, the opposite effect was seen with the sham group, which is represented by the line closest to the gray arrow. Panel A of FIG. 40 shows the relative change based on the duration of active periods, and Panel B of FIG. 40 shows the normalized nighttime active durations, calculated by dividing the duration of each active period by the duration of the matching entire nighttime period.

FIG. 41 shows the effect of gamma oscillation inducing non-invasive sensory stimulation therapy on maintenance of daytime activities, as assessed by Activities of Daily Living (ADCS-ADL) scope. The graph shows that changes in daytime activities significantly improved in the treatment group and declined in the sham group. The X-axis compares the period from Week 1-12 and the period from Week 13-24. The Y-axis demonstrates the change in ADCS-ADL score during Weeks 13-24 relative to Weeks 1-12.

FIG. 42 provides a flow chart demonstrating the proposed relationship between Alzheimer's disease and sleep dysfunction. This was adapted from Wang, C. and D. M. Holtzman (2020). “Bidirectional relationship between sleep and Alzheimer's disease: role of amyloid, tau, and other factors.” Neuropsychopharmacology 45 (1): 104-120.

FIG. 43 provides an exemplary embodiment of a hand-held controller for adjusting parameters of the stimulus delivered by an operably coupled stimulus apparatus.

FIG. 44 provides the results on matter volume change from baseline (%) for treatment and control groups who received 40 Hz gamma oscillation inducting sensory stimulation therapy and sham sensory stimulation therapy, respectively, for a 6-month period.

The dark gray boxes correspond to the Treatment group participants, and the light gray boxes correspond to the Placebo group participants. Error bars indicate standard error (SE).

FIG. 45 provides the T1-weighted image to T2-weighted images (Tlw/T2w) ratio change in white matter (% change from baseline) for Placebo group participants (light gray) and Treatment group participants (dark gray) after receiving sham and 40 Hz gamma oscillation inducing sensory stimulation therapy, respectively, for a 6-month period.

FIGS. 46A and 46B provide measurements of volume change in white matter structures as a percent change relative to baseline. The Treatment group participants are indicated by dark gray, and the Placebo group participants' results are indicated in light gray. FIG. 46A provides the results for entorhinal region, left cingulate lobe, pars triangularis region, cuneus region, lateral occipital region, postcentral region, left occipital lobe, left frontal lobe, left parietal lobe, occipital lobe, left temporal lobe and caudal middle frontal region (sorted in ascending order by p value) for the treatment group after 6 months of treatment. FIG. 46B provides the results for the precentral region, paracentral region, lingual region, fusiform region, frontal lobe, rostral anterior cingulate region, inferior temporal region, right occipital lobe, parietal lobe, rostral middle frontal, precuneus region, medial orbitofrontal region, and temporal lobe (sorted in ascending order by p value).

FIGS. 47A and 47B provide the T1w/T2w ratio change in white matter structures (% change from baseline) for Placebo and Treatment group participants after receiving sham and 40 Hz gamma oscillation inducing sensory stimulation therapy, respectively, for a 6-month period favors the treatment group. FIG. 47A provides the results for the entorhinal region, pars triangularis region, postcentral region, left parietal lobe, lateral occipital region, paracentral region, rostral middle frontal region, supramarginal region, precentral region, parietal lobe, right occipital lobe, fusiform region, occipital lobe, left frontal lobe, cuneus region, precuneus region, inferior parietal region, frontal lobe, lingual region, left occipital lobe, left temporal lobe, right parietal lobe and pars orbitalis region, with white matter structures sorted in ascending order by p value. FIG. 47B provides the results for the right frontal lobe, caudal middle frontal region, rostral anterior cingulate region, superior frontal region, temporal lobe, medial orbitofrontal region, posterior cingulate region, superior parietal region, left cingulate lobe, superior temporal region, cingulate lobe, and temporal pole region, with white matter structures sorted in ascending order by p value.

FIG. 48 provides an example of a participant's usage of a 40 Hz auditory and visual stimulation device throughout a 6-month period. The participant selected different visual and audio settings (first and second rows from the top) while the frequency of the device was set to 40 Hz (third row from the top). The time of the day the device was used was recorded by the device (fourth row from the top). Independently, the participant entered the therapy times into a diary (fifth row from the top). This participant shows close to 100% adherence (bottom row).

FIG. 49 shows changes in the Alzheimer's Disease Assessment Scale-Cognitive Subscale (ADAS-Cog) score as a function of baseline coherence after six months of active treatment using a 40 Hz auditory and visual stimulation device. An overall negative correlation between baseline coherence and ADAS-Cog score is observed.

FIG. 50 provides changes in the Alzheimer's Disease Cooperative Study-Activities of Daily Living (ADCS-ADL) score as a function of baseline coherence after six months of active treatment using a 40 Hz auditory and visual stimulation device. An overall positive correlation between baseline coherence and ADCS-ADL score is observed.

FIG. 51 provides changes in the Alzheimer's Disease Cooperative Study-Activities of Daily Living (ADCS-ADL), Attentive Participation in Conversations score as a function of baseline coherence after six months of active treatment using a 40 Hz auditory and visual stimulation device. An overall positive correlation between baseline coherence and ADCS-ADL, Attentive Participation in Conversations score is observed.

FIG. 52 provides changes in the Alzheimer's Disease Cooperative Study-Activities of Daily Living (ADCS-ADL), Finding Belongings score as a function of baseline coherence after six months of active treatment using a 40 Hz auditory and visual stimulation device. An overall positive correlation between baseline coherence and ADCS-ADL, Finding Belongings score is observed.

FIG. 53 provides changes in the Clinical Dementia Rating (CDR) scale, Memory score as a function of baseline coherence after six months of active treatment using a 40 Hz auditory and visual stimulation device. An overall negative correlation between baseline coherence and CDR, Memory score is observed.

FIG. 54 provides changes in the Clinical Dementia Rating (CDR) scale, Orientation score as a function of baseline coherence after six months of active treatment using a 40 Hz auditory and visual stimulation device. An overall negative correlation between baseline coherence and CDR, Orientation score is observed.

FIG. 55 provides changes in the Clinical Dementia Rating scale, Sum of Boxes (CDR SB) score as a function of baseline coherence after six months of active treatment using a 40 Hz auditory and visual stimulation device. An overall negative correlation between baseline coherence and CDR SB score is observed.

FIG. 56 provides changes in the Mini-Mental State Examination (MMSE) score as a function of baseline coherence after six months of active treatment using a 40 Hz auditory and visual stimulation device. An overall positive correlation between baseline coherence and MMSE score is observed.

FIG. 57 shows changes in the magnetic resonance imaging (MRI) lateral ventricle volume as a percentage of total intracranial volume (vMRI-LV as % in TIV) as a function of baseline coherence after six months of active treatment using a 40 Hz auditory and visual stimulation device. An overall negative correlation between baseline coherence and lateral ventricle (LV) volume is observed.

FIG. 58 shows changes in the MRI temporal cortex thickness (mm) as a function of baseline coherence after six months of active treatment using a 40 Hz auditory and visual stimulation device. An overall positive correlation between baseline coherence and temporal thickness is observed.

FIG. 59 shows changes in the Neuropsychiatric Inventory Questionnaire (NPIQ) Severity score as a function of baseline coherence after six months of active treatment using a 40 Hz auditory and visual stimulation device. An overall negative correlation between baseline coherence and NPIQ Severity score is observed.

FIG. 60 shows changes in the positron emission tomography (PET) Composite amyloid standardized uptake value ratio (SUVR) as a function of baseline coherence after six months of active treatment using a 40 Hz auditory and visual stimulation device. An overall negative correlation between baseline coherence and PET Composite SUVR is observed.

FIG. 61 shows changes in the PET Occipital amyloid SUVR as a function of baseline coherence after six months of active treatment using a 40 Hz auditory and visual stimulation device. An overall negative correlation between baseline coherence and PET Occipital SUVR is observed.

The features and advantages of the present solution will become more apparent from the detailed description set forth below when taken in conjunction with the drawings, in which like reference characters identify corresponding elements throughout. In the drawings, like reference numbers generally indicate like elements.

### DETAILED DESCRIPTION

Neurological conditions that impact the nervous systems of humans and animals can be difficult to diagnose, treat, and evaluate, because of often overlapping or similar symptoms between diseases, lack of accurate quantitative assays based on biomarkers, or long preclinical and prodromal phases.

Alzheimer's Disease (AD) is one example neurological condition, where many of these issues exist. AD may progress for years or decades before any symptoms become apparent. AD lacks a widely available and accepted quantitative assays based on biomarkers that provides certainty in diagnosis. Diagnosis of AD may involve a multidimensional analysis of a patient and the patient's familial medical history, the patient's subjective reports of symptoms, MRIs, lab work, and evaluations by a multitude of medical experts. Even still, AD is associated with a high rate of misdiagnosis (10%-20%). Some of the misdiagnosis may be attributed to different neurodegenerative or psychiatric disorders mistaken for AD.

Some neurodegenerative illnesses, like AD, may be associated with long preclinical and prodromal phases, that can lead to symptoms such as cognitive dysfunction, behavioral abnormalities, and impaired performance of activity of daily living. Symptoms arising from neurodegenerative illnesses can onset over a long duration of time, and when detected, the causal illness may have developed significantly into moderate or severe stages of the disease, with little expectation of amelioration. For example, preclinical stages (before any physical symptoms may become apparent) of Alzheimer's disease may last for years or for decades.

Even once initial symptoms start to become apparent, the disease may progress slowly such that the symptoms are easy to ignore or dismiss. Before onset of clinical dementia, there can be several stages of cognitive decline. In some cases, one of the first stages may be subjective cognitive decline (SCD). SCD can refer to a self-reported experience of worsening or more frequent confusion or memory loss; within this stage, individuals can be identified as “SCD plus” referring to patients which have both cognitive complaints and concurrent AD-associated pathological changes. In some cases, the patients that are classified as “SCD plus” can have the following high-risk features for further cognitive decline: a subjective decline in memory, onset of SCD within the last 5 years, >60 years of age at SCD onset, concerns (worries) associated with SCD, feelings of worse performance than others in the same age group, or confirmation of cognitive decline by an informant. In some cases, the next stage of cognitive decline after SCD may be Mild Cognitive Impairment (MCI); MCI can be characterized in patients that have problems with memory, language, thinking, or judgement. In some cases, it can be difficult to dissociate the element of subjectivity (e.g., “self-reported”) in clinical evaluations of AD diagnosis and monitoring of AD progression.

Prion diseases are another set of neurological conditions where the issues exist. Prion disease, also known as transmissible spongiform encephalopathies, can refer to a group of fatal neurodegenerative diseases which can include Creutzfeldt-Jakob Disease (CJD), Variant Creutzfeldt-Jakob Disease (vCJD), Gerstmann-Sträussler-Scheinker Syndrome, Fatal Familial Insomnia, Kuru, and others. In some cases, prion diseases can have similar symptoms with other and/or with AD. In some cases, different types of prion diseases can cause brain damage that exhibit similar features such as: extensive spongiform degeneration, widespread neuronal loss, synaptic alterations, atypical brain inflammation, and accumulation of protein aggregates. In some cases, prion diseases such as CJD, Kuru, and Gerstmann-Sträussler-Scheinker disease, may form amyloid plaques that is similar to those observed in AD.

Recognized herein is a need for evaluating neurological conditions using a quantitative assay. For example, several resting-state EEG markers are identified as potential biomarkers for monitoring decline in integrity of neuronal network activities in AD. EEG biomarkers are considered for detecting stages of neurological disorders (e.g., preclinical AD or early-stage AD), and for differentiating between neurological disorders (e.g., AD and a prion disease). Recognized herein is that neurophysiological responses to gamma oscillation inducing non-invasive sensory stimulation may predict clinical outcome in subjects with AD or other neurological disorders.

In some aspects, the present disclosure describes systems and methods for stimulating at least a portion of a brain region of a subject with gamma oscillation inducing non-invasive sensory stimulus and measuring a response from the brain region. In some cases, the response may be used to differentiate between different neurological conditions, for example, between a neurodegenerative disorder and a psychiatric disorder. In some cases, the response may be used to differentiate between neurodegenerative disorders. In some cases, the response may be used to differentiate between psychiatric disorders.

In some cases, the response may be used to predict an expected treatment outcome. In some cases, the expected treatment outcome may be a speed at which a disease may progress or a survival rate. In some cases, the expected treatment outcome may be an improvement in survival rate, slowing the progression of a disease, or a recommended treatment. Various other possible treatment outcomes are disclosed herein.

In some aspects, the present disclosure describes a method of predicting an expected treatment outcome of a subject. In some cases, the method comprises administering a gamma oscillation inducing non-invasive sensory stimulus to the subject. In some cases, the method comprises measuring a response from the subject. In some cases, the method comprises using a machine learning algorithm to predict the expected treatment outcome of the subject based at least partially on the measured response.

In some aspects, the present disclosure provides methods of generating a plurality of datasets based on a response of a plurality of individuals, wherein the individuals are assigned to a distinct population based on shared characteristics. In some aspects, the plurality of datasets may be used to predict a treatment response of a subject having one or more of the shared characteristics. For example, the plurality of data sets may comprise EEG response profiles to varying stimuli. A distinct population of individuals may comprise healthy controls. Another distinct population of individuals may comprise a group of subjects with a neurological disorder. A subject's response to gamma oscillation inducing non-invasive sensory stimulus may be evaluated and compared to the responses provided in the datasets. This comparison may reveal, for example, that the subject is a healthy individual. This comparison may reveal, alternatively, that the subject shared response characteristics associated with a certain disease or disorder. Accordingly, the comparison may be used to diagnose the subject as having the disease or disorder.

In some aspects, the present disclosure describes a computer-implemented method of predicting a response to a treatment for a subject diagnosed with or at a risk of developing a neurodegenerative disorder associated with cognitive decline. In some cases, the computer-implemented method comprises providing a visual gamma oscillation inducing non-invasive sensory stimulus to the subject. In some cases, the computer-implemented method comprises performing an encephalogram on a brain region of the subject to measure a plurality of bioelectrical signals.

In some aspects, the present disclosure describes a computer-implemented method of administering a treatment and computing an expected clinical outcome score of the treatment of a subject diagnosed with or at a risk of developing Alzheimer's Disease. In some cases, the computer-implemented method comprises administering a therapeutic dose of gamma oscillation inducing non-invasive sensory stimulus to a brain of the subject. In some cases, the computer-implemented method comprises performing an encephalogram on the brain of the subject to measure a plurality of bioelectrical signals. In some cases, the computer-implemented method comprises computing, using a machine learning algorithm, the expected clinical outcome score of the subject based on the plurality of bioelectrical signals. In some cases, the computer-implemented method comprises adjusting the therapeutic dose based at least partially on the expected clinical outcome score.

Predicting an expected treatment outcome may comprise using any one of various methods, qualitative or quantitative evaluations. In some cases, predicting may comprise computing one or more prediction scores using a computer program. In some cases, a prediction score may be logical value, a categorical value, a probability value, or any combination thereof. In some cases, predicting may comprise comparing the prediction score against a predetermined threshold value. In some cases, the predetermined threshold value may be a value above which the expected treatment outcome is favorable to the subject. In some cases, the predetermined threshold value may be a value under which the expected treatment outcome is not favorable to the subject. In some cases, the predetermined threshold value may be a value above which the expected treatment outcome is categorized as a specific type of outcome. In some cases, the predetermined threshold value may be a value under which the expected treatment outcome is not categorized as a specific type of outcome. In some cases, predicting may comprise predicting by a medical professional.

Predicting the expected treatment outcome of the subject may comprise predicting at various time points or time ranges in the future. In some cases, predicting the expected treatment outcome of the subject may be predicting at least 1, 2, 3, 4, 5, 10, 15, 20, 25, 30, 35, 40, 45, 50, 55, or 60 minutes in advance. In some cases, predicting the expected treatment outcome of the subject may be predicting at least 1, 2, 3, 4, 5, 6, 7, 8, 9, 10, 11, 12, 13, 14, 15, 16, 17, 18, 19, 20, 21, 22, 23, or 24 hours in advance. In some cases, predicting the expected treatment outcome of the subject may be predicting at least 1, 2, 3, 4, 5, 6, or 7 days in advance. In some cases, predicting the expected treatment outcome of the subject may be predicting at least 1, 2, 3, or 4 weeks in advance. In some cases, predicting the expected treatment outcome of the subject may be predicting at least 1, 2, 3, 4, 5, 6, 7, 8, 9, 10, 11, or 12 months in advance. In some cases, predicting the expected treatment outcome of the subject may be predicting at least 1, 2, 3, 4, 5, 6, 7, 8, 9, or 10 years in advance. In some cases, predicting the expected treatment outcome of the subject may be predicting at most 1, 2, 3, 4, 5, 10, 15, 20, 25, 30, 35, 40, 45, 50, 55, or 60 minutes in advance. In some cases, predicting the expected treatment outcome of the subject may be predicting at most 1, 2, 3, 4, 5, 6, 7, 8, 9, 10, 11, 12, 13, 14, 15, 16, 17, 18, 19, 20, 21, 22, 23, or 24 hours in advance. In some cases, predicting the expected treatment outcome of the subject may be predicting at most 1, 2, 3, 4, 5, 6, or 7 days in advance. In some cases, predicting the expected treatment outcome of the subject may be predicting at most 1, 2, 3, or 4 weeks in advance. In some cases, predicting the expected treatment outcome of the subject may be predicting at most 1, 2, 3, 4, 5, 6, 7, 8, 9, 10, 11, or 12 months in advance. In some cases, predicting the expected treatment outcome of the subject may be predicting at most 1, 2, 3, 4, 5, 6, 7, 8, 9, or 10 years in advance. In some cases, predicting the expected treatment outcome of the subject may be predicting in a range formed by any combination of the above.

In some cases, the expected treatment outcome may be an outcome for a gamma oscillation inducing non-invasive sensory stimulus treatment. The expected treatment outcome may comprise a likelihood to response to treatment. The treatment may be for any one of the diseases or disorders disclosed herein. In some cases, the treatment may be for Creutzfeldt-Jakob disease (CJD), variant CJD, Gerstmann-Sträussler Scheinker Syndrome, Fatal Familial Insomnia, Kuru, or any combination thereof. In some cases, the expected treatment outcome may be an episode associated with the psychiatric or neurological disorder. In some cases, the episode may be a bipolar episode or a schizophrenic episode.

In some cases, the treatment is for a microglial-mediated disease or disorder. The microglial-mediated disease or disorder may comprise a neurodegenerative disease associated with tauopathy, including but not limited to Alzheimer's disease, frontotemporal dementia, chronic traumatic encephalopathy (CTE), and corticobasilar degeneration. In some cases, the subject has an inherited ataxia. Hereditary ataxias frequently cause atrophy of the cerebellum as a result of impaired circuitry and function of the cerebellar cortex, a result of neurodegeneration of cellular afferents and the Purkinje cells, which have long axonal projections that comprise the only sources of output from the cerebellar cortex to deep cerebellar nuclei.

In some cases, the treatment is for a neuropsychiatric disorder associated with brain atrophy, which is mediated by microglial cells. For example, individuals with schizophrenia often show reduced postmortem cortical tissue. This phenomenon is caused by synaptic pruning, which reflects abnormalities in microglia-like cells and synaptic function. In other embodiments, the present disclosure provides methods and systems for alleviating symptoms of depression. Stress, impaired neurogenesis, and defects in synaptic plasticity are associated with depression. Chronic stress promotes microglial hyper-ramification and astroglial atrophy. Thus, in some embodiments, the system and methods disclosed may alleviate symptoms associated with chronic stress or depression by improving synaptic plasticity and stimulating neural networking, along with improving microglial-mediated clearance.

In some cases, the treatment is for symptoms associated with a stroke. For example, the stroke may be an ischemic stroke, which causes a neuroinflammatory response and activates microglia to help repair the brain. Ischemic stroke is associated with disappearance of synaptic activity. As a result, brain tissue within the penumbra during an ischemic stroke is structurally intact, but functionally silent.

In some cases, the treatment is for a demyelinating disease. Demyelinating disease may comprise Multiple Sclerosis or Acute disseminated encephalomyelitis, both of which may cause neuroinflammation and cerebral atrophy. In multiple sclerosis (MS), brain or cerebral atrophy is common due to demyelination and destruction of nerve cells. Widespread myelin damage occurs, causing damage to the myelin-rich white matter of the brain, occurs as a result of a number of attacks which occur over time. In acute disseminated encephalomyelitis, similar symptoms are seen, but the onset of widespread myelin damage is often due to a single episode or attack.

The outcome may be any one of various clinically relevant outcomes. In some cases, the outcome may be survival. In some cases, the outcome may be prolonged survival. In some cases, the outcome may be an improvement in symptoms associated with a disease or disorder, such as a neurodegenerative disease. For example, in some cases, the outcome comprises an improvement in the behavioral and psychological symptoms of dementia (BPSD). For example, the symptoms associated with the disease or disorder may comprise apathy, anxiety, or depression. In some cases, the outcome may be higher quality of life. In some cases, the outcome may be an improvement in a pathological process, such as better sleep. In some cases, the outcome may be lower quality of life. In some cases, the outcome may be being cured. In some cases, the outcome may be being uncured. In some cases, the outcome may be improved cognitive function. In some cases, the outcome may be worsened cognitive function. In some cases, the outcome may be better memory. In some cases, the outcome may be worse memory. In some cases, the outcome may be happiness. In some cases, the outcome may be depression. In some cases, the outcome may be death. The outcome may be a quantitatively or a qualitatively measurable or a determinable value or a state for any of the aforementioned examples of clinically relevant outcomes. In some cases, the outcome may be a quantitative or qualitative evaluation that can be made by a medical professional. In some cases, the outcome may be a self-evaluation that can be made by a subject, a patient report, or a care partner report prepared on the subject. In some cases, the outcome may be an outcome of a treatment for sleep fragmentation. In some cases, the outcome maybe a quantitative or qualitative evaluation that can be made by an instrument or a computer.

A subject can be any animal having nerves. In some cases, the subject may be mammal. In some cases, the subject may be a human being. The subject may be of various ages, genders, sex, height, weight, or any other clinically relevant biometrics.

A subject may be diagnosed with, be suspected of being afflicted with, or be at a risk of being afflicted with any one of the diseases disclosed herein. In some cases, the disease may be a neurodegenerative disorder associated with cognitive decline. In some cases, the subject may be afflicted, be suspected of being afflicted, or be at a risk of being afflicted with a prion disease or transmissible spongiform encephalopathy. In some cases, the subject may be afflicted, be suspected of being afflicted, or be at a risk of being afflicted with Alzheimer's disease, Creutzfeldt-Jakob disease (CJD), variant CJD, Gerstmann-Sträussler Scheinker Syndrome, Fatal Familial Insomnia, Kuru, or any combination thereof. In some cases, the disease may be a psychiatric or neurological disorder associated with cognitive decline. In some cases, the psychiatric or neurological disorder is a mood disorder, depression, bipolar disorder, anxiety, addiction, neurosis, anorexia, bulimia, dementia, mild cognitive impairment, subjective cognitive decline, Lewy body dementia, Parkinson's disease, sleep fragmentation, schizophrenia, or any combination thereof.

In some cases, administering may be a non-invasive procedure. In some cases, administering may be indirect (e.g., without physical contact) stimulation of optical nerves. In some cases, administering may use light. In some cases, administering may be indirect stimulation of auditory nerves. In some cases, administering may be indirect stimulation of any one of the nerves disclosed herein. In some cases, administering may use sound. In some cases, administering may be direct stimulation. In some cases, administering may be electricity delivered to a region in the subject's body. In some cases, administering may be vibration delivered to a region in the subject's body. In some cases, the region in the subject's body may be skin on the head of the subject, surface of the skull of the subject, surface of a membrane surrounding the subject's brain, the subject's brain, a region in the subject's brain, a retinal nerve of the subject, or a cochlear nerve of the subject. In some cases, the gamma oscillation inducing non-invasive sensory stimulus may be a visual stimulus, an auditory stimulus, a kinesthetic stimulus, or any combination thereof. The gamma oscillation inducing non-invasive sensory stimulus may be provided using any one of the devices or methods disclosed herein.

In some cases, administering may be performed continuously for a predetermined duration of time. In some cases, the predetermined duration of time may be between 10 minutes and 2 hours. In some cases, the predetermined duration of time may be at least 1, 2, 3, 4, 5, 10, 15, 20, 25, 30, 35, 40, 45, 50, 55, or 60 minutes. In some cases, the predetermined duration of time may be at least 1, 2, 3, 4, 5, 6, 7, 8, 9, 10, 11, 12, 13, 14, 15, 16, 17, 18, 19, 20, 21, 22, 23, or 24 hours. In some cases, the predetermined duration of time may be at least 1, 2, 3, 4, 5, 6, or 7 days. In some cases, the predetermined duration of time may be at most 1, 2, 3, 4, 5, 10, 15, 20, 25, 30, 35, 40, 45, 50, 55, or 60 minutes. In some cases, the predetermined duration of time may be at most 1, 2, 3, 4, 5, 6, 7, 8, 9, 10, 11, 12, 13, 14, 15, 16, 17, 18, 19, 20, 21, 22, 23, or 24 hours. In some cases, the predetermined duration of time may be at most 1, 2, 3, 4, 5, 6, or 7 days.

In some cases, administering may be performed a number of discrete times. In some cases, administering may be performed about once a day for 6 months. In some cases, the administering may be performed at least about 1, 2, 3, 4, 5, 6, 7, 8, 9, or 10 times a day. In some cases, the administering may be performed at most about 1, 2, 3, 4, 5, 6, 7, 8, 9, or 10 times a day. In some cases, the administering may be performed a number of times over a period of at least about 1, 2, 3, or 4 weeks. In some cases, the administering may be performed a number of times over a period of at least about 1, 2, 3, 4, 5, 6, 7, 8, 9, 10, 11, or 12 months. In some cases, the administering may be performed a number of times over a period of at most about 1, 2, 3, or 4 weeks. In some cases, the administering may be performed a number of times over a period of at most about 1, 2, 3, 4, 5, 6, 7, 8, 9, 10, 11, or 12 months.

In some cases, the prediction score may be presented to a medical professional. In some cases, the medical professional may be a patient, a nurse, a doctor, or an analyst at an insurance company.

The gamma oscillation inducing non-invasive stimulus may be any type of non-invasive stimulus with a component that induces gamma brainwaves (also referred to as neural activity). The gamma oscillation inducing non-invasive sensory stimulus may be any stimulus that can be administered to a subject, such that a gamma oscillation is induced in a nerve of the subject. In some cases, the gamma oscillation inducing non-invasive sensory stimulus may be a gamma oscillation inducing non-invasive sensory stimulus. In some cases, the gamma oscillation inducing non-invasive sensory stimulus may be a non-invasive sensory stimulus. In some embodiments, the gamma oscillation inducing non-invasive stimulus may induce neural oscillations in frequency ranges other than the gamma range. In some embodiments, the gamma oscillation inducing non-invasive stimulus may induce neural oscillations in the theta frequency range. In some embodiments, the gamma oscillation inducing non-invasive stimulus may induce neural oscillations in the gamma frequency range that alternate with neural oscillations in other frequency ranges (e.g., theta, alpha, or beta frequency ranges). The gamma oscillation inducing non-invasive sensory stimulus may have characteristics of any stimulus disclosed herein.

The response of the subject may be any clinically relevant response from the subject. In some cases, the response may be correlated with the expected treatment outcome for the subject. In some cases, the response may be a change in brain activity. In some cases, the brain activity may be measured with EEG. In some cases, the response may be change in EEG coherence. In some cases, the EEG coherence may be correlated with one or more measures of the clinical outcome of the subject. In some cases, the response may be a change in the subject's ability to carry out a cognitive engaging task (e.g., identifying objects, drawing, speaking, listening, tasting, smelling, counting, playing a game, or playing an instrument). In some cases, the response may be a change in a biometric signal, including but not limited to, heart rate, blood pressure, breathing rate, body temperature, or an electrical signal from any region of the subject's body. In some cases, the response may be measured with Mini-Mental State Examination (MMSE), Clinical Dementia Rating (CDR), Alzheimer's Disease Cooperative Study-Activities of Daily Living (ADCS-ADL), magnetic resonance imaging (MRI) volumetric data from lateral ventricles, or any combination thereof. In some cases, the biometric signal may be sleep data.

Various machine learning algorithms may be used in the method. In some cases, the machine learning algorithm may employ any one of machine learning elements disclosed herein. In some cases, the machine learning algorithm may be configured to receive the response measured from the subject. In some cases, the machine learning algorithm may be configured to receive one or more EEG signals measured from the subject. In some cases, the machine learning algorithm may be configured to receive one or more signals having a frequency from about 4 Hz and 400 Hz. In some cases, the machine learning algorithm may be configured to receive one or more signals having a frequency from about 20 Hz and 200 Hz. In some cases, the machine learning algorithm may be configured to receive one or more signals having a frequency from about 20 Hz and 80 Hz. In some cases, the machine learning algorithm may be configured to output one or more values for the expected treatment outcome.

In some cases, the method may further consider network information of the subject to predict the expected treatment outcome of the subject. In some cases, the network information may comprise cell phone data, GPS data, social network data, or any combination thereof. Social network data can be used, for example, to monitor a patient's behavior and predict future behavior. In an exemplary embodiment, for a patient with bipolar disorder, future behavior may comprise manic or depressive episode.

In some cases, the subject may be selected for a treatment plan or a clinical study based at least partially on the expected treatment outcome. In some cases, the treatment plan may be a treatment plan for any one of the diseases disclosed herein. In some cases, the treatment plan may be a gamma oscillation inducing non-invasive sensory stimulation treatment plan. In some cases, the clinical study may be a study for gamma oscillation inducing non-invasive sensory stimulation therapy.

In some cases, the method may comprise adjusting the treatment for the subject based at least partially on the expected treatment outcome of the subject. In some cases, the adjusting may be an adjustment of the therapeutic dose of the treatment. In some cases, the adjustment of the therapeutic dose may be an adjustment of the amount (e.g., the volume or the weight) of a drug or pharmaceutical for a drug-based or pharmaceutical-based treatment.

In some cases, the adjustment of the therapeutic dose may be an adjustment of a parameter of the gamma oscillation inducing non-invasive sensory stimulus administered for a gamma oscillation inducing non-invasive sensory stimulus treatment. In some cases, the adjustment of the therapeutic dose may be an adjustment of a parameter of the gamma oscillation inducing non-invasive sensory stimulus administered for a gamma oscillation inducing non-invasive sensory periodic stimulus treatment. In some cases, the adjustment of the therapeutic dose may be an adjustment of a parameter of the gamma oscillation inducing non-invasive sensory stimulus administered for a gamma oscillation inducing non-invasive sensory stimulus with periodic components treatment. In some cases, the adjustment of the therapeutic dose may be an adjustment of a parameter of the gamma oscillation inducing non-invasive sensory stimulus administered for a gamma oscillation inducing non-invasive sensory non-periodic stimulus treatment. In some cases, the adjustment of the therapeutic dose may be an adjustment of a parameter of the gamma oscillation inducing non-invasive sensory stimulus administered for a gamma oscillation inducing non-invasive sensory stimulus with non-periodic components treatment. In some cases, the adjustment of the therapeutic dose may be an adjustment of a parameter of the gamma oscillation inducing non-invasive sensory stimulus administered for a periodic or a non-periodic component of a gamma oscillation inducing non-invasive sensory stimulus treatment. In some cases, the adjustment of a parameter of the gamma oscillation inducing non-invasive sensory stimulus is an adjustment of the duration of the stimulus. In some cases, the adjustment of a parameter of the gamma oscillation inducing non-invasive sensory stimulus is an adjustment of the intensity of the stimulus. In some cases, the adjustment of a parameter of the gamma oscillation inducing non-invasive sensory stimulus is an adjustment of the amplitude of the stimulus. In some cases, the adjustment of a parameter of the gamma oscillation inducing non-invasive sensory stimulus is an adjustment of the wavelength of the stimulus. In some cases, the adjustment of a parameter of the gamma oscillation inducing non-invasive sensory stimulus is an adjustment of the frequency of the stimulus. In some cases, the adjustment of a parameter of the gamma oscillation inducing non-invasive sensory stimulus is an adjustment of the waveform of the stimulus. In some cases, the adjustment of a parameter of the gamma oscillation inducing non-invasive sensory stimulus is an adjustment of the duty cycle of the stimulus. In some cases, the adjustment of a parameter of the gamma oscillation inducing non-invasive sensory stimulus is an adjustment of the interstimulus interval of the stimulus. In some cases, adjustment of a parameter of the gamma oscillation inducing non-invasive sensory stimulus is an adjustment of the spectrum of the stimulus. In some cases, adjustment of a parameter of the gamma oscillation inducing non-invasive sensory stimulus is an adjustment of the envelope of the stimulus. In some cases, the adjustment of a parameter of the gamma oscillation inducing non-invasive sensory stimulus is an adjustment of the modulation of the stimulus. In some cases, the adjustment of a parameter of the gamma oscillation inducing non-invasive sensory stimulus is an adjustment of the frequency of the modulation of the stimulus. In some cases, the adjustment of a parameter of the gamma oscillation inducing non-invasive sensory stimulus is an adjustment of the amplitude of the modulation of the stimulus. In some cases, the adjustment of a parameter of the gamma oscillation inducing non-invasive sensory stimulus is an adjustment of the harmonic structure of the stimulus. In some cases, the adjustment of a parameter of the gamma oscillation inducing non-invasive sensory stimulus is an adjustment of the phase of the stimulus.

In some cases, the adjustment of the therapeutic dose may be an adjustment of the parameters of a uni-directional wave stimulus. In some cases, the adjustment of the therapeutic dose may be an adjustment of the parameters of a bi-directional wave stimulus. In some cases, the adjustment of the therapeutic dose may be an adjustment of the parameters of a square-wave stimulus. In some cases, the adjustment of the therapeutic dose may be an adjustment of the parameters of a rectangular-wave stimulus. In certain cases, the adjustment of the therapeutic dose may be an adjustment of the parameters of a pulse stimulus. In some cases, the adjustment of the therapeutic dose may be an adjustment of the parameters of a sinusoidal wave stimulus. In some cases, the adjustment of the therapeutic dose may be an adjustment of the parameters of a triangle-wave stimulus. In some cases, the adjustment of the therapeutic dose may be an adjustment of the parameters of a sawtooth-wave stimulus. In some cases, the adjustment of the therapeutic dose may be an adjustment of the parameters of a ramp-wave stimulus. In some cases, the adjustment of the therapeutic dose may be an adjustment of the parameters of a noise stimulus. In certain cases, the adjustment of the therapeutic dose may be an adjustment of the parameters of a white noise stimulus. In certain cases, the adjustment of the therapeutic dose may be an adjustment of the parameters of a pink noise stimulus. In certain cases, the adjustment of the therapeutic dose may be an adjustment of the parameters of a red noise stimulus. In certain cases, the adjustment of the therapeutic dose may be an adjustment of the parameters of a purple noise stimulus. In certain cases, the adjustment of the therapeutic dose may be an adjustment of the parameters of a grey noise stimulus. In some cases, the adjustment of the therapeutic dose may be an adjustment of the parameters of a sweep stimulus. In some cases, the adjustment of the therapeutic dose may be an adjustment of the parameters of a chirp stimulus. In certain cases, the adjustment of the therapeutic dose may be an adjustment of the parameters of an O-chirp stimulus. In certain cases, the adjustment of the therapeutic dose may be an adjustment of the parameters of a linear chirp stimulus. In certain cases, the adjustment of the therapeutic dose may be an adjustment of the parameters of an exponential chirp stimulus. In certain cases, the adjustment of the therapeutic dose may be an adjustment of the parameters of a hyperbolic chirp stimulus. In some cases, the adjustment of the therapeutic dose may be an adjustment of the parameters of a click stimulus. In certain cases, the adjustment of the therapeutic dose may be an adjustment of the parameters of a frequency modulated wave stimulus. In certain cases, the adjustment of the therapeutic dose may be an adjustment of the parameters of an amplitude modulated wave stimulus.

In some cases, the adjustment of the therapeutic dose may be an adjustment of a delivery parameter of the gamma oscillation inducing non-invasive sensory stimulus administered for a gamma oscillation inducing non-invasive sensory stimulus treatment. In some cases, the adjustment of a delivery parameter of the gamma oscillation inducing non-invasive sensory stimulus is an adjustment of the time course of delivery of the stimulus. In some cases, the adjustment of a delivery parameter of the gamma oscillation inducing non-invasive sensory stimulus is an adjustment of the intensity of delivery of the stimulus. In some cases, the adjustment of a delivery parameter of the gamma oscillation inducing non-invasive sensory stimulus is an adjustment of the duration of delivery of the stimulus. In some cases, the adjustment of a delivery parameter of the gamma oscillation inducing non-invasive sensory stimulus is an adjustment of the modality of delivery of the stimulus.

In some cases, the therapeutic dose may be any adjustable dose for a treatment for any one of the diseases disclosed herein.

### Methods and Systems for Identifying EEG Biomarkers

In some aspects, the present disclosure describes a method of identifying a biomarker. In some cases, the method comprises training a machine learning algorithm to identify a statistical relationship between (i) a first dataset comprising a plurality of response measurements for a plurality of subjects, wherein the plurality of response measurements comprises a response to a gamma oscillation inducing non-invasive sensory stimulus for each subject in the plurality of subjects, and (ii) a second dataset comprising a plurality of clinical measurements for the plurality of subjects. In some cases, the method comprises identifying, using the machine learning algorithm, a biomarker that is associated with a distinct clinical outcome.

In some cases, identifying the biomarker may comprise finding one or more patterns in a response data that is correlated with a clinical measurement. In some cases, the response data may comprise bioelectrical measurements. In some cases, the response data may be EEG data. In some cases, the one or more characteristics may comprise a range of amplitudes in the EEG data. In some cases, the one or more characteristics may comprise a range of frequencies in the EEG data. In some cases, the one or more characteristics may comprise of higher order relations between two or more different brain regions observed through EEG as preserved phase relations at a particular frequency (coherence) or frequencies in the frequency domain, or preserved phase relations (synchronous activity up to a phase delay in the waveforms) in the time domain.

In some cases, the response measurements may be any one of response measurements disclosed herein. In some cases, the clinical measurements may be any one of clinical measurements disclosed herein. In some cases, the measurements may be taken from a diverse group of subjects of different genders, ages, demographics, genetics, or health. In some cases, the plurality of subjects may comprise subjects afflicted with a disease disclosed herein and subjects not afflicted with the disease.

In some cases, stimulus induced EEG responses may predict one or more clinical outcomes.

In some cases, stimulus induced EEG responses may predict a unified measure of clinical outcome that consists of combinations of multiple outcomes that may add at different proportions.

In some cases, a unified outcome that we relate EEG to can be obtained from singular value decomposition or principal component analysis of multiple outcomes.

In some cases, EEG responses to a specific stimulus can predict the direction of maximum change where each axis may represent different clinical outcome.

### Delivery Methods and Systems

The present disclosure provides a method directed at determining an expected treatment outcome at least by evoking gamma wave oscillations in a subject, the method comprising delivering a gamma oscillation inducing non-invasive sensory stimulus and/or evoking gamma wave oscillations in a subject. In some embodiments, the gamma oscillation inducing non-invasive sensory stimulus induces a signal that is indicative of an expected treatment outcome.

In some embodiments, the gamma oscillation inducing non-invasive sensory stimulus is delivered through one or more of: visual, auditory, tactile, olfactory stimulation, or bone conduction. In some embodiments combined audio-visual stimulation is delivered for an hour each day for a 3 to 6 month or longer period. In some embodiments, stimulation is delivered for two hours each day. In some embodiments, stimulation is delivered for multiple periods over the course of a day. In some embodiments, combined audio-visual stimulation is delivered over an extended open-ended period of time. In some embodiments, stimulus is delivered in periods of varying durations. In some embodiments, the gamma oscillation inducing non-invasive sensory stimulus is delivered at least in part through glasses, goggles, a mask, or other worn apparatus that provide visual stimulation.

In some embodiments, the gamma oscillation inducing non-invasive sensory stimulus is delivered at least in part through one or more devices in the user's environment, such as a speaker, lighting fixtures, bed attachment, wall mounted screen, or other household device. In some embodiments, the one or more devices are controlled by a further device, such as a phone, tablet, or home automation hub, configured to manage the delivery of the gamma oscillation inducing non-invasive sensory stimulus through the one or more devices in the user's environment.

In some embodiments, the gamma oscillation inducing non-invasive sensory stimulus is delivered through a pair of opaque or partially transparent glasses worn by the subject with illuminating elements on the interior providing a visual signal. In some embodiments, the gamma oscillation inducing non-invasive sensory stimulus is delivered through headphones or earbuds worn by the subject providing an auditory signal. In some embodiments, combined visual and auditory signals are provided by such headphones and glasses worn together at the same time. In some embodiments visual and auditory signals are delivered separately by glasses or headphones worn at different times. An exemplary embodiment includes a pair of glasses, with LEDs on the interior of the glasses providing visual stimulation and headphones providing auditory stimulation.

In some embodiments, the gamma oscillation inducing non-invasive sensory stimulus is delivered through vibrotactile stimulation via an article of clothing or body attachment. In some embodiments, the gamma oscillation inducing non-invasive sensory stimulus may be delivered through the user's nostrils.

In some embodiments, the gamma oscillation inducing non-invasive sensory stimulus is delivered through transcranial magnetic stimulation (TMS). In some embodiments, the gamma oscillation inducing non-invasive sensory stimulus is delivered through transcranial alternating current stimulation (tACS). In some embodiments, the gamma oscillation inducing non-invasive sensory stimulus is delivered through transcranial pulse current stimulation (tPCS).

In some embodiments, the gamma oscillation inducing non-invasive sensory stimulus is administered at least in part by a device as specified in one or more of US Patents U.S. Pat. No. 10,307,611 B2, U.S. Pat. No. 10,293,177 B2, or U.S. Pat. No. 10,279,192 B2.

In some embodiments, gamma oscillation inducing non-invasive sensory stimulus is delivered to more than one subject present in a space. In an exemplary embodiment, gamma oscillation inducing non-invasive sensory stimulus is delivered to more than one subject in a space through devices present in the space, such devices delivering the same stimulus to all present subjects, or customized stimulus to individual subjects, or a combination thereof.

### Program Parameters and Parameter Values

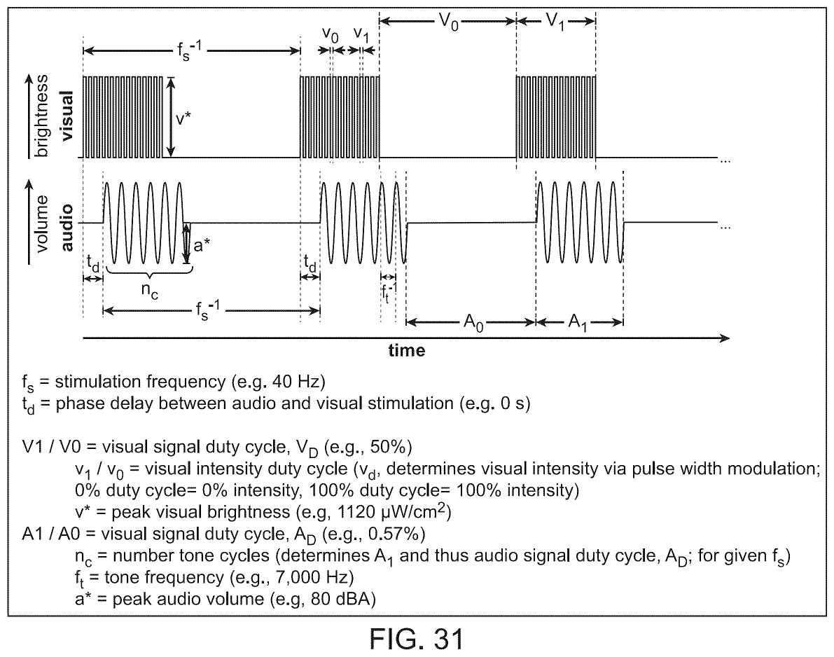

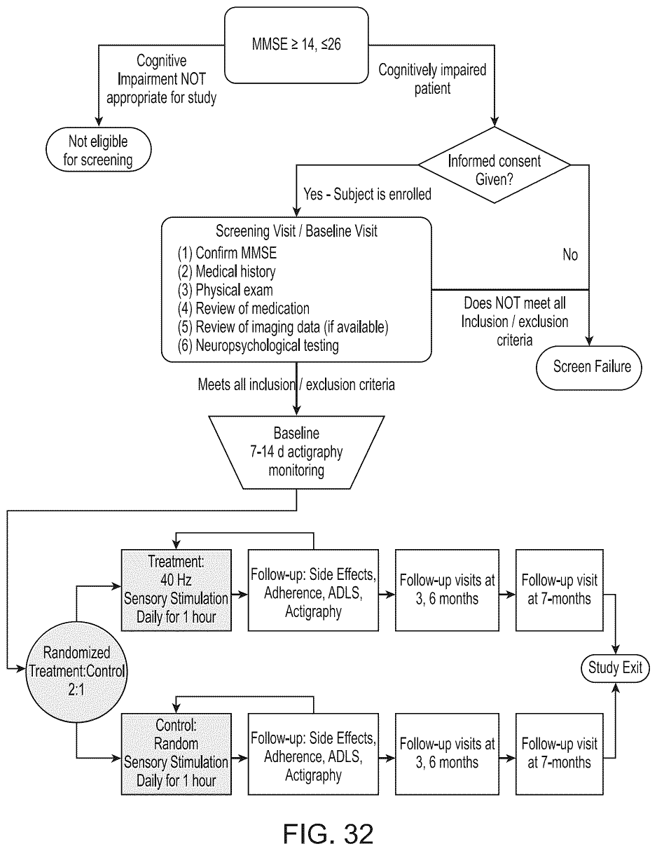

In some embodiments, gamma oscillation inducing non-invasive sensory stimulus parameters are configured with a stimulus frequency (e.g., fs in FIG. 31) of approximately 30 Hz to approximately 50 Hz for both audio and visual signals. In some embodiments, audio and visual signals are offset relative to each other by a delay (e.g., td in FIG. 31). In exemplary embodiment audio and visual signals are synchronized (td=0 s).

In some embodiments, gamma oscillation inducing non-invasive sensory stimulus parameters are configured with a variety of timing and intensity parameters. In an exemplary embodiment, these parameters include those illustrated in FIG. 31. In some embodiments, these parameters are preconfigured; in some embodiments they are adjusted at least in part by a third party such as a caregiver or healthcare provider; in some embodiments one or more parameters are adjusted responsive to measurements or analysis of one or more of: user context, measured sleep quality related parameters associated with the user, observed, or detected use of the stimulation device. In some embodiments, gamma oscillation inducing non-invasive sensory stimulus parameters are adjusted to detected or analyzed neurogenerative disease symptom progression. Various frequencies and various intensities may be used as parameters for the gamma oscillation inducing non-invasive sensory stimulus.

In some embodiments, one or more stimulus parameters are based at least partially on various clinical measures of treatment outcomes of cognitive function disclosed herein. In some embodiments, varying combinations stimulus parameters are used during different time periods and subsequent stimulation parameters are selected at least in part based on comparison of clinical measures of treatment outcomes for cognitive function among at least some of those periods.

In some embodiments, the present disclosure delivers 40 Hz non-invasive audio, visual, or combined audio-visual stimulation. In some embodiments, stimulus is delivered at one or more stimulation frequencies (e.g., fs in FIG. 31). In some embodiments, stimulus is delivered at one or more stimulation frequencies (e.g., fs in FIG. 31) in the approximate range of 30-50 Hz. In some embodiments, “gamma” refers to frequencies in the range 30-50 Hz. In some embodiments, the stimulus is periodic. In some embodiment, the stimulus is non-periodic. In some embodiments, the stimulus is a periodic stimulus with non-periodic components. In some embodiments, the stimulus is intermittent. In some embodiments stimulus is delivered based at least in part on a user's detected, reported, or demographically or individually associated or dominant alpha wave frequency.

In some embodiments, specific visual parameters include one or more of: stimulation frequency, intensity (brightness), hue, visual patterns, spatial frequency, contrast, and duty-cycle. In an exemplary embodiment, visual stimulation is provided at a stimulation frequency of 40 Hz, brightness between 0 μW/cm2 to 1120 μW/cm2, and 50% visual signal duty-cycle.

In some embodiments, non-invasive stimulation is delivered as combined visual and auditory stimulation, delivered at 40 Hz frequency. In some embodiments, visual and auditory stimulation is synchronized to begin each cycle simultaneously. In some embodiments, the beginning of each auditory and visual stimulation cycle is offset by a configured time. In some embodiments, visual and auditory signals are delivered at an intensity clearly recognized by subjects and adjusted to their tolerance level.

In some embodiments, at least some of the parameters or characteristics of the non-invasive signal administered to a subject correspond to those specified in one or more of US Patents U.S. Pat. No. 10,307,611 B2, U.S. Pat. No. 10,293,177 B2, or U.S. Pat. No. 10,279,192 B2. In some embodiments, at least some of the parameters or characteristics of the non-invasive signal administered to a subject correspond to those specified in one or more of US Patents U.S. Pat. No. 10,159,816 B2 or U.S. Pat. No. 10,265,497 B2.

In some embodiments specific audio parameters include one or more of: stimulation frequency, intensity (volume), and duty-cycle. In some embodiments, audio frequency is adjusted responsive to a subject's hearing characteristics, for example to frequencies that a subject is better at hearing. In an exemplary embodiment, audio stimulation is provided at an audio tone frequency of 7,000 Hz, volume level between 0 dBA to 80 dBA, and 0.57% audio signal duty-cycle.

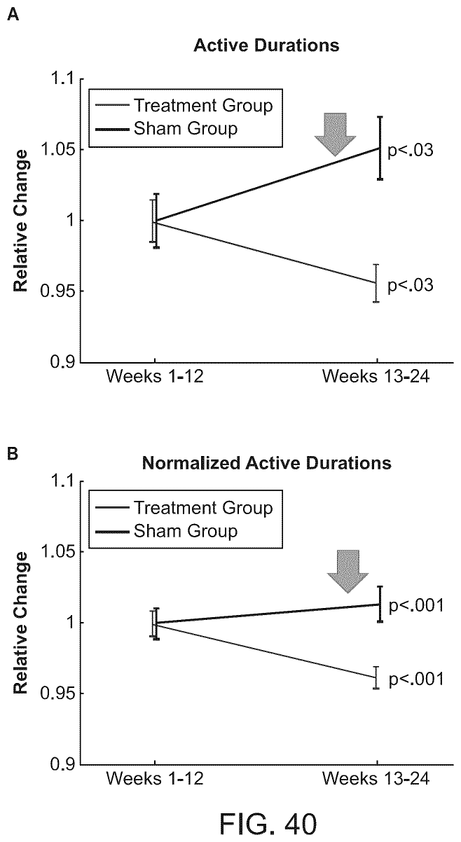

In some embodiments, non-invasive stimulation parameters are selected directed at evoking gamma wave oscillations in the brains of human subjects. In some embodiments, non-invasive stimulation parameters are selected directed at inducing alpha waves in human subjects (FIG. 40). In some embodiments, the non-invasive stimulation parameters are directed at inducing beta waves in human subjects. In some embodiments, the non-invasive stimulation parameters are directed at inducing gamma waves in human subjects.

In some embodiments light levels and hue are adjusted to avoid fatiguing the subject. In some embodiments light levels and hue are adjusted to provide motivation to the subject. In some embodiments, parameters to each ear or eye are adjusted in a similar manner. In some embodiments, parameters to each ear or eye are adjusted differently. In an exemplary embodiment, audio, and visual parameters such as tone and hue are varied to provide engagement or motivation to the subject to continue applying the stimulus or monitoring.

### Neural Stimulation Via Visual Stimulation

In some embodiments, systems and methods of the present disclosure are directed to controlling frequencies of neural oscillations using visual signals and, in doing so, causing a detectable signal indicative of an expected treatment outcome. The visual stimulation can adjust, control, or otherwise affect the frequency of the neural oscillations to provide beneficial effects to one or more cognitive states or cognitive functions of the brain, or the immune system, while mitigating or preventing adverse consequences on a cognitive state or cognitive function. The visual stimulation can result in sensory evoked neural oscillations that can produce detectable signals that may be correlated with potentially beneficial effects to one or more cognitive states of the brain, cognitive functions of the brain, the immune system, or inflammation. In some cases, the visual stimulation can result in local effect, such as in the visual cortex and associate regions. In some cases, the visual stimulation can result in a more expansive effect and cause alterations in physiology in more than just the nervous system. The sensory evoked neural oscillations may produce detectable signals that may be correlated with expected treatment outcomes for disorders, maladies, diseases, inefficiencies, injuries, or other issues related to a cognitive function of the brain, cognitive state of the brain, the immune system, or inflammation.

Neural oscillation occurs in humans or animals and includes rhythmic or repetitive neural activity in the central nervous system. Neural tissue can generate oscillatory activity by mechanisms within individual neurons or by interactions between neurons. Oscillations can appear as either oscillations in membrane potential or as rhythmic patterns of action potentials, which can produce oscillatory activation of post-synaptic neurons. Synchronized activity of a group of neurons can give rise to macroscopic oscillations, which, for example, can be observed by electroencephalography (“EEG”), magnetoencephalography (“MEG”), functional magnetic resonance imaging (“fMRI”), or electrocorticography (“ECOG”). Neural oscillations can be characterized by their frequency, amplitude, and phase. These signal properties can be observed from neural recordings using time-frequency analysis.

For example, an EEG can measure oscillatory activity among a group of neurons, and the measured oscillatory activity can be categorized into frequency bands as follows: delta activity corresponds to a frequency band from 0-4 Hz; theta activity corresponds to a frequency band from 4-8 Hz; alpha activity corresponds to a frequency band from 8-12 Hz; beta activity corresponds to a frequency band from 13-30 Hz; and gamma activity corresponds to a frequency band from 30-100 Hz.

The frequency and presence or activity of neural oscillations can be associated with cognitive states or cognitive functions such as information transfer, perception, motor control and memory. In some cases, the frequency and presence or activity of neural oscillations can be associated with deficiencies in cognitive states or cognitive functions. Based on the cognitive state or cognitive function, the frequency of neural oscillations can vary. Further, certain frequencies of neural oscillations can have beneficial effects or adverse consequences on one or more cognitive states or function. In some cases, characteristics of neural oscillations may be indicative of an expected treatment outcome.

Sensory evoked or sensory induced neural oscillations occur when an external stimulation of a particular frequency is encoded by neurons and triggers neural activity in the brain that results in neurons oscillating at a frequency corresponding to the particular frequency of the external stimulation. Thus, sensory evoked neural oscillations can refer to synchronizing neural oscillations in the brain using external stimulation such that the neural oscillations occur at a frequency that corresponds to a particular frequency component of the external stimulation. In some cases, sensory evoked neural oscillations may comprise additional neural oscillations having a frequency different from the frequency of the external stimulation. In some cases, the additional neural oscillations may be correlated with an expected treatment outcome.

Systems and methods of the present disclosure can provide external visual stimulation to achieve sensory induction of neural oscillations. For example, external signals, such as light pulses or high-contrast visual patterns, can be perceived by the brain. The brain, responsive to observing or perceiving the light pulses, can adjust, manage, or control the frequency of neural oscillations. The light pulses generated at a predetermined frequency and perceived by ocular means via a direct visual field or a peripheral visual field can trigger neural activity in the brain to induce neural oscillations in a particular frequency range. The frequency of neural oscillations can be affected at least in part by the frequency of light pulses. While high-level cognitive function may gate or interfere with sensory induction of neural oscillations in some regions, the brain can react to the visual stimulation at the sensory cortices. Thus, systems and methods of the present disclosure can provide sensory induction of neural oscillations using external visual stimulus such as light pulses emitted at a predetermined frequency to synchronize electrical activity among groups of neurons based on the frequency of light pulses. The sensory induction of neural oscillations in one or more portions or regions of the brain can be observed based on the aggregate frequency of oscillations produced by the synchronous electrical activity in ensembles of cortical neurons. The frequency of the light pulses can cause or adjust this synchronous electrical activity in the ensembles of cortical neurons to oscillate at a frequency corresponding to the frequency of the light pulses. In some cases, the brain may respond to the external visual stimulations to produce a detectable signal, wherein the detectable signal has characteristics that are correlated with an expected treatment outcome.

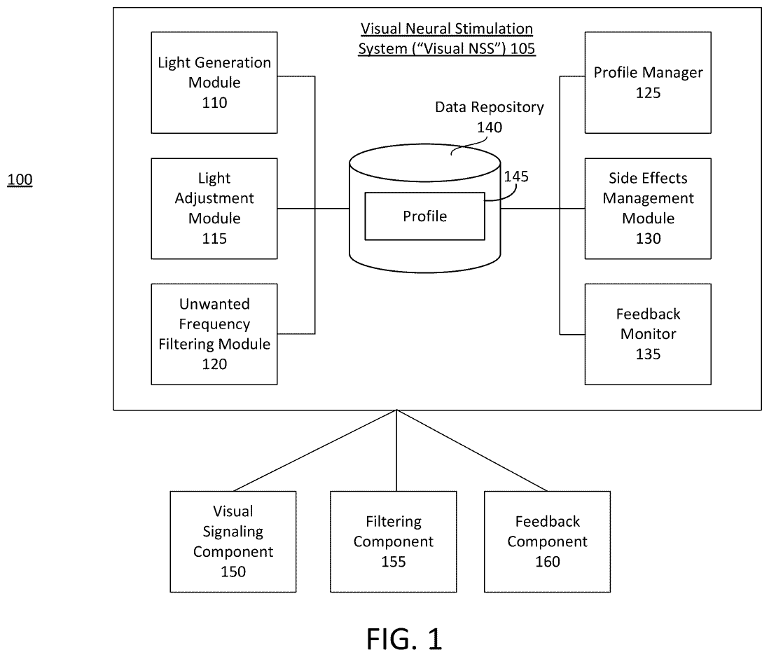

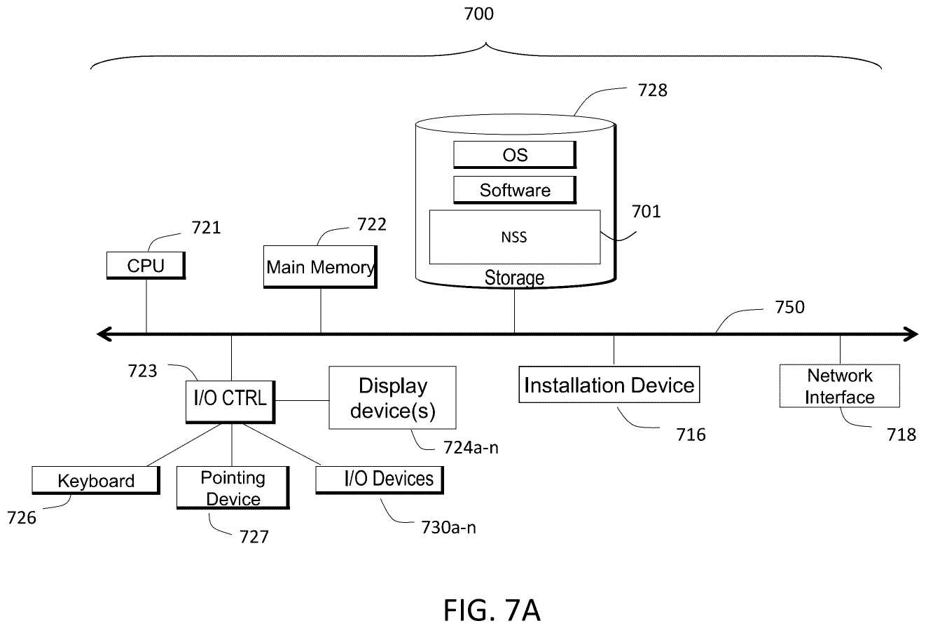

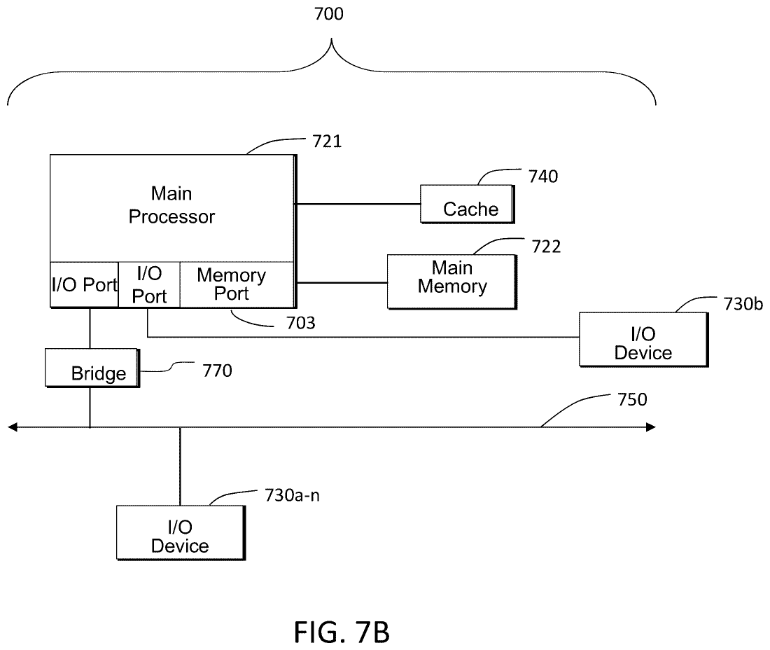

FIG. 1 is a block diagram depicting a system to perform visual stimulus induction of neural oscillations in accordance with an embodiment. The system 100 can include a neural stimulation system (“NSS”) 105. The NSS 105 can be referred to as visual NSS 105 or NSS 105. In brief overview, the NSS 105 can include, access, interface with, or otherwise communicate with one or more of a light generation module 110, light adjustment module 115, unwanted frequency filtering module 120, profile manager 125, side effects management module 130, feedback monitor 135, data repository 140, visual signaling component 150, filtering component 155, or feedback component 160. The light generation module 110, light adjustment module 115, unwanted frequency filtering module 120, profile manager 125, side effects management module 130, feedback monitor 135, visual signaling component 150, filtering component 155, or feedback component 160 can each include at least one processing unit or other logic device such as programmable logic array engine, or module configured to communicate with the database repository 150. The light generation module 110, light adjustment module 115, unwanted frequency filtering module 120, profile manager 125, side effects management module 130, feedback monitor 135, visual signaling component 150, filtering component 155, or feedback component 160 can be separate components, a single component, or part of the NSS 105. The system 100 and its components, such as the NSS 105, may include hardware elements, such as one or more processors, logic devices, or circuits. The system 100 and its components, such as the NSS 105, can include one or more hardware or interface component depicted in system 700 in FIG. 7A and FIG. 7B. For example, a component of system 100 can include or execute on one or more processors 721, access storage 728 or memory 722, and communicate via network interface 718.

Still referring to FIG. 1, and in further detail, the NSS 105 can include at least one light generation module 110. The light generation module 110 can be designed and constructed to interface with a visual signaling component 150 to provide instructions or otherwise cause or facilitate the generation of a visual signal, such as a light pulse or flash of light, having one or more predetermined parameter. The light generation module 110 can include hardware or software to receive and process instructions or data packets from one or more module or component of the NSS 105. The light generation module 110 can generate instructions to cause the visual signaling component 150 to generate a visual signal. The light generation module 110 can control or enable the visual signaling component 150 to generate the visual signal having one or more predetermined parameters.

The light generation module 110 can be communicatively coupled to the visual signaling component 150. The light generation module 110 can communicate with the visual signaling component 150 via a circuit, electrical wire, data port, network port, power wire, ground, electrical contacts, or pins. The light generation module 110 can wirelessly communicate with the visual signaling component 150 using one or more wireless protocols such as BlueTooth, BlueTooth Low Energy, Zigbee, Z-Wave, IEEE 802.11, WIFI, 3G, 4G, LTE, near field communications (“NFC”), or other short, medium, or long-range communication protocols, etc. The light generation module 110 can include or access network interface 718 to communicate wirelessly or over a wire with the visual signaling component 150.

The light generation module 110 can interface, control, or otherwise manage various types of visual signaling components 150 in order to cause the visual signaling component 150 to generate, block, control, or otherwise provide the visual signal having one or more predetermined parameters. The light generation module 110 can include a driver configured to drive a light source of the visual signaling component 150. For example, the light source can include a light emitting diode (“LED”), and the light generation module 110 can include an LED driver, chip, microcontroller, operational amplifiers, transistors, resistors, or diodes configured to drive the LED light source by providing electricity or power having certain voltage and current characteristics.

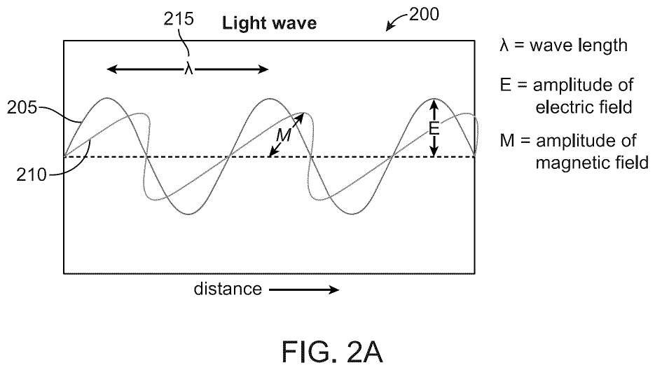

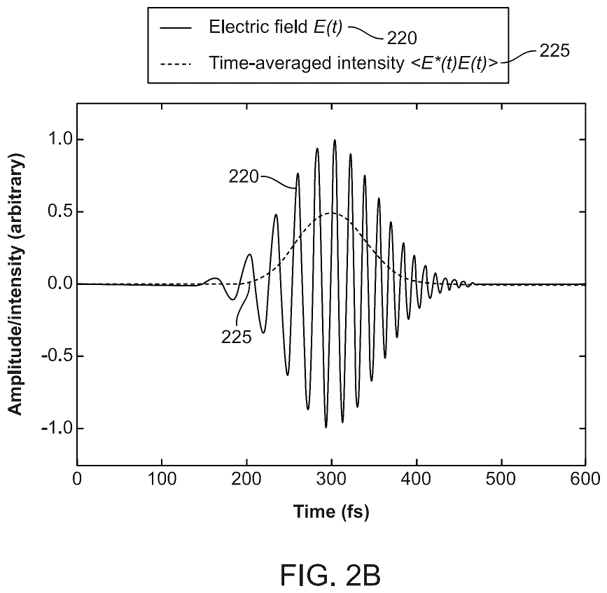

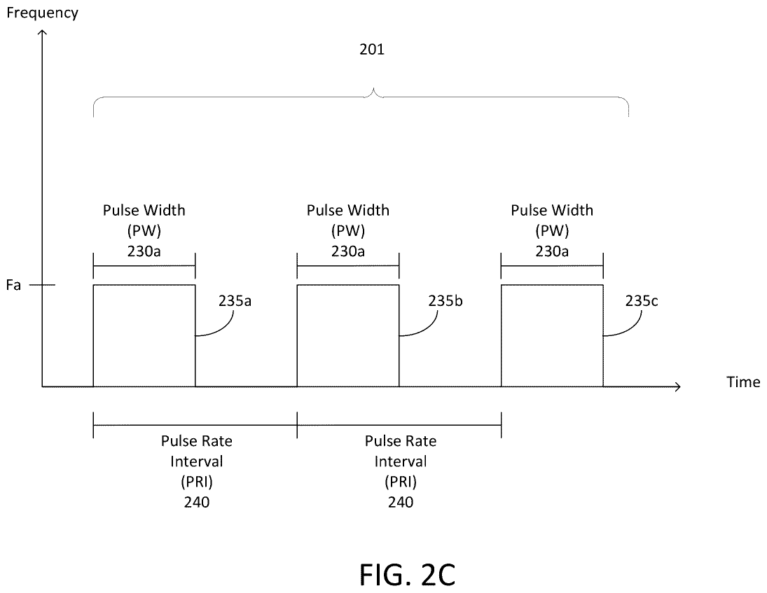

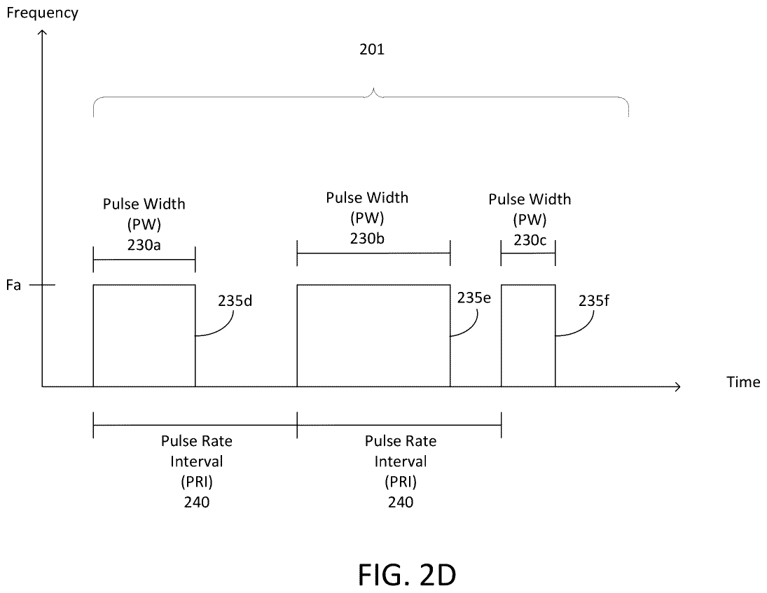

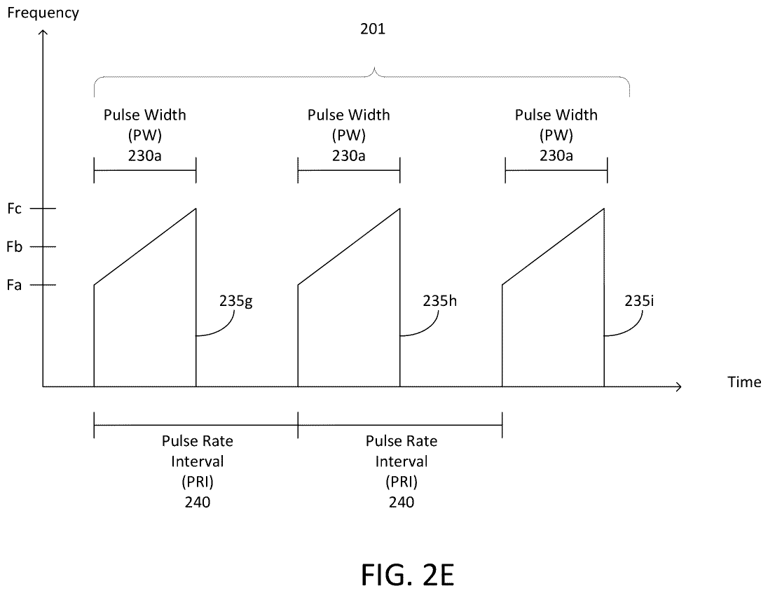

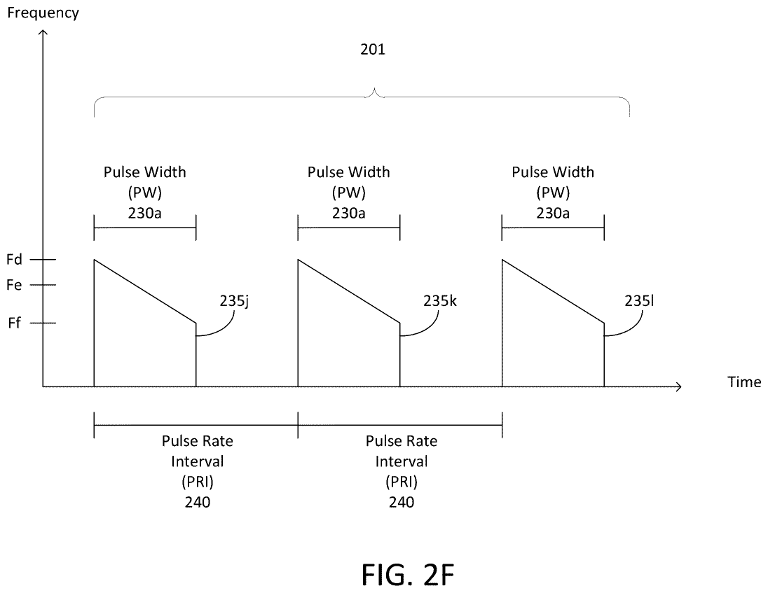

In some embodiments, the light generation module 110 can instruct the visual signaling component 150 to provide a visual signal that include a light wave 200 as depicted in FIG. 2A. The light wave 200 can include or be formed of electromagnetic waves. The electromagnetic waves of the light wave can have respective amplitudes and travel orthogonal to one another as depicted by the amplitude of the electric field 205 versus time and the amplitude of the magnetic field 210 versus time. The light wave 200 can have a wavelength 215. The light wave can also have a frequency. The product of the wavelength 215 and the frequency can be the speed of the light wave. For example, the speed of the light wave can be approximately 299,792,458 meters per second in a vacuum.

The light generation module 110 can instruct the visual signaling component 150 to generate light waves having one or more predetermined wavelength or intensity. The wavelength of the light wave can correspond to the visible spectrum, ultraviolet spectrum, infrared spectrum, or some other wavelength of light. For example, the wavelength of the light wave within the visible spectrum range can range from 390 to 700 nanometers (“nm”). Within the visible spectrum, the light generation module 110 can further specify one or more wavelengths corresponding to one or more colors. For example, the light generation module 110 can instruct the visual signaling component 150 to generate visual signals comprising one or more light waves having one or more wavelength corresponding to one or more of ultra-violet (e.g., 10-380 nm); violet (e.g., 380-450 nm), blue (e.g., 450-495 nm), green (e.g., 495-570 nm), yellow (e.g., 570-590 nm), orange (e.g., 590-620 nm), red (e.g., 620-750 nm); or infrared (e.g., 750-1000000 nm). The wavelength can range from 10 nm to 100 micrometers. In some embodiments, the wavelength can be in the range of 380 to 750 nm.

The light generation module 110 can determine to provide visual signals that include light pulses. The light generation module 110 can instruct or otherwise cause the visual signaling component 150 to generate light pulses. A light pulse can refer to a burst of light waves. For example, FIG. 2B illustrates a burst of a light wave. The burst of light wave can refer to a burst of an electric field 250 generated by the light wave. The burst of the electric field 250 of the light wave can be referred to as a light pulse or a flash of light. For example, a light source that is intermittently turned on and off can create bursts, flashes, or pulses of light.

FIG. 2C illustrates pulses of light 235a-c in accordance with an embodiment. The light pulses 235a-c can be illustrated via a graph in the frequency spectrum. The y-axis indicates frequency of the light wave (e.g., the speed of the light wave divided by the wavelength), and the x-axis represents time. The visual signal can include modulations of light wave between a frequency of Fₐ and frequency different from Fₐ. For example, the NSS 105 can modulate a light wave between a frequency in the visible spectrum, such as Fa, and a frequency outside the visible spectrum. The NSS 105 can modulate the light wave between two or more frequencies, between an on state and an off state, or between a high-power state and a low power state.

In some cases, the frequency of the light wave used to generate the light pulse can be constant at Fₐ, thereby generating a square wave in the frequency spectrum. In some embodiments, each of the three pulses 235a-c can include light waves having a same frequency, Fₐ.

The width of each of the light pulses (e.g., the duration of the burst of the light wave) can correspond to a pulse width 230a. The pulse width 230a can refer to the length or duration of the burst. The pulse width 230a can be measured in units of time or distance. In some embodiments, the pulses 235a-c can include lights waves having different frequencies from one another. In some embodiments, the pulses 235a-c can have different pulse widths 230a from one another, as illustrated in FIG. 2D. For example, a first pulse 235d of FIG. 2D can have a pulse width 230a, while a second pulse 235e has a second pulse width 230b that is greater than the first pulse width 230a. A third pulse 235f can have a third pulse width 230c that is less than the second pulse width 230b. The third pulse width 230c can also be less than the first pulse width 230a. While the pulse widths 230a-c of the pulses 235d-f of the pulse train may vary, the light generation module 110 can maintain a constant pulse rate interval 240 for the pulse train.

The pulses 235a-c can form a pulse train having a pulse rate interval 240. The pulse rate interval 240 can be quantified using units of time. The pulse rate interval 240 can be based on a frequency of the pulses of the pulse train 201. The frequency of the pulses of the pulse train 201 can be referred to as a modulation frequency. For example, the light generation module 110 can provide a pulse train 201 with a predetermined frequency corresponding to gamma activity, such as 40 Hz. To do so, the light generation module 110 can determine the pulse rate interval 240 by taking the multiplicative inverse (or reciprocal) of the frequency (e.g., 1 divided by the predetermined frequency for the pulse train). For example, the light generation module 110 can take the multiplicative inverse of 40 Hz by dividing 1 by 40 Hz to determine the pulse rate interval 240 as 0.025 seconds. The pulse rate interval 240 can remain constant throughout the pulse train. In some embodiments, the pulse rate interval 240 can vary throughout the pulse train or from one pulse train to a subsequent pulse train. In some embodiments, the number of pulses transmitted during a second can be fixed, while the pulse rate interval 240 varies.

In some embodiments, the light generation module 110 can generate a light pulse having a light wave that varies in frequency. For example, the light generation module 110 can generate up-chirp pulses where the frequency of the light wave of the light pulse increases from the beginning of the pulse to the end of the pulse as illustrated in FIG. 2E. For example, the frequency of a light wave at the beginning of pulse 235g can be Fₐ. The frequency of the light wave of the pulse 235g can increase from Fₐ to Fb in the middle of the pulse 235g, and then to a maximum of Fc at the end of the pulse 235g. Thus, the frequency of the light wave used to generate the pulse 235g can range from Fₐ to F_{c}. The frequency can increase linearly, exponentially, or based on some other rate or curve.

The light generation module 110 can generate down-chirp pulses, as illustrated in FIG. 2F, where the frequency of the light wave of the light pulse decreases from the beginning of the pulse to the end of the pulse. For example, the frequency of a light wave at the beginning of pulse 235j can be F_{d}. The frequency of the light wave of the pulse 235j can decrease from F_{d} to Fₑ in the middle of the pulse 235j, and then to a minimum of F_{f} at the end of the pulse 235j. Thus, the frequency of the light wave used to generate the pulse 235j can range from F_{d} to F_{f}. The frequency can decrease linearly, exponentially, or based on some other rate or curve.

Visual signaling component 150 can be designed and constructed to generate the light pulses responsive to instructions from the light generation module 110. The instructions can include, for example, parameters of the light pulse such as a frequency or wavelength of the light wave, intensity, duration of the pulse, frequency of the pulse train, pulse rate interval, or duration of the pulse train (e.g., a number of pulses in the pulse train or the length of time to transmit a pulse train having a predetermined frequency). The light pulse can be perceived, observed, or otherwise identified by the brain via ocular means such as eyes. The light pulses can be transmitted to the eye via direct visual field or peripheral visual field.

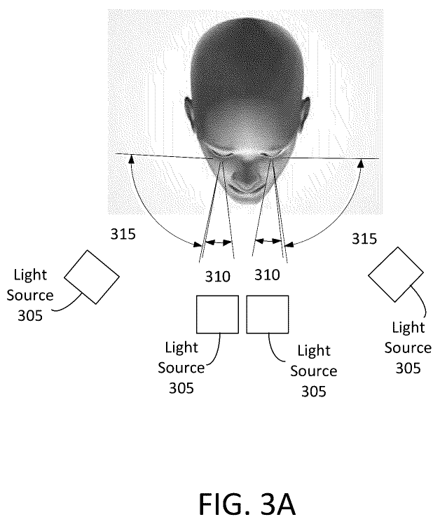

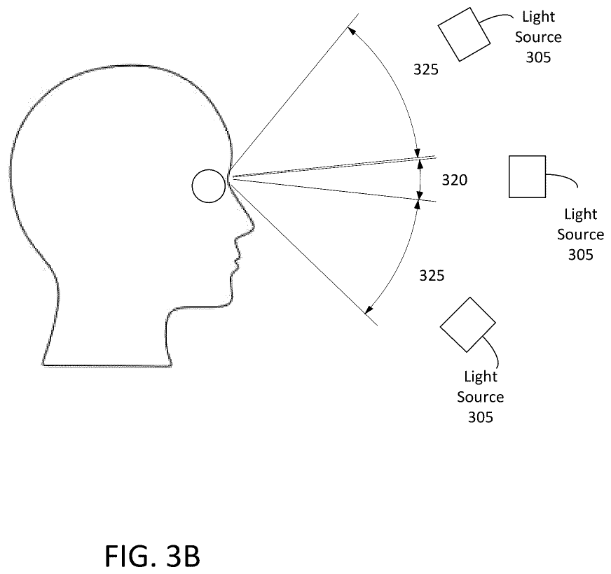

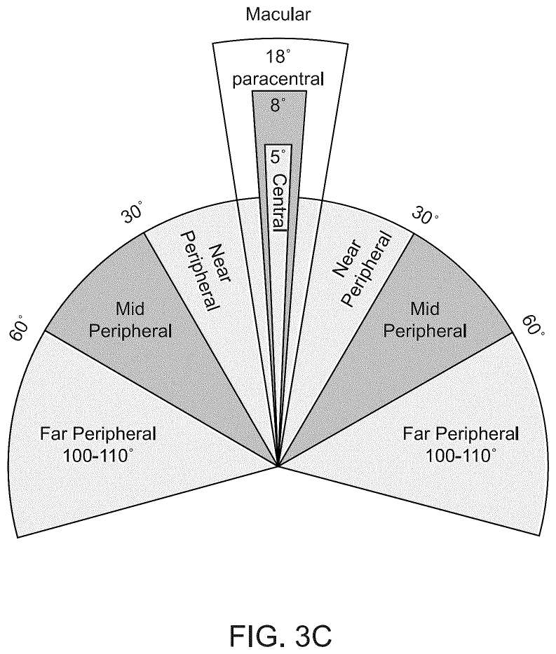

FIG. 3A illustrates a horizontal direct visual field 310 and a horizontal peripheral visual field. FIG. 3B illustrates a vertical direct visual field 320 and a vertical peripheral visual field 325. FIG. 3C illustrates degrees of direct visual fields and peripheral visual fields, including relative distances at which visual signals might be perceived in the different visual fields. The visual signaling component 150 can include a light source 305. The light source 305 can be positioned to transmit light pulses into the direct visual field 310 or 320 of a person's eyes. The NSS 105 can be configured to transmit light pulses into the direct visual field 310 or 320 because this may facilitate sensory induction of neural oscillations as the person may pay more attention to the light pulses. The level of attention can be quantitatively measured directly in the brain, indirectly through the person's eye behavior, or by active feedback (e.g., mouse tracking).

The light source 305 can be positioned to transmit light pulses into a peripheral visual field 315 or 325 of a person's eyes. For example, the NSS 105 can transmit light pulses into the peripheral visual field 315 or 325 as these light pulses may be less distracting to the person who might be performing other tasks, such as reading, walking, driving, etc. Thus, the NSS 105 can provide subtle, on-going visual brain stimulation by transmitting light pulses via the peripheral visual field.

In some embodiments, the light source 305 can be head-worn, while in other embodiments the light source 305 can be held by a subject's hands, placed on a stand, hung from a ceiling, or connected to a chair or otherwise positioned to direct light towards the direct or peripheral visual fields. For example, a chair or externally supported system can include or position the light source 305 to provide the visual input while maintaining a fixed/pre-specified relationship between the subject's visual field and the visual stimulus. The system can provide an immersive experience. For example, the system can include an opaque or partially opaque dome that includes the light source. The dome can be positioned over the subject's head while the subject sits or reclines in chair. The dome can cover portions of the subject's visual field, thereby reducing external distractions and facilitating sensory induction of neural oscillations of regions of the brain.

The light source 305 can include any type of light source or light emitting device. The light source can include a coherent light source, such as a laser. The light source 305 can include a light emitting diode (LED), Organic LED, fluorescent light source, incandescent light, or any other light emitting device. The light source can include a lamp, light bulb, or one or more light emitting diodes of various colors (e.g., white, red, green, blue). In some embodiments, the light source includes a semiconductor light emitting device, such as a light emitting diode of any spectral or wavelength range. In some embodiments, the light source 305 includes a broadband lamp or a broadband light source. In some embodiments, the light source includes a black light. In some embodiments, light source 305 includes a hollow cathode lamp, a fluorescent tube light source, a neon lamp, an argon lamp, a plasma lamp, a xenon flash lamp, a mercury lamp, a metal halide lamp, or a sulfur lamp. In some embodiments, the light source 305 includes a laser, or a laser diode. In some embodiments, light source 305 includes an OLED, PHOLED, QDLED, or any other variation of a light source utilizing an organic material. In some embodiments, light source 305 includes a monochromatic light source. In some embodiments, light source 305 includes a polychromatic light source. In some embodiments, the light source 305 includes a light source emitting light partially in the spectral range of ultraviolet light. In some embodiments, light source 305 includes a device, product or a material emitting light partially in the spectral range of visible light. In some embodiments, light source 305 is a device, product or a material partially emanating or emitting light in the spectral range of the infrared light. In some embodiments, light source 305 includes a device, product or a material emanating or emitting light in the visible spectral range. In some embodiments, light source 305 includes a light guide, an optical fiber or a waveguide through which light is emitted from the light source.

In some embodiments, light source 305 includes one or more mirrors for reflecting or redirecting of light. For example, the mirrors can reflect or redirect light towards the direct visual field 310 or 320, or the peripheral visual field 315 or 325. The light source 305 can include interact with microelectromechanical devices (“MEMS”). The light source 305 can include or interact with a digital light projector (“DLP”). In some embodiments, the light source 305 can include ambient light or sunlight. The ambient light or sunlight can be focused by one or more optical lenses and directed towards the direct visual field or peripheral field. The ambient light or sunlight can be directed by one or more mirrors towards the directed visual field or peripheral visual field.

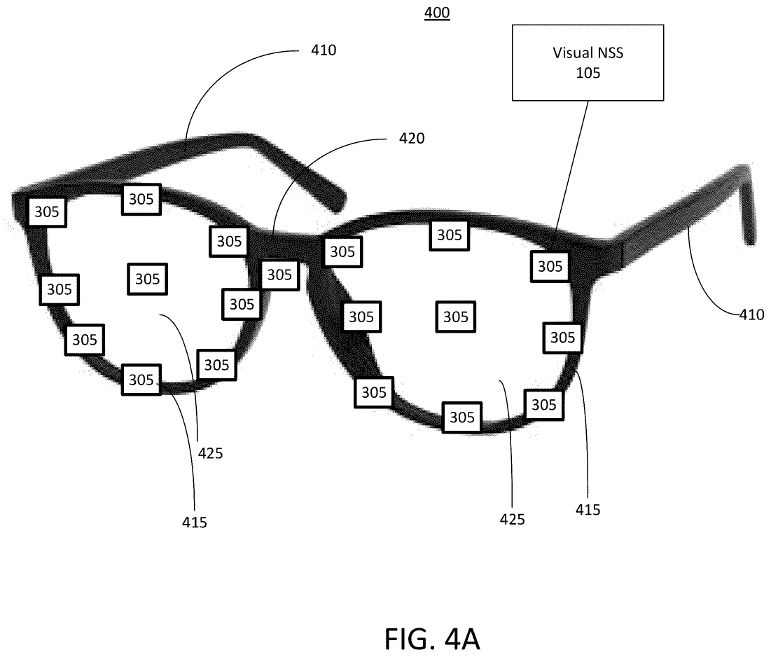

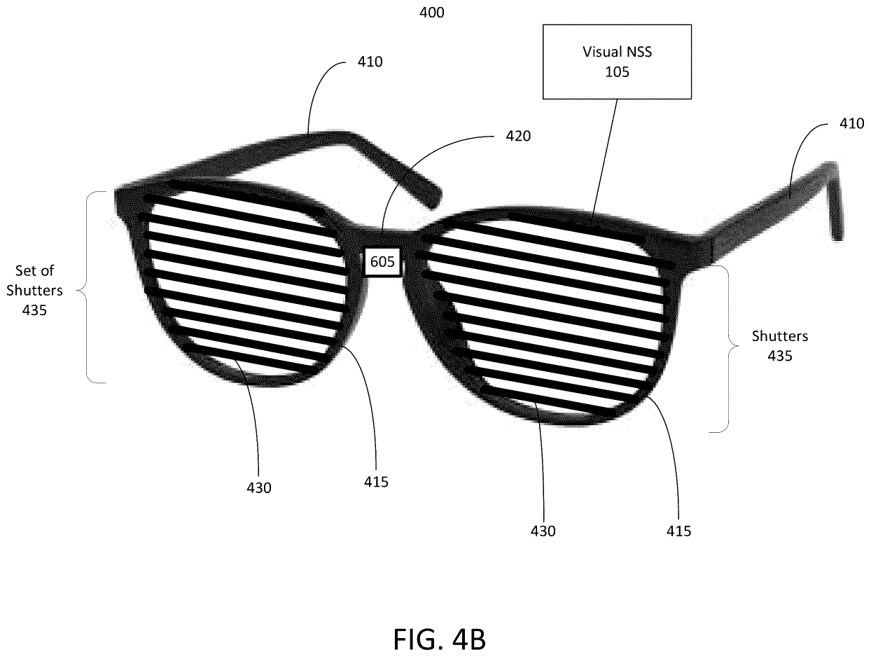

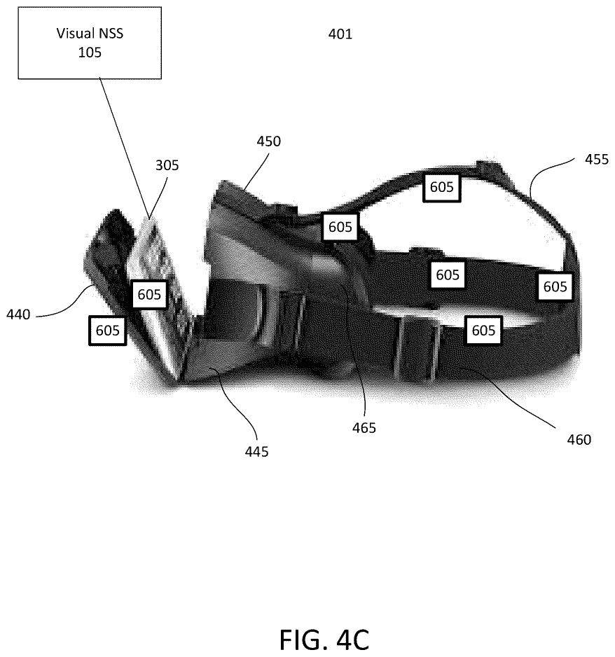

In cases where the light source is ambient light, the ambient light is not positioned but the ambient light can enter the eye via a direct visual field or peripheral visual field. In some embodiments, the light source 305 can be positioned to direct light pulses towards the direct visual field or peripheral field. For example, one or more light sources 305 can be attached, affixed, coupled, mechanically coupled, or otherwise provided with a frame 400 as illustrated in FIG. 4A. In some embodiments, the visual signaling component 150 can include the frame 400. Additional details of the operation of the NSS 105 in conjunction with the frame 400 including one or more light sources 305 are provided below, in the section labelled as “NSS Operating with A Frame”. Thus, the light source can include any type of light source such as an optical light source, mechanical light source, or chemical light source. The light source can include any material or object that is reflective or opaque that can generate, emit, or reflect oscillating patterns of light, such as a fan rotating in front of a light, or bubbles. In some embodiments, the light source can include optical illusions that are invisible, physiological phenomena that are within the eye (e.g., pressing the eyeball), or chemicals applied to the eye.

### Systems and Devices Configured for Neural Stimulation Via Visual Stimulation

Referring now to FIG. 4A, the frame 400 can be designed and constructed to be placed or positioned on a person's head. The frame 400 can be configured to be worn by the person. The frame 400 can be designed and constructed to stay in place. The frame 400 can be configured to be worn and stay in place as a person sits, stands, walks, runs, or lays down flat. The light source 305 can be configured on the frame 400 to project light pulses towards the person's eyes during these various positions. In some embodiments, the light source 305 can be configured to project light pulses towards the person's eyes if their eyelids are closed such that the light pulse penetrates the eyelid to be perceived by the retina. The frame 400 can include a bridge 420. The frame 400 can include one or more eye wires 415 coupled to the bridge 420. The bridge 420 can be positioned in between the eye wires 415. The frame 400 can include one or more temples extending from the one or more eye wires 415. In some embodiments, the eye wires 415 can include or hold a lens 425. In some embodiments, the eye wires 415 can include or hold a solid material 425 or cover 425. The lens, solid material, or cover 425 can be transparent, semi-transparent, opaque, or completely block out external light.

One or more light sources 305 can be positioned on or adjacent to the eye wire 415, lens or other solid material 425, or bridge 420. For example, a light source 305 can be positioned in the middle of the eye wire 415 on a solid material 425 in order to transmit light pulses into the direct visual field. In some embodiments, a light source 305 can be positioned at a corner of the eye wire 415, such as a corner of the eye wire 415 coupled to the temple 410, in order to transmit light pulses towards a peripheral field.

The NSS 105 can perform visual stimulus induction of neural oscillations via a single eye or both eyes. For example, the NSS 105 can direct light pulses to a single eye or both eyes. The NSS 105 can interface with a visual signaling component 150 that includes a frame 400 and two eye wires 415. However, the visual signaling component 150 may include a single light source 305 configured and positioned to direct light pulses to a first eye. The visual signaling component 150 can further include a light blocking component that keeps out or blocks the light pulses generated from the light source 305 from entering a second eye. The visual signaling component 150 can block or prevent light from entering the second eye during the sensory induction of neural oscillations.

In some embodiments, the visual signaling component 150 can alternatively transmit or direct light pulses to the first eye and the second eye. For example, the visual signaling component 150 can direct light pulses to the first eye for a first time interval. The visual signaling component 150 can direct light pulses to the second eye for a second time interval. The first time interval and the second time interval can be a same time interval, overlapping time intervals, mutually exclusive time intervals, or subsequent time intervals.

FIG. 4B illustrates a frame 400 comprising a set of shutters 435 that can block at least a portion of light that enters through the eye wire 415. The set of shutters 435 can intermittently block ambient light or sunlight that enters through the eye wire 415. The set of shutters 435 can open to allow light to enter through the eye wire 415, and close to at least partially block light that enters through the eye wire 415. Additional details of the operation of the NSS 105 in conjunction with the frame 400 including one or more shutters 430 are provided below, in the section labelled as “NSS Operating with A Frame”.

The set of shutters 435 can include one or more shutter 430 that is opened and closed by one or more actuator. The shutter 430 can be formed from one or more materials. The shutter 430 can include one or more materials. The shutter 430 can include or be formed from materials that are capable of at least partially blocking or attenuating light.

The frame 400 can include one or more actuators configured to at least partially open or close the set of shutters 435 or an individual shutter 430. The frame 400 can include one or more types of actuators to open and close the shutters 435. For example, the actuator can include a mechanically driven actuator. The actuator can include a magnetically driven actuator. The actuator can include a pneumonic actuator. The actuator can include a hydraulic actuator. The actuator can include a piezoelectric actuator. The actuator can include a micro-electromechanical systems (“MEMS”).

The set of shutters 435 can include one or more shutter 430 that is opened and closed via electrical or chemical techniques. For example, the shutter 430 or set of shutters 435 can be formed from one or more chemicals. The shutter 430 or set of shutters can include one or more chemicals. The shutter 430 or set of shutters 435 can include or be formed from chemicals that are capable of at least partially blocking or attenuating light.

For example, the shutter 430 or set of shutters 435 can include photochromic lenses configured to filter, attenuate, or block light. The photochromic lenses can automatically darken when exposed to sunlight. The photochromic lens can include molecules that are configured to darken the lens. The molecules can be activated by light waves, such as ultraviolet radiation or other light wavelengths. Thus, the photochromic molecules can be configured to darken the lens in response to a predetermined wavelength of light.

The shutter 430 or set of shutters 435 can include electrochromic glass or plastic. Electrochromic glass or plastic can change from light to dark (e.g., clear to opaque) in response to an electrical voltage or current. Electrochromic glass or plastic can include metal-oxide coatings that are deposited on the glass or plastic, multiple layers, and lithium ions that travel between two electrodes between a layer to lighten or darken the glass.

The shutter 430 or set of shutters 435 can include micro shutters. Micro shutters can include tiny windows that measure 100 by 200 microns. The micro shutters can be arrayed in the eye frame 415 in a waffle-like grid. The individual micro shutters can be opened or closed by an actuator. The actuator can include a magnetic arm that sweeps past the micro shutter to open or close the micro shutter. An open micro shutter can allow light to enter through the eye frame 415, while a closed micro shutter can block, attenuate, or filter the light.

The NSS 105 can drive the actuator to open and close one or more shutters 430 or the set of shutters 435 at a predetermined frequency, such as 40 Hz. By opening and closing the shutter 430 at the predetermined frequency, the shutter 430 can allow flashes of light to pass through the eye wire 415 at the predetermined frequency. Thus, the frame 400 including a set of shutters 435 may not include or use separate light source coupled to the frame 400, such as a light source 305 coupled to frame 400 depicted in FIG. 4A.

In some embodiments, the visual signaling component 150 or light source 305 can refer to or be included in a virtual reality headset 401, as depicted in FIG. 4C. For example, the virtual reality headset 401 can be designed and constructed to receive a light source 305. The light source 305 can include a computing device having a display device, such as a smartphone or mobile telecommunications device. The virtual reality headset 401 can include a cover 440 that opens to receive the light source 305. The cover 440 can close to lock or hold the light source 305 in place. When closed, the cover 440 and case 450 and 445 can form an enclosure for the light source 305. This enclosure can provide an immersive experience that minimize or eliminates unwanted visual distractions. The virtual reality headset can provide an environment to maximize sensory induction of neural oscillations. The virtual reality headset can provide an augmented reality experience. In some embodiments, the light source 305 can form an image on another surface such that the image is reflected off the surface and towards a subject's eye (e.g., a heads up display that overlays on the screen a flickering object or an augmented portion of reality). Additional details of the operation of the NSS 105 in conjunction with the virtual reality headset 401 are provided below, in the section labeled as “Systems and Devices Configured for Neural Stimulation Via Visual Stimulation”.

The virtual reality headset 401 includes straps 455 and 460 configured to secure the virtual reality headset 401 to a person's head. The virtual reality headset 401 can be secured via straps 455 and 460 such to minimize movement of the headset 401 worn during physical activity, such as walking or running. The virtual reality headset 401 can include a skull cap formed from 460 or 455.

The feedback sensor 605 can include an electrode, dry electrode, gel electrode, saline soaked electrode, or adhesive-based electrodes.

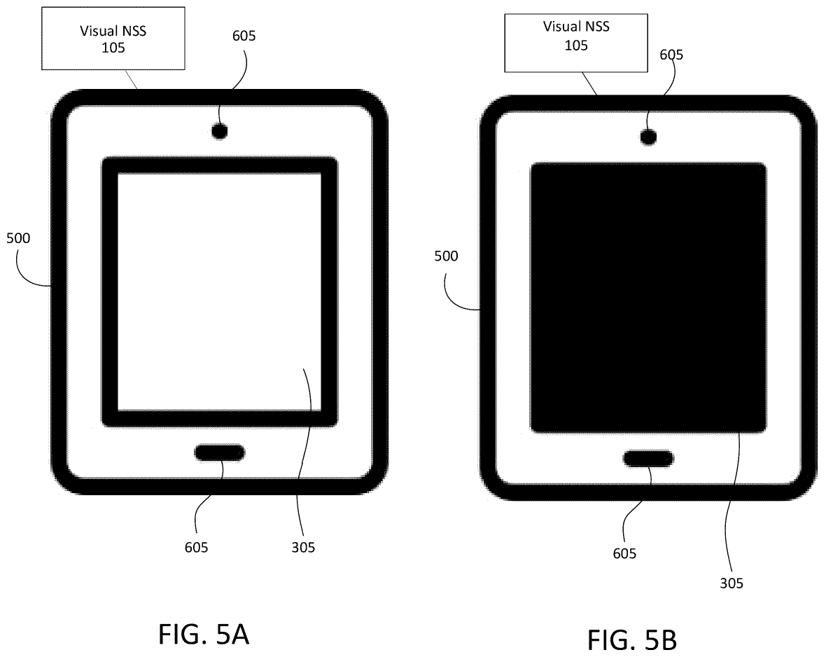

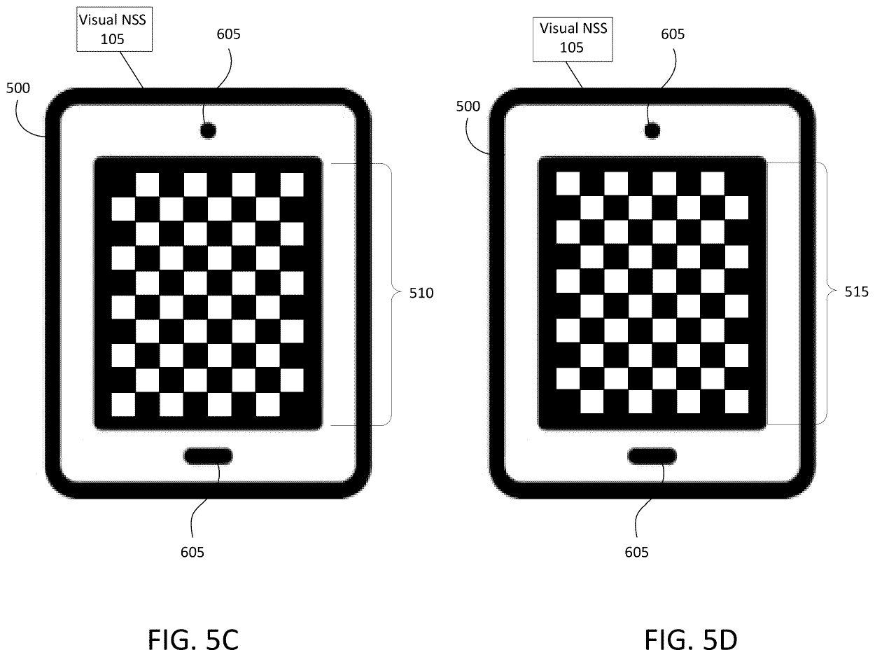

FIGS. 5A-5D illustrate embodiments of the visual signaling component 150 that can include a tablet computing device 500 or other computing device 500 having a display screen 305 as the light source 305. The visual signaling component 150 can transmit light pulses, light flashes, or patterns of light via the display screen 305 or light source 305.

FIG. 5A illustrates a display screen 305 or light source 305 that transmits light. The light source 305 can transmit light comprising a wavelength in the visible spectrum. The NSS 105 can instruct the visual signaling component 150 to transmit light via the light source 305. The NSS 105 can instruct the visual signaling component 150 to transmit flashes of light or light pulses having a predetermined pulse rate interval. For example, FIG. 5B illustrates the light source 305 turned off or disabled such that the light source does not emit light or emits a minimal or reduced amount of light. The visual signaling component 150 can cause the tablet computing device 500 to enable (e.g., FIG. 5A) and disable (e.g., FIG. 5B) the light source 305 such that flashes of light have a predetermined frequency, such as 40 Hz. The visual signaling component 150 can toggle or switch the light source 305 between two or more states to generate flashes of light or light pulses with the predetermined frequency.

In some embodiments, the light generation module 110 can instruct or cause the visual signaling component 150 to display a pattern of light via display device 305 or light source 305, as depicted in FIGS. 5C and 5D. The light generation module 110 can cause the visual signaling component 150 can flicker, toggle or switch between two or more patterns to generate flashes of light or light pulses. Patterns can include, for example, alternating checkerboard patterns 510 and 515. The pattern can include symbols, characters, or images that can be toggled or adjusted from one state to another state. For example, the color of a character or text relative to a background color can be inverted to cause a switch between a first state 510 and a second state 515. Inverting a foreground color and background color at a predetermined frequency can generate light pulses by way of indicating visual changes that can facilitate adjusting or managing a frequency of neural oscillations. Additional details of the operation of the NSS 105 in conjunction with the tablet 500 are provided below, in the section labeled as “NSS Operating with a Tablet”.

In some embodiments, the light generation module 110 can instruct or cause the visual signaling component 150 to flicker, toggle, or switch between images configured to stimulate specific or predetermined portions of the brain or a specific cortex. The presentation, form, color, motion, and other aspects of the light or image-based stimuli can dictate which cortex or cortices are recruited to process the stimuli. The visual signaling component 150 can stimulate discrete portions of the cortex by modulating the presentation of the stimuli to target specific or general regions of interest. The relative position in the field of view, the color of the input, or the motion and speed of the light stimuli can dictate which region of the cortex is stimulated.

For example, the brain can include at least two portions that process predetermined types of visual stimuli: the primary visual cortex on the left side of the brain, and the calcarine fissure on the right side of the brain. Each of these two portions can have one or more multiple sub-portions that process predetermined types of visual stimuli. For example, the calcarine fissure can include a sub-portion referred to as area V5 that can include neurons that respond strongly to motion but may not register stationary objects. Subjects with damage to area V5 may have motion blindness, but otherwise normal vision. In another example, the primary visual cortex can include a sub-portion referred to as area V4 that can include neurons that are specialized for color perception. Subjects with damage to area V4 may have color blindness and only perceive objects in shades of gray. In another example, the primary visual cortex can include a sub-portion referred to as area V1 that includes neurons that respond strongly to contrast edges and helps segment the image into separate objects.

Thus, the light generation module 110 can instruct or cause the visual signaling component 150 to form a type of still image or video, or generate a flicker, or toggle between images that configured to stimulate specific or predetermined portions of the brain or a specific cortex. For example, the light generation module 110 can instruct or cause the visual signaling component 150 to generate images of human faces to stimulate a fusiform face area, which can facilitate sensory induction of neural oscillations for subjects having prosopagnosia or face blindness. The light generation module 110 can instruct or cause the visual signaling component 150 to generate images of faces flickering to target this area of the subject's brain. In another example, the light generation module 110 can instruct the visual signaling component 150 to generate images that include edges or line drawings to stimulate neurons of the primary visual cortex that respond strongly to contrast edges.

The NSS 105 can include, access, interface with, or otherwise communicate with at least one light adjustment module 115. The light adjustment module 115 can be designed and constructed to measure or verify an environmental variable (e.g., light intensity, timing, incident light, ambient light, eye lid status, etc.) to adjust a parameter associated with the visual signal, such as a frequency, amplitude, wavelength, intensity pattern or other parameter of the visual signal. The light adjustment module 115 can automatically vary a parameter of the visual signal based on profile information or feedback. The light adjustment module 115 can receive the feedback information from the feedback monitor 135. The light adjustment module 115 can receive instructions or information from a side effects management module 130. The light adjustment module 115 can receive profile information from profile manager 125.

The NSS 105 can include, access, interface with, or otherwise communicate with at least one unwanted frequency filtering module 120. The unwanted frequency filtering module 120 can be designed and constructed to block, mitigate, reduce, or otherwise filter out frequencies of visual signals that are undesired to prevent or reduce an amount of such visual signals from being perceived by the brain. The unwanted frequency filtering module 120 can interface, instruct, control, or otherwise communicate with a filtering component 155 to cause the filtering component 155 to block, attenuate, or otherwise reduce the effect of the unwanted frequency on the neural oscillations.

The NSS 105 can include, access, interface with, or otherwise communicate with at least one profile manager 125. The profile manager 125 can be designed or constructed to store, update, retrieve or otherwise manage information associated with one or more subjects associated with visual stimulus induced neural oscillations. Profile information can include, for example, historical treatment information, historical sensory induced of neural oscillations information, dosing information, parameters of light waves, feedback, physiological information, environmental information, or other data associated with the systems and methods of sensory induction of neural oscillations.

The NSS 105 can include, access, interface with, or otherwise communicate with at least one side effects management module 130. The side effects management module 130 can be designed and constructed to provide information to the light adjustment module 115 or the light generation module 110 to change one or more parameter of the visual signal in order to reduce a side effect. Side effects can include, for example, nausea, migraines, fatigue, seizures, eye strain, or loss of sight.

The side effects management module 130 can automatically instruct a component of the NSS 105 to alter or change a parameter of the visual signal. The side effects management module 130 can be configured with predetermined thresholds to reduce side effects. For example, the side effects management module 130 can be configured with a maximum duration of a pulse train, maximum intensity of light waves, maximum amplitude, maximum duty cycle of a pulse train (e.g., the pulse width multiplied by the frequency of the pulse train), maximum number of treatments for sensory induction of neural oscillations in a time period (e.g., 1 hour, 2 hours, 12 hours, or 24 hours).

The side effects management module 130 can cause a change in the parameter of the visual signal in response to feedback information. The side effect management module 130 can receive feedback from the feedback monitor 135. The side effects management module 130 can determine to adjust a parameter of the visual signal based on the feedback. The side effects management module 130 can compare the feedback with a threshold to determine to adjust the parameter of the visual signal.

The side effects management module 130 can be configured with or include a policy engine that applies a policy or a rule to the current visual signal and feedback to determine an adjustment to the visual signal. For example, if feedback indicates that a patient receiving visual signals has a heart rate or pulse rate above a threshold, the side effects management module 130 can turn off the pulse train until the pulse rate stabilizes to a value below the threshold, or below a second threshold that is lower than the threshold.

The NSS 105 can include, access, interface with, or otherwise communicate with at least one feedback monitor 135. The feedback monitor can be designed and constructed to receive feedback information from a feedback component 160. Feedback component 160 can include, for example, a feedback sensor 605 such as a temperature sensor, heart or pulse rate monitor, physiological sensor, ambient light sensor, ambient temperature sensor, sleep status via actigraphy, blood pressure monitor, respiratory rate monitor, brain wave sensor, EEG probe, electrooculography (“EOG”) probes configured to measure the corneo-retinal standing potential that exists between the front and the back of the human eye, accelerometer, gyroscope, motion detector, proximity sensor, camera, microphone, or photo detector.

In some embodiments, a computing device 500 can include the feedback component 160 or feedback sensor 605, as depicted in FIGS. 5C and 5D. For example, the feedback sensor on tablet 500 can include a front-facing camera that can capture images of a person viewing the light source 305.

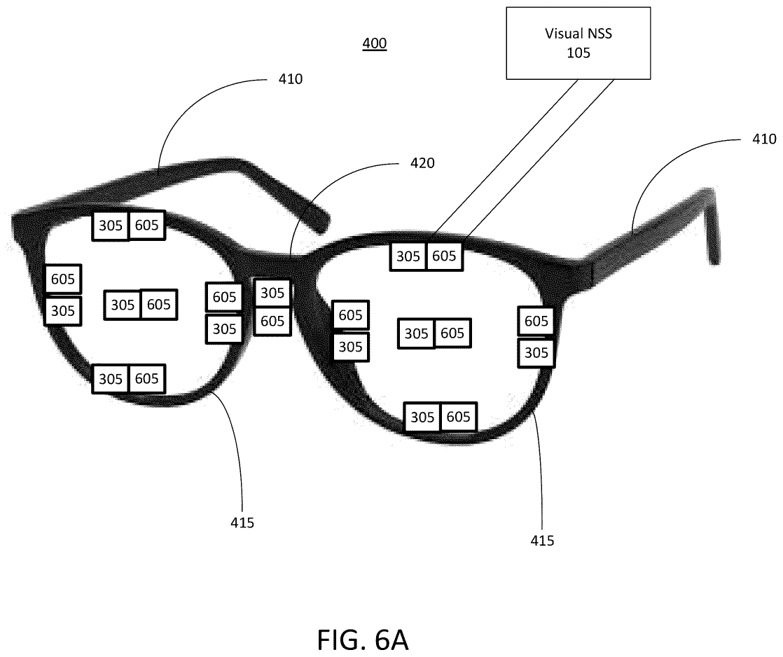

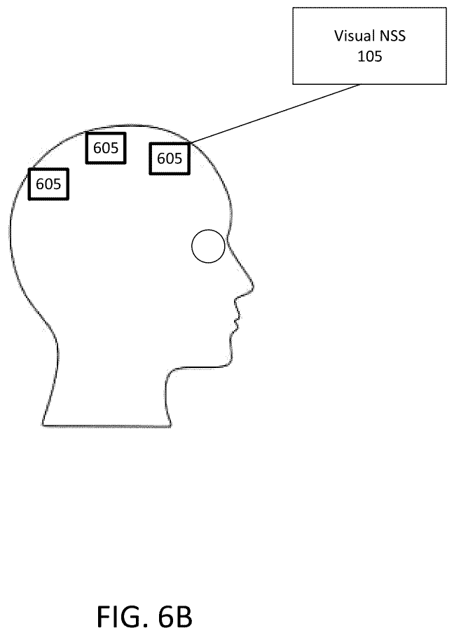

FIG. 6A depicts one or more feedback sensors 605 provided on a frame 400. In some embodiments, a frame 400 can include one or feedback sensors 605 provided on a portion of the frame, such as the bridge 420 or portion of the eye wire 415. The feedback sensor 605 can be provided with or coupled to the light source 305. The feedback sensor 605 can be separate from the light source 305.

The feedback sensor 605 can interact with or communicate with NSS 105. For example, the feedback sensor 605 can provide detected feedback information or data to the NSS 105 (e.g., feedback monitor 135). The feedback sensor 605 can provide data to the NSS 105 in real-time, for example as the feedback sensor 605 detects or senses or information. The feedback sensor 605 can provide the feedback information to the NSS 105 based on a time interval, such as 1 minute, 2 minutes, 5 minutes, 10 minutes, hourly, 2 hours, 4 hours, 12 hours, or 24 hours. The feedback sensor 605 can provide the feedback information to the NSS 105 responsive to a condition or event, such as a feedback measurement exceeding a threshold or falling below a threshold. The feedback sensor 605 can provide feedback information responsive to a change in a feedback parameter. In some embodiments, the NSS 105 can ping, query, or send a request to the feedback sensor 605 for information, and the feedback sensor 605 can provide the feedback information in response to the ping, request, or query.

FIG. 6B illustrates feedback sensors 605 placed or positioned at, on, or near a person's head. Feedback sensors 605 can include, for example, EEG probes that detect brain wave activity.

The feedback monitor 135 can detect, receive, obtain, or otherwise identify feedback information from the one or more feedback sensors 605. The feedback monitor 135 can provide the feedback information to one or more component of the NSS 105 for further processing or storage. For example, the profile manager 125 can update profile data structure 145 stored in data repository 140 with the feedback information. Profile manager 125 can associate the feedback information with an identifier of the patient or person undergoing the visual brain stimulation, as well as a time stamp and date stamp corresponding to receipt or detection of the feedback information.

The feedback monitor 135 can determine a level of attention. The level of attention can refer to the focus provided to the light pulses used for brain stimulation. The feedback monitor 135 can determine the level of attention using various hardware and software techniques. The feedback monitor 135 can assign a score to the level of attention (e.g., 1 to 10 with 1 being low attention and 10 being high attention, or vice versa, 1 to 100 with 1 being low attention and 100 being high attention, or vice versa, 0 to 1 with 0 being low attention and 1 being high attention, or vice versa), categorize the level of attention (e.g., low, medium, high), grade the attention (e.g., A, B, C, D, or F), or otherwise provide an indication of a level of attention.

In some cases, the feedback monitor 135 can track a person's eye movement to identify a level of attention. The feedback monitor 135 can interface with a feedback component 160 that includes an eye-tracker. The feedback monitor 135 (e.g., via feedback component 160) can detect and record eye movement of the person and analyze the recorded eye movement to determine an attention span or level of attention. The feedback monitor 135 can measure eye gaze which can indicate or provide information related to covert attention. For example, the feedback monitor 135 (e.g., via feedback component 160) can be configured with electro-oculography (“EOG”) to measure the skin electric potential around the eye, which can indicate a direction the eye faces relative to the head. In some embodiments, the EOG can include a system or device to stabilize the head so it cannot move, in order to determine the direction of the eye relative to the head. In some embodiments, the EOG can include or interface with a head tracker system to determine the position of the heads, and then determine the direction of the eye relative to the head.

In some embodiments, the feedback monitor 135 and feedback component 160 can determine or track the direction of the eye or eye movement using video detection of the pupil or corneal reflection. For example, the feedback component 160 can include one or more camera or video camera. The feedback component 160 can include an infra-red source that sends light pulses towards the eyes. The light can be reflected by the eye. The feedback component 160 can detect the position of the reflection. The feedback component 160 can capture or record the position of the reflection. The feedback component 160 can perform image processing on the reflection to determine or compute the direction of the eye or gaze direction of the eye.

The feedback monitor 135 can compare the eye direction or movement to historical eye direction or movement of the same person, nominal eye movement, or other historical eye movement information to determine a level of attention. For example, if the eye is focused on the light pulses during the pulse train, then the feedback monitor 135 can determine that the level of attention is high. If the feedback monitor 135 determines that the eye moved away from the pulse train for 25% of the pulse train, then the feedback monitor 135 can determine that the level of attention is medium. If the feedback monitor 135 determines that the eye movement occurred for more than 50% of the pulse train or the eye was not focused on the pulse train for greater than 50%, then the feedback monitor 135 can determine that the level of attention is low.

In some embodiments, the system 100 can include a filter (e.g., filtering component 155) to control the spectral range of the light emitted from the light source. In some embodiments, light source includes a light reactive material affecting the light emitted, such as a polarizer, filter, prism or a photochromic material, or electrochromic glass or plastic. The filtering component 155 can receive instructions from the unwanted frequency filtering module 120 to block or attenuate one or more frequencies of light.

The filtering component 155 can include an optical filter that can selectively transmit light in a particular range of wavelengths or colors, while blocking one or more other ranges of wavelengths or colors. The optical filter can modify the magnitude or phase of the incoming light wave for a range of wavelengths. The optical filter can include an absorptive filter, or an interference or dichroic filter. An absorptive filter can take energy of a photon to transform the electromagnetic energy of a light wave into internal energy of the absorber (e.g., thermal energy). The reduction in intensity of a light wave propagating through a medium by absorption of a part of its photons can be referred to as attenuation.

An interference filter or dichroic filter can include an optical filter that reflects one or more spectral bands of light, while transmitting other spectral bands of light. An interference filter or dichroic filter may have a nearly zero coefficient of absorption for one or more wavelengths. Interference filters can be high-pass, low-pass, bandpass, or band-rejection. An interference filter can include one or more thin layers of a dielectric material or metallic material having different refractive indices.

In an illustrative implementation, the NSS 105 can interface with a visual signaling component 150, a filtering component 155, and a feedback component 160. The visual signaling component 150 can include hardware or devices, such as glass frames 400 and one or more light sources 305. The filtering component 155 can include hardware or devices, such as a feedback sensor 605. The filtering component 155 can include hardware, materials, or chemicals, such as a polarizing lens, shutters, electrochromic materials, or photochromic materials.

### Computing Environment

FIGS. 7A and 7B depict block diagrams of a computing device 700. As shown in FIGS. 7A and 7B, each computing device 700 includes a central processing unit 721, and a main memory unit 722. As shown in FIG. 7A, a computing device 700 can include a storage device 728, an installation device 716, a network interface 718, an I/O controller 723, display devices 724a-724n, a keyboard 726 and a pointing device 727, e.g., a mouse. The storage device 728 can include, without limitation, an operating system, software, and software of a neural stimulation system (“NSS”) 701. The NSS 701 can include or refer to one or more of NSS 105, NSS 905, or NSOS 1605. As shown in FIG. 7B, each computing device 700 can also include additional optional elements, e.g., a memory port 703, a bridge 770, one or more input/output devices 730a-730n (generally referred to using reference numeral 730), and a cache memory 740 in communication with the central processing unit 721.

The central processing unit 721 is any logic circuitry that responds to and processes instructions fetched from the main memory unit 722. In many embodiments, the central processing unit 721 is provided by a microprocessor unit, e.g.: those manufactured by Intel Corporation of Mountain View, California; those manufactured by Motorola Corporation of Schaumburg, Illinois; the ARM processor (from, e.g., ARM Holdings and manufactured by ST, TI, ATMEL, etc.) and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California; or field programmable gate arrays (“FPGAs”) from Altera in San Jose, CA, Intel Corporation, Xlinix in San Jose, CA, or MicroSemi in Aliso Viejo, CA, etc. The computing device 700 can be based on any of these processors, or any other processor capable of operating as described herein. The central processing unit 721 can utilize instruction level parallelism, thread level parallelism, different levels of cache, and multi-core processors. A multi-core processor can include two or more processing units on a single computing component. Examples of multi-core processors include the AMD PHENOM IIX2, INTEL CORE i5 and INTEL CORE i7.

Main memory unit 722 can include one or more memory chips capable of storing data and allowing any storage location to be directly accessed by the microprocessor 721. Main memory unit 722 can be volatile and faster than storage 728 memory. Main memory units 722 can be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 722 or the storage 728 can be non-volatile, e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 722 can be based on any of the above-described memory chips, or any other available memory chips capable of operating as described herein. In the embodiment shown in FIG. 7A, the processor 721 communicates with main memory 722 via a system bus 750 (described in more detail below). FIG. 7B depicts an embodiment of a computing device 700 in which the processor communicates directly with main memory 722 via a memory port 703. For example, in FIG. 7B the main memory 722 can be DRDRAM.

FIG. 7B depicts an embodiment in which the main processor 721 communicates directly with cache memory 740 via a secondary bus, sometimes referred to as a backside bus. In other embodiments, the main processor 721 communicates with cache memory 740 using the system bus 750. Cache memory 740 typically has a faster response time than main memory 722 and is typically provided by SRAM, BSRAM, or EDRAM. In the embodiment shown in FIG. 7B, the processor 721 communicates with various I/O devices 730 via a local system bus 750. Various buses can be used to connect the central processing unit 721 to any of the I/O devices 730, including a PCI bus, a PCI-X bus, or a PCI-Express bus, or a NuBus. For embodiments in which the I/O device is a video display 724, the processor 721 can use an Advanced Graphics Port (AGP) to communicate with the display 724 or the I/O controller 723 for the display 724. FIG. 7B depicts an embodiment of a computer 700 in which the main processor 721 communicates directly with I/O device 730b or other processors 721′ via HYPERTRANSPORT, RAPIDIO, or INFINIBAND communications technology. FIG. 7B also depicts an embodiment in which local busses and direct communication are mixed: the processor 721 communicates with I/O device 730a using a local interconnect bus while communicating with I/O device 730b directly.

A wide variety of I/O devices 730a-730n can be present in the computing device 700. Input devices can include keyboards, mice, trackpads, trackballs, touchpads, touch mice, multi-touch touchpads and touch mice, microphones (analog or MEMS), multi-array microphones, drawing tablets, cameras, single-lens reflex camera (SLR), digital SLR (DSLR), CMOS sensors, CCDs, accelerometers, inertial measurement units, infrared optical sensors, pressure sensors, magnetometer sensors, angular rate sensors, depth sensors, proximity sensors, ambient light sensors, gyroscopic sensors, or other sensors. Output devices can include video displays, graphical displays, speakers, headphones, inkjet printers, laser printers, and 3D printers.

Devices 730a-730n can include a combination of multiple input or output devices, including, e.g., Microsoft KINECT, Nintendo Wiimote for the WII, Nintendo WII U GAMEPAD, or Apple IPHONE. Some devices 730a-730n allow gesture recognition inputs through combining some of the inputs and outputs. Some devices 730a-730n provides for facial recognition which can be utilized as an input for different purposes including authentication and other commands. Some devices 730a-730n provides for voice recognition and inputs, including, e.g., Microsoft KINECT, SIRI for IPHONE by Apple, Google Now or Google Voice Search.

Additional devices 730a-730n have both input and output capabilities, including, e.g., haptic feedback devices, touchscreen displays, or multi-touch displays. Touchscreen, multi-touch displays, touchpads, touch mice, or other touch sensing devices can use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices can allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, can have larger surfaces, such as on a table-top or on a wall, and can also interact with other electronic devices. Some I/O devices 730a-730n, display devices 724a-724n or group of devices can be augmented reality devices. The I/O devices can be controlled by an I/O controller 721 as shown in FIG. 7A. The I/O controller 721 can control one or more I/O devices, such as, e.g., a keyboard 126 and a pointing device 727, e.g., a mouse or optical pen. Furthermore, an I/O device can also provide storage and/or an installation medium 116 for the computing device 700. In still other embodiments, the computing device 700 can provide USB connections (not shown) to receive handheld USB storage devices. In further embodiments, an I/O device 730 can be a bridge between the system bus 750 and an external communication bus, e.g., a USB bus, a SCSI bus, a FireWire bus, an Ethernet bus, a Gigabit Ethernet bus, a Fibre Channel bus, or a Thunderbolt bus.

In some embodiments, display devices 724a-724n can be connected to I/O controller 721. Display devices can include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays can use, e.g., stereoscopy, polarization filters, active shutters, or autostereoscopy. Display devices 724a-724n can also be a head-mounted display (HMD). In some embodiments, display devices 724a-724n or the corresponding I/O controllers 723 can be controlled through or have hardware support for OPENGL or DIRECTX API or other graphics libraries.

In some embodiments, the computing device 700 can include or connect to multiple display devices 724a-724n, which each can be of the same or different type and/or form. As such, any of the I/O devices 730a-730n and/or the I/O controller 723 can include any type and/or form of suitable hardware, software, or combination of hardware and software to support, enable or provide for the connection and use of multiple display devices 724a-724n by the computing device 700. For example, the computing device 700 can include any type and/or form of video adapter, video card, driver, and/or library to interface, communicate, connect, or otherwise use the display devices 724a-724n. In one embodiment, a video adapter can include multiple connectors to interface to multiple display devices 724a-724n. In other embodiments, the computing device 700 can include multiple video adapters, with each video adapter connected to one or more of the display devices 724a-724n. In some embodiments, any portion of the operating system of the computing device 700 can be configured for using multiple displays 724a-724n. In other embodiments, one or more of the display devices 724a-724n can be provided by one or more other computing devices 700a or 700b connected to the computing device 700, via the network 140. In some embodiments, software can be designed and constructed to use another computer's display device as a second display device 724a for the computing device 700. For example, in one embodiment, an Apple iPad can connect to a computing device 700 and use the display of the device 700 as an additional display screen that can be used as an extended desktop.

Referring again to FIG. 7A, the computing device 700 can comprise a storage device 728 (e.g., one or more hard disk drives or redundant arrays of independent disks) for storing an operating system or other related software, and for storing application software programs such as any program related to the software for the NSS. Examples of storage device 728 include, e.g., hard disk drive (HDD); optical drive including CD drive, DVD drive, or BLU-RAY drive; solid-state drive (SSD); USB flash drive; or any other device suitable for storing data. Some storage devices can include multiple volatile and non-volatile memories, including, e.g., solid state hybrid drives that combine hard disks with solid state cache. Some storage devices 728 can be non-volatile, mutable, or read-only. Some storage devices 728 can be internal and connect to the computing device 700 via a bus 750. Some storage devices 728 can be external and connect to the computing device 700 via a I/O device 730 that provides an external bus. Some storage devices 728 can connect to the computing device 700 via the network interface 718 over a network, including, e.g., the Remote Disk for MACBOOK AIR by Apple. Some client devices 700 cannot require a non-volatile storage device 728 and can be thin clients or zero clients 202. Some storage devices 728 can also be used as an installation device 716 and can be suitable for installing software and programs. Additionally, the operating system and the software can be run from a bootable medium, for example, a bootable CD, e.g., KNOPPIX, a bootable CD for GNU/Linux that is available as a GNU/Linux distribution from knoppix.net.

Computing device 700 can also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc.

Furthermore, the computing device 700 can include a network interface 718 to interface to the network 140 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 700 communicates with other computing devices 700′ via any type and/or form of gateway or tunneling protocol e.g., Secure Socket Layer (SSL) or Transport Layer Security (TLS), or the Citrix Gateway Protocol manufactured by Citrix Systems, Inc. of Ft. Lauderdale, Florida. The network interface 118 can comprise a built-in network adapter, network interface card, PCMCIA network card, EXPRESSCARD network card, card bus network adapter, wireless network adapter, USB network adapter, modem, or any other device suitable for interfacing the computing device 700 to any type of network capable of communication and performing the operations described herein.

A computing device 700 of the sort depicted in FIG. 7A can operate under the control of an operating system, which controls scheduling of tasks and access to system resources. The computing device 700 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 7000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Washington; MAC OS and iOS, manufactured by Apple, Inc. of Cupertino, California; and Linux, a freely-available operating system, e.g., Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, can be used on zero clients or thin clients, including, e.g., CHROMEBOOKS.

The computer system 700 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 700 has sufficient processor power and memory capacity to perform the operations described herein. In some embodiments, the computing device 700 can have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 700 is a gaming system. For example, the computer system 700 can comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, or an XBOX 360 device manufactured by the Microsoft Corporation of Redmond, Washington, or an OCULUS RIFT or OCULUS VR device manufactured BY OCULUS VR, LLC of Menlo Park, California.

In some embodiments, the computing device 700 is a digital audio player such as the Apple IPOD, IPOD Touch, and IPOD NANO lines of devices, manufactured by Apple Computer of Cupertino, California. Some digital audio players can have other functionality, including, e.g., a gaming system or any functionality made available by an application from a digital application distribution platform. For example, the IPOD Touch can access the Apple App Store. In some embodiments, the computing device 700 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 700 is a tablet e.g., the IPAD line of devices by Apple; GALAXY TAB family of devices by Samsung; or KINDLE FIRE, by Amazon.com, Inc. of Seattle, Washington. In other embodiments, the computing device 700 is an eBook reader, e.g., the KINDLE family of devices by Amazon.com, or NOOK family of devices by Barnes & Noble, Inc. of New York City, New York.

In some embodiments, the communications device 700 includes a combination of devices, e.g., a smartphone combined with a digital audio player or portable media player. For example, one of these embodiments is a smartphone, e.g., the IPHONE family of smartphones manufactured by Apple, Inc.; a Samsung GALAXY family of smartphones manufactured by Samsung, Inc.; or a Motorola DROID family of smartphones. In yet another embodiment, the communications device 700 is a laptop or desktop computer equipped with a web browser and a microphone and speaker system, e.g., a telephony headset. In these embodiments, the communications devices 700 are web-enabled and can receive and initiate phone calls. In some embodiments, a laptop or desktop computer is also equipped with a webcam or other video capture device that enables video chat and video call.

In some embodiments, the status of one or more machines 700 in the network are monitored, generally as part of network management. In one of these embodiments, the status of a machine can include an identification of load information (e.g., the number of processes on the machine, CPU, and memory utilization), of port information (e.g., the number of available communication ports and the port addresses), or of session status (e.g., the duration and type of processes, and whether a process is active or idle). In another of these embodiments, this information can be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### A Method for Neural Stimulation

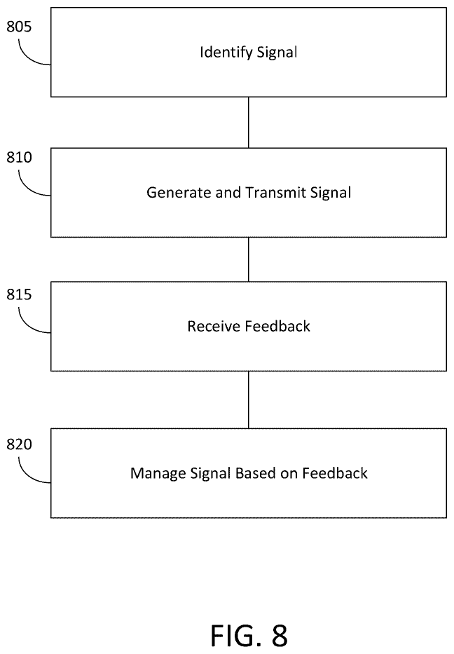

In FIG. 8 is a flow diagram of a method of performing visual stimulus induction of neural oscillations in accordance with an embodiment. The method 800 can be performed by one or more system, component, module, or element depicted in FIGS. 1-7B, including, for example, a neural stimulation system (NSS). In brief overview, the NSS can identify a visual signal to provide at block 805. At block 810, the NSS can generate and transmit the identified visual signal. At 815 the NSS can receive or determine feedback associated with neural activity, physiological activity, environmental parameters, or device parameters. At 820 the NSS can manage, control, or adjust the visual signal based on the feedback.

NSS Operating with A Frame

The NSS 105 can operate in conjunction with the frame 400 including a light source 305 as depicted in FIG. 4A. The NSS 105 can operate in conjunction with the frame 400 including a light source 30 and a feedback sensor 605 as depicted in FIG. 6A. The NSS 105 can operate in conjunction with the frame 400 including at least one shutter 430 as depicted in FIG. 4B. The NSS 105 can operate in conjunction with the frame 400 including at least one shutter 430 and a feedback sensor 605.

In operation, a user of the frame 400 can wear the frame 400 on their head such that eye wires 415 encircle or substantially encircle their eyes. In some cases, the user can provide an indication to the NSS 105 that the glass frames 400 have been worn and that the user is ready to undergo sensory induction of neural oscillations. The indication can include an instruction, command, selection, input, or other indication via an input/output interface, such as a keyboard 726, pointing device 727, or other I/O devices 730a-n. The indication can be a motion-based indication, visual indication, or voice-based indication. For example, the user can provide a voice command that indicates that the user is ready to undergo sensory induction of brainwave oscillation.

In some cases, the feedback sensor 605 can determine that the user is ready to undergo sensory induction of neural oscillations. The feedback sensor 605 can detect that the glass frames 400 have been placed on a user's head. The NSS 105 can receive motion data, acceleration data, gyroscope data, temperature data, or capacitive touch data to determine that the frames 400 have been placed on the user's head. The received data, such as motion data, can indicate that the frames 400 were picked up and placed on the user's head. The temperature data can measure the temperature of or proximate to the frames 400, which can indicate that the frames are on the user's head. In some cases, the feedback sensor 605 can perform eye tracking to determine a level of attention a user is paying to the light source 305 or feedback sensor 605. The NSS 105 can detect that the user is ready responsive to determining that the user is paying a high level of attention to the light source 305 or feedback sensor 605. For example, staring at, gazing, or looking in the direction of the light source 305 or feedback sensor 605 can provide an indication that the user is ready to undergo sensory induction of neural oscillations.

Thus, the NSS 105 can detect or determine that the frames 400 have been worn and that the user is in a ready state, or the NSS 105 can receive an indication or confirmation from the user that the user has worn the frames 400 and the user is ready to undergo sensory induction of neural oscillations. Upon determining that the user is ready, the NSS 105 can initialize the sensory induction of neural oscillations process. In some embodiments, the NSS 105 can access a profile data structure 145. For example, a profile manager 125 can query the profile data structure 145 to determine one or more parameter for the external visual stimulation used for the sensory induction of neural oscillations process. Parameters can include, for example, a type of visual stimulation, an intensity of the visual stimulation, frequency of the visual stimulation, duration of the visual stimulation, or wavelength of the visual stimulation. The profile manager 125 can query the profile data structure 145 to obtain historical sensory induction of neural oscillations information, such as prior visual stimulation sessions. The profile manager 125 can perform a lookup in the profile data structure 145. The profile manager 125 can perform a look-up with a username, user identifier, location information, fingerprint, biometric identifier, retina scan, voice recognition and authentication, or other identifying technique.

The NSS 105 can determine a type of external visual stimulation based on the hardware 400. The NSS 105 can determine the type of external visual stimulation based on the type of light source 305 available. For example, if the light source 305 includes a monochromatic LED that generates light waves in the red spectrum, the NSS 105 can determine that the type of visual stimulation includes pulses of light transmitted by the light source. However, if the frames 400 do not include an active light source 305, but, instead, include one or more shutters 430, the NSS 105 can determine that the light source is sunlight or ambient light that is to be modulated as it enters the user's eye via a plane formed by the eye wire 415.

In some embodiments, the NSS 105 can determine the type of external visual stimulation based on historical sensory induction of neural oscillations sessions. For example, the profile data structure 145 can be pre-configured with information about the type of visual signaling component 150.

The NSS 105 can determine, via the profile manager 125, a modulation frequency for the pulse train or the ambient light. For example, NSS 105 can determine, from the profile data structure 145, that the modulation frequency for the external visual stimulation may be set to 40 Hz. Depending on the type of visual stimulation, the profile data structure 145 can further indicate a pulse length, intensity, wavelength of the light wave forming the light pulse, or duration of the pulse train.

In some cases, the NSS 105 can determine or adjust one or more parameter of the external visual stimulation. For example, the NSS 105 (e.g., via feedback component 160 or feedback sensor 605) can determine a level or amount of ambient light. The NSS 105 (e.g., via light adjustment module 115 or side effects management module 130) can establish, initialize, set, or adjust the intensity or wavelength of the light pulse. For example, the NSS 105 can determine that there is a low level of ambient light. Due to the low level of ambient light, the user's pupils may be dilated. The NSS 105 can determine, based on detecting a low level of ambient light, that the user's pupils are likely dilated. In response to determining that the user's pupils are likely dilated, the NSS 105 can set a low level of intensity for the pulse train. The NSS 105 can further use a light wave having a longer wavelength (e.g., red), which may reduce strain on the eyes.

In some embodiments, the NSS 105 can monitor (e.g., via feedback monitor 135 and feedback component 160) the level of ambient light throughout the sensory induction of neural oscillations process to automatically and periodically adjust the intensity or color of light pulses. For example, if the user began the sensory induction of neural oscillations process when there was a high level of ambient light, the NSS 105 can initially set a higher intensity level for the light pulses and use a color that includes light waves having lower wavelengths (e.g., blue). However, in some embodiments in which the ambient light level decreases throughout the sensory induction of neural oscillations process, the NSS 105 can automatically detect the decrease in ambient light and, in response to the detection, adjust or lower the intensity while increasing the wavelength of the light wave. The NSS 105 can adjust the light pulses to provide a high contrast ratio to facilitate induction of neural oscillations.

In some embodiments, the NSS 105 (e.g., via feedback monitor 135 and feedback component 160) can monitor or measure physiological conditions to set or adjust a parameter of the light wave. For example, the NSS 105 can monitor or measure a level of pupil dilation to adjust or set a parameter of the light wave. In some embodiments, the NSS 105 can monitor or measure heart rate, pulse rate, blood pressure, body temperature, perspiration, or brain activity to set or adjust a parameter of the light wave.

In some embodiments, the NSS 105 can be preconfigured to initially transmit light pulses having a lowest setting for light wave intensity (e.g., low amplitude of the light wave or high wavelength of the light wave) and gradually increase the intensity (e.g., increase the amplitude of the light wave or decrease the wavelength of the light wave) while monitoring feedback until an optimal light intensity is reached. An optimal light intensity can refer to a highest intensity without adverse physiological side effects, such as blindness, seizures, heart attack, migraines, or other discomfort. The NSS 105 (e.g., via side effects management module 130) can monitor the physiological symptoms to identify the adverse side effects of the external visual stimulation, and adjust (e.g., via light adjustment module 115) the external visual stimulation accordingly to reduce or eliminate the adverse side effects.

In some embodiments, the NSS 105 (e.g., via light adjustment module 115) can adjust a parameter of the light wave or light pulse based on a level of attention. For example, during the sensory induction of neural oscillations process, the user may get bored, lose focus, fall asleep, or otherwise not pay attention to the light pulses. Not paying attention to the light pulses may reduce the efficacy of the sensory induction of neural oscillations process, resulting in neurons oscillating at a frequency different from the desired modulation frequency of the light pulses.

NSS 105 can detect the level of attention the user is paying to the light pulses using the feedback monitor 135 and one or more feedback component 160. The NSS 105 can perform eye tracking to determine the level of attention the user is providing to the light pulses based on the gaze direction of the retina or pupil. The NSS 105 can measure eye movement to determine the level of attention the user is paying to the light pulses. The NSS 105 can provide a survey or prompt asking for user feedback that indicates the level of attention the user is paying to the light pulses. Responsive to determining that the user is not paying a satisfactory amount of attention to the light pulses (e.g., a level of eye movement that is greater than a threshold or a gaze direction that is outside the direct visual field of the light source 305), the light adjustment module 115 can change a parameter of the light source to gain the user's attention. For example, the light adjustment module 115 can increase the intensity of the light pulse, adjust the color of the light pulse, or change the duration of the light pulse. The light adjustment module 115 can randomly vary one or more parameters of the light pulse. The light adjustment module 115 can initiate an attention seeking light sequence configured to regain the user's attention. For example, the light sequence can include a change in color or intensity of the light pulses in a predetermined, random, or pseudo-random pattern. The attention seeking light sequence can enable or disable different light sources if the visual signaling component 150 includes multiple light sources. Thus, the light adjustment module 115 can interact with the feedback monitor 135 to determine a level of attention the user is providing to the light pulses and adjust the light pulses to regain the user's attention if the level of attention falls below a threshold.

In some embodiments, the light adjustment module 115 can change or adjust one or more parameter of the light pulse or light wave at predetermined time intervals (e.g., every 5 minutes, 10 minutes, 15 minutes, or 20 minutes) to regain or maintain the user's attention level.

In some embodiments, the NSS 105 (e.g., via unwanted frequency filtering module 120) can filter, block, attenuate, or remove unwanted visual external stimulation. Unwanted visual external stimulation can include, for example, unwanted modulation frequencies, unwanted intensities, or unwanted wavelengths of light waves. The NSS 105 can deem a modulation frequency to be unwanted if the modulation frequency of a pulse train is different or substantially different (e.g., 1%, 2%, 5%, 10%, 15%, 20%, 25%, or more than 25%) from a desired frequency.

For example, the desired modulation frequency for sensory induction of neural oscillations can be 40 Hz. However, for example, a modulation frequency of 15 Hz or 90 Hz can hinder sensory induction of neural oscillations. Thus, the NSS 105 can filter out the light pulses or light waves corresponding to the 15 Hz or 90 Hz modulation frequency.

In some embodiments, the NSS 105 can detect, via feedback component 160, that there are light pulses from an ambient light source that corresponds to an unwanted modulation frequency of 20 Hz. The NSS 105 can further determine the wavelength of the light waves of the light pulses corresponding to the unwanted modulation frequency. The NSS 105 can instruct the filtering component 155 to filter out the wavelength corresponding to the unwanted modulation frequency. For example, the wavelength corresponding to the unwanted modulation frequency can correspond to the color blue. The filtering component 155 can include an optical filter that can selectively transmit light in a particular range of wavelengths or colors, while blocking one or more other ranges of wavelengths or colors. The optical filter can modify the magnitude or phase of the incoming light wave for a range of wavelengths. For example, the optical filter can be configured to block, reflect, or attenuate the blue light wave corresponding to the unwanted modulation frequency. The light adjustment module 115 can change the wavelength of the light wave generated by the light generation module 110 and light source 305 such that the desired modulation frequency is not blocked or attenuated by the unwanted frequency filtering module 120.

NSS Operating with a Virtual Reality Headset

The NSS 105 can operate in conjunction with the virtual reality headset 401 including a light source 305 as depicted in FIG. 4C. The NSS 105 can operate in conjunction with the virtual reality headset 401 including a light source 305 and a feedback sensor 605 as depicted in FIG. 4C. In some embodiments, the NSS 105 can determine that the visual signaling component 150 hardware includes a virtual reality headset 401. Responsive to determining that the visual signaling component 150 includes a virtual reality headset 401, the NSS 105 can determine that the light source 305 includes a display screen of a smartphone or other mobile computing device.

The virtual reality headset 401 can provide an immersive, non-disruptive visual stimulation experience. The virtual reality headset 401 can provide an augmented reality experience. The feedback sensors 605 can capture pictures or video of the physical, real world to provide the augmented reality experience. The unwanted frequency filtering module 120 can filter out unwanted modulation frequencies prior to projecting, displaying, or providing the augmented reality images via the display screen 305.

In operation, a user of the frame 401 can wear the frame 401 on their head such that the virtual reality headset eye sockets 465 cover the user's eyes. The virtual reality headset eye sockets 465 can encircle or substantially encircle their eyes. The user can secure the virtual reality headset 401 to the user's headset using one or more straps 455 or 460, a skull cap, or other fastening mechanism. In some cases, the user can provide an indication to the NSS 105 that the virtual reality headset 401 has been placed and secured to the user's head and that the user is ready to undergo sensory induction of neural oscillations. The indication can include an instruction, command, selection, input, or other indication via an input/output interface, such as a keyboard 726, pointing device 727, or other I/O devices 730a-n. The indication can be a motion-based indication, visual indication, or voice-based indication. For example, the user can provide a voice command that indicates that the user is ready to undergo sensory induction of neural oscillations.

In some cases, the feedback sensor 605 can determine that the user is ready to undergo sensory induction of neural oscillations. The feedback sensor 605 can detect that the virtual reality headset 401 has been placed on a user's head. The NSS 105 can receive motion data, acceleration data, gyroscope data, temperature data, or capacitive touch data to determine that the virtual reality headset 401 has been placed on the user's head. The received data, such as motion data, can indicate that the virtual reality headset 401 was picked up and placed on the user's head. The temperature data can measure the temperature of or proximate to the virtual reality headset 401, which can indicate that the virtual reality headset 401 is on the user's head. In some cases, the feedback sensor 605 can perform eye tracking to determine a level of attention a user is paying to the light source 305 or feedback sensor 605. The NSS 105 can detect that the user is ready responsive to determining that the user is paying a high level of attention to the light source 305 or feedback sensor 605. For example, staring at, gazing, or looking in the direction of the light source 305 or feedback sensor 605 can provide an indication that the user is ready to undergo sensory induction of neural oscillations.

In some embodiments, a sensor 605 on the straps 455, straps 460 or eye socket 605 can detect that the virtual reality headset 401 is secured, placed, or positioned on the user's head. The sensor 605 can be a touch sensor that senses or detects the touch of the user's head.

Thus, the NSS 105 can detect or determine that the virtual reality headset 401 has been worn and that the user is in a ready state, or the NSS 105 can receive an indication or confirmation from the user that the user has worn the virtual reality headset 401 and the user is ready to undergo sensory induction of neural oscillations. Upon determining that the user is ready, the NSS 105 can initialize the sensory induction of neural oscillations process. In some embodiments, the NSS 105 can access a profile data structure 145. For example, a profile manager 125 can query the profile data structure 145 to determine one or more parameter for the external visual stimulation used for the sensory induction of neural oscillations process. Parameters can include, for example, a type of visual stimulation, an intensity of the visual stimulation, frequency of the visual stimulation, duration of the visual stimulation, or wavelength of the visual stimulation. The profile manager 125 can query the profile data structure 145 to obtain historical sensory induced neural oscillations information, such as prior visual stimulation sessions. The profile manager 125 can perform a lookup in the profile data structure 145. The profile manager 125 can perform a look-up with a username, user identifier, location information, fingerprint, biometric identifier, retina scan, voice recognition and authentication, or other identifying technique.

The NSS 105 can determine a type of external visual stimulation based on the hardware 401. The NSS 105 can determine the type of external visual stimulation based on the type of light source 305 available. For example, if the light source 305 includes a smartphone or display device, the visual stimulation can include turning on and off the display screen of the display device. The visual stimulation can include displaying a pattern on the display device 305, such as a checkered pattern, that can alternate in accordance with the desired frequency modulation. The visual stimulation can include light pulses generated by a light source 305 such as an LED that is placed within the virtual reality headset 401 enclosure.

In cases where the virtual reality headset 401 provides an augmented reality experience, the visual stimulation can include overlaying content on the display device and modulating the overlaid content at the desired modulation frequency. For example, the virtual reality headset 401 can include a camera 605 that captures the real, physical world. While displaying the captured image of the real, physical world, the NSS 105 can also display content that is modulated at the desired modulation frequency. The NSS 105 can overlay the content modulated at the desired modulation frequency. The NSS 105 can otherwise modify, manipulate, modulation, or adjust a portion of the display screen or a portion of the augmented reality to generate or provide the desired modulation frequency.

For example, the NSS 105 can modulate one or more pixels based on the desired modulation frequency. The NSS 105 can turn pixels on and off based on the modulation frequency. The NSS 105 can turn of pixels on any portion of the display device. The NSS 105 can turn on and off pixels in a pattern. The NSS 105 can turn on and off pixels in the direct visual field or peripheral visual field. The NSS 105 can track or detect a gaze direction of the eye and turn on and off pixels in the gaze direction, so the light pulses (or modulation) are in the direct vision field. Thus, modulating the overlaid content or otherwise manipulated the augmented reality display or other image provided via a display device in the virtual reality headset 401 can generate light pulses or light flashes having a modulation frequency configured to facilitate sensory induction of neural oscillations.

The NSS 105 can determine, via the profile manager 125, a modulation frequency for the pulse train or the ambient light. For example, NSS 105 can determine, from the profile data structure 145, that the modulation frequency for the external visual stimulation may be set to 40 Hz. Depending on the type of visual stimulation, the profile data structure 145 can further indicate a number of pixels to modulate, intensity of pixels to modulate, pulse length, intensity, wavelength of the light wave forming the light pulse, or duration of the pulse train.

In some cases, the NSS 105 can determine or adjust one or more parameter of the external visual stimulation. For example, the NSS 105 (e.g., via feedback component 160 or feedback sensor 605) can determine a level or amount of light in captured image used to provide the augmented reality experience. The NSS 105 (e.g., via light adjustment module 115 or side effects management module 130) can establish, initialize, set, or adjust the intensity or wavelength of the light pulse based on the light level in the image data corresponding to the augmented reality experience. For example, the NSS 105 can determine that there is a low level of light in the augmented reality display because it may be dark outside. Due to the low level of light in the augmented reality display, the user's pupils may be dilated. The NSS 105 can determine, based on detecting a low level of light, that the user's pupils are likely dilated. In response to determining that the user's pupils are likely dilated, the NSS 105 can set a low level of intensity for the light pulses or light source providing the modulation frequency. The NSS 105 can further use a light wave having a longer wavelength (e.g., red), which may reduce strain on the eyes.

In some embodiments, the NSS 105 can monitor (e.g., via feedback monitor 135 and feedback component 160) the level of light throughout the sensory induction of neural oscillations process to automatically and periodically adjust the intensity or color of light pulses. For example, if the user began the sensory induction of neural oscillations process when there was a high level of ambient light, the NSS 105 can initially set a higher intensity level for the light pulses and use a color that includes light waves having lower wavelengths (e.g., blue). However, as the light level decreases throughout the sensory induction of neural oscillations process, the NSS 105 can automatically detect the decrease in light and, in response to the detection, adjust or lower the intensity while increasing the wavelength of the light wave. The NSS 105 can adjust the light pulses to provide a high contrast ratio to facilitate sensory induction of neural oscillations.

In some embodiments, the NSS 105 (e.g., via feedback monitor 135 and feedback component 160) can monitor or measure physiological conditions to set or adjust a parameter of the light pulses while the user is wearing the virtual reality headset 401. For example, the NSS 105 can monitor or measure a level of pupil dilation to adjust or set a parameter of the light wave. In some embodiments, the NSS 105 can monitor or measure, via one or more feedback sensor of the virtual reality headset 401 or other feedback sensor, a heart rate, pulse rate, blood pressure, body temperature, perspiration, or brain activity to set or adjust a parameter of the light wave.

In some embodiments, the NSS 105 can be preconfigured to initially transmit, via display device 305, light pulses having a lowest setting for light wave intensity (e.g., low amplitude of the light wave or high wavelength of the light wave) and gradually increase the intensity (e.g., increase the amplitude of the light wave or decrease the wavelength of the light wave) while monitoring feedback until an optimal light intensity is reached. An optimal light intensity can refer to a highest intensity without adverse physiological side effects, such as blindness, seizures, heart attack, migraines, or other discomfort. The NSS 105 (e.g., via side effects management module 130) can monitor the physiological symptoms to identify the adverse side effects of the external visual stimulation, and adjust (e.g., via light adjustment module 115) the external visual stimulation accordingly to reduce or eliminate the adverse side effects.

In some embodiments, the NSS 105 (e.g., via light adjustment module 115) can adjust a parameter of the light wave or light pulse based on a level of attention. For example, during the sensory induction of neural oscillations process, the user may get bored, lose focus, fall asleep, or otherwise not pay attention to the light pulses generated via the display screen 305 of the virtual reality headset 401. Not paying attention to the light pulses may reduce the efficacy of the sensory induction of neural oscillations process, resulting in neurons oscillating at a frequency different from the desired modulation frequency of the light pulses.

NSS 105 can detect the level of attention the user is paying or providing to the light pulses using the feedback monitor 135 and one or more feedback component 160 (e.g., including feedback sensors 605). The NSS 105 can perform eye tracking to determine the level of attention the user is providing to the light pulses based on the gaze direction of the retina or pupil. The NSS 105 can measure eye movement to determine the level of attention the user is paying to the light pulses. The NSS 105 can provide a survey or prompt asking for user feedback that indicates the level of attention the user is paying to the light pulses. Responsive to determining that the user is not paying a satisfactory amount of attention to the light pulses (e.g., a level of eye movement that is greater than a threshold or a gaze direction that is outside the direct visual field of the light source 305), the light adjustment module 115 can change a parameter of the light source 305 or display device 305 to gain the user's attention. For example, the light adjustment module 115 can increase the intensity of the light pulse, adjust the color of the light pulse, or change the duration of the light pulse. The light adjustment module 115 can randomly vary one or more parameters of the light pulse. The light adjustment module 115 can initiate an attention seeking light sequence configured to regain the user's attention. For example, the light sequence can include a change in color or intensity of the light pulses in a predetermined, random, or pseudo-random pattern. The attention seeking light sequence can enable or disable different light sources if the visual signaling component 150 includes multiple light sources. Thus, the light adjustment module 115 can interact with the feedback monitor 135 to determine a level of attention the user is providing to the light pulses and adjust the light pulses to regain the user's attention if the level of attention falls below a threshold.

In some embodiments, the light adjustment module 115 can change or adjust one or more parameter of the light pulse or light wave at predetermined time intervals (e.g., every 5 minutes, 10 minutes, 15 minutes, or 20 minutes) to regain or maintain the user's attention level.

In some embodiments, the NSS 105 (e.g., via unwanted frequency filtering module 120) can filter, block, attenuate, or remove unwanted visual external stimulation. Unwanted visual external stimulation can include, for example, unwanted modulation frequencies, unwanted intensities, or unwanted wavelengths of light waves. The NSS 105 can deem a modulation frequency to be unwanted if the modulation frequency of a pulse train is different or substantially different (e.g., 1%, 2%, 5%, 10%, 15%, 20%, 25%, or more than 25%) from a desired frequency.

For example, the desired modulation frequency for sensory induction of neural oscillations can be 40 Hz. However, for example, a modulation frequency of 15 Hz or 90 Hz can hinder sensory induction of neural oscillations. Thus, the NSS 105 can filter out the light pulses or light waves corresponding to the 15 Hz or 90 Hz modulation frequency. For example, the virtual reality headset 401 can detect unwanted modulation frequencies in the physical, real world and eliminate, attenuate, filter out or otherwise remove the unwanted frequencies providing to generating the or providing the augmented reality experience. The NSS 105 can include an optical filter configured to perform digital signal processing or digital image processing to detect the unwanted modulation frequency in the real world captured by the feedback sensor 605. The NSS 105 can detect other content, image or motion having an unwanted parameter (e.g., color, brightness, contrast ratio, modulation frequency), and eliminate same from the augmented reality experience projected to the user via the display screen 305. The NSS 105 can apply a color filter to adjust the color or remove a color of the augmented reality display. The NSS 105 can adjust, modify, or manipulate the brightness, contrast ratio, sharpness, tint, hue, or other parameter of the image or video displayed via the display device 305.

In some embodiments, the NSS 105 can detect, via feedback component 160, that there is captured image or video content from the real, physical world that corresponds to an unwanted modulation frequency of 20 Hz. The NSS 105 can further determine the wavelength of the light waves of the light pulses corresponding to the unwanted modulation frequency. The NSS 105 can instruct the filtering component 155 to filter out the wavelength corresponding to the unwanted modulation frequency. For example, the wavelength corresponding to the unwanted modulation frequency can correspond to the color blue. The filtering component 155 can include a digital optical filter that can digitally remove content or light in a particular range of wavelengths or colors, while allowing one or more other ranges of wavelengths or colors. The digital optical filter can modify the magnitude or phase of the image for a range of wavelengths. For example, the digital optical filter can be configured to attenuate, erase, replace or otherwise alter the blue light wave corresponding to the unwanted modulation frequency. The light adjustment module 115 can change the wavelength of the light wave generated by the light generation module 110 and display device 305 such that the desired modulation frequency is not blocked or attenuated by the unwanted frequency filtering module 120.

NSS Operating with a Tablet

The NSS 105 can operate in conjunction with the tablet 500 as depicted in FIGS. 5A-5D. In some embodiments, the NSS 105 can determine that the visual signaling component 150 hardware includes a tablet device 500 or other display screen that is not affixed or secured to a user's head. The tablet 500 can include a display screen that has one or more component or function of the display screen 305 or light source 305 depicted in conjunction with FIGS. 4A and 4C. The light source 305 in a tablet can be the display screen. The tablet 500 can include one or more feedback sensor that includes one or more component or function of the feedback sensor depicted in conjunction with FIGS. 4B, 4C and 6A.

The tablet 500 can communicate with the NSS 105 via a network, such as a wireless network or a cellular network. The NSS 105 can, in some embodiments, execute the NSS 105 or a component thereof. For example, the tablet 500 can launch, open or switch to an application or resource configured to provide at least one functionality of the NSS 105. The tablet 500 can execute the application as a background process or a foreground process. For example, the graphical user interface for the application can be in the background while the application causes the display screen 305 of the tablet to overlay content or light that changes or modulates at a desired frequency for sensory induction of neural oscillations (e.g., 40 Hz).

The tablet 500 can include one or more feedback sensors 605. In some embodiments, the tablet can use the one or more feedback sensors 605 to detect that a user is holding the tablet 500. The tablet can use the one or more feedback sensors 605 to determine a distance between the light source 305 and the user. The tablet can use the one or more feedback sensors 605 to determine a distance between the light source 305 and the user's head. The tablet can use the one or more feedback sensors 605 to determine a distance between the light source 305 and the user's eyes.

In some embodiments, the tablet 500 can use a feedback sensor 605 that includes a receiver to determine the distance. The tablet can transmit a signal and measure the amount of time it takes for the transmitted signal to leave the tablet 500, bounce on the object (e.g., user's head) and be received by the feedback sensor 605. The tablet 500 or NSS 105 can determine the distance based on the measured amount of time and the speed of the transmitted signal (e.g., speed of light).

In some embodiments, the tablet 500 can include two feedback sensors 605 to determine a distance. The two feedback sensors 605 can include a first feedback sensor 605 that is the transmitter and a second feedback sensor that is the receiver.

In some embodiments, the tablet 500 can include two or more feedback sensors 605 that include two or more cameras. The two or more cameras can measure the angles and the position of the object (e.g., the user's head) on each camera, and use the measured angles and position to determine or compute the distance between the tablet 500 and the object.

In some embodiments, the tablet 500 (or application thereof) can determine the distance between the tablet and the user's head by receiving user input. For example, user input can include an approximate size of the user's head. The tablet 500 can then determine the distance from the user's head based on the inputted approximate size.

The tablet 500, application, or NSS 105 can use the measured or determined distance to adjust the light pulses or flashes of light emitted by the light source 305 of the tablet 500. The tablet 500, application, or NSS 105 can use the distance to adjust one or more parameter of the light pulses, flashes of light or other content emitted via the light source 305 of the tablet 500. For example, the tablet 500 can adjust the intensity of the light pulses emitted by light source 305 based on the distance. The tablet 500 can adjust the intensity based on the distance in order to maintain a consistent or similar intensity at the eye irrespective of the distance between the light source 305 and the eye. The tablet can increase the intensity proportional to the square of the distance.

The tablet 500 can manipulate one or more pixels on the display screen 305 to generate the light pulses or modulation frequency for sensory induction of neural oscillations. The tablet 500 can overlay light sources, light pulses, or other patterns to generate the modulation frequency for sensory induction of neural oscillations. Similar to the virtual reality headset 401, the tablet can filter out or modify unwanted frequencies, wavelengths, or intensity.

Similar to the frames 400, the tablet 500 can adjust a parameter of the light pulses or flashes of light generated by the light source 305 based on ambient light, environmental parameters, or feedback.

In some embodiments, the tablet 500 can execute an application that is configured to generate the light pulses or modulation frequency for sensory induction of neural oscillations.

The application can execute in the background of the tablet such that all content displayed on a display screen of the tablet are displayed as light pulses at the desired frequency. The tablet can be configured to detect a gaze direction of the user. In some embodiments, the tablet may detect the gaze direction by capturing an image of the user's eye via the camera of the tablet. The tablet 500 can be configured to generate light pulses at particular locations of the display screen based on the gaze direction of the user. In embodiments where direct vision field is to be employed, the light pulses can be displayed at locations of the display screen that correspond to the user's gaze. In embodiments where peripheral vision field is to be employed, the light pulses can be displayed at locations of the displays screen that are outside the portion of the display screen corresponding to the user's gaze.

### Neural Stimulation Via Auditory Stimulation

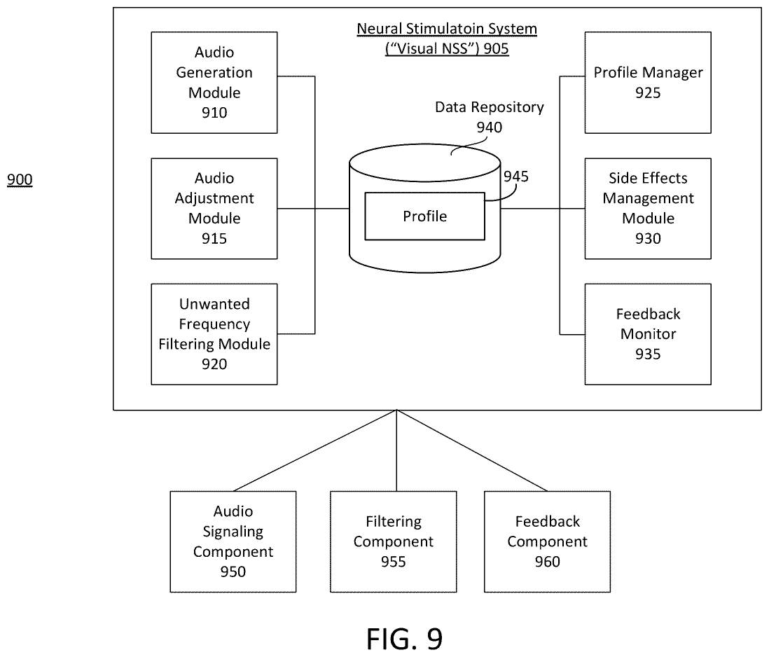

FIG. 9 is a block diagram depicting a system for neural stimulation via auditory stimulation in accordance with an embodiment. The system 900 can include a neural stimulation system (“NSS”) 905. The NSS 905 can be referred to as an auditory NSS 905 or NSS 905. In brief overview, the auditory neural stimulation system (“NSS”) 905 can include, access, interface with, or otherwise communicate with one or more of an audio generation module 910, audio adjustment module 915, unwanted frequency filtering module 920, profile manager 925, side effects management module 930, feedback monitor 935, data repository 940, audio signaling component 950, filtering component 955, or feedback component 960. The audio generation module 910, audio adjustment module 915, unwanted frequency filtering module 920, profile manager 925, side effects management module 930, feedback monitor 935, audio signaling component 950, filtering component 955, or feedback component 960 can each include at least one processing unit or other logic device such as programmable logic array engine, or module configured to communicate with the database repository 950. The audio generation module 910, audio adjustment module 915, unwanted frequency filtering module 920, profile manager 925, side effects management module 930, feedback monitor 935, audio signaling component 950, filtering component 955, or feedback component 960 can be separate components, a single component, or part of the NSS 905. The system 100 and its components, such as the NSS 905, may include hardware elements, such as one or more processors, logic devices, or circuits. The system 100 and its components, such as the NSS 905, can include one or more hardware or interface component depicted in system 700 in FIGS. 7A and 7B. For example, a component of system 100 can include or execute on one or more processors 721, access storage 728 or memory 722, and communicate via network interface 718.

Still referring to FIG. 9, and in further detail, the NSS 905 can include at least one audio generation module 910. The audio generation module 910 can be designed and constructed to interface with an audio signaling component 950 to provide instructions or otherwise cause or facilitate the generation of an audio signal, such as an audio burst, audio pulse, audio chirp, audio sweep, or other acoustic wave having one or more predetermined parameters. The audio generation module 910 can include hardware or software to receive and process instructions or data packets from one or more module or component of the NSS 905. The audio generation module 910 can generate instructions to cause the audio signaling component 950 to generate an audio signal. The audio generation module 910 can control or enable the audio signaling component 950 to generate the audio signal having one or more predetermined parameters.

The audio generation module 910 can be communicatively coupled to the audio signaling component 950. The audio generation module 910 can communicate with the audio signaling component 950 via a circuit, electrical wire, data port, network port, power wire, ground, electrical contacts, or pins. The audio generation module 910 can wirelessly communicate with the audio signaling component 950 using one or more wireless protocols such as BlueTooth, BlueTooth Low Energy, Zigbee, Z-Wave, IEEE 802, WIFI, 3G, 4G, LTE, near field communications (“NFC”), or other short, medium or long-range communication protocols, etc. The audio generation module 910 can include or access network interface 718 to communicate wirelessly or over a wire with the audio signaling component 950.

The audio generation module 910 can interface, control, or otherwise manage various types of audio signaling components 950 in order to cause the audio signaling component 950 to generate, block, control, or otherwise provide the audio signal having one or more predetermined parameters. The audio generation module 910 can include a driver configured to drive an audio source of the audio signaling component 950. For example, the audio source can include a speaker, and the audio generation module 910 (or the audio signaling component) can include a transducer that converts electrical energy to sound waves or acoustic waves. The audio generation module 910 can include a computing chip, microchip, circuit, microcontroller, operational amplifiers, transistors, resistors, or diodes configured to provide electricity or power having certain voltage and current characteristics to drive the speaker to generate an audio signal with desired acoustic characteristics.

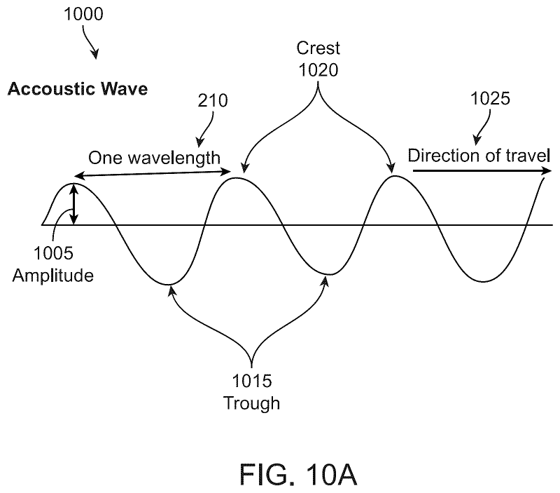

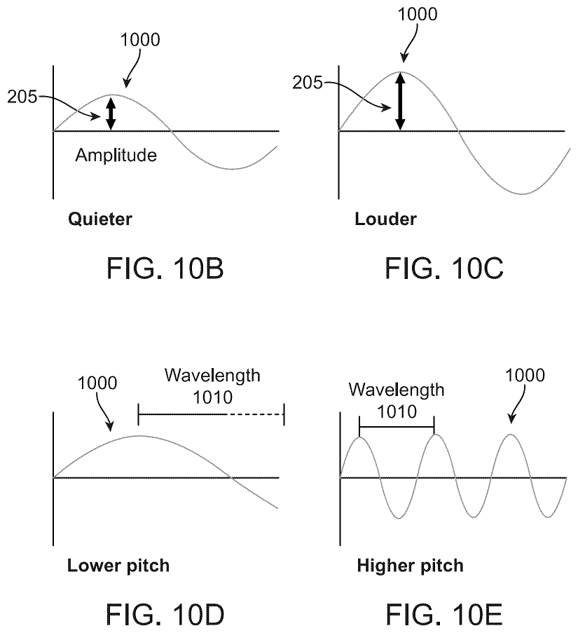

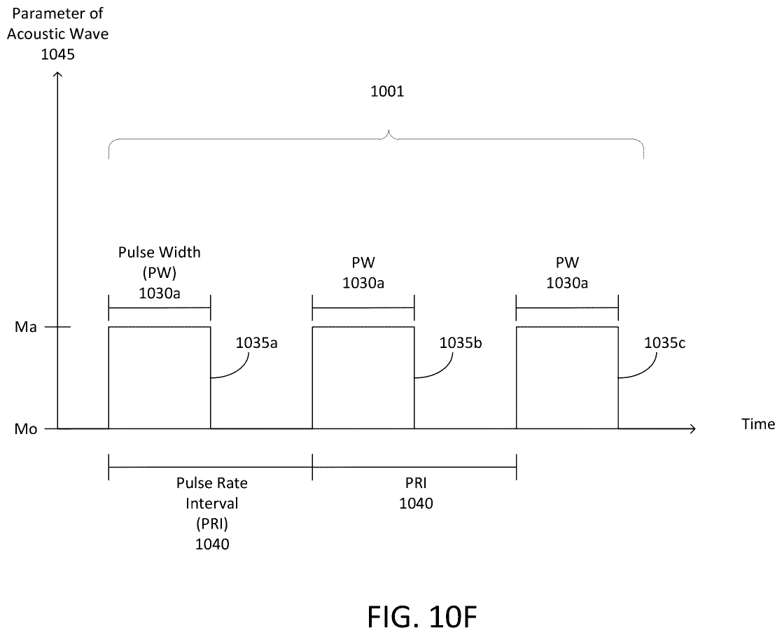

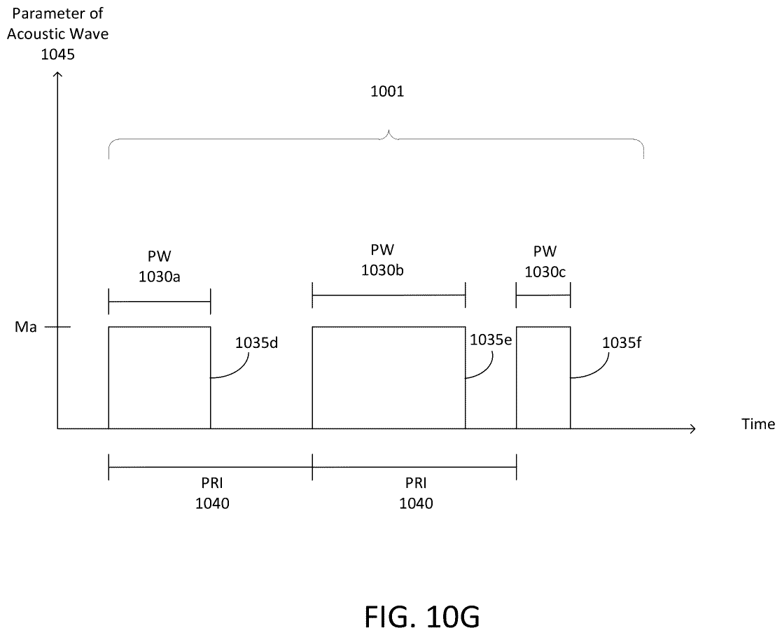

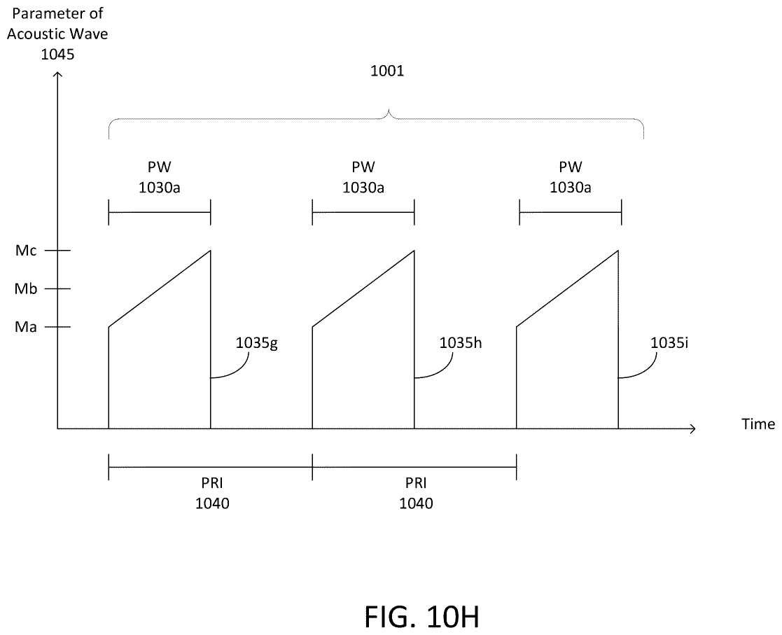

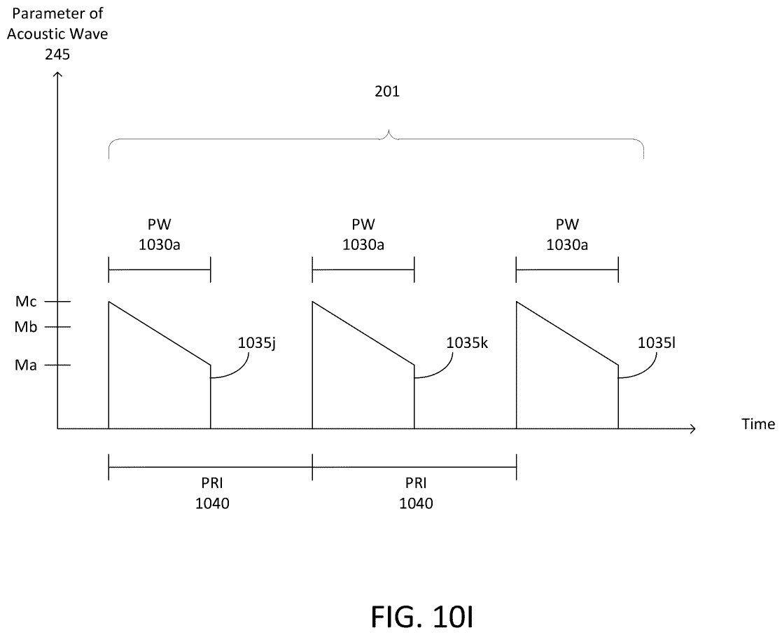

In some embodiments, the audio generation module 910 can instruct the audio signaling component 950 to provide an audio signal. For example, the audio signal can include an acoustic wave 1000 as depicted in FIG. 10A. The audio signal can include multiple acoustic waves. The audio signal can generate one or more acoustic waves. The acoustic wave 1000 can include or be formed of a mechanical wave of pressure and displacement that travels through media such as gases, liquids, and solids. The acoustic wave can travel through a medium to cause vibration, sound, ultrasound, or infrasound. The acoustic wave can propagate through air, water, or solids as longitudinal waves. The acoustic wave can propagate through solids as a transverse wave.

The acoustic wave can generate sound due to the oscillation in pressure, stress, particle displacement, or particle velocity propagated in a medium with internal forces (e.g., elastic or viscous), or the superposition of such propagated oscillation. Sound can refer to the auditory sensation evoked by this oscillation. For example, sound can refer to the reception of acoustic waves and their perception by the brain.

The audio signaling component 950 or audio source thereof can generate the acoustic waves by vibrating a diaphragm of the audio source. For example, the audio source can include a diaphragm such as a transducer configured to inter-convert mechanical vibrations to sounds. The diaphragm can include a thin membrane or sheet of various materials, suspended at its edges. The varying pressure of sound waves imparts mechanical vibrations to the diaphragm which can then create acoustic waves or sound.

The acoustic wave 1000 illustrated in FIG. 10A includes a wavelength 1010. The wavelength 1010 can refer to a distance between successive crests 1020 of the wave. The wavelength 1010 can be related to the frequency of the acoustic wave and the speed of the acoustic wave. For example, the wavelength can be determined as the quotient of the speed of the acoustic wave divided by the frequency of the acoustic wave. The speed of the acoustic wave can the product of the frequency and the wavelength. The frequency of the acoustic wave can be the quotient of the speed of the acoustic wave divided by the wavelength of the acoustic wave. Thus, the frequency and the wavelength of the acoustic wave can be inversely proportional. The speed of sound can vary based on the medium through which the acoustic wave propagates. For example, the speed of sound in air can be 343 meters per second.

A crest 1020 can refer to the top of the wave or point on the wave with the maximum value. The displacement of the medium is at a maximum at the crest 1020 of the wave. The trough 1015 is the opposite of the crest 1020. The trough 1015 is the minimum or lowest point on the wave corresponding to the minimum amount of displacement.

The acoustic wave 1000 can include an amplitude 1005. The amplitude 1005 can refer to a maximum extent of a vibration or oscillation of the acoustic wave 1000 measured from a position of equilibrium. The acoustic wave 1000 can be a longitudinal wave if it oscillates or vibrates in the same direction of travel 1025. In some cases, the acoustic wave 1000 can be a transverse wave that vibrates at right angles to the direction of its propagation.

The audio generation module 910 can instruct the audio signaling component 950 to generate acoustic waves or sound waves having one or more predetermined amplitude or wavelength. Wavelengths of the acoustic wave that are audible to the human ear range from approximately 17 meters to 17 millimeters (or 20 Hz to 20 kHz). The audio generation module 910 can further specify one or more properties of an acoustic wave within or outside the audible spectrum. For example, the frequency of the acoustic wave can range from 0 to 50 kHz. In some embodiments, the frequency of the acoustic wave can range from 8 to 12 kHz. In some embodiments, the frequency of the acoustic wave can be 10 kHz.

The NSS 905 can modulate, modify, change, or otherwise alter properties of the acoustic wave 1000. For example, the NSS 905 can modulate the amplitude or wavelength of the acoustic wave. As depicted in FIG. 10B and FIG. 10C, the NSS 905 can adjust, manipulate, or otherwise modify the amplitude 1005 of the acoustic wave 1000. For example, the NSS 905 can lower the amplitude 1005 to cause the sound to be quieter, as depicted in FIG. 10B, or increase the amplitude 1005 to cause the sound to be louder, as depicted in FIG. 10C.

In some cases, the NSS 905 can adjust, manipulate, or otherwise modify the wavelength 1010 of the acoustic wave. As depicted in FIG. 10D and FIG. 10E, the NSS 905 can adjust, manipulate, or otherwise modify the wavelength 1010 of the acoustic wave 1000. For example, the NSS 905 can increase the wavelength 1010 to cause the sound to have a lower pitch, as depicted in FIG. 10D, or reduce the wavelength 1010 to cause the sound to have a higher pitch, as depicted in FIG. 10E.

The NSS 905 can modulate the acoustic wave. Modulating the acoustic wave can include modulating one or more properties of the acoustic wave. Modulating the acoustic wave can include filtering the acoustic wave, such as filtering out unwanted frequencies or attenuating the acoustic wave to lower the amplitude. Modulating the acoustic wave can include adding one or more additional acoustic waves to the original acoustic wave. Modulating the acoustic wave can include combining the acoustic wave such that there is constructive or destructive interference where the resultant, combined acoustic wave corresponds to the modulated acoustic wave.

The NSS 905 can modulate or change one or more properties of the acoustic wave based on a time interval. The NSS 905 can change the one or more properties of the acoustic at the end of the time interval. For example, the NSS 905 can change a property of the acoustic wave every 30 seconds, 1 minute, 2 minutes, 3 minutes, 5 minutes, 7 minutes, 10 minutes, or 15 minutes. The NSS 905 can change a modulation frequency of the acoustic wave, where the modulation frequency refers to the repeated modulations or inverse of the pulse rate interval of the acoustic pulses. The modulation frequency can be a predetermined or desired frequency. The modulation frequency can correspond to a desired stimulation frequency of neural oscillations. The modulation frequency can be set to facilitate or cause sensory induction of neural oscillations. The NSS 905 can set the modulation frequency to a frequency in the range of 0.1 Hz to 10,000 Hz. For example, the NSS 905 can set the modulation frequency to about. 1 Hz, 1 Hz, 5 Hz, 10 Hz, 20 Hz, 25 Hz, 30 Hz, 31 Hz, 32 Hz, 33 Hz, 34 Hz, 35 Hz, 36 Hz, 37 Hz, 38 Hz, 39 Hz, 40 Hz, 41 Hz, 42 Hz, 43 Hz, 44 Hz, 45 Hz, 46 Hz, 47 Hz, 48 Hz, 49 Hz, 50 Hz, 60 Hz, 70 Hz, 80 Hz, 90 Hz, 100 Hz, 150 Hz, 160 Hz, 200 Hz, 240 Hz, 250 Hz, 300 Hz, 320 Hz, 400 Hz, 480 Hz, 500 Hz, 640 Hz, 1000 Hz, 1,280 Hz, 2000 Hz, 3000 Hz, 4,000 Hz, 5000 Hz, 6,000 Hz, 7,000 Hz, 8,000 Hz, 9,000 Hz, or 10,000 Hz.

The audio generation module 910 can determine to provide audio signals that include bursts of acoustic waves, audio pulses, or modulations to acoustic waves. The audio generation module 910 can instruct or otherwise cause the audio signaling component 950 to generate acoustic bursts or pulses. An acoustic pulse can refer to a burst of acoustic waves or a modulation to a property of an acoustic wave that is perceived by the brain as a change in sound. For example, an audio source that is intermittently turned on and off can create audio bursts or changes in sound. The audio source can be turned on and off based on a predetermined or fixed pulse rate interval, such as every 0.025 seconds, to provide a pulse repetition frequency of 40 Hz. The audio source can be turned on and off to provide a pulse repetition frequency in the range of 0.1 Hz to 10 kHz or more.

For example, FIGS. 10F-10I illustrates bursts of acoustic waves or bursts of modulations that can be applied to acoustic waves. The bursts of acoustic waves can include, for example, audio tones, beeps, or clicks. The modulations can refer to changes in the amplitude of the acoustic wave, changes in frequency or wavelength of the acoustic wave, overlaying another acoustic wave over the original acoustic wave, presenting the acoustic wave at intervals of desired frequency, or otherwise modifying or changing the acoustic wave.

For example, FIG. 10F illustrates acoustic bursts 1035a-c (or modulation pulses 1035a-c) in accordance with an embodiment. The acoustic bursts 1035a-c can be illustrated via a graph where the y-axis represents a parameter of the acoustic wave (e.g., frequency, wavelength, or amplitude) of the acoustic wave. The x-axis can represent time (e.g., seconds, milliseconds, or microseconds).

The audio signal can include a modulated acoustic wave that is modulated between different frequencies, wavelengths, or amplitudes. For example, the NSS 905 can modulate an acoustic wave between a frequency in the audio spectrum, such as Ma, and a frequency outside the audio spectrum, such as Mo. The NSS 905 can modulate the acoustic wave between two or more frequencies, between an on state and an off state, or between a high-power state and a low power state.

The acoustic bursts 1035a-c can have an acoustic wave parameter with value Ma that is different from the value Mo of the acoustic wave parameter. The modulation Ma can refer to a frequency or wavelength, or amplitude. The pulses 1035a-c can be generated with a pulse rate interval (PRI) 1040.

For example, the acoustic wave parameter can be the frequency of the acoustic wave. The first value Mo can be a low frequency or carrier frequency of the acoustic wave, such as 10 kHz. The second value, Ma, can be different from the first frequency Mo. The second frequency Ma can be lower or higher than the first frequency Mo. For example, the second frequency Ma can be 11 kHz. The difference between the first frequency and the second frequency can be determined or set based on a level of sensitivity of the human ear. The difference between the first frequency and the second frequency can be determined or set based on profile information 945 for the subject. The difference between the first frequency Mo and the second frequency Ma can be determined such that the modulation or change in the acoustic wave facilitate sensory induction of neural oscillations.

In some cases, the parameter of the acoustic wave used to generate the acoustic burst 1035a can be constant at Ma, thereby generating a square wave as illustrated in FIG. 10F. In some embodiments, each of the three pulses 1035a-c can include acoustic waves having a same frequency Ma.

The width of each of the acoustic bursts or pulses (e.g., the duration of the burst of the acoustic wave with the parameter Ma) can correspond to a pulse width 1030a. The pulse width 1030a can refer to the length or duration of the burst. The pulse width 1030a can be measured in units of time or distance. In some embodiments, the pulses 1035a-c can include acoustic waves having different frequencies from one another. In some embodiments, the pulses 1035a-c can have different pulse widths 1030a from one another, as illustrated in FIG. 10G. For example, a first pulse 1035d of FIG. 10G can have a pulse width 1030a, while a second pulse 1035e has a second pulse width 1030b that is greater than the first pulse width 1030a. A third pulse 1035f can have a third pulse width 1030c that is less than the second pulse width 1030b. The third pulse width 1030c can also be less than the first pulse width 1030a. While the pulse widths 1030a-c of the pulses 1035d-f of the pulse train may vary, the audio generation module 910 can maintain a constant pulse rate interval 1040 for the pulse train.

The pulses 1035a-c can form a pulse train having a pulse rate interval 1040. The pulse rate interval 1040 can be quantified using units of time. The pulse rate interval 1040 can be based on a frequency of the pulses of the pulse train 201. The frequency of the pulses of the pulse train 201 can be referred to as a modulation frequency. For example, the audio generation module 910 can provide a pulse train 201 with a predetermined frequency, such as 40 Hz. To do so, the audio generation module 910 can determine the pulse rate interval 1040 by taking the multiplicative inverse (or reciprocal) of the frequency (e.g., 1 divided by the predetermined frequency for the pulse train). For example, the audio generation module 910 can take the multiplicative inverse of 40 Hz by dividing 1 by 40 Hz to determine the pulse rate interval 1040 as 0.025 seconds. The pulse rate interval 1040 can remain constant throughout the pulse train. In some embodiments, the pulse rate interval 1040 can vary throughout the pulse train or from one pulse train to a subsequent pulse train. In some embodiments, the number of pulses transmitted during a second can be fixed, while the pulse rate interval 1040 varies.

In some embodiments, the audio generation module 910 can generate an audio burst or audio pulse having an acoustic wave that varies in frequency, amplitude, or wavelength. For example, the audio generation module 910 can generate up-chirp pulses where the frequency, amplitude, or wavelength of the acoustic wave of the audio pulse increases from the beginning of the pulse to the end of the pulse as illustrated in FIG. 10H. For example, the frequency, amplitude, or wavelength of the acoustic wave at the beginning of pulse 1035g can be Ma. The frequency, amplitude, or wavelength of the acoustic wave of the pulse 1035g can increase from Ma to Mb in the middle of the pulse 1035g, and then to a maximum of Mc at the end of the pulse 1035g. Thus, the frequency, amplitude or wavelength of the acoustic wave used to generate the pulse 1035g can range from Ma to Mc. The frequency, amplitude or wavelength can increase linearly, exponentially, or based on some other rate or curve. One or more of the frequency, amplitude or wavelength of the acoustic wave can change from the beginning of the pulse to the end of the pulse.

The audio generation module 910 can generate down-chirp pulses, as illustrated in FIG. 10I, where the frequency, amplitude, or wavelength of the acoustic wave of the acoustic pulse decreases from the beginning of the pulse to the end of the pulse. For example, the frequency, amplitude, or wavelength of an acoustic wave at the beginning of pulse 1035j can be Mc. The frequency, amplitude, or wavelength of the acoustic wave of the pulse 1035j can decrease from Mc to Mb in the middle of the pulse 1035j, and then to a minimum of Ma at the end of the pulse 1035j. Thus, the frequency, amplitude or wavelength of the acoustic wave used to generate the pulse 1035j can range from Mc to Ma. The frequency, amplitude or wavelength can decrease linearly, exponentially, or based on some other rate or curve. One or more of the frequency, amplitude, or wavelength of the acoustic wave can change from the beginning of the pulse to the end of the pulse.

In some embodiments, the audio generation module 910 can instruct or cause the audio signaling component 950 to generate audio pulses to stimulate specific or predetermined portions of the brain or a specific cortex. The frequency, wavelength, modulation frequency, amplitude and other aspects of the audio pulse, tone or music-based stimuli can dictate which cortex or cortices are recruited to process the stimuli. The audio signaling component 950 can stimulate discrete portions of the cortex by modulating the presentation of the stimuli to target specific or general regions of interest. The modulation parameters or amplitude of the audio stimuli can dictate which region of the cortex is stimulated. For example, different regions of the cortex are recruited to process different frequencies of sound, called their characteristic frequencies. Further, ear laterality of stimulation can influence cortex response since some subjects can be treated by stimulating one ear as opposed to both ears.

Audio signaling component 950 can be designed and constructed to generate the audio pulses responsive to instructions from the audio generation module 910. The instructions can include, for example, parameters of the audio pulse such as a frequency, wavelength or of the acoustic wave, duration of the pulse, frequency of the pulse train, pulse rate interval, or duration of the pulse train (e.g., a number of pulses in the pulse train or the length of time to transmit a pulse train having a predetermined frequency). The audio pulse can be perceived, observed, or otherwise identified by the brain via cochlear means such as ears. The audio pulses can be transmitted to the ear via an audio source speaker in close proximity to the ear, such as headphones, earbuds, bone conduction transducers, or cochlear implants. The audio pulses can be transmitted to the ear via an audio source or speaker not in close proximity to the ear, such as a surround sound speaker system, bookshelf speakers, or other speaker not directly or indirectly in contact with the ear.

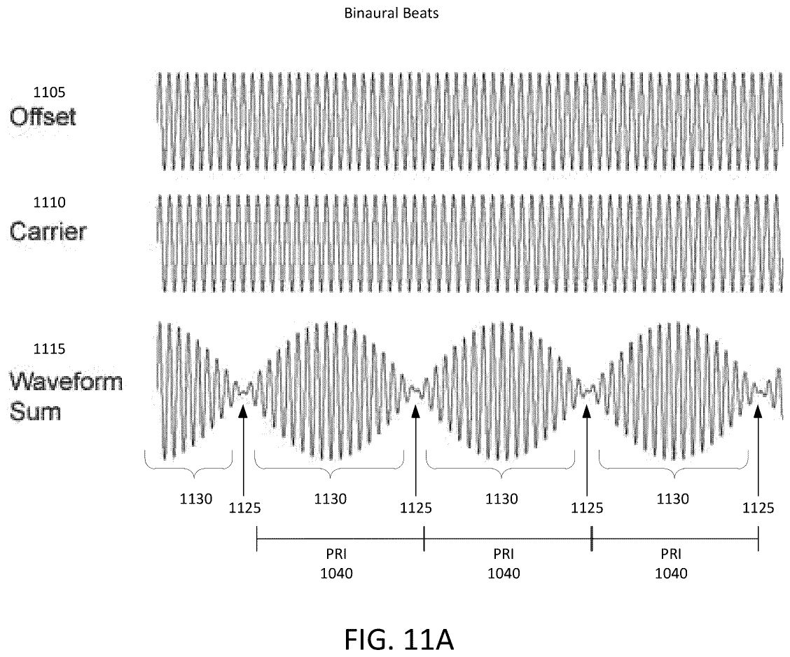

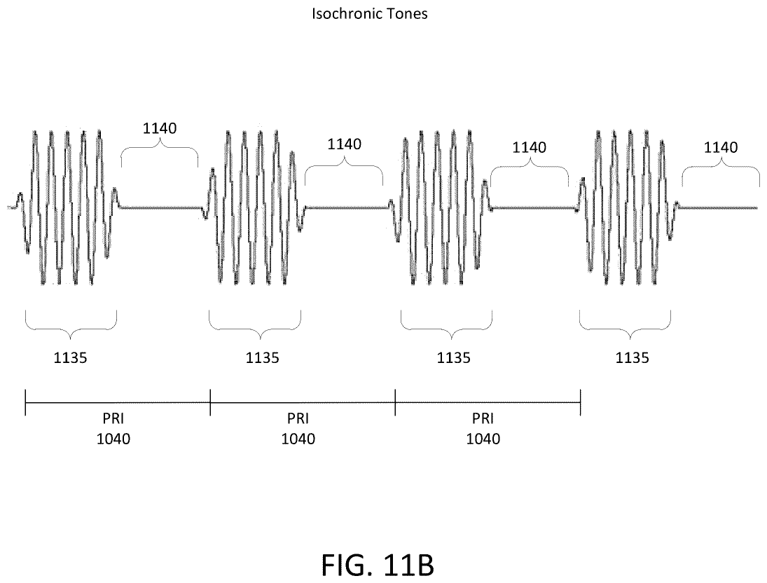

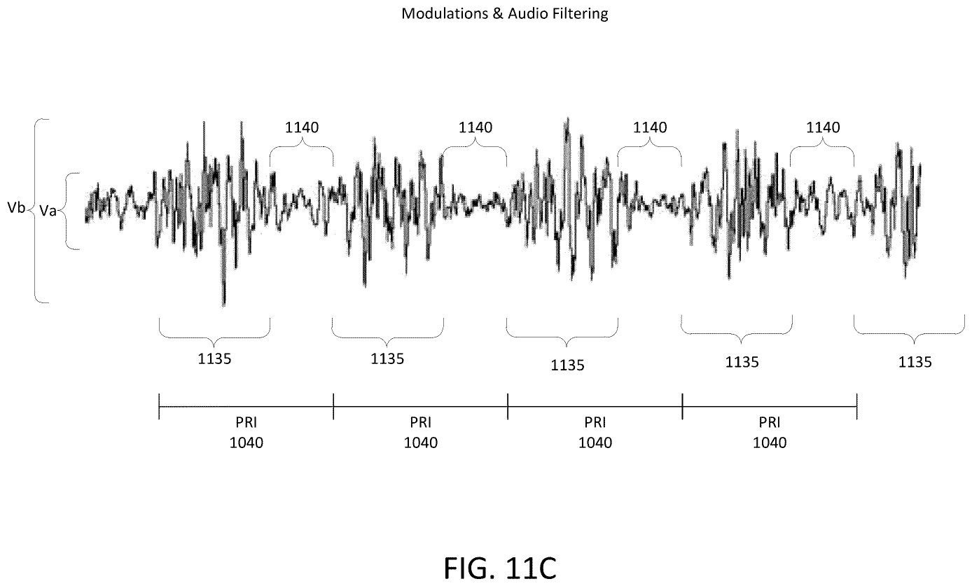

FIG. 11A illustrates audio signals using binaural beats or binaural pulses, in accordance with an embodiment. In brief summary, binaural beats refers to providing a different tone to each ear of the subject. When the brain perceives the two different tones, the brain mixes the two tones together to create a pulse. The two different tones can be selected such that the sum of the tones creates a pulse train having a desired pulse rate interval 1040.

The audio signaling component 950 can include a first audio source that provides an audio signal to the first ear of a subject, and a second audio source that provides a second audio signal to the second ear of a subject. The first audio source and the second audio source can be different. The first ear may only perceive the first audio signal from the first audio source, and the second ear may only receive the second audio signal from the second audio source. Audio sources can include, for example, headphones, earbuds, or bone conduction transducers. The audio sources can include stereo audio sources.

The audio generation component 910 can select a first tone for the first ear and a different second tone for the second ear. A tone can be characterized by its duration, pitch, intensity (or loudness), or timbre (or quality). In some cases, the first tone and the second tone can be different if they have different frequencies. In some cases, the first tone and the second tone can be different if they have different phase offsets. The first tone and the second tone can each be pure tones. A pure tone can be a tone having a sinusoidal waveform with a single frequency.

As illustrated in FIG. 11A, the first tone or offset wave 1105 is slightly different from the second tone 1110 or carrier wave 1110. The first tone 1105 has a higher frequency than the second tone 1110. The first tone 1105 can be generated by a first earbud that is inserted into one of the subject's ears, and the second tone 1110 can be generated by a second earbud that is inserted into the other of the subject's ears. When the auditory cortex of the brain perceives the first tone 1105 and the second tone 1110, the brain can sum the two tones. The brain can sum the acoustic waveforms corresponding to the two tones. The brain can sum the two waveforms as illustrated by waveform sum 1115. Due to the first and second tones having a different parameter (such as a different frequency or phase offset), portions of the waves can add and subtract from another to result in waveform 1115 having one or more pulses 1130 (or beats 1130). The pulses 1130 can be separated by portions 1125 that are at equilibrium. The pulses 1130 perceived by the brain by mixing these two different waveforms together can produce sensory induction of neural oscillations.

In some embodiments, the NSS 905 can generate binaural beats using a pitch panning technique. For example, the audio generation module 910 or audio adjustment module 915 can include or use a filter to modulate the pitch of a sound file or single tone up and down, and at the same time pan the modulation between stereo sides, such that one side will have a slightly higher pitch while the other side has a pitch that is slightly lower. The stereo sides can refer to the first audio source that generates and provides the audio signal to the first ear of the subject, and the second audio source that generates and provides the audio signal to the second ear of the subject. A sound file can refer to a file format configured to store a representation of, or information about, an acoustic wave. Example sound file formats can include .mp3, .wav, .aac, .m4a, .smf, etc.

The NSS 905 can use this pitch panning technique to generate a type of spatial positioning that, when listened to through stereo headphones, is perceived by the brain in a manner similar to binaural beats. The NSS 905 can, therefore, use this pitch panning technique to generate pulses or beats using a single tone or a single sound file.

In some cases, the NSS 905 can generate monaural beats or monaural pulses. Monaural beats or pulses are similar to binaural beats in that they are also generated by combining two tones to form a beat. The NSS 905 or component of system 100 can form monaural beats by combining the two tones using a digital or analog technique before the sound reaches the ears, as opposed to the brain combining the waveforms as in binaural beats. For example, the NSS 905 (or audio generation component 910) can identify and select two different waveforms that, when combined, produce beats or pulses having a desired pulse rate interval. The NSS 905 can identify a first digital representation of a first acoustic waveform and identify a second digital representation of a second acoustic waveform having a different parameter than the first acoustic waveform. The NSS 905 can combine the first and second digital waveforms to generate a third digital waveform different from the first digital waveform and the second digital waveform. The NSS 905 can then transmit the third digital waveform in a digital form to the audio signaling component 950. The NSS 905 can translate the digital waveform to an analog format and transmit the analog format to the audio signaling component 950. The audio signaling component 950 can then, via an audio source, generate the sound to be perceived by one or both ears. The same sound can be perceived by both ears. The sound can include the pulses or beats spaced at the desired pulse rate interval 1040.

FIG. 11B illustrates acoustic pulses having isochronic tones, in accordance with an embodiment. Isochronic tones are evenly spaced tone pulses. Isochronic tones can be created without having to combine two different tones. The NSS 905 or other component of system 100 can create the isochronic tone by turning a tone on and off. The NSS 905 can generate the isochronic tones or pulses by instructing the audio signaling component to turn on and off. The NSS 905 can modify a digital representation of an acoustic wave to remove or set digital values of the acoustic wave such that sound is generated during the pulses 1135 and no sound is generated during the null portions 1140.

By turning on and off the acoustic wave, the NSS 905 can establish acoustic pulses 1135 that are spaced apart by a pulse rate interval 1040 that corresponds to a desired stimulation frequency, such as 40 Hz. The isochronic pulses spaced part at the desired PRI 1040 can produce sensory induction of neural oscillations.

FIG. 11C illustrates audio pulses generated by the NSS 905 using a soundtrack, in accordance with an embodiment. A soundtrack can include or refer to a complex acoustical wave that includes multiple different frequencies, amplitudes, or tones. For example, a soundtrack can include a voice track, a musical instrument track, a musical track having both voice and musical instruments, nature sounds, or white noise.

The NSS 905 can modulate the soundtrack to produce sensory induction of neural oscillations by rhythmically adjusting a component in the sound. For example, the NSS 905 can modulate the volume by increasing and decreasing the amplitude of the acoustic wave or soundtrack to create the rhythmic stimulus corresponding to the stimulation frequency for producing sensory induction of neural oscillations. Thus, the NSS 905 can embed, into a sound track acoustic pulses having a pulse rate interval corresponding to the desired stimulation frequency to produce sensory induction of neural oscillations. The NSS 905 can manipulate the soundtrack to generate a new, modified soundtrack having acoustic pulses with a pulse rate interval corresponding to the desired stimulation frequency to produce sensory induction of neural oscillations.

As illustrated in FIG. 11C, pulses 1135 are generated by modulating the volume from a first level Va to a second level Vb. During portions 1140 of the acoustic wave 345, the NSS 905 can set or keep the volume at Va. The volume Va can refer to an amplitude of the wave, or a maximum amplitude or crest of the wave 345 during the portion 1140. The NSS 905 can then adjust, change, or increase the volume to Vb during portion 1135. The NSS 905 can increase the volume by a predetermined amount, such as a percentage, a number of decibels, a subject-specified amount, or other amount. The NSS 905 can set or maintain the volume at Vb for a duration corresponding to a desired pulse length for the pulse 1135.

In some embodiments, the NSS 905 can include an attenuator to attenuate the volume from level Vb to level Va. In some embodiments, the NSS 905 can instruct an attenuator (e.g., an attenuator of audio signaling component 950) to attenuate the volume from level Vb to level Va. In some embodiments, the NSS 905 can include an amplifier to amplify or increase the volume from Va to Vb. In some embodiments, the NSS 905 can instruct an amplifier (e.g., an amplifier of the audio signaling component 950) to amplify or increase the volume from Va to Vb.

Referring back to FIG. 9, the NSS 905 can include, access, interface with, or otherwise communicate with at least one audio adjustment module 915. The audio adjustment module 915 can be designed and constructed to adjust a parameter associated with the audio signal, such as a frequency, amplitude, wavelength, pattern, or other parameter of the audio signal. The audio adjustment module 915 can automatically vary a parameter of the audio signal based on profile information or feedback. The audio adjustment module 915 can receive the feedback information from the feedback monitor 935. The audio adjustment module 915 can receive instructions or information from a side effects management module 930. The audio adjustment module 915 can receive profile information from profile manager 925.

The NSS 905 can include, access, interface with, or otherwise communicate with at least one unwanted frequency filtering module 920. The unwanted frequency filtering module 920 can be designed and constructed to block, mitigate, reduce, or otherwise filter out frequencies of audio signals that are undesired to prevent or reduce an amount of such audio signals from being perceived by the brain. The unwanted frequency filtering module 920 can interface, instruct, control, or otherwise communicate with a filtering component 955 to cause the filtering component 955 to block, attenuate, or otherwise reduce the effect of the unwanted frequency on the neural oscillations.

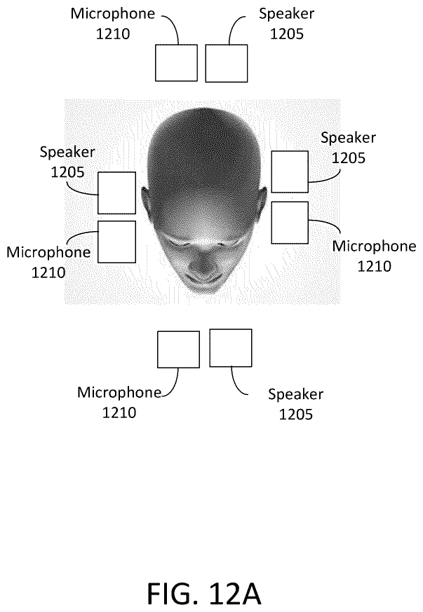

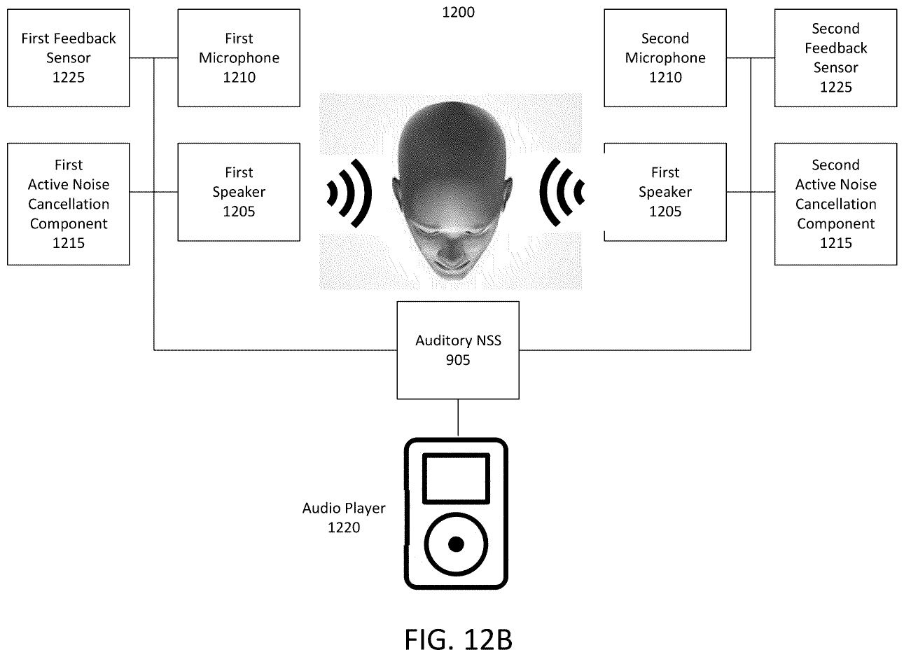

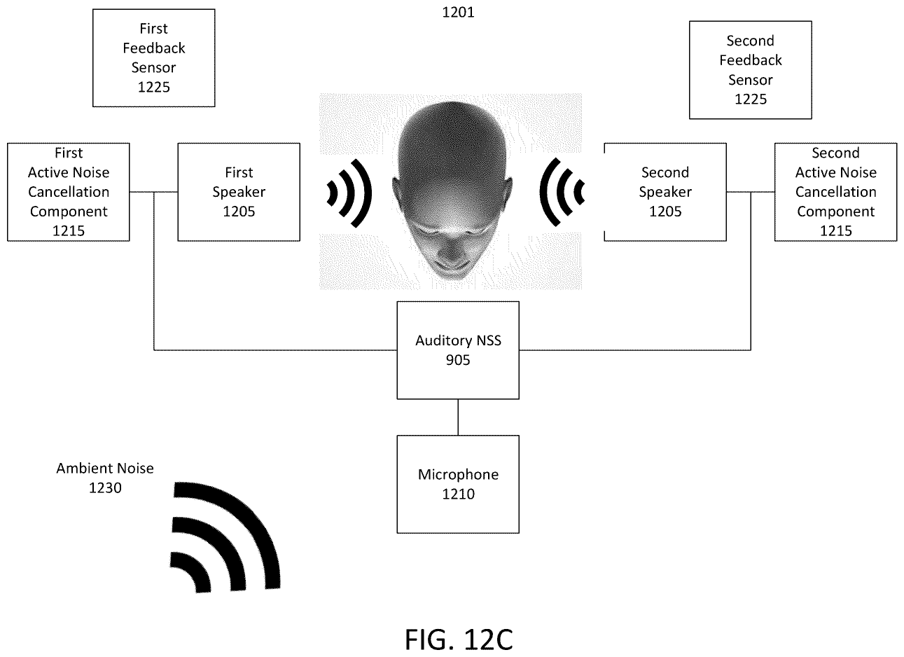

The unwanted frequency filtering module 920 can include an active noise control component (e.g., active noise cancellation component 1215 depicted in FIG. 12B). Active noise control can be referred to or include active noise cancellation or active noise reduction. Active noise control can reduce an unwanted sound by adding a second sound having a parameter specifically selected to cancel or attenuate the first sound. In some cases, the active noise control component can emit a sound wave with the same amplitude but with an inverted phase (or antiphase) to the original unwanted sound. The two waves can combine to form a new wave, and effectively cancel each other out by destructive interference.

The active noise control component can include analog circuits or digital signal processing. The active noise control component can include adaptive techniques to analyze waveforms of the background aural or non-aural noise. Responsive to the background noise, the active noise control component can generate an audio signal that can either phase shift or invert the polarity of the original signal. This inverted signal can be amplified by a transducer or speaker to create a sound wave directly proportional to the amplitude of the original waveform, creating destructive interference. This can reduce the volume of the perceivable noise.

In some embodiments, a noise-cancellation speaker can be co-located with a sound source speaker. In some embodiments, a noise cancellation speaker can be co-located with a sound source that is to be attenuated.

The unwanted frequency filtering module 920 can filter out unwanted frequencies that can adversely impact auditory induction of neural oscillations. For example, an active noise control component can identify that audio signals include acoustic bursts having the desired pulse rate interval, as well as acoustic bursts having an unwanted pulse rate interval. The active noise control component can identify the waveforms corresponding to the acoustic bursts having the unwanted pulse rate interval and generate an inverted phase waveform to cancel out or attenuate the unwanted acoustic bursts.

The NSS 905 can include, access, interface with, or otherwise communicate with at least one profile manager 925. The profile manager 925 can be designed or constructed to store, update, retrieve or otherwise manage information associated with one or more subjects associated with the auditory induction of neural oscillations. Profile information can include, for example, historical treatment information, historical brain sensory induced neural oscillations information, dosing information, parameters of acoustic waves, feedback, physiological information, environmental information, or other data associated with the systems and methods of sensory induction of neural oscillations.

The NSS 905 can include, access, interface with, or otherwise communicate with at least one side effects management module 930. The side effects management module 930 can be designed and constructed to provide information to the audio adjustment module 915 or the audio generation module 910 to change one or more parameter of the audio signal in order to reduce a side effect. Side effects can include, for example, nausea, migraines, fatigue, seizures, ear strain, deafness, ringing, or tinnitus.

The side effects management module 930 can automatically instruct a component of the NSS 905 to alter or change a parameter of the audio signal. The side effects management module 930 can be configured with predetermined thresholds to reduce side effects. For example, the side effects management module 930 can be configured with a maximum duration of a pulse train, maximum amplitude of acoustic waves, maximum volume, maximum duty cycle of a pulse train (e.g., the pulse width multiplied by the frequency of the pulse train), maximum number of treatments for sensory induction of neural oscillations in a time period (e.g., 1 hour, 2 hours, 12 hours, or 24 hours).

The side effects management module 930 can cause a change in the parameter of the audio signal in response to feedback information. The side effect management module 930 can receive feedback from the feedback monitor 935. The side effects management module 930 can determine to adjust a parameter of the audio signal based on the feedback. The side effects management module 930 can compare the feedback with a threshold to determine to adjust the parameter of the audio signal.

The side effects management module 930 can be configured with or include a policy engine that applies a policy or a rule to the current audio signal and feedback to determine an adjustment to the audio signal. For example, if feedback indicates that a patient receiving audio signals has a heart rate or pulse rate above a threshold, the side effects management module 930 can turn off the pulse train until the pulse rate stabilizes to a value below the threshold, or below a second threshold that is lower than the threshold.

The NSS 905 can include, access, interface with, or otherwise communicate with at least one feedback monitor 935. The feedback monitor can be designed and constructed to receive feedback information from a feedback component 960. Feedback component 960 can include, for example, a feedback sensor 1405 such as a temperature sensor, heart or pulse rate monitor, physiological sensor, ambient noise sensor, microphone, ambient temperature sensor, blood pressure monitor, brain wave sensor, EEG probe, electrooculography (“EOG”) probes configured measure the corneo-retinal standing potential that exists between the front and the back of the human eye, accelerometer, gyroscope, motion detector, proximity sensor, camera, microphone, or photo detector.

### Systems and Devices Configured for Neural Stimulation Via Auditory Stimulation

FIG. 12A illustrates a system for auditory induction of neural oscillations in accordance with an embodiment. The system 1200 can include one or more speakers 1205. The system 1200 can include one or more microphones. In some embodiments, the system can include both speakers 1205 and microphones 1210. In some embodiments, the system 1200 includes speakers 1205 and may not include microphones 1210. In some embodiments, the system 1200 includes microphones 1210 and may not include speakers 1210.

The speakers 1205 can be integrated with the audio signaling component 950. The audio signaling component 950 can include speakers 1205. The speakers 1205 can interact or communicate with audio signaling component 950. For example, the audio signaling component 950 can instruct the speaker 1205 to generate sound.

The microphones 1210 can be integrated with the feedback component 960. The feedback component 960 can include microphones 1210. The microphones 1210 can interact or communicate with feedback component 960. For example, the feedback component 960 can receive information, data, or signals from microphone 1210.

In some embodiments, the speaker 1205 and the microphone 1210 can be integrated together or a same device. For example, the speaker 1205 can be configured to function as the microphone 1210. The NSS 905 can toggle the speaker 1205 from a speaker mode to a microphone mode.

In some embodiments, the system 1200 can include a single speaker 1205 positioned at one of the ears of the subject. In some embodiments, the system 1200 can include two speakers. A first speaker of the two speakers can be positioned at a first ear, and the second speaker of the two speakers can be positioned at the second ear. In some embodiments, additional speakers can be positioned in front of the subject's head, or behind the subject's head. In some embodiments, one or more microphones 1210 can be positioned at one or both ears, in front of the subject's head, or behind the subject's head.

The speaker 1205 can include a dynamic cone speaker configured to produce sound from an electrical signal. The speaker 1205 can include a full-range driver to produce acoustic waves with frequencies over some or all of the audible range (e.g., 60 Hz to 20,000 Hz). The speaker 1205 can include a driver to produce acoustic waves with frequencies outside the audible range, such as 0 to 60 Hz, or in the ultrasonic range such as 20 kHz to 4 GHz. The speaker 1205 can include one or more transducers or drivers to produce sounds at varying portions of the audible frequency range. For example, the speaker 1205 can include tweeters for high range frequencies (e.g., 2,000 Hz to 20,000 Hz), mid-range drivers for middle frequencies (e.g., 250 Hz to 2000 Hz), or woofers for low frequencies (e.g., 60 Hz to 250 Hz).

The speaker 1205 can include one or more types of speaker hardware, components, or technology to produce sound. For example, the speaker 1205 can include a diaphragm to produce sound. The speaker 1205 can include a moving-iron loudspeaker that uses a stationary coil to vibrate a magnetized piece of metal. The speaker 1205 can include a piezoelectric speaker. A piezoelectric speaker can use the piezoelectric effect to generate sound by applying a voltage to a piezoelectric material to generate motion, which is converted into audible sound using diaphragms and resonators.

The speaker 1205 can include various other types of hardware or technology, such as magnetostatic loudspeakers, magnetostrictive speakers, electrostatic loudspeakers, a ribbon speaker, planar magnetic loudspeakers, bending wave loudspeakers, coaxial drivers, horn loudspeakers, Heil air motion transducers, or transparent ionic conductions speaker.

In some cases, the speaker 1205 may not include a diaphragm. For example, the speaker 1205 can be a plasma arc speaker that uses electrical plasma as a radiating element. The speaker 1205 can be a thermoacoustic speakers that uses carbon nanotube thin film. The speaker 1205 can be a rotary woofer that includes a fan with blades that constantly change their pitch.

In some embodiments, the speaker 1205 can include a headphone or a pair of headphones, earspeakers, earphones, or earbuds. Headphones can be relatively small speakers as compared to loudspeakers. Headphones can be designed and constructed to be placed in the ear, around the ear, or otherwise at or near the ear. Headphones can include electroacoustic transducers that convert an electrical signal to a corresponding sound in the subject's ear. In some embodiments, the headphones 1205 can include or interface with a headphone amplifier, such as an integrated amplifier or a standalone unit.

In some embodiments, the speaker 1205 can include headphones that can include an air jet that pushes air into the auditory canal, pushing the tympanum in a manner similar to that of a sound wave. The compression and rarefaction of the tympanic membrane through bursts of air (with or without any discernible sound) can control frequencies of neural oscillations similar to auditory signals. For example, the speaker 1205 can include air jets or a device that resembles in-ear headphones that either push, pull or both push and pull air into and out of the ear canal in order to compress or pull the tympanic membrane to affect the frequencies of neural oscillations. The NSS 905 can instruct, configure, or cause the air jets to generate bursts of air at a predetermined frequency.

In some embodiments, the headphones can connect to the audio signaling component 950 via a wired or wireless connection. In some embodiments, the audio signaling component 950 can include the headphones. In some embodiments, the headphones 1205 can interface with one or more components of the NSS 905 via a wired or wireless connection. In some embodiments, the headphones 1205 can include one or more components of the NSS 905 or system 100, such as the audio generation module 910, audio adjustment module 915, unwanted frequency filtering module 920, profile manager 925, side effects management module 930, feedback monitor 935, audio signaling component 950, filtering component 955, or feedback component 960.

The speaker 1205 can include or be integrated into various types of headphones. For example, the headphones can include, for example, circumaural headphones (e.g., full size headphones) that include circular or ellipsoid earpads that are designed and constructed to seal against the head to attenuate external noise. Circumaural headphones can facilitate providing an immersive auditory brainwave wave stimulation experience, while reducing external distractions. In some embodiments, headphones can include supra-aural headphones, which include pads that press against the ears rather than around them. Supra-aural headphones may provide less attenuation of external noise.

Both circumaural headphones and supra-aural headphones can have an open back, closed back, or semi open back. An open back leaks more noise and allows more ambient sounds to enter but provides a more natural or speaker-like sound. Closed back headphones block more of the ambient noise as compared to open back headphones, thus providing a more immersive auditory brainwave stimulation experience while reducing external distractions.

In some embodiments, headphones can include ear-fitting headphones, such as earphones or in-ear headphones. Earphones (or earbuds) can refer to small headphones that are fitted directly in the outer ear, facing but not inserted in the ear canal. Earphones, however, provide minimal acoustic isolation and allow ambient noise to enter. In-ear headphones (or in-ear monitors or canalphones) can refer to small headphones that can be designed and constructed for insertion into the ear canal. In-ear headphones engage the ear canal and can block out more ambient noise as compared to earphones, thus providing a more immersive auditory brainwave stimulation experience. In-ear headphones can include ear canal plugs made or formed from one or more material, such as silicone rubber, elastomer, or foam. In some embodiments, in-ear headphones can include custom-made castings of the ear canal to create custom-molded plugs that provide added comfort and noise isolation to the subject, thereby further improving the immersiveness of the auditory brainwave stimulation experience.

In some embodiments, one or more microphones 1210 can be used to detect sound. A microphone 1210 can be integrated with a speaker 1205. The microphone 1210 can provide feedback information to the NSS 905 or other component of system 100. The microphone 1210 can provide feedback to a component of the speaker 1205 to cause the speaker 1205 to adjust a parameter of audio signal.

The microphone 1210 can include a transducer that converts sound into an electrical signal. The Microphone 1210 can use electromagnetic induction, capacitance change, or piezoelectricity to produce the electrical signal from air pressure variations. In some cases, the microphone 1210 can include or be connected to a pre-amplifier to amplify the signal before it is recorded or processed. The microphone 1210 can include one or more type of microphone, including, for example, a condenser microphone, RF condenser microphone, electret condenser, dynamic microphone, moving-coil microphone, ribbon microphone, carbon microphone, piezoelectric microphone, crystal microphone, fiber optic microphone, laser microphone, liquid or water microphone, microelectromechanical systems (“MEMS”) microphone, or speakers as microphones.

The feedback component 960 can include or interface with the microphone 1210 to obtain, identify, or receive sound. The feedback component 960 can obtain ambient noise. The feedback component 960 can obtain sound from the speakers 1205 to facilitate the NSS 905 adjusting a characteristic of the audio signal generated by the speaker 1205. The microphone 1210 can receive voice input from the subject, such as audio commands, instructions, requests, feedback information, or responses to survey questions.

In some embodiments, one or more speakers 1205 can be integrated with one or more microphones 1210. For example, the speaker 1205 and microphone 1210 can form a headset, be placed in a single enclosure, or may even be the same device since the speaker 1205 and the microphone 1210 may be structurally designed to toggle between a sound generation mode and a sound reception mode.

FIG. 12B illustrates a system configuration for auditory induction of neural oscillations in accordance with an embodiment. The system 1200 can include at least one speaker 1205. The system 1200 can include at least microphone 1210. The system 1200 can include at least one active noise cancellation component 1215. The system 1200 can include at least one feedback sensor 1225. The system 1200 can include or interface with the NSS 905. The system 1200 can include or interface with an audio player 1220.

The system 1200 can include a first speaker 1205 positioned at a first ear. The system 1200 can include a second speaker 1205 positioned at a second year. The system 1200 can include a first active noise cancellation component 1215 communicatively coupled with the first microphone 1210. The system 1200 can include a second active noise cancellation component 1215 communicatively coupled with the second microphone 1210. In some cases, the active noise cancellation component 1215 can communicate with both the first speaker 1205 and the second speaker 1205, or both the first microphone 1210 and the second microphone 1210. The system 1200 can include a first microphone 1210 communicatively coupled with the active noise cancellation component 1215. The system 1200 can include a second microphone 1210 communicatively coupled with the active noise cancelation component 1215. In some embodiments, each of the microphone 1210, speaker 1205 and active noise cancellation component can communicate or interface with the NSS 905. In some embodiments, the system 1200 can include a feedback sensor 1225 and a second feedback sensor 1225 communicatively coupled to the NSS 905, the speaker 1205, microphone 1210, or active noise cancellation component 1215.

In operation, and in some embodiments, the audio player 1220 can play a musical track. The audio player 1220 can provide the audio signal corresponding to the musical track via a wired or wireless connection to the first and second speakers 1205. In some embodiments, the NSS 905 can intercept the audio signal from the audio player. For example, the NSS 905 can receive the digital or analog audio signal from the audio player 1220. The NSS 905 can be intermediary to the audio player 1220 and a speaker 1205. The NSS 905 can analyze the audio signal corresponding to the music in order to embed an auditory brainwave stimulation signal. For example, the NSS 905 can adjust the volume of the auditory signal from the audio player 1220 to generate acoustic pulses having a pulse rate interval as depicted in FIG. 11C. In some embodiments, the NSS 905 can use a binaural beats technique to provide different auditory signals to the first and second speakers that, when perceived by the brain, is combined to have the desired stimulation frequency.

In some embodiments, the NSS 905 can adjust for any latency between first and second speakers 1205 such that the brain perceives the audio signals at the same or substantially same time (e.g., within 1 millisecond, 2 milliseconds, 5 milliseconds, or 10 milliseconds). The NSS 905 can buffer the audio signals to account for latency such that audio signals are transmitted from the speakers at the same time.

In some embodiments, the NSS 905 may not be intermediary to the audio player 1220 and the speaker. For example, the NSS 905 can receive the musical track from a digital music repository. The NSS 905 can manipulate or modify the musical track to embed acoustic pulses in accordance with the desired PRI. The NSS 905 can then provide the modified musical track to the audio player 1220 to provide the modified audio signal to the speaker 1205.

In some embodiments, an active noise cancellation component 1215 can receive ambient noise information from the microphone 1210, identify unwanted frequencies or noise, and generate an inverted phase waveform to cancel out or attenuate the unwanted waveforms. In some embodiments, the system 1200 can include an additional speaker that generates the noise canceling waveform provided by the noise cancellation component 1215. The noise cancellation component 1215 can include the additional speaker.

The feedback sensor 1225 of the system 1200 can detect feedback information, such as environmental parameters or physiological conditions. The feedback sensor 1225 can provide the feedback information to NSS 905. The NSS 905 can adjust or change the audio signal based on the feedback information. For example, the NSS 905 can determine that a pulse rate of the subject exceeds a predetermined threshold, and then lower the volume of the audio signal. The NSS 905 can detect that the volume of the auditory signal exceeds a threshold and decrease the amplitude. The NSS 905 can determine that the pulse rate interval is below a threshold, which can indicate that a subject is losing focus or not paying a satisfactory level of attention to the audio signal, and the NSS 905 can increase the amplitude of the audio signal or change the tone or music track. In some embodiments, the NSS 905 can vary the tone or the music track based on a time interval. Varying the tone or the music track can cause the subject to pay a greater level of attention to the auditory stimulation, which can facilitate sensory induction of neural oscillations.

In some embodiments, the NSS 905 can receive neural oscillation information from EEG probes 1225, and adjust the auditory stimulation based on the EEG information. For example, the NSS 905 can determine, from the probe information, that neurons are oscillating at an undesired frequency. The NSS 905 can then identify the corresponding undesired frequency in ambient noise using the microphone 1210. The NSS 905 can then instruct the active noise cancellation component 1215 to cancel out the waveforms corresponding to the ambient noise having the undesired frequency.

In some embodiments, the NSS 905 can enable a passive noise filter. A pass noise filter can include a circuit having one or more or a resistor, capacitor or an inductor that filters out undesired frequencies of noise. In some cases, a passive filter can include a sound insulating material, sound proofing material, or sound absorbing material.

FIG. 4C illustrates a system configuration for auditory induction of neural oscillations in accordance with an embodiment. The system 401 can provide auditory brainwave stimulation using ambient noise source 1230. For example, system 401 can include the microphone 1210 that detects the ambient noise 1230. The microphone 1210 can provide the detected ambient noise to NSS 905. The NSS 905 can modify the ambient noise 1230 before providing it to the first speaker 1205 or the second speaker 1205. In some embodiments, the system 401 can be integrated or interface with a hearing aid device. A hearing aid can be a device designed to improve hearing.

The NSS 905 can increase or decrease the amplitude of the ambient noise 1230 to generate acoustic bursts having the desired pulse rate interval. The NSS 905 can provide the modified audio signals to the first and second speakers 1205 to facilitate auditory induction of neural oscillations.

In some embodiments, the NSS 905 can overlay a click train, tones, or other acoustic pulses over the ambient noise 1230. For example, the NSS 905 can receive the ambient noise information from the microphone 1210, apply an auditory stimulation signal to the ambient noise information, and then present the combined ambient noise information and auditory stimulation signal to the first and second speakers 1205. In some cases, the NSS 905 can filter out unwanted frequencies in the ambient noise 1230 prior to providing the auditory stimulation signal to the speakers 1205.

Thus, using the ambient noise 1230 as part of the auditory stimulation, a subject can observe the surroundings or carry on with their daily activities while receiving auditory stimulation to facilitate sensory induction of neural oscillations.

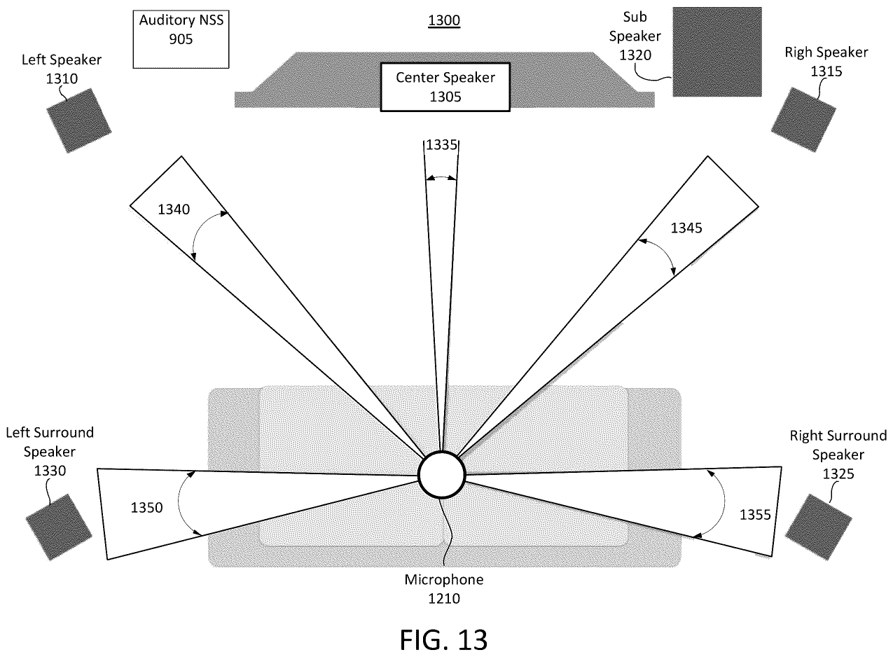

FIG. 13 illustrates a system configuration for auditory induction of neural oscillations in accordance with an embodiment. The system 1300 can provide auditory stimulation for sensory induction of neural oscillations using a room environment. The system 1300 can include one or more speakers. The system 1300 can include a surround sound system. For example, the system 1300 includes a left speaker 1310, right speaker 1315, center speaker 1305, right surround speaker 1325, and left surround speaker 1330. System 1300 an include a sub-woofer 1320. The system 1300 can include the microphone 1210. The system 1300 can include or refer to a 5.1 surround system. In some embodiments, the system 1300 can have 1, 2, 3, 4, 5, 6, 7 or more speakers.

When providing auditory stimulation using a surround system, the NSS 905 can provide the same or different audio signals to each of the speakers in the system 1300. The NSS 905 can modify or adjust audio signals provided to one or more of the speakers in system 1300 in order to facilitate sensory induction of neural oscillations. For example, the NSS 905 can receive feedback from microphone 1210 and modify, manipulate, or otherwise adjust the audio signal to optimize the auditory stimulation provided to a subject located at a position in the room that corresponds to the location of the microphone 1210. The NSS 905 can optimize or improve the auditory stimulation perceived at the location corresponding to microphone 1210 by analyzing the acoustic beams or waves generated by the speakers that propagate towards the microphone 1210.

The NSS 905 can be configured with information about the design and construction of each speaker. For example, speaker 1305 can generate sound in a direction that has an angle of 1335; speaker 1310 can generate sound that travels in a direction having an angle of 1340; speaker 1315 can generate sound that travels in a direction having an angle of 1345; speaker 1325 can generate sound that travels in a direction having an angle of 1355; and speaker 1330 can generate sound that travels in a direction having an angle of 1350. These angles can be the optimal or predetermined angles for each of the speakers. These angles can refer to the optimal angle of each speaker such that a person positioned at location corresponding to microphone 1210 can receive the optimum auditory stimulation. Thus, the speakers in system 1300 can be oriented to transmit auditory stimulation towards the subject.

In some embodiments, the NSS 905 can enable or disable one or more speakers. In some embodiments, the NSS 905 can increase or decrease the volume of the speakers to facilitate sensory induction of neural oscillations. The NSS 905 can intercept musical tracks, television audio, movie audio, internet audio, audio output from a set top box, or other audio source. The NSS 905 can adjust or manipulate the received audio and transmit the adjusted audio signals to the speakers in system 1300 to produce sensory induction of neural oscillations.

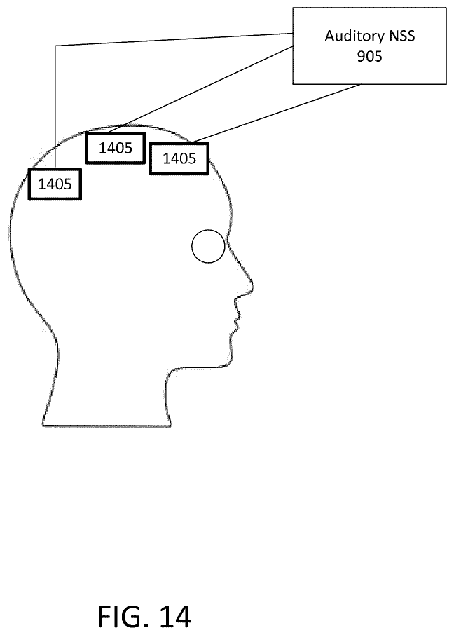

FIG. 14 illustrates feedback sensors 1405 placed or positioned at, on, or near a person's head. Feedback sensors 1405 can include, for example, EEG probes that detect brain wave activity.

The feedback monitor 935 can detect, receive, obtain, or otherwise identify feedback information from the one or more feedback sensors 1405. The feedback monitor 935 can provide the feedback information to one or more component of the NSS 905 for further processing or storage. For example, the profile manager 925 can update profile data structure 945 stored in data repository 940 with the feedback information. Profile manager 925 can associate the feedback information with an identifier of the patient or person undergoing the auditory brain stimulation, as well as a time stamp and date stamp corresponding to receipt or detection of the feedback information.

The feedback monitor 935 can determine a level of attention. The level of attention can refer to the focus provided to the acoustic pulses used for brain stimulation. The feedback monitor 935 can determine the level of attention using various hardware and software techniques. The feedback monitor 935 can assign a score to the level of attention (e.g., 1 to 10 with 1 being low attention and 10 being high attention, or vice versa, 1 to 100 with 1 being low attention and 100 being high attention, or vice versa, 0 to 1 with 0 being low attention and 1 being high attention, or vice versa), categorize the level of attention (e.g., low, medium, high), grade the attention (e.g., A, B, C, D, or F), or otherwise provide an indication of a level of attention.

In some cases, the feedback monitor 935 can track a person's eye movement to identify a level of attention. The feedback monitor 935 can interface with a feedback component 960 that includes an eye-tracker. The feedback monitor 935 (e.g., via feedback component 960) can detect and record eye movement of the person and analyze the recorded eye movement to determine an attention span or level of attention. The feedback monitor 935 can measure eye gaze which can indicate or provide information related to covert attention. For example, the feedback monitor 935 (e.g., via feedback component 960) can be configured with electro-oculography (“EOG”) to measure the skin electric potential around the eye, which can indicate a direction the eye faces relative to the head. In some embodiments, the EOG can include a system or device to stabilize the head so it cannot move in order to determine the direction of the eye relative to the head. In some embodiments, the EOG can include or interface with a head tracker system to determine the position of the heads, and then determine the direction of the eye relative to the head.

In some embodiments, the feedback monitor 935 and feedback component 960 can determine a level of attention the subject is paying to the auditory stimulation based on eye movement. For example, increased eye movement may indicate that the subject is focusing on visual stimuli, as opposed to the auditory stimulation. To determine the level of attention the subject is paying to visual stimuli as opposed to the auditory stimulation, the feedback monitor 935 and feedback component 960 can determine or track the direction of the eye or eye movement using video detection of the pupil or corneal reflection. For example, the feedback component 960 can include one or more camera or video camera. The feedback component 960 can include an infra-red source that sends light pulses towards the eyes. The light can be reflected by the eye. The feedback component 960 can detect the position of the reflection. The feedback component 960 can capture or record the position of the reflection. The feedback component 960 can perform image processing on the reflection to determine or compute the direction of the eye or gaze direction of the eye.

The feedback monitor 935 can compare the eye direction or movement to historical eye direction or movement of the same person, nominal eye movement, or other historical eye movement information to determine a level of attention. For example, the feedback monitor 935 can determine a historical amount of eye movement during historical auditory stimulation sessions. The feedback monitor 935 can compare the current eye movement with the historical eye movement to identify a deviation. The NSS 905 can determine, based on the comparison, an increase in eye movement and further determine that the subject is paying less attention to the current auditory stimulation based on the increase in eye movement. In response to detecting the decrease in attention, the feedback monitor 935 can instruct the audio adjustment module 915 to change a parameter of the audio signal to capture the subject's attention. The audio adjustment module 915 can change the volume, tone, pitch, or music track to capture the subject's attention or increase the level of attention the subject is paying to the auditory stimulation. Upon changing the audio signal, the NSS 905 can continue to monitor the level of attention. For example, upon changing the audio signal, the NSS 905 can detect a decrease in eye movement which can indicate an increase in a level of attention provided to the audio signal.

The feedback sensor 1405 can interact with or communicate with NSS 905. For example, the feedback sensor 1405 can provide detected feedback information or data to the NSS 905 (e.g., feedback monitor 935). The feedback sensor 1405 can provide data to the NSS 905 in real-time, for example as the feedback sensor 1405 detects or senses or information. The feedback sensor 1405 can provide the feedback information to the NSS 905 based on a time interval, such as 1 minute, 2 minutes, 5 minutes, 10 minutes, hourly, 2 hours, 4 hours, 12 hours, or 24 hours. The feedback sensor 1405 can provide the feedback information to the NSS 905 responsive to a condition or event, such as a feedback measurement exceeding a threshold or falling below a threshold. The feedback sensor 1405 can provide feedback information responsive to a change in a feedback parameter. In some embodiments, the NSS 905 can ping, query, or send a request to the feedback sensor 1405 for information, and the feedback sensor 1405 can provide the feedback information in response to the ping, request, or query.

### Method for Neural Stimulation Via Auditory Stimulation

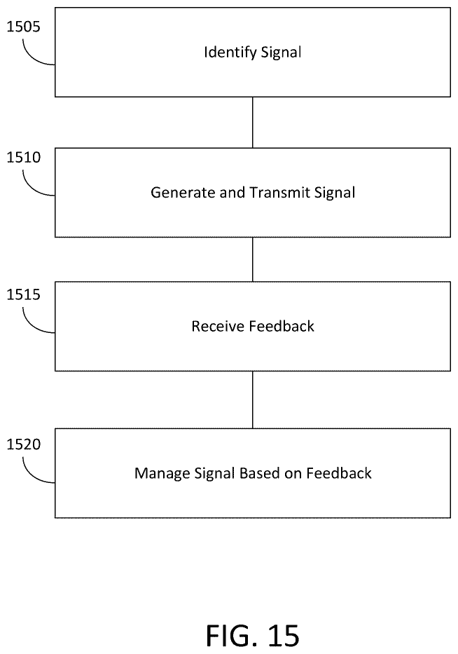

FIG. 15 is a flow diagram of a method of performing auditory induction of neural oscillations in accordance with an embodiment. The method 800 can be performed by one or more system, component, module, or element depicted in FIGS. 7A, 7B, and 9-14, including, for example, a neural stimulation system (NSS). In brief overview, the NSS can identify an audio signal to provide at block 1505. At block 1510, the NSS can generate and transmit the identified audio signal. At 1515 the NSS can receive or determine feedback associated with neural activity, physiological activity, environmental parameters, or device parameters. At 1520 the NSS can manage, control, or adjust the audio signal based on the feedback.

NSS Operating with Headphones

The NSS 905 can operate in conjunction with the speakers 1205 as depicted in FIG. 12A. The NSS 905 can operate in conjunction with earphones or in-ear phones including the speaker 1205 and a feedback sensor 1405.

In operation, a subject using the headphones can wear the headphones on their head such that speakers or placed at or in the ear canals. In some cases, the subject can provide an indication to the NSS 905 that the headphones have been worn and that the subject is ready to undergo sensory induction of neural oscillations. The indication can include an instruction, command, selection, input, or other indication via an input/output interface, such as a keyboard 726, pointing device 727, or other I/O devices 730a-n. The indication can be a motion-based indication, visual indication, or voice-based indication. For example, the subject can provide a voice command that indicates that the subject is ready to undergo sensory induction of neural oscillations.

In some cases, the feedback sensor 1405 can determine that the subject is ready to undergo sensory induction of neural oscillations. The feedback sensor 1405 can detect that the headphones have been placed on a subject's head. The NSS 905 can receive motion data, acceleration data, gyroscope data, temperature data, or capacitive touch data to determine that the headphones have been placed on the subject's head. The received data, such as motion data, can indicate that the headphones were picked up and placed on the subject's head. The temperature data can measure the temperature of or proximate to the headphones, which can indicate that the headphones are on the subject's head. The NSS 905 can detect that the subject is ready responsive to determining that the subject is paying a high level of attention to the headphones or feedback sensor 1405.

Thus, the NSS 905 can detect or determine that the headphones have been worn and that the subject is in a ready state, or the NSS 905 can receive an indication or confirmation from the subject that the subject has worn the headphones and the subject is ready to undergo sensory induction of neural oscillations. Upon determining that the subject is ready, the NSS 905 can initialize the sensory induction of neural oscillations process. In some embodiments, the NSS 905 can access a profile data structure 945. For example, a profile manager 925 can query the profile data structure 945 to determine one or more parameter for the external auditory stimulation used for the sensory induction of neural oscillations process. Parameters can include, for example, a type of audio stimulation technique, an intensity or volume of the audio stimulation, frequency of the audio stimulation, duration of the audio stimulation, or wavelength of the audio stimulation. The profile manager 925 can query the profile data structure 945 to obtain historical sensory induced neural oscillations information, such as prior auditory stimulation sessions. The profile manager 925 can perform a lookup in the profile data structure 945. The profile manager 925 can perform a look-up with a username, user identifier, location information, fingerprint, biometric identifier, retina scan, voice recognition and authentication, or other identifying technique.

The NSS 905 can determine a type of external auditory stimulation based on the components connected to the headphones. The NSS 905 can determine the type of external auditory stimulation based on the type of speakers 1205 available. For example, if the headphones are connected to an audio player, the NSS 905 can determined to embed acoustic pulses. If the headphones are not connected to an audio player, but only the microphone, the NSS 905 can determine to inject a pure tone or modify ambient noise.

In some embodiments, the NSS 905 can determine the type of external auditory stimulation based on historical sensory induction of neural oscillations sessions. For example, the profile data structure 945 can be pre-configured with information about the type of audio signaling component 950.

The NSS 905 can determine, via the profile manager 925, a modulation frequency for the pulse train or the audio signal. For example, NSS 905 can determine, from the profile data structure 945, that the modulation frequency for the external auditory stimulation should be set to 40 Hz. Depending on the type of auditory stimulation, the profile data structure 945 can further indicate a pulse length, intensity, wavelength of the acoustic wave forming the audio signal, or duration of the pulse train.

In some cases, the NSS 905 can determine or adjust one or more parameter of the external auditory stimulation. For example, the NSS 905 (e.g., via feedback component 960 or feedback sensor 1405) can determine an amplitude of the acoustic wave or volume level for the sound. The NSS 905 (e.g., via audio adjustment module 915 or side effects management module 930) can establish, initialize, set, or adjust the amplitude or wavelength of the acoustic waves or acoustic pulses. For example, the NSS 905 can determine that there is a low level of ambient noise. Due to the low level of ambient noise, subject's hearing may not be impaired or distracted. The NSS 905 can determine, based on detecting a low level of ambient noise, that it may not be necessary to increase the volume, or that it may be possible to reduce the volume to maintain the efficacy of sensory induction of neural oscillations.

In some embodiments, the NSS 905 can monitor (e.g., via feedback monitor 935 and feedback component 960) the level of ambient noise throughout the sensory induction of neural oscillations process to adjust the amplitude of the acoustic pulses automatically and periodically. For example, if the subject began the brainwave entrainment process when there was a high level of ambient noise, the NSS 905 can initially set a higher amplitude for the acoustic pulses and use a tone that includes frequencies that are easier to perceive, such as 10 kHz. However, in some embodiments in which the ambient noise level decreases throughout the sensory induction of neural oscillations process, the NSS 905 can automatically detect the decrease in ambient noise and, in response to the detection, adjust or lower the volume while decreasing the frequency of the acoustic wave. The NSS 905 can adjust the acoustic pulses to provide a high contrast ratio with respect to ambient noise to facilitate sensory induction of neural oscillations.

In some embodiments, the NSS 905 (e.g., via feedback monitor 935 and feedback component 960) can monitor or measure physiological conditions to set or adjust a parameter of the acoustic wave. In some embodiments, the NSS 905 can monitor or measure heart rate, pulse rate, blood pressure, body temperature, perspiration, or brain activity to set or adjust a parameter of the acoustic wave.

In some embodiments, the NSS 905 can be preconfigured to initially transmit acoustic pulses having a lowest setting for the acoustic wave intensity (e.g., low amplitude or high wavelength) and gradually increase the intensity (e.g., increase the amplitude of the or decrease the wavelength) while monitoring feedback until an optimal audio intensity is reached. An optimal audio intensity can refer to a highest intensity without adverse physiological side effects, such as deafness, seizures, heart attack, migraines, or other discomfort. The NSS 905 (e.g., via side effects management module 930) can monitor the physiological symptoms to identify the adverse side effects of the external auditory stimulation, and adjust (e.g., via audio adjustment module 915) the external auditory stimulation accordingly to reduce or eliminate the adverse side effects.

In some embodiments, the NSS 905 (e.g., via audio adjustment module 915) can adjust a parameter of the audio wave or acoustic pulse based on a level of attention. For example, during the sensory induction of neural oscillations process, the subject may get bored, lose focus, fall asleep, or otherwise not pay attention to the acoustic pulses. Not paying attention to the acoustic pulses may reduce the efficacy of the sensory induction of neural oscillations process, resulting in neurons oscillating at a frequency different from the desired modulation frequency of the acoustic pulses.

NSS 905 can detect the level of attention the subject is paying to the acoustic pulses using the feedback monitor 935 and one or more feedback component 960. Responsive to determining that the subject is not paying a satisfactory amount of attention to the acoustic pulses, the audio adjustment module 915 can change a parameter of the audio signal to gain the subject's attention. For example, the audio adjustment module 915 can increase the amplitude of the acoustic pulse, adjust the tone of the acoustic pulse, or change the duration of the acoustic pulse. The audio adjustment module 915 can randomly vary one or more parameters of the acoustic pulse. The audio adjustment module 915 can initiate an attention seeking acoustic sequence configured to regain the subject's attention. For example, the audio sequence can include a change in frequency, tone, amplitude, or insert words or music in a predetermined, random, or pseudo-random pattern. The attention seeking audio sequence can enable or disable different acoustic sources if the audio signaling component 950 includes multiple audio sources or speakers. Thus, the audio adjustment module 915 can interact with the feedback monitor 935 to determine a level of attention the subject is providing to the acoustic pulses and adjust the acoustic pulses to regain the subject's attention if the level of attention falls below a threshold.

In some embodiments, the audio adjustment module 915 can change or adjust one or more parameter of the acoustic pulse or acoustic wave at predetermined time intervals (e.g., every 5 minutes, 10 minutes, 15 minutes, or 20 minutes) to regain or maintain the subject's attention level.

In some embodiments, the NSS 905 (e.g., via unwanted frequency filtering module 920) can filter, block, attenuate, or remove unwanted auditory external stimulation. Unwanted auditory external stimulation can include, for example, unwanted modulation frequencies, unwanted intensities, or unwanted wavelengths of sound waves. The NSS 905 can deem a modulation frequency to be unwanted if the modulation frequency of a pulse train is different or substantially different (e.g., 1%, 2%, 5%, 10%, 15%, 20%, 25%, or more than 25%) from a desired frequency.

For example, the desired modulation frequency for sensory induction of neural oscillations can be 40 Hz. However, a modulation frequency of 20 Hz or 80 Hz can reduce the beneficial effects to cognitive functioning of the brain, a cognitive state of the brain, the immune system, or inflammation that can result from sensory induction of neural oscillations at other frequencies, such as 40 Hz. Thus, the NSS 905 can filter out the acoustic pulses corresponding to the 20 Hz or 80 Hz modulation frequency.

In some embodiments, the NSS 905 can detect, via feedback component 960, that there are acoustic pulses from an ambient noise source that corresponds to an unwanted modulation frequency of 20 Hz. The NSS 905 can further determine the wavelength of the acoustic waves of the acoustic pulses corresponding to the unwanted modulation frequency. The NSS 905 can instruct the filtering component 955 to filter out the wavelength corresponding to the unwanted modulation frequency.

### Neural Stimulation Via Peripheral Nerve Stimulation

In some embodiments, systems and methods of the present disclosure can provide peripheral nerve stimulation to cause or induce neural oscillations. For example, haptic stimulation on the skin around sensory nerves forming part of or connected to the peripheral nervous system can cause or induce electrical activity in the sensory nerves, causing a transmission to the brain via the central nervous system, which can be perceived by the brain or can cause or induce electrical and neural activity in the brain, including activity resulting in neural oscillations. Similarly, electric currents on or through the skin around sensory nerves forming part of or connected to the peripheral nervous system can cause or induce electrical activity in the sensory nerves, causing a transmission to the brain via the central nervous system, which can be perceived by the brain or can cause or induce electrical and neural activity in the brain, including activity resulting in neural oscillations. The brain, responsive to receiving the peripheral nerve stimulations, can adjust, manage, or control the frequency of neural oscillations. The electric currents can result in depolarization of neural cells, such as due to electric current stimuli such as time-varying pulses. The electric current pulse may directly cause depolarization. Secondary effects in other regions of the brain may be gated or controlled by the brain in response to the depolarization. The peripheral nerve stimulations generated at a predetermined frequency can trigger neural activity in the brain to cause or induce neural oscillations. The frequency of neural oscillations can be based on or correspond to the frequency of the peripheral nerve stimulations, or a modulation frequency associated with the peripheral nerve stimulations. Thus, systems and methods of the present disclosure can cause or induce neural oscillations using peripheral nerve stimulations such as electric current pulses modulated at a predetermined frequency to synchronize electrical activity among groups of neurons based on the frequency of the peripheral nerve stimulations. Sensory induction of neural oscillations can be observed based on the aggregate frequency of neural oscillations produced by the synchronous electrical activity in ensembles of cortical neurons. The frequency of the modulation of the electric currents, or pulses thereof, can cause or adjust this synchronous electrical activity in the ensembles of cortical neurons to oscillate at a frequency corresponding to the frequency of the peripheral nerve stimulation pulses.

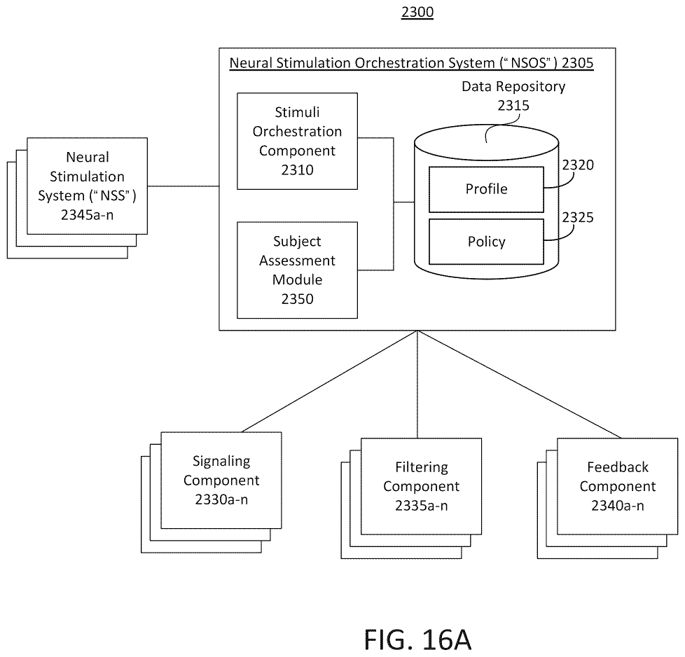

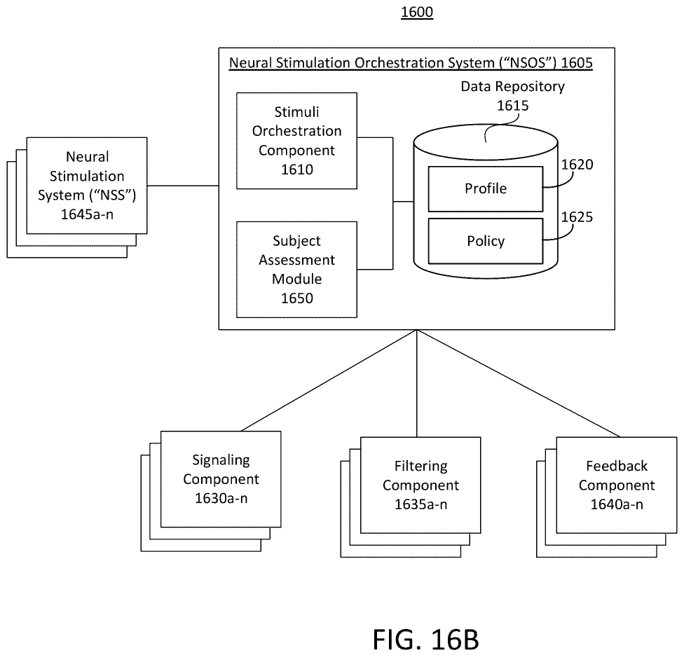

FIG. 16A is a block diagram depicting a system to perform peripheral nerve stimulation to cause or induce neural oscillations, such as to cause brain entrainment, in accordance with an embodiment. The system 1600 can include a peripheral nerve stimulation system 1605. In brief overview, the peripheral nerve stimulation system (or peripheral nerve stimulation neural stimulation system) (“NSS”) 1605 can include, access, interface with, or otherwise communicate with one or more of a nerve stimulus generation module 1610, nerve stimulus adjustment module 1615, profile manager 1625, side effects management module 1630, feedback monitor 1635, data repository 1640, nerve stimulus generator component 1650, shielding component 1655, feedback component 1660, or nerve stimulus amplification component 1665. The nerve stimulus generation module 1610, nerve stimulus adjustment module 1615, profile manager 1625, side effects management module 1630, feedback monitor 1635, nerve stimulus generator component 1650, shielding component 1655, feedback component 1660, or nerve stimulus amplification component 1665 can each include at least one processing unit or other logic device such as programmable logic array engine, or module configured to communicate with the database repository 1650. The nerve stimulus generation module 1610, nerve stimulus adjustment module 1615, profile manager 1625, side effects management module 1630, feedback monitor 1635, nerve stimulus generator component 1650, shielding component 1655, feedback component 1660, or nerve stimulus amplification component 1665 can be separate components, a single component, or part of the NSS 1605. The system 1600 and its components, such as the NSS 1605, may include hardware elements, such as one or more processors, logic devices, or circuits. The system 1600 and its components, such as the NSS 1605, can include one or more hardware or interface component depicted in system 700 in FIGS. 7A and 7B. For example, a component of system 1600 can include or execute on one or more processors 721, access storage 728 or memory 722, and communicate via network interface 718.

### Neural Stimulation Via Multiple Modes of Stimulation

FIG. 16B is a block diagram depicting a system for neural stimulation via multiple modes of stimulation in accordance with an embodiment. The system 1600 can include a neural stimulation orchestration system (“NSOS”) 1605. The NSOS 1605 can provide multiple modes of stimulation. For example, the NSOS 1605 can provide a first mode of stimulation that includes visual stimulation, and a second mode of stimulation that includes auditory stimulation. For each mode of stimulation, the NSOS 1605 can provide a type of signal. For example, for the visual mode of stimulation, the NSOS 1605 can provide the following types of signals: light pulses, image patterns, flicker of ambient light, or augmented reality. NSOS 1605 can orchestrate, manage, control, or otherwise facilitate providing multiple modes of stimulation and types of stimulation.

In brief overview, the NSOS 1605 can include, access, interface with, or otherwise communicate with one or more of a stimuli orchestration component 1610, a subject assessment module 1650, a data repository 1615, one or more signaling components 1630a-n, one or more filtering components 1635a-n, one or more feedback components 1640a-n, and one or more neural stimulation systems (“NSS”) 1645a-n. The data repository 1615 can include or store a profile data structure 1620 and a policy data structure 1625. The stimuli orchestration component 1610 and subject assessment module 1650 can include at least one processing unit or other logic device such as programmable logic array engine, or module configured to communicate with the database repository 1615. The stimuli orchestration component 1610 and subject assessment module 1650 can be a single component, include separate components, or be part of the NSOS 1605. The system 1600 and its components, such as the NSOS 1605, may include hardware elements, such as one or more processors, logic devices, or circuits. The system 1600 and its components, such as the NSOS 1605, can include one or more hardware or interface component depicted in system 700 in FIGS. 7A and 7B. For example, a component of system 1600 can include or execute on one or more processors 721, access storage 728 or memory 722, and communicate via network interface 718. The system 1600 can include one or more component or functionality depicted in FIGS. 1-15, including, for example, system 100, system 900, visual NSS 105, or auditory NSS 905. For example, at least one of the signaling components 1630a-n can include one or more component or functionality of visual signaling component 150 or audio signaling component 950. At least one of the filtering components 1635a-n can include one or more component or functionality of filtering component 155 or filtering component 955. At least one of the feedback components 1640a-n can include one or more component or functionality of feedback component 160 or feedback component 960. At least one of the NSS 1645a-n can include one or more component or functionality of visual NSS 105 or auditory NSS 905.

Still referring to FIG. 16B, and in further detail, the NSOS 1605 can include at least stimuli orchestration component 1610. The stimuli orchestration component 1610 can be designed and constructed to perform neural stimulation using multiple modalities of stimulation. The stimuli orchestration component 1610 or NSOS 1605 can interface with at least one of the signaling components 1630a-n, at least one of the filtering components 1635a-n or at least one of the feedback components 1640a-n. One or more of the signaling components 1630a-n can be a same type of signaling component or a different type of signaling component. The type of signaling component can correspond to a mode of stimulation. For example, multiple types of signaling components 1630a-n can correspond to visual signaling components or auditory signaling components. In some cases, at least one of the signaling components 1630a-n includes a visual signaling component 150 such as a light source, LED, laser, tablet computing device, or virtual reality headset. At least one of the signaling components includes an audio signaling component 950, such as headphones, speakers, cochlear implants, or air jets.

One or more of the filtering components 1635a-n can be a same type of filtering component or a different type of filtering component. One or more of the feedback components 1640a-n can be a same type of feedback component or a different type of feedback component. For example, the feedback components 1640a-n can include an electrode, dry electrode, gel electrode, saline soaked electrode, adhesive-based electrodes, a temperature sensor, heart or pulse rate monitor, physiological sensor, ambient light sensor, ambient temperature sensor, sleep status via actigraphy, blood pressure monitor, respiratory rate monitor, brain wave sensor, EEG probe, EOG probes configured measure the corneo-retinal standing potential that exists between the front and the back of the human eye, accelerometer, gyroscope, motion detector, proximity sensor, camera, microphone, or photo detector.

The stimuli orchestration component 1610 can include or be configured with an interface to communicate with different types of signaling components 1630a-n, filtering components 1635a-n or feedback components 1640a-n. The NSOS 1605 or stimuli orchestration component 1610 can interface with system intermediary to one of the signaling components 1630a-n, filtering components 1635a-n, or feedback components 1640a-n. For example, the stimuli orchestration component 1610 can interface with the visual NSS 105 depicted in FIG. 1 or auditory NSS 905 depicted in FIG. 9. Thus, in some embodiments, the stimuli orchestration component 1610 or NSOS 1605 can indirectly interface with at least one of the signaling components 1630a-n, filtering components 1635a-n, or feedback components 1640a-n.

The stimuli orchestration component 1610 (e.g., via the interface) can ping each of the signaling components 1630a-n, filtering components 1635a-n, and feedback components 1640a-n to determine information about the components. The information can include a type of the component (e.g., visual, auditory, attenuator, optical filter, temperature sensor, or light sensor), configuration of the component (e.g., frequency range, amplitude range), or status information (e.g., standby, ready, online, enabled, error, fault, offline, disabled, warning, service needed, availability, or battery level).

The stimuli orchestration component 1610 can instruct or cause at least one of the signaling components 1630a-n to generate, transmit or otherwise provide a signal that can be perceived, received, or observed by the brain and affect a frequency of neural oscillations in at least one region or portion of a subject's brain. The signal can be perceived via various means, including, for example, optical nerves or cochlear cells.

The stimuli orchestration component 1610 can access the data repository 1615 to retrieve profile information 1620 and a policy 1625. The profile information 1620 can include profile information 145 or profile information 945. The policy 1625 can include a multi-modal stimulation policy. The policy 1625 can indicate a multi-modal stimulation program. The stimuli orchestration component 1610 can apply the policy 1625 to profile information to determine a type of stimulation (e.g., visual or auditory) and determine a value for a parameter for each type of stimulation (e.g., amplitude, frequency, wavelength, color, etc.). The stimuli orchestration component 1610 can apply the policy 1625 to the profile information 1620 and feedback information received from one or more feedback components 1640a-n to determine or adjust the type of stimulation (e.g., visual or auditory) and determine or adjust the value parameter for each type of stimulation (e.g., amplitude, frequency, wavelength, color, etc.). The stimuli orchestration component 1610 can apply the policy 1625 to profile information to determine a type of filter to be applied by at least one of the filtering components 1635a-n (e.g., audio filter or visual filter) and determine a value for a parameter for the type of filter (e.g., frequency, wavelength, color, sound attenuation, etc.). The stimuli orchestration component 1610 can apply the policy 1625 to profile information and feedback information received from one or more feedback components 1640a-n to determine or adjust the type of filter to be applied by at least one of the filtering components 1635a-n (e.g., audio filter or visual filter) and determine or adjust the value for the parameter for filter (e.g., frequency, wavelength, color, sound attenuation, etc.).

The NSOS 1605 can obtain the profile information 1620 via a subject assessment module 1650. The subject assessment module 1650 can be designed and constructed to determine, for one or more subjects, information that can facilitate neural stimulation via one or more modes of stimulation. The subject assessment module 1650 can receive, obtain, detect, determine, or otherwise identify the information via feedback components 1640a-n, surveys, queries, questionnaires, prompts, remote profile information accessible via a network, diagnostic tests, or historical treatments.

The subject assessment module 1650 can receive the information prior to initiating neural stimulation, during neural stimulation, or after neural stimulation. For example, the subject assessment module 1650 can provide a prompt with a request for information prior to initiating the neural stimulation session. The subject assessment module 1650 can provide a prompt with a request for information during the neural stimulation session. The subject assessment module 1650 can receive feedback from feedback component 1640a-n (e.g., an EEG probe) during the neural stimulation session. The subject assessment module 1650 can provide a prompt with a request for information subsequent to termination of the neural stimulation session. The subject assessment module 1650 can receive feedback from feedback component 1640a-n subsequent to termination of the neural stimulation session.

The subject assessment module 1650 can use the information to determine an effectiveness of a modality of stimulation (e.g., visual stimulation or auditory stimulation) or a type of signal (e.g., light pulse from a laser or LED source, ambient light flicker, or image pattern displayed by a tablet computing device). For example, the subject assessment module 1650 can determine that the desired neural stimulation resulted from a first mode of stimulation or first type of signal, while the desired neural stimulation did not occur or took longer to occur with the second mode of stimulation or second type of signal. The subject assessment module 1650 can determine that the desired neural stimulation was less pronounced from the second mode of stimulation or second type of signal relative to the first mode of stimulation or first type of signal based on feedback information from a feedback component 1640a-n.

The subject assessment module 1650 can determine the level of effectiveness of each mode or type of stimulation independently or based on a combination of modes or types of stimulation. A combination of modes of stimulation can refer to transmitting signals from different modes of stimulation at the same or substantially similar time. A combination of modes of stimulation can refer to transmitting signals from different modes of stimulation in an overlapping manner. A combination of modes of stimulation can refer to transmitting signals from different modes of stimulation in a non-overlapping manner, but within a time interval from one another (e.g., transmit a signal pulse train from a second mode of stimulation within 0.5 seconds, 1 second, 1.5 seconds, 2 seconds, 2.5 seconds, 3 seconds, 5 seconds, 7 seconds, 10 seconds, 12 seconds, 15 seconds, 20 seconds, 30 seconds, 45 seconds, 60 seconds, 1 minute, 2 minutes 3 minutes 5 minutes, 10 minutes, or other time interval where the effect on the frequency of neural oscillation by a first mode can overlap with the second mode).

The subject assessment module 1650 can aggregate or compile the information and update the profile data structure 1620 stored in data repository 1615. In some cases, the subject assessment module 1650 can update or generate a policy 1625 based on the received information. The policy 1625 or profile information 1620 can indicate which modes or types of stimulation are more likely to have a desired effect on neural stimulation, while reducing side effects.

The stimuli orchestration component 1610 can instruct or cause multiple signaling components 1630a-n to generate, transmit or otherwise provide different types of stimulation or signals pursuant to the policy 1625, profile information 1620 or feedback information detected by feedback components 1640a-n. The stimuli orchestration component 1610 can cause multiple signaling components 1630a-n to generate, transmit or otherwise provide different types of stimulation or signals simultaneously or at substantially the same time. For example, a first signaling component 1630a can transmit a first type of stimulation at the same time as a second signaling component 1630b transmits a second type of stimulation. The first signaling component 1630a can transmit or provide a first set of signals, pulses, or stimulation at the same time the second signaling component 1630b transmits or provides a second set of signals, pulses, or stimulation. For example, a first pulse from a first signaling component 1630a can begin at the same time or substantially the same time (e.g., 1%, 2%, 3%, 4%, 5%, 6%, 7%, 10%, 15%, 20%) as a second pulse from a second signaling component 1630b. First and second pulses can end at the same time or substantially same time. In another example, a first pulse train can be transmitted by the first signaling component 1630a at the same or substantially similar time as a second pulse train transmitted by the second signaling component 1630b.

The stimuli orchestration component 1610 can cause multiple signaling components 1630a-n to generate, transmit or otherwise provide different types of stimulation or signals in an overlapping manner. The different pulses or pulse trains may overlap one another but may not necessary being or end at a same time. For example, at least one pulse in the first set of pulses from the first signaling component 1630a can at least partially overlap, in time, with at least one pulse from the second set of pulses from the second signaling component 1630b. For example, the pulses can straddle one another. In some cases, a first pulse train transmitted or provided by the first signaling component 1630a can at least partially overlap with a second pulse train transmitted or provided by the second signaling component 1630b. The first pulse train can straddle the second pulse train.

The stimuli orchestration component 1610 can cause multiple signaling components 1630a-n to generate, transmit or otherwise provide different types of stimulation or signals such that they are received, perceived, or otherwise observed by one or more regions or portions of the brain at the same time, simultaneously or at substantially the same time. The brain can receive different modes of stimulation or types of signals at different times. The duration of time between transmission of the signal by a signaling component 1630a-n and reception or perception of the signal by the brain can vary based on the type of signal (e.g., visual, auditory), parameter of the signal (e.g., velocity or speed of the wave, amplitude, frequency, wavelength), or distance between the signaling component 1630a-n and the nerves or cells of the subject configured to receive the signal (e.g., eyes or ears). The stimuli orchestration component 1610 can offset or delay the transmission of signals such that the brain perceives the different signals at the desired time. The stimuli orchestration component 1610 can offset or delay the transmission of a first signal transmitted by a first signaling component 1630a relative to transmission of a second signal transmitted by a second signaling component 1630b. The stimuli orchestration component 1610 can determine an amount of an offset for each type of signal or each signaling component 1630a-n relative to a reference clock or reference signal. The stimuli orchestration component 1610 can be preconfigured or calibrated with an offset for each signaling component 1630a-n.

The stimuli orchestration component 1610 can determine to enable or disable the offset based on the policy 1625. For example, the policy 1625 may indicate to transmit multiple signals at the same time, in which case the stimuli orchestration component 1610 may disable or not use an offset. In another example, the policy 1625 may indicate to transmit multiple signals such that they are perceived by the brain at the same time, in which case the stimuli orchestration component 1610 may enable or use the offset.

In some embodiments, the stimuli orchestration component 1610 can stagger signals transmitted by different signaling components 1630a-n. For example, the stimuli orchestration component 1610 can stagger the signals such that the pulses from different signaling components 1630a-n are non-overlapping. The stimuli orchestration component 1610 can stagger pulse trains from different signaling components 1630a-n such that they are non-overlapping. The stimuli orchestration component 1610 can set parameters for each mode of stimulation or signaling component 1630a-n such that the signals they are non-overlapping.

Thus, the stimuli orchestration component 1610 can set parameters for signals transmitted by one or more signaling components 1630a-n such that the signals are transmitted in a synchronously or asynchronously or perceived by the brain synchronously or asynchronously. The stimuli orchestration component 1610 can apply the policy 1625 to available signaling components 1630a-n to determine the parameters to set for each signaling component 1630a-n for the synchronous or asynchronous transmission. The stimuli orchestration component 1610 can adjust parameters such as a time delay, phase offset, frequency, pulse rate interval, or amplitude to synchronize the signals.

In some embodiments, the NSOS 1605 can adjust or change the mode of stimulation, or a type of signal based on feedback received from a feedback component 1640a-n. The stimuli orchestration component 1610 can adjust the mode of stimulation or type of signal based on feedback on the subject, feedback on the environment, or a combination of feedback on the subject and the environment. Feedback on the subject can include, for example, physiological information, temperature, attention level, level of fatigue, activity (e.g., sitting, laying down, walking, biking, or driving), vision ability, hearing ability, side effects (e.g., pain, migraine, ringing in ear, or blindness), or frequency of neural oscillation at a region or portion of the brain (e.g., EEG probes). Feedback information on the environment can include, for example, ambient temperature, ambient light, ambient sound, battery information, or power source.

The stimuli orchestration component 1610 can determine to maintain or change an aspect of the stimulation treatment based on the feedback. For example, the stimuli orchestration component 1610 can determine that the neurons are not oscillating at the desired frequency in response to the first mode of stimulation. Responsive to determining that the neurons are not oscillating at the desired frequency, the stimuli orchestration component 1610 can disable the first mode of stimulation and enable a second mode of stimulation. The stimuli orchestration component 1610 can again determine (e.g., via feedback component 1640a) that the neurons are not oscillating at the desired frequency in response to the second mode of stimulation. Responsive to determining that the neurons are still not oscillating at the desired frequency, the stimuli orchestration component 1610 can increase an amplitude of the signal corresponding to the second mode of stimulation. The stimuli orchestration component 1610 can determine that the neurons are oscillating at the desired frequency in response to increasing the amplitude of a signal corresponding to the second mode of stimulation.

The stimuli orchestration component 1610 can monitor the frequency of neural oscillations at a region or portion of the brain. The stimuli orchestration component 1610 can determine that neurons in a first region of the brain are oscillating at the desired frequency, whereas neurons in a second region of the brain are not oscillating at the desired frequency. The stimuli orchestration component 1610 can perform a lookup in the profile data structure 1620 to determine a mode of stimulation or type of signal that maps to the second region of the brain. The stimuli orchestration component 1610 can compare the results of the lookup with the currently enabled mode of stimulation to determine that a third mode of stimulation is more likely to cause the neurons in the second region of the brain to oscillate at the desired frequency. Responsive to the determination, the stimuli orchestration component 1610 can identify a signaling component 1630a-n configured to generate and transmit signals corresponding to the selected third mode of stimulation and instruct or cause the identified signaling component 1630a-n to transmit the signals.

In some embodiments, the stimuli orchestration component 1610 can determine, based on feedback information, that a mode of stimulation is likely to affect the frequency of neural oscillation, or unlikely to affect the frequency of neural oscillation. The stimuli orchestration component 1610 can select a mode of stimulation from a plurality of modes of stimulation that is most likely to affect the frequency of neural stimulation or result in a desired frequency of neural oscillation. If the stimuli orchestration component 1610 determines, based on the feedback information, that a mode of stimulation is unlikely to affect the frequency of neural oscillation, the stimuli orchestration component 1610 can disable the mode of stimulation for a predetermined duration or until the feedback information indicates that the mode of stimulation would be effective.

The stimuli orchestration component 1610 can select one or more modes of stimulation to conserve resources or minimize resource utilization. For example, the stimuli orchestration component 1610 can select one or more modes of stimulation to reduce or minimize power consumption if the power source is a battery or if the battery level is low. In another example, the stimuli orchestration component 1610 can select one or more modes of stimulation to reduce heat generation if the ambient temperature is above a threshold or the temperature of the subject is above a threshold. In another example, the stimuli orchestration component 1610 can select one or more modes of stimulation to increase the level of attention if the stimuli orchestration component 1610 determines that the subject is not focusing on the stimulation (e.g., based on eye tracking or an undesired frequency of neural oscillations).

### Neural Stimulation Via Visual Stimulation and Auditory Stimulation

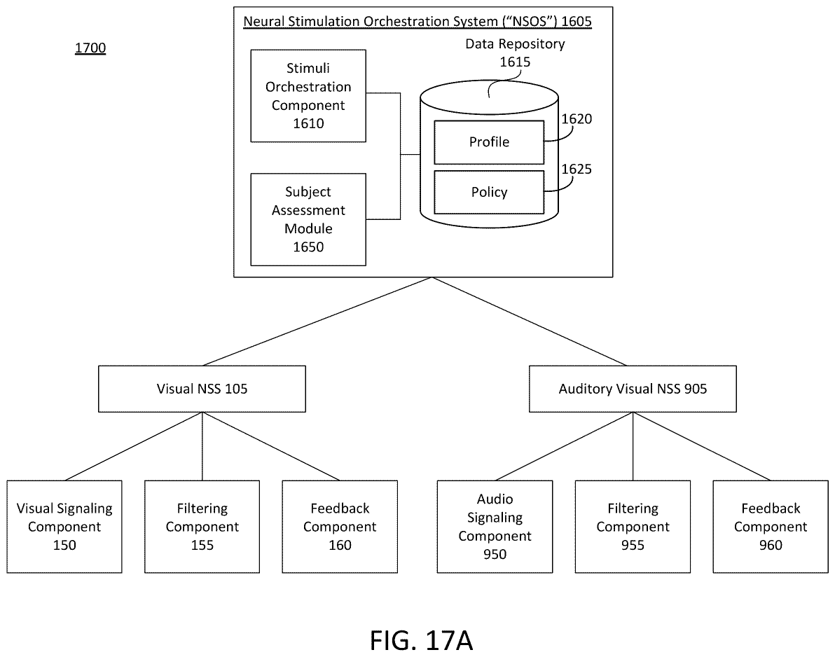

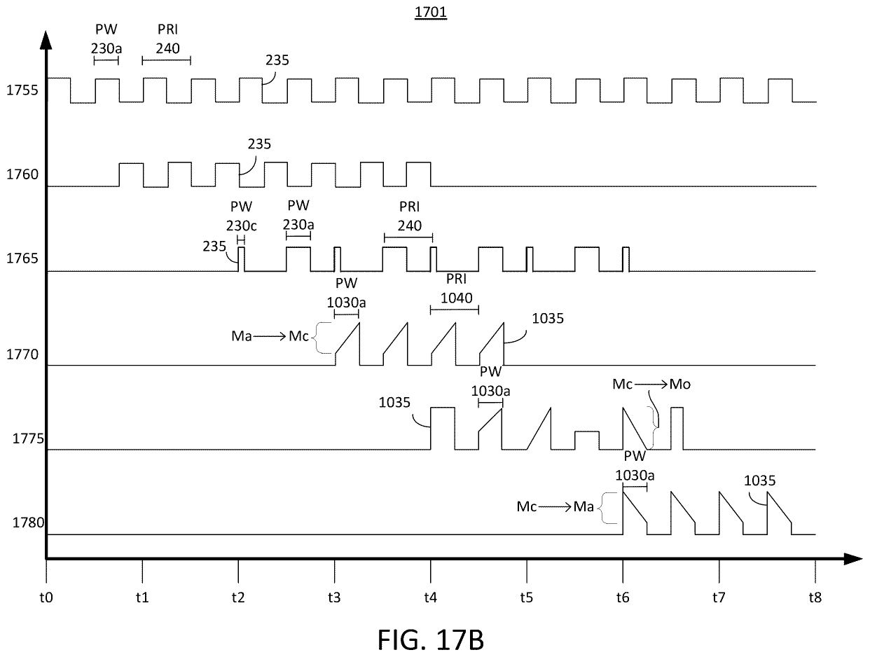

FIG. 17A is a block diagram depicting an embodiment of a system for neural stimulation via visual stimulation and auditory stimulation. The system 1700 can include the NSOS 1605. The NSOS 1605 can interface with the visual NSS 105 and the auditory NSS 905. The visual NSS 105 can interface or communicate with the visual signaling component 150, filtering component 155, and feedback component 160. The auditory NSS 905 can interface or communicate with the audio signaling component 950, filtering component 955, and feedback component 960.

To provide neural stimulation via visual stimulation and auditory stimulation, the NSOS 1605 can identify the types of available components for the neural stimulation session. The NSOS 1605 can identify the types of visual signals the visual signaling component 150 is configured to generate. The NSOS 1605 can also identify the type of audio signals the audio signaling component 950 is configured to generate. The NSOS 1605 can be configured about the types of visual signals and audio signals the components 150 and 950 are configured to generate. The NSOS 1605 can ping the components 150 and 950 for information about the components 150 and 950. The NSOS 1605 can query the components, send an SNMP request, broadcast a query, or otherwise determine information about the available visual signaling component 150 and audio signaling component 950.

For example, the NSOS 1605 can determine that the following components are available for neural stimulation: the visual signaling component 150 includes the virtual reality headset 401 depicted in FIG. 4C; the audio signaling component 950 includes the speaker 1205 depicted in FIG. 12B; the feedback component 160 includes an ambient light sensor 605, an eye tracker 605 and an EEG probe depicted in FIG. 4C; the feedback component 960 includes a microphone 1210 and feedback sensor 1225 depicted in FIG. 12B; and the filtering component 955 includes a noise cancellation component 1215. The NSOS 1605 can further determine an absence of filtering component 155 communicatively coupled to the visual NSS 105. The NSOS 1605 can determine the presence (available or online) or absence (offline) of components via visual NSS 105 or auditory NSS 905. The NSOS 1605 can further obtain identifiers for each of the available or online components.

The NSOS 1605 can perform a lookup in the profile data structure 1620 using an identifier of the subject to identify one or more types of visual signals and audio signals to provide to the subject. The NSOS 1605 can perform a lookup in the profile data structure 1620 using identifiers for the subject and each of the online components to identify one or more types of visual signals and audio signals to provide to the subject. The NSOS 1605 can perform a lookup up in the policy data structure 1625 using an identifier of the subject to obtain a policy for the subject. The NSOS 1605 can perform a lookup in the policy data structure 1625 using identifiers for the subject and each of the online components to identify a policy for the types of visual signals and audio signals to provide to the subject.

FIG. 17B is a diagram depicting waveforms used for neural stimulation via visual stimulation and auditory stimulation in accordance with an embodiment. FIG. 17B illustrates example sequences or a set of sequences 1701 that the stimuli orchestration component 1610 can generate or cause to be generated by one or more visual signaling components 150 or audio signal components 950. The stimuli orchestration component 1610 can retrieve the sequences from a data structure stored in data repository 1615 of NSOS 1605, or a data repository corresponding to NSS 105 or NSS 905. The sequences can be stored in a table format, such as TABLE 1 below. In some embodiments, the NSOS 1605 can select predetermined sequences to generate a set of sequences for a treatment session or time period, such as the set of sequences in TABLE 1. In some embodiments, the NSOS 1605 can obtain a predetermined or preconfigured set of sequences. In some embodiments, the NSOS 1605 can construct or generate the set of sequences, or each sequence based on information obtained from the subject assessment module 1650. In some embodiments, the NSOS 1605 can remove or delete sequences from the set of sequences based on feedback, such as adverse side effects. The NSOS 1605, via subject assessment module 1650, can include sequences that are more likely to stimulate neurons in a predetermined region of the brain to oscillate at a desired frequency.

The NSOS 1605 can determine, based on the profile information, policy, and available components, to use the following sequences illustrated in example TABLE 1 provide neural stimulation using both visual signals and auditory signals.

TABLE 1
Audio and Video Stimulation Sequences

| | | | | Stimu- | | |
|---|---|---|---|---|---|---|
| Sequence | | | Signal | lation | | Timing |
| Identifier | Mode | Signal Type | Parameter | Frequency | | Schedule |
| 1755 | visual | light pulses | Color: red; | 40 | Hz | {t0:t8) |
| | | from a laser | Intensity: | | | |
| | | light source | low; | | | |
| | | | PW: 230a | | | |
| 1760 | visual | checkerboard | color: | 40 | Hz | {t1:t4} |
| | | pattern | black/white; | | | |
| | | image from a | intensity: | | | |
| | | tablet display | high; | | | |
| | | screen light | PW: 230a | | | |
| | | source | | | | |
| 1765 | visual | modulated | PW: | 40 | Hz | {t2:t6} |
| | | ambient light | 230c/230a; | | | |
| | | by a frame | | | | |
| | | with actuated | | | | |
| | | shutters | | | | |
| 1770 | audio | music from | amplitude | 40 | Hz | {t3:t5} |
| | | headphones | variation | | | |
| | | or speakers | from Mₐ to | | | |
| | | connected to | M_{c}; | | | |
| | | an audio | PW: 1030a | | | |
| | | player | | | | |
| 1775 | audio | acoustic or | PW: 1030a; | 39.8 | Hz | {t4:t7} |
| | | audio bursts | frequency | | | |
| | | provided by | variation | | | |
| | | headphones | from M_{c} to | | | |
| | | or speakers | Mₒ; | | | |
| 1780 | audio | air pressure | PW: 1030a; | 40 | Hz | {t6:t8} |
| | | generated by | pressure | | | |
| | | a cochlear air | varies from | | | |
| | | jet | M_{c} to Mₐ | | | |

As illustrated in TABLE 1, each waveform sequence can include one or more characteristics, such as a sequence identifier, a mode, a signal type, one or more signal parameters, a modulation or stimulation frequency, and a timing schedule. As illustrated in FIG. 17B and TABLE 1, the sequence identifiers are 1755, 1760, 1765, 1765, 1770, 1775, and 1760.

The stimuli orchestration component 1610 can receive the characteristics of each sequence. The stimuli orchestration component 1610 can transmit, configure, load, instruct or otherwise provide the sequence characteristics to a signaling component 1630a-n. In some embodiments, the stimuli orchestration component 1610 can provide the sequence characteristics to the visual NSS 105 or the auditory NSS 905, while in some cases the stimuli orchestration component 1610 can directly provide the sequence characteristics to the visual signaling component 150 or audio signaling component 950.

The NSOS 1605 can determine, from the TABLE 1 data structure, that the mode of stimulation for sequences 1755, 1760 and 1765 is visual by parsing the table and identifying the mode. The NSOS 1605, responsive to determine the mode is visual, can provide the information or characteristics associated with sequences 1755, 1760 and 1765 to the visual NSS 105. The NSS 105 (e.g., via the light generation module 110) can parse the sequence characteristics and then instruct the visual signaling component 150 to generate and transmit the corresponding visual signals. In some embodiments, the NSOS 1605 can directly instruct the visual signaling component 150 to generate and transmit visual signals corresponding to sequences 1755, 1760 and 1765.

The NSOS 1605 can determine, from the TABLE 1 data structure, that the mode of stimulation for sequences 1770, 1775 and 1780 is audio by parsing the table and identifying the mode. The NSOS 1605, responsive to determine the mode is audio, can provide the information or characteristics associated with sequences 1770, 1775 and 1780 to the auditory NSS 905. The NSS 905 (e.g., via the light generation module 110) can parse the sequence characteristics and then instruct the audio signaling component 950 to generate and transmit the corresponding audio signals. In some embodiments, the NSOS 1605 can directly instruct the visual signaling component 150 to generate and transmit visual signals corresponding to sequences 1770, 1775 and 1780.

For example, the first sequence 1755 can include a visual signal. The signal type can include light pulses 235 generated by a light source 305 that includes a laser. The light pulses can include light waves having a wavelength corresponding to the color red in the visible spectrum. The intensity of the light can be set to low. An intensity level of low can correspond to a low contrast ratio (e.g., relative to the level of ambient light) or a low absolute intensity. The pulse width for the light burst can correspond to pulse width 230a depicted in FIG. 2C. The stimulation frequency can be 40 Hz, or the stimulation frequency can correspond to a pulse rate interval (“PRI”) of 0.025 seconds. The first sequence 1655 can run from t₀ to t₈. The first sequence 1655 can run for the duration of the session or treatment. The first sequence 1655 can run while one or more other sequences are other running. The time intervals can refer to absolute times, time periods, number of cycles, or some other event. The time interval from t₀ to t₈ can be, for example, 1 minute, 2 minutes, 3 minutes, 4 minutes, 5 minutes, 7 minutes, 10 minutes, 12 minutes, 15 minutes, 20 minutes, or more or less. The time interval can be cut short or terminated by the subject or responsive to feedback information. The time intervals can be adjusted based on profile information or by the subject via an input device.

The second sequence 1760 can be another visual signal that begins at t₁ and ends at t₄. The second sequence 1760 can include a signal type of a checkerboard image pattern that is provided by a display screen of a tablet. The signal parameters can include the colors black and white such that the checkerboard alternates black and white squares. The intensity can be high, which can correspond to a high contrast ratio relative to ambient light; or there can be a high contrast between the objects in the checkerboard pattern. The pulse width for the checkerboard pattern can be the same as the pulse width 230a as in sequence 1755. Sequence 1760 can begin and end at a different time than sequence 1755. For example, sequence 1760 can begin at t₁, which can be offset from to by 5 seconds, 10 seconds, 15 seconds, 20 seconds, 20 seconds, 30 seconds, 1 minute, 2 minutes, 3 minutes, or more or less. The visual signaling component 150 can initiate the second sequence 1760 at t₁, and it can terminate the second sequence at t₄. Thus, the second sequence 1760 can overlap with the first sequence 1755.

While pulse trains or sequences 1755 and 1760 can overlap with one another, the pulses 235 of the second sequence 1760 may not overlap with the pulses 235 of the first sequence 1755. For example, the pulses 235 of the second sequence 1760 can be offset from the pulses 235 of the first sequence 1755 such that they are non-overlapping.

The third sequence 1765 can include a visual signal. The signal type can include ambient light that is modulated by actuated shutters configured on frames (e.g., frames 400 depicted in FIG. 4B). The pulse width can vary from 230c to 230a in the third sequence 1765. The stimulation frequency can still be 40 Hz, such that the PRI is the same as the PRI in sequence 1760 and 1755. The pulses 235 of the third sequence 1765 can at least partially overlap with the pulses 235 of sequence 1755, but they may not overlap with the pulses 235 of the sequence 1760. Further, the pulse 235 can refer to block ambient light or allowing ambient light to be perceived by the eyes. In some embodiments, pulse 235 can correspond to blocking ambient light, in which case the laser light pulses 1755 may appear to have a higher contrast ratio. In some cases, the pulses 235 of sequence 1765 can correspond to allowing ambient light to enter the eyes, in which case the contrast ratio for pulses 235 of sequence 1755 may be lower, which may mitigate adverse side effects.

The fourth sequence 1770 can include an auditory stimulation mode. The fourth sequence 1770 can include up-chirp pulses 1035. The audio pulses can be provided via headphones or speakers 1205 of FIG. 12B. For example, the pulses 1035 can correspond to modulating music played by an audio player 1220 as depicted in FIG. 12B. The modulation can range from Ma to Mc. The modulation can refer to modulating the amplitude of the music. The amplitude can refer to the volume. Thus, the NSOS 1605 can instruct the audio signaling component 950 to increase the volume from a volume level Mₐ to a volume level M_{c} during a duration PW 1030a, and then return the volume to a baseline level or muted level in between pulses 1035. The PRI 240 can be 0.025, the PRI can or correspond to a 40 Hz stimulation frequency. The NSOS 1605 can instruct the fourth sequence 1770 to begin at t₃, which overlaps with visual stimulation sequences 1755, 1760 and 1765.

The fifth sequence 1775 can include another audio stimulation mode. The fifth sequence 1775 can include acoustic bursts. The acoustic bursts can be provided by the headphones or speakers 1205 of FIG. 12B. The sequence 1775 can include pulses 1035. The pulses 1035 can vary from one pulse to another pulse in the sequence. The fifth waveform 1775 can be configured to re-focus the subject to increase the subject's attention level to the neural stimulation. The fifth sequence 1775 can increase the subject's attention level by varying parameters of the signal from one pulse to the other pulse. The fifth sequence 1775 can vary the frequency from one pulse to the other pulse. For example, the first pulse 1035 in sequence 1775 can have a higher frequency than the previous sequences. The second pulse can be an up-chirp pulse having a frequency that increases from a low frequency to a high frequency. The third pulse can be a sharper up-chirp pulse that has frequency that increases from an even lower frequency to the same high frequency. The fifth pulse can have a low stable frequency. The sixth pulse can be a down-chirp pulse going from a high frequency to the lowest frequency. The seventh pulse can be a high frequency pulse with a small pulse width. The fifth sequence 1775 can being at t₄ and end at t₇. The fifth sequence can overlap with sequence 1755; and partially overlap with sequence 1765 and 1770. The fifth sequence may not overlap with sequence 1760. The stimulation frequency can be 39.8 Hz.

The sixth sequence 1780 can include an audio stimulation mode. The signal type can include pressure or air provided by an air jet. The sixth sequence can begin at t₆ and end at t₈. The sixth sequence 1780 can overlap with sequence 1755, and partially overlap with sequences 1765 and 1775. The sixth sequence 1780 can end the neural stimulation session along with the first sequence 1755. The air jet can provide pulses 1035 with pressure ranging from a high-pressure M_{c} to a low-pressure Mₐ. The pulse width can be 1030a, and the stimulation frequency can be 40 Hz.

The NSOS 1605 can adjust, change, or otherwise modify sequences or pulses based on feedback. In some embodiments, the NSOS 1605 can determine, based on the profile information, policy, and available components, to provide neural stimulation using both visual signals and auditory signals. The NSOS 1605 can determine to synchronize the transmit time of the first visual pulse train and the first audio pulse train. The NSOS 1605 can transmit the first visual pulse train and the first audio pulse train for a first duration (e.g., 1 minute, 2 minutes, or 3 minutes). At the end of the first duration, the NSOS 1605 can ping an EEG probe to determine a frequency of neural oscillation in a region of the brain. If the frequency of oscillation is not at the desired frequency of oscillation, the NSOS 1605 can select a sequence out of order or change the timing schedule of a sequence.

For example, the NSOS 1605 can ping a feedback sensor at t₁. The NSOS 1605 can determine, at t₁, that neurons of the primary visual cortex are oscillating at the desired frequency. Thus, the NSOS 1605 can determine to forego transmitting sequences 1760 and 1765 because neurons of the primary visual cortex are already oscillating at the desired frequency. The NSOS 1605 can determine to disable sequences 1760 and 1765. The NSOS 1605, responsive to the feedback information, can disable the sequences 1760 and 1765. The NSOS 1605, responsive to the feedback information, can modify a flag in the data structure corresponding to TABLE 1 to indicate that the sequences 1760 and 1765 are disabled.

The NSOS 1605 can receive feedback information at t₂. At t₂, the NSOS 1605 can determine that the frequency of neural oscillation in the primary visual cortex is different from the desired frequency. Responsive to determining the difference, the NSOS 1605 can enable or re-enable sequence 1765 in order to stimulate the neurons in the primary visual cortex such that the neurons may oscillate at the desired frequency.

Similarly, the NSOS 1605 can enable or disable audio stimulation sequences 1770, 1775 and 1780 based on feedback related to the auditory cortex. In some cases, the NSOS 1605 can determine to disable all audio stimulation sequences if the visual sequence 1755 is successfully affecting the frequency of neural oscillations in the brain at each time period t₁, t₂, t₃, t₄, t₅, t₆, t₇, and t₈. In some cases, the NSOS 1605 can determine that the subject is not paying attention at t₄, and it can go from only enabling visual sequence 1755 directly to enabling audio sequence 1755 to re-focus the user using a different stimulation mode.

### Method for Neural Stimulation Via Visual Stimulation and Auditory Stimulation

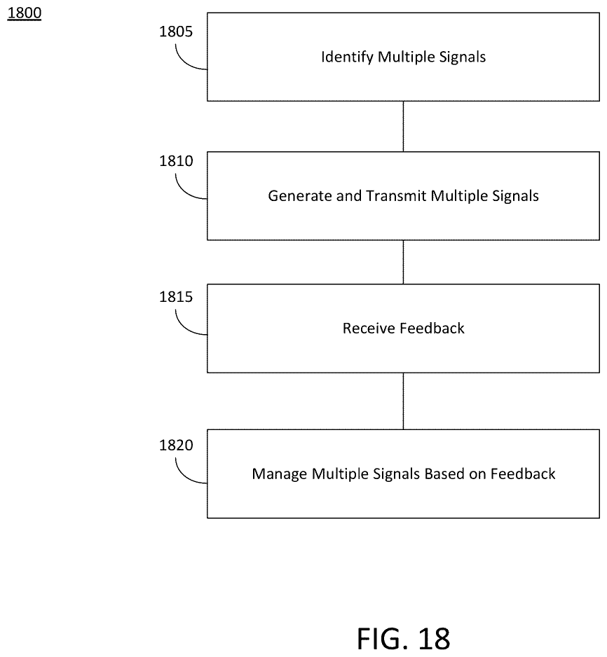

FIG. 18 is a flow diagram of a method for neural stimulation via visual stimulation and auditory stimulation in accordance with an embodiment. The method 180 can be performed by one or more system, component, module, or element depicted in FIGS. 1-17B, including, for example, a neural stimulation orchestration component or neural stimulations system. In brief overview, the NSOS can identify multiple modes of signals to provide at block 1805. At block 1810, the NSOS can generate and transmit the identified signals corresponding to the multiple modes. A block 1815, the NSOS can receive or determine feedback associated with neural activity, physiological activity, environmental parameters, or device parameters. At block 1820, the NSOS can manage, control, or adjust the one or more signals based on the feedback.

While this specification contains many specific implementation details, these should not be construed as limitations on the scope of any inventions or of what can be claimed, but rather as descriptions of features specific to particular embodiments of particular aspects. Certain features described in this specification in the context of separate embodiments can also be implemented in combination in a single embodiment. Conversely, various features described in the context of a single embodiment can also be implemented in multiple embodiments separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the embodiments described above should not be understood as requiring such separation in all embodiments, and it should be understood that the described program components and systems can generally be integrated in a single software product or packaged into multiple software products.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’.

Thus, particular exemplary embodiments of the subject matter have been described. In some cases, the actions recited in the claims can be performed in a different order and still achieve desirable results. In addition, the processes depicted in the accompanying figures do not necessarily require the particular order shown, or sequential order, to achieve desirable results.

The present technology, including the systems, methods, devices, components, modules, elements, or functionality described or illustrated in, or in association with, the figures can treat, prevent, protect against, or otherwise affect brain atrophy and disorders, conditions and diseases associated with brain atrophy.

Neural Stimulation System with Sleep-Related Monitoring Modules

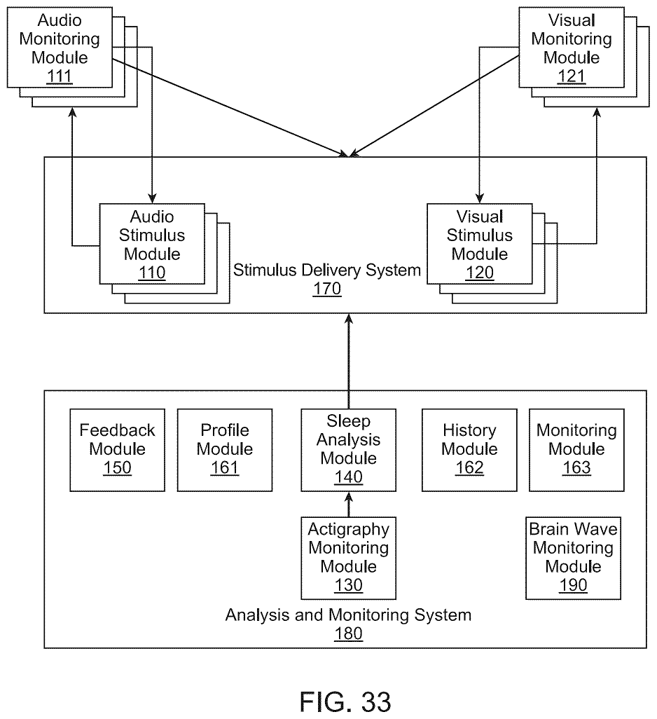

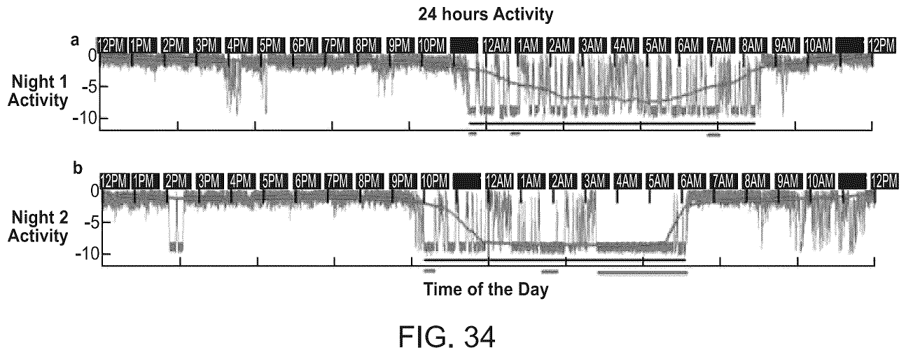

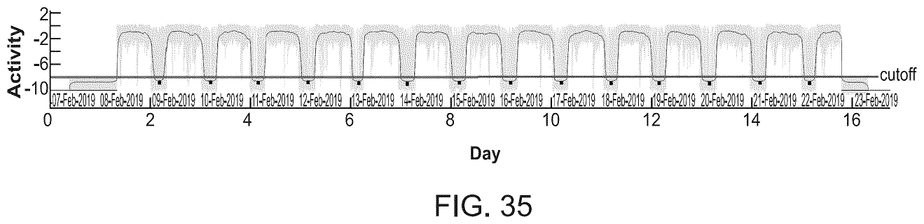

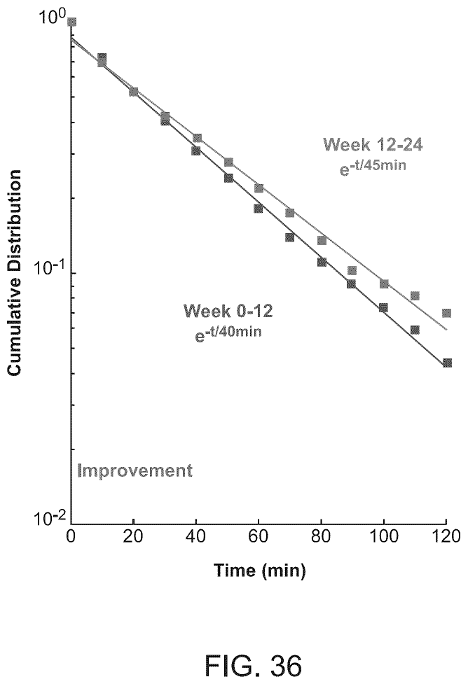

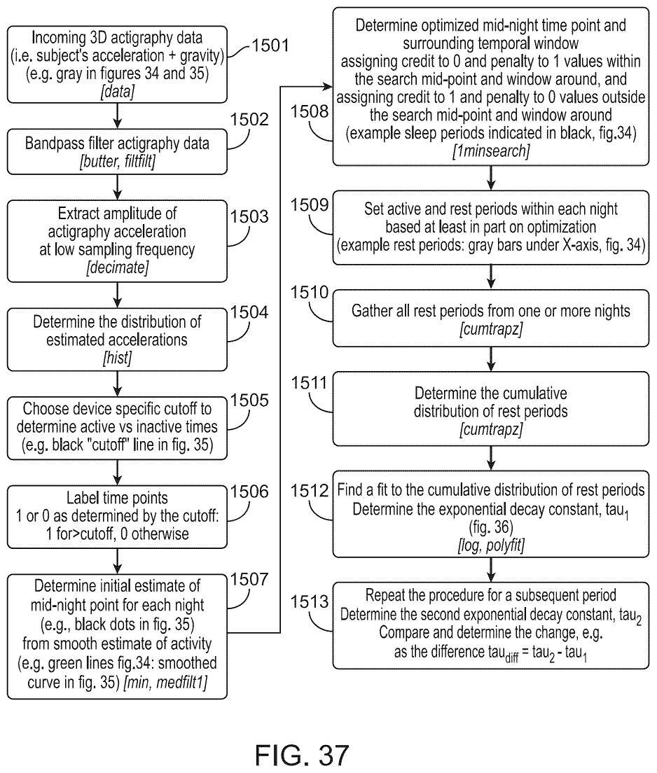

FIG. 33 provides a neural stimulation system comprising a stimulus delivery system coupled to an analysis and monitoring system. In some embodiments, the present technological solution comprises a stimulus delivery system which includes one or more of: one or more Audio Stimulus Module (110), one or more Visual Stimulus Module (120). These modules may be in addition to tactile or other stimulus modules (not shown). These modules provide the delivery of audio or visual stimulus at specific parameter values. In some embodiments the values of these parameters are responsive to one or more of: one or more Audio Monitoring Module (111), one or more Visual Monitoring Module (121).

In some embodiments, the present technological solution includes one or more of: one or more Feedback Module (150) collecting, storing, or processing feedback from users or third parties; one or more Profile Module (161) storing and processing profile or demographic information related to one or more users or third parties, or of populations of users or third parties; one or more History Module (162) storing or processing history and logs related to one or more users or third parties, or of populations of users or third parties; one or more Monitoring Module (163), collecting, storing, logging, and/or analyzing aspects of one or more users or third parties, including but not limited to: aspects of the environment, state, behavior, input, responses, diagnosis, disease progression, compliance, engagement, mood, adherence. In some embodiments the present technological solution includes one or more Brain Wave Monitoring Module (190) measuring and analyzing brain wave activity in one or more users, including but not limited to detecting and characterizing gamma wave power and sensory induction of gamma neural oscillations.

In some embodiments, the present technological solution includes one or more of: one or more Actigraphy Monitoring Module (130), one or more Sleep Analysis Module (140). In some embodiments, one or more Sleep Analysis Module is responsive, at least in part, to information communicated from one or more Actigraphy Monitoring Module. In some embodiments, a Sleep Analysis Module performs sleep analysis based at least in part on actigraphy information collected at least in part by an Actigraphy Monitoring Module. In some embodiments, Sleep Analysis Module performs one or more analysis steps described in FIG. 37.

In some embodiments, one or more of an Audio Stimulus Module, a Visual Stimulus Module, and/or a Stimulus Delivery System (170) managing or incorporating one or more stimulus modules, may be responsive to one or more of: one or more Analysis and Monitoring System (130) and/or monitoring modules, including but not limited to: one or more Feedback Module (150), one or more Profile Module (161), one or more History Module (162), one or more Monitoring Module (163), one or more Sleep Analysis Module (140), one or more Actigraphy Monitoring Module (130), one or more Brain Wave Monitoring Module (190), and/or one or more Stimulus Delivery System (170) managing or incorporating one or more analysis and monitoring module.

Neural Stimulation System with Imperceptible Stimulus

The present disclosure describes a method for neuromodulating a subject, comprising displaying a cognitively engaging content and providing a gamma oscillation inducing non-invasive sensory stimulus via a display device, wherein the gamma oscillation inducing non-invasive sensory stimulus: (a) comprises an average amplitude, a duty cycle, or both that renders the gamma oscillation inducing non-invasive sensory stimulus imperceptible to the subject; and (b) causes a therapeutic improvement in a cognitive function, thereby neuromodulating the subject.

The advantage of providing the gamma oscillation inducing non-invasive sensory stimulus together with a cognitively engaging content can be that the therapeutic benefits from the gamma oscillation inducing non-invasive sensory stimulus may be experienced through daily activities of the subject. The subject can receive the gamma oscillation inducing non-invasive sensory stimulus while experiencing various cognitively engaging content, for instance, a movie. In some embodiments, the cognitively engaging content may comprise a picture. In some embodiments, the cognitively engaging content may comprise a video. In some embodiments, the cognitively engaging content may comprise a game. In some embodiments, the cognitively engaging content may comprise a writing. In some embodiments, the cognitively engaging content may comprise a story. In some embodiments, the cognitively engaging content may comprise a song. In some embodiments, the cognitively engaging content may comprise music. In some embodiments, the cognitively engaging content may comprise ambient noise.

The advantage of providing the gamma oscillation inducing non-invasive sensory stimulus such that it is imperceptible to the subject can be that the cognitively engaging content will not be perceptually different, changed, or disrupted by the gamma oscillation inducing non-invasive sensory stimulus. Another advantage of providing the gamma oscillation inducing non-invasive sensory stimulus such that it is imperceptible can be that when the subject is participating in the cognitively engaging content with other people (e.g., friends and family), the cognitively engaging content will not be perceptually different, changed, or disrupted by the gamma oscillation inducing non-invasive sensory stimulus to the other people.

In some embodiments, displaying may comprise displaying a visual stimulus. In some embodiments, displaying may comprise displaying an auditory stimulus. In some embodiments, displaying may comprise displaying a haptic stimulus. In some embodiments, the gamma oscillation inducing non-invasive sensory stimulus may be imperceptible to the subject. In some embodiments, the gamma oscillation inducing non-invasive sensory stimulus may be imperceptible when displayed in combination with the cognitively engaging content. In some embodiments, the gamma oscillation inducing non-invasive sensory stimulus may be imperceptible to the subject's vision. In some embodiments, the gamma oscillation inducing non-invasive sensory stimulus may be imperceptible to the subject's sense of hearing. In some embodiments, the gamma oscillation inducing non-invasive sensory stimulus may be imperceptible to the subject's sense of touch.

In some embodiments, the intensity of the gamma oscillation inducing non-invasive sensory stimulus may be at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 candelas per square meter. In some embodiments, the intensity of the gamma oscillation inducing non-invasive sensory stimulus may be at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 candelas per square meter.

In some embodiments, the intensity of the gamma oscillation inducing non-invasive sensory stimulus may be at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 nits. In some embodiments, the intensity of the gamma oscillation inducing non-invasive sensory stimulus may be at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 nits.

In some embodiments, the intensity of the gamma oscillation inducing non-invasive sensory stimulus may be at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 decibels. In some embodiments, the intensity of the gamma oscillation inducing non-invasive sensory stimulus may be at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 decibels.

In some embodiments, the duty cycle of the gamma oscillation inducing non-invasive sensory stimulus may be at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 91, 92, 93, 94, 95, 96, 97, 98, 99, 99.1, 99.2, 99.3, 99.4, 99.5, 99.6, 99.7, 99.8, or 99.9 percent duty cycle. In some embodiments, the duty cycle of the gamma oscillation inducing non-invasive sensory stimulus may be at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 91, 92, 93, 94, 95, 96, 97, 98, 99, 99.1, 99.2, 99.3, 99.4, 99.5, 99.6, 99.7, 99.8, or 99.9 percent duty cycle.

In some embodiments, the average amplitude of the gamma oscillation inducing non-invasive sensory stimulus may be at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 91, 92, 93, 94, 95, 96, 97, 98, 99, 99.1, 99.2, 99.3, 99.4, 99.5, 99.6, 99.7, 99.8, or 99.9 percent of the average amplitude of the cognitively engaging content. In some embodiments, the average amplitude of the gamma oscillation inducing non-invasive sensory stimulus may be at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 91, 92, 93, 94, 95, 96, 97, 98, 99, 99.1, 99.2, 99.3, 99.4, 99.5, 99.6, 99.7, 99.8, or 99.9 percent of the average amplitude of the cognitively engaging content.

In some embodiments, neuromodulating may comprise neuromodulating at least a portion of the subject's nervous system. In some embodiments, neuromodulating may comprise neuromodulating at least a portion of the subject's retina. In some embodiments, neuromodulating may comprise neuromodulating at least a portion of the subject's cone cells. In some embodiments, neuromodulating may comprise neuromodulating at least a portion of the subject's rod cells. In some embodiments, neuromodulating may comprise neuromodulating at least a portion of the subject's retinal ganglion cell axons. In some embodiments, neuromodulating may comprise neuromodulating at least a portion of the subject's glial cells. In some embodiments, neuromodulating may comprise neuromodulating at least a portion of the subject's optic nerve. In some embodiments, neuromodulating may comprise neuromodulating at least a portion of the subject's optic chiasma. In some embodiments, neuromodulating may comprise neuromodulating at least a portion of the subject's optic tract. In some embodiments, neuromodulating may comprise neuromodulating at least a portion of the subject's lateral geniculate nucleus. In some embodiments, neuromodulating may comprise neuromodulating at least a portion of the subject's pretectal nuclei. In some embodiments, neuromodulating may comprise neuromodulating at least a portion of the subject's superior colliculus. In some embodiments, neuromodulating may comprise neuromodulating at least a portion of the subject's optic nerve. In some embodiments, neuromodulating may comprise neuromodulating at least a portion of the subject's Commissure of Gudden. In some embodiments, neuromodulating may comprise neuromodulating at least a portion of the subject's pulvinar. In some embodiments, neuromodulating may comprise neuromodulating at least a portion of the subject's medial geniculate body. In some embodiments, neuromodulating may comprise neuromodulating at least a portion of the subject's nucleus of oculomotor nucleus. In some embodiments, neuromodulating may comprise neuromodulating at least a portion of the subject's trochlar nerve nucleus. In some embodiments, neuromodulating may comprise neuromodulating at least a portion of the subject's abducent nerve nucleus. In some embodiments, neuromodulating may comprise neuromodulating at least a portion of the subject's occipital lobes.

In some embodiments, neuromodulating may comprise neuromodulating at least a portion of the subject's cochlear nerve. In some embodiments, neuromodulating may comprise neuromodulating at least a portion of the subject's auditory nerve. In some embodiments, neuromodulating may comprise neuromodulating at least a portion of the subject's vestibular nerve. In some embodiments, neuromodulating may comprise neuromodulating at least a portion of the subject's vestibulocochlear nerve. In some embodiments, neuromodulating may comprise neuromodulating at least a portion of the subject's pons. In some embodiments, neuromodulating may comprise neuromodulating at least a portion of the subject's auditory cortex.

In some embodiments, neuromodulating may comprise neuromodulating at least a portion of the subject's somatosensory system. In some embodiments, neuromodulating may comprise neuromodulating at least a portion of the subject's posterior nerve roots. In some embodiments, neuromodulating may comprise neuromodulating at least a portion of the subject's nucleus gracilis. In some embodiments, neuromodulating may comprise neuromodulating at least a portion of the subject's nucleus cunealus. In some embodiments, neuromodulating may comprise neuromodulating at least a portion of the subject's fasciculus gracilis. In some embodiments, neuromodulating may comprise neuromodulating at least a portion of the subject's fasciculus cunealus. In some embodiments, neuromodulating may comprise neuromodulating at least a portion of the subject's medial lemniscus.

In some embodiments, neuromodulating may comprise neuromodulating at least a portion of the subject's brain. In some embodiments, neuromodulating may comprise neuromodulating at least a portion of the subject's frontal lobe. In some embodiments, neuromodulating may comprise neuromodulating at least a portion of the subject's cerebral cortex. In some embodiments, neuromodulating may comprise neuromodulating at least a portion of the subject's parietal lobe. In some embodiments, neuromodulating may comprise neuromodulating at least a portion of the subject's temporal lobe. In some embodiments, neuromodulating may comprise neuromodulating at least a portion of the subject's cerebellum. In some embodiments, neuromodulating may comprise neuromodulating at least a portion of the subject's brain stem. In some embodiments, neuromodulating may comprise neuromodulating at least a portion of the subject's medulla oblongata. In some embodiments, neuromodulating may comprise neuromodulating at least a portion of the subject's oculomotor nerve. In some embodiments, neuromodulating may comprise neuromodulating at least a portion of the subject's hypophysis. In some embodiments, neuromodulating may comprise neuromodulating at least a portion of the subject's posterior lobe. In some embodiments, neuromodulating may comprise neuromodulating at least a portion of the subject's mammillary. In some embodiments, neuromodulating may comprise neuromodulating at least a portion of the subject's anterior lobe body. In some embodiments, neuromodulating may comprise neuromodulating at least a portion of the subject's infundibulum. In some embodiments, neuromodulating may comprise neuromodulating at least a portion of the subject's rostrum of corpus callosum. In some embodiments, neuromodulating may comprise neuromodulating at least a portion of the subject's genu of corpus callosum. In some embodiments, neuromodulating may comprise neuromodulating at least a portion of the subject's column of fornix. In some embodiments, neuromodulating may comprise neuromodulating at least a portion of the subject's body of fornix. In some embodiments, neuromodulating may comprise neuromodulating at least a portion of the subject's body of corpus callosum. In some embodiments, neuromodulating may comprise neuromodulating at least a portion of the subject's thalamus. In some embodiments, neuromodulating may comprise neuromodulating at least a portion of the subject's intermediate mass of thalamus. In some embodiments, neuromodulating may comprise neuromodulating at least a portion of the subject's posterior commissure. In some embodiments, neuromodulating may comprise neuromodulating at least a portion of the subject's pineal body. In some embodiments, neuromodulating may comprise neuromodulating at least a portion of the subject's trigeminal nerve. In some embodiments, neuromodulating may comprise neuromodulating at least a portion of the subject's facial nerve. In some embodiments, neuromodulating may comprise neuromodulating at least a portion of the subject's acoustic nerve. In some embodiments, neuromodulating may comprise neuromodulating at least a portion of the subject's glossopharyngeal nerve. In some embodiments, neuromodulating may comprise neuromodulating at least a portion of the subject's vagus nerve. In some embodiments, neuromodulating may comprise neuromodulating at least a portion of the subject's hypoglossal nerve. In some embodiments, neuromodulating may comprise neuromodulating at least a portion of the subject's root filaments of cervical nerve. In some embodiments, neuromodulating may comprise neuromodulating at least a portion of the subject's tonsil of cerebellum. In some embodiments, neuromodulating may comprise neuromodulating at least a portion of the subject's abducent nerve. In some embodiments, neuromodulating may comprise neuromodulating at least a portion of the subject's interpeduncular fossa. In some embodiments, neuromodulating may comprise neuromodulating at least a portion of the subject's trochlear nerve. In some embodiments, neuromodulating may comprise neuromodulating at least a portion of the subject's semilunar ganglion. In some embodiments, neuromodulating may comprise neuromodulating at least a portion of the subject's mandibular nerve. In some embodiments, neuromodulating may comprise neuromodulating at least a portion of the subject's ophthalmic nerve. In some embodiments, neuromodulating may comprise neuromodulating at least a portion of the subject's maxillary nerve. In some embodiments, neuromodulating may comprise neuromodulating at least a portion of the subject's olfactory trigone. In some embodiments, neuromodulating may comprise neuromodulating at least a portion of the subject's tuber cinereum. In some embodiments, neuromodulating may comprise neuromodulating at least a portion of the subject's olfactory tract. In some embodiments, neuromodulating may comprise neuromodulating at least a portion of the subject's olfactory bulb.

In some embodiments, the subject may have a cognitive disorder. In some embodiments, the subject may have Alzheimer's disease. In some embodiments, the subject may have behavioral variant frontotemporal dementia. In some embodiments, the subject may have corticobasal degeneration. In some embodiments, the subject may have Huntington's disease. In some embodiments, the subject may have Lewy body dementia. In some embodiments, the subject may have mild cognitive impairment. In some embodiments, the subject may have primary progressive aphasia. In some embodiments, the subject may have progressive supranuclear palsy. In some embodiments, the subject may have vascular dementia. In some embodiments, the subject may have Parkinson's disease. In some embodiments, the subject may have a concussion. In some embodiments, the subject may have attention deficit disorder. In some embodiments, the subject may have early onset dementia. In some embodiments, the subject may have epilepsy. In some embodiments, the subject may have normal pressure hydrocephalus. In some embodiments, the subject may have posterior cortical atrophy. In some embodiments, the subject may have a stroke. In some embodiments, the subject may have a traumatic brain injury. In some embodiments, the subject may have multiple sclerosis. In some embodiments, the subject may have chemotherapy related cognitive impairment. In some embodiments, the subject may be afflicted with neurosis. In some embodiments, the subject may be afflicted with anxiety. In some embodiments, the subject may be afflicted with depression. In some embodiments, the subject may be afflicted with an addiction. In some embodiments, the subject may be afflicted with an eating disorder. In some embodiments, the subject may be afflicted with a sleeping disorder. In some embodiments, the subject may be afflicted insomnia. In some embodiments, the subject may be afflicted with sleep-fragmentation. In some embodiments, the subject may be afflicted Alzheimer's associated sleep-fragmentation.

Various expected treatment outcomes may be predicted by a method of the present disclosure method. In some embodiments, the expected treatment outcome may comprise an improvement in or a deterioration in neurotic behavior. In some embodiments, the expected treatment outcome may comprise an improvement in or a deterioration in anxious behavior. In some embodiments, the expected treatment outcome may comprise an improvement in or a deterioration in depressive behavior. In some embodiments, the expected treatment outcome may comprise an improvement in or a deterioration in addictive behavior. In some embodiments, the expected treatment outcome may comprise an improvement in or a deterioration in food-seeking behavior. In some embodiments, the cognitive function may comprise sleeping behavior.

The expected treatment outcome in the subject's cognitive function may be measured by various clinically relevant measure for the cognitive function. In some embodiments, the expected treatment outcome may comprise an improvement in the subject's cognitive function as measured by self-reported levels of improvement of the cognitive function. In some embodiments, the expected treatment outcome may comprise an improvement in the subject's cognitive function as measured by a change in the average heart rate of the subject over the course of a minute, an hour, a day, a week, a month, or a year.

In some embodiments, the expected treatment outcome in the anxious behavior of the subject may comprise an expected improvement in the subject's anxious behavior as measured by the State-Trait Anxiety Inventory, Beck Anxiety Inventory, or Hospital Anxiety and Depression Scale-Anxiety.

In some embodiments, the expected treatment outcome in the depressive behavior of the subject may comprise an expected improvement in the subject's depressive behavior as measured by Beck Depression Inventory, Center For Epidemiological Study-Depression Scale, Geriatric Depression Scale, Hamilton Rating Scale For Depression, Montgomery Åsberg Depression Rating Scale, Patient Health Questionnaire, Patient Health Questionnaire-9, Quick Inventory of Depressive Symptomatology-Clinician Rated 16, or Quick Inventory of Depressive Symptomatology-Self Reported 16.

In some embodiments, the expected treatment outcome in the addictive behavior of the subject may comprise an expected improvement in the subject's addictive behavior as measured by Addiction Severity Instrument, Alcohol Dependence Scale, Benzodiazepine Questionnaire, Chemical Use Abuse and Dependence Scale, Drug Use Screening Inventory, Global Appraisal of Individual Needs, Severity of Alcohol Dependence Questionnaire, Severity of Amphetamine Dependence, Severity of Dependence Scale, Severity of Opiate Dependence Questionnaire, Substance Dependence Severity Scale, or Substance Use Involvement Index.

In some embodiments, the expected treatment outcome in the food-seeking behavior of the subject may comprise an expected improvement in the subject's food-seeking behavior as measured by Eating Disorder Examination, Eating Disorder Examination Questionnaire, Clinical Impairment Assessment, Clinical Perfectionism Questionnaire, Eating Problem Check List, or Starvation Symptoms Inventory.

In some embodiments, the expected treatment outcome may comprise an expected improvement in the subject's sleeping behavior as measured by reduction in the frequency of sleep fragmentation. In some embodiments, the expected treatment outcome may comprise an expected improvement in the subject's sleeping behavior as measured by increase in the regularity of sleep cycles. In some embodiments, the expected treatment outcome may comprise an expected improvement in the subject's sleeping behavior as measured by self-reported quality of sleep by the subject.

In some embodiments, the expected treatment outcome in the sleeping behavior of the subject may comprise an expected improvement in the subject's sleeping behavior as measured by Multiple Sleep Latency Test, International Restless Legs Scale, Johns Hopkins Restless Legs Severity Scale, Restless Legs Syndrome-6 Measure, Pediatric Restless Legs Syndrome Severity Scale, Augmentation Severity Rating Scale, Epworth Sleepiness Scale, Stanford Sleepiness Scale, or the Parkinson's Disease-Sleep-Daytime Sleepiness Subscale, or Inappropriate Sleep Composite Score.

In some embodiments, the expected treatment outcome may comprise an expected improvement in the subject's sleeping behavior as measured using a polysomnography device. In some embodiments, the expected treatment outcome may comprise an expected improvement in the subject's sleeping behavior as measured using an actigraphy device. In some embodiments, the expected treatment outcome may comprise an expected improvement in the subject's sleeping behavior as measured using a cyclic alternating pattern device.

In some embodiments, the gamma oscillation inducing non-invasive sensory stimulus changes the levels of a chemical in the subject. In some embodiments, the chemical is a sugar. In some embodiments, the chemical is glucose. In some embodiments, the chemical is a hormone. In some embodiments, the chemical is dopamine, serotonin, cortisol, oxytocin, endorphin, cortisol, or insulin. In some embodiments, the chemical is glutamate or norepinephrine. In some embodiments, the chemical is adrenaline. In some embodiments, changing the levels of the chemical is localized in a brain region of the subject.

In some embodiments, the expected treatment outcome may comprise an expectation of increasing slow wave activity during non-REM sleep of the subject. In some embodiments, the expected treatment outcome may comprise an expected improvement in the subject's sensitivity to a chemical in the subject's brain region. In some embodiments, the expected treatment outcome may comprise an expected change in the metabolism of the subject. In some embodiments, the expected treatment outcome may comprise an expected promotion of brain oscillatory activity. In some embodiments, the expected treatment outcome may comprise an expected slowing the progression of cognitive decline. In some embodiments, the expected treatment outcome may comprise an expected slowing the progression of age-related cognitive decline. In some embodiments, the expected treatment outcome may comprise an expected slowing the progression of Alzheimer's Disease associated cognitive decline.

In some embodiments, the expected treatment outcome may comprise an expected change in the bioactivity of one or more cells in the subject. In some embodiments, the one or more cells may comprise a microglial cell. In some embodiments, the one or more cells may comprise an astrocyte. In some embodiments, the one or more cells may comprise a myeloid cell. In some embodiments, the one or more cells may comprise a monocyte. In some embodiments, the one or more cells may comprise a macrophage. In some embodiments, the one or more cells may comprise a dendritic cell. In some embodiments, the one or more cells may comprise a T cell. In some embodiments, the one or more cells may comprise a B cell. In some embodiments, the one or more cells may comprise a natural killer cell.

In some embodiments, the bioactivity may comprise the clearing activity of microglial cells. In some embodiments, the clearing activity may comprise clearing cellular debris in a brain region. In some embodiments, the clearing activity may comprise clearing a chemical in a brain region. In some embodiments, the clearing activity may comprise clearing protein in a brain region. In some embodiments, the clearing activity may comprise clearing amyloid beta protein in a brain region. In some embodiments, the clearing activity may comprise clearing amyloid beta protein precursors in a brain region. In some embodiments, the clearing activity may comprise clearing amyloid beta protein metabolites in a brain region.

In some embodiments, the bioactivity may comprise the lifecycle of microglial cells. In some embodiments, the bioactivity may comprise increasing the number of microglial cells in a brain region. In some embodiments, the bioactivity may comprise reducing the number of microglial cells in a brain region. In some embodiments, the bioactivity may comprise increasing the life expectancy of microglial cells in a brain region. In some embodiments, the bioactivity may comprise reducing the number of microglial cells in a brain region.

In some embodiments, the bioactivity may comprise an immune response. In some embodiments, the bioactivity may comprise increasing the magnitude of the immune response. In some embodiments, the bioactivity may comprise reducing the magnitude of the immune response. In some embodiments, the bioactivity may comprise reducing inflammation caused by an immune response. In some embodiments, the bioactivity may be present in a brain region.

In some embodiments, the method further comprises administering a therapeutically effective amount of a pharmaceutical after determining an expected treatment outcome. In some embodiments, the pharmaceutical may be an anti-depressant, an anxiolytic, an antipsychotic, a selective serotonin reuptake inhibitor, a sedative, or a pharmaceutical for treating Alzheimer's disease. In some embodiments, the pharmaceutical may comprise donepezil, galantamine, memantine, rivastigmine, aducanumab, fluoxetine, escitalopram, sertraline, fluvoxamine, citalopram, paroxetine, amitriptyline, mirtazapine, imipramine, bupropion, nortriptyline, trazodone, duloxetine, desvenlafaxine, venlafaxine, selegiline, buspirone, aripiprazole, diazepam, lorazepam, clonazepam, oxazepam, clomipramine, pregabalin, methadone, buprenorphine, or varenicline.

In some embodiments, the display device may comprise a display made using one or more of a light emitting diode. In some embodiments, the display device may comprise an electroluminescent display. In some embodiments, the display device may comprise a liquid crystal display. In some embodiments, the display device may comprise a backlit liquid crystal display. In some embodiments, the display device may comprise an organic light emitting diode display. In some embodiments, the display device may comprise a plasma display. In some embodiments, the display device may comprise a quantum dot display. In some embodiments, the display device may comprise a thin-film transistor display. In some embodiments, the display device may comprise a digital light processing display. In some embodiments, the display device may comprise a laser display. In some embodiments, the display device may comprise a microLED display.

In some embodiments, the light emitting diode may comprise an organic electroluminescent diode. In some embodiments, the light emitting diode may comprise a quantum dot diode. In some embodiments, the light emitting diode may comprise an active-matrix organic light-emitting diode. In some embodiments, the light emitting diode may comprise an electroluminescent diode.

In some embodiments, the display device may comprise a tablet. In some embodiments, the display device may comprise a phone. In some embodiments, the display device may comprise a computer. In some embodiments, the display device may comprise a television. In some embodiments, the display device may comprise a watch.

### Neural Stimulation System to Increase Gamma Oscillations

The present disclosure describes a device comprising: (a) a display configured to display a cognitively engaging content; (b) a stimulation source operatively coupled to the display, the stimulation source configured to emit a stimulus having a frequency that causes an increase in gamma oscillations, wherein the stimulus is displayed in association with the cognitively engaging content.

In some embodiments, the device may comprise a refresh rate from about 55 Hz and 65 Hz. In some embodiments, the device may comprise a refresh rate from about 115 Hz and 125 Hz. In some embodiments, the device may comprise a refresh rate from about 175 Hz and 185 Hz. In some embodiments, the device may comprise a frame rate from about 55 Hz and 65 Hz. In some embodiments, the device may comprise a frame rate from about 115 Hz and 125 Hz. In some embodiments, the device may comprise a frame rate from about 175 Hz and 185 Hz.

In some embodiments, the stimulation source may form at least a portion of a perimeter surrounding the cognitively engaging content. In some embodiments, the portion of the perimeter may be adjacent to the cognitively engaging content. In some embodiments, the portion of the perimeter may be separated from the cognitively engaging content by at least about 1 mm. In some embodiments, the portion of the perimeter may be separated from the cognitively engaging content by at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mm.

The perimeter may comprise various shapes and sizes. In some embodiments, the perimeter may be approximately rectangular, circular, triangular or any other shape. In some embodiments, the perimeter may form a section of a shape, e.g., an arc of a circle, one side of a rectangle, two sides of a rectangle, or three sides of a rectangle.

In some embodiments, the simulation source may be configured to be mounted on or near the display. In some embodiments, the stimulation source may be configured to be mounted on a tablet, a television, a phone, or a watch. In some embodiments, the stimulation source may be configured to be mounted on a light fixture. In some embodiments, the stimulation source is configured to be wearable by the subject. The device may be various household objects. In some embodiments, the device may be a light fixture, a lamp, a laptop screen, a computer screen, a speaker, an earbug, or an electronic picture frame.

In some embodiments, the stimulation source may comprise a filter, wherein the filter displays the gamma-oscillation inducing non-invasive sensory stimulus by blocking out at least a portion of other stimuli at a gamma-oscillation inducing frequency, wherein the other stimuli originate from the cognitively engaging content. In some embodiments, the stimulation source may comprise a cover, wherein the cover displays the gamma-oscillation inducing non-invasive sensory stimulus by adding out the gamma-oscillation inducing to other stimuli, wherein the other stimuli originate from the cognitively engaging content.

In some embodiments, the stimulation source may form at least a portion of the display. The portion may comprise various shapes and sizes of the display. In some embodiments, the portion may comprise a rectangular shape, a triangular shape, or any other shape.

In some embodiments, the stimulation source may comprise at least a portion made of a transparent material. In some embodiments, the transparent material may comprise a glass or a polymer. In some embodiments, the transparent material may be an electrochromic material. In some embodiments, the transparent material may be polypyrrole, poly(3,4-ethylenedioxythiophene) (PEDOT), polyaniline, or viologen.

The stimulation source may be configured to display the gamma inducing non-invasive sensory stimulus for various amounts of time. In some embodiments, the stimulation source is configured to display the waveform for a duration of at least about 1 second. In some embodiments, the stimulation source is configured to display the stimulus for a duration of at least about 1 minute. In some embodiments, the stimulation source is configured to display the stimulus for a duration of at least about 1 hour.

### Combination Therapies

In one aspect, the present disclosure provides combination therapies comprising the administration of one or more additional therapeutic regimens in conjunction with methods described herein. In some embodiments, the additional therapeutic regimens are directed to the treatment or prevention of the disease or disorder targeted by methods of the present technology.

In some embodiments, the additional therapeutic regimens comprise administration of one or more pharmacological agents that are used to treat or prevent disorders targeted by methods of the present technology. In some embodiments, methods of the present technology facilitate the use of lower doses of pharmacological agents to treat or prevent targeted disorders.

In some embodiments, the additional therapeutic regimens comprise non-pharmacological therapies that are used to treat or prevent disorders targeted by methods of the present technology such as, but not limited to, cognitive or physical therapeutic regimens.

In some embodiments, a pharmacological agent is administered in conjunction with therapeutic methods described herein. In some embodiments, the pharmacological agent is directed to inducing a relaxed state in a subject administered methods of the present technology. In some embodiments, the pharmacological agent is directed to inducing a heightened state of awareness in a subject administered methods of the present technology. In some embodiments, the pharmacological agent is directed to modulating neuronal and/or synaptic activity. In some embodiments, the agent promotes neuronal and/or synaptic activity. In some embodiments, the agent targets a cholinergic receptor. In some embodiments, the agent is a cholinergic receptor agonist. In some embodiments, the agent is acetylcholine or an acetylcholine derivative. In some embodiments, the agent is an acetylcholinesterase inhibitor.

In some embodiments, the agent inhibits neuronal and/or synaptic activity. In some embodiments, the agent is a cholinergic receptor antagonist. In some embodiments, the agent is an acetylcholine inhibitor or an acetylcholine derivative inhibitor. In some embodiments, the agent is acetylcholinesterase or an acetylcholinesterase derivative.

### Machine Learning Elements

In some cases, machine learning, artificial intelligence, and machine learning algorithms can refer to a model or a method of training the model that captures statistics or patterns provided in a dataset to a learning algorithm.

In some cases, a machine learning algorithm may use dimensionality reduction. The terms reducing, dimensionality reduction, projection, component analysis, feature space reduction, latent space engineering, feature space engineering, representation engineering, or latent space embedding can refer to a method of transforming a given input data with an initial number of dimensions to another form of data that has fewer dimensions than the initial number of dimensions. In some cases, the terms can refer to the principle of reducing a set of input dimensions to a smaller set of output dimensions.

In some cases, a machine learning algorithm may use normalization. The term normalizing can refer to a collection of methods for adjusting a dataset to align the dataset to a common scale. In some cases, a normalizing method can comprise multiplying a portion or the entirety of a dataset by a factor. In some cases, a normalizing method can comprise adding or subtracting a constant from a portion or the entirety of a dataset. In some cases, a normalizing method can comprise adjusting a portion or the entirety of a dataset to a known statistical distribution. In some cases, a normalizing method can comprise adjusting a portion or the entirety of a dataset to a normal distribution. In some cases, a normalizing method can comprise adjusting the dataset so that the signal strength of a portion or the entirety of a dataset is about the same.

In some cases, a machine learning algorithm may use data conversion. Converting can comprise one or more steps of various of conversions of data. In some cases, converting can comprise normalizing data. In some cases, converting can comprise performing a mathematical operation that computes a score based on a distance between 2 points in the data. In some embodiments, the distance can comprise a distance between two edges in a graph. In some embodiments, the distance can comprise a distance between two nodes in a graph. In some embodiments, the distance can comprise a distance between a node and an edge in a graph. In some embodiments, the distance can comprise a Euclidean distance. In some embodiments, the distance can comprise a non-Euclidean distance. In some embodiments, the distance can be computed in a frequency space. In some embodiments, the distance can be computed in Fourier space. In some embodiments, the distance can be computed in Laplacian space. In some embodiments, the distance can be computed in spectral space. In some embodiments, the mathematical operation can be a monotonic function based on the distance. In some embodiments, the mathematical operation can be a non-monotonic function based on the distance. In some embodiments, the mathematical operation can be an exponential decay function. In some embodiments, the mathematical operation can be a learned function.

In some embodiments, converting can comprise transforming a data in one representation to another representation. In some embodiments, converting can comprise transforming data into another form of data with less dimensions. In some embodiments, converting can comprise linearizing one or more curved paths in the data. In some embodiments, converting can be performed on data comprising data in Euclidean space. In some embodiments, converting can be performed on data comprising data in graph space. In some embodiments, converting can be performed on data in a discrete space. In some embodiments, converting can be performed on data comprising data in frequency space. In some embodiments, converting can transform data in discrete space to continuous space, continuous space to discrete space, graph space to continuous space, continuous space to graph space, graph space to discrete space, discrete space to graph space, or any combination thereof. In some embodiments, converting can comprise transforming data in discrete space into a frequency domain. In some embodiments, converting can comprise transforming data in continuous space into a frequency domain. In some embodiments, converting can comprise transforming data in graph space into a frequency domain.

In some embodiments, reducing can comprise transforming a given input data with any initial number of dimensions to another form of data that has any number of dimensions fewer than the initial number of dimensions. In some embodiments, reducing can comprise transforming input data into another form of data with fewer dimensions. In some embodiments, reducing can comprise linearizing one or more curved paths in the input data to the output data. In some embodiments, reducing can be performed on data comprising data in Euclidean space. In some embodiments, reducing can be performed on data comprising data in graph space. In some embodiments, reducing can be performed on data in a discrete space. In some embodiments, reducing can transform data in discrete space to continuous space, continuous space to discrete space, graph space to continuous space, continuous space to graph space, graph space to discrete space, discrete space to graph space, or any combination thereof.

In some cases, a machine learning algorithm may use clustering. The terms clustering, cluster analysis, or generating modules can refer to a method of grouping samples in a dataset by some measure of similarity. Samples can be grouped in a set space, for example, element ‘a’ is in set ‘A’. Samples can be grouped in a continuous space, for example, element ‘a’ is a point in Euclidean space with distance ‘l’ away from the centroid of elements comprising cluster ‘A’. Samples can be grouped in a graph space, for example, element ‘a’ is highly connected to elements comprising cluster ‘A’. These terms can refer to the principle of organizing a plurality of elements into groups in some mathematical space based on some measure of similarity.

Clustering can comprise grouping any number of samples in a dataset by any quantitative measure of similarity. In some embodiments, clustering can comprise K-means clustering. In some embodiments, clustering can comprise hierarchical clustering. In some embodiments, clustering can comprise using random forest models. In some embodiments, clustering can comprise boosted tree models. In some embodiments, clustering can comprise using support vector machines. In some embodiments, clustering can comprise calculating one or more N−1 dimensional surfaces in N-dimensional space that partitions a dataset into clusters. In some embodiments, clustering can comprise distribution-based clustering. In some embodiments, clustering can comprise fitting a plurality of prior distributions over the data distributed in N-dimensional space. In some embodiments, clustering can comprise using density-based clustering. In some embodiments, clustering can comprise using fuzzy clustering. In some embodiments, clustering can comprise computing probability values of a data point belonging to a cluster. In some embodiments, clustering can comprise using constraints. In some embodiments, clustering can comprise using supervised learning. In some embodiments, clustering can comprise using unsupervised learning.

In some embodiments, clustering can comprise grouping samples based on similarity. In some embodiments, clustering can comprise grouping samples based on quantitative similarity. In some embodiments, clustering can comprise grouping samples based on one or more features of each sample. In some embodiments, clustering can comprise grouping samples based on one or more labels of each sample. In some embodiments, clustering can comprise grouping samples based on Euclidean coordinates. In some embodiments, clustering can comprise grouping samples based the features of the nodes and edges of each sample.

In some embodiments, comparing can comprise comparing between a first group and different second group. In some embodiments, a first or a second group can each independently be a cluster. In some embodiments, a first or a second group can each independently be a group of clusters. In some embodiments, comparing can comprise comparing between one cluster with a group of clusters. In some embodiments, comparing can comprise comparing between a first group of clusters with second group of clusters different than the first group. In some embodiments, one group can be one sample. In some embodiments, one group can be a group of samples. In some embodiments, comparing can comprise comparing between one sample versus a group of samples. In some embodiments, comparing can comprise comparing between a group of samples versus a group of samples.

A machine learning model can comprise one or more of various machine learning models. In some embodiments, the machine learning model can comprise one machine learning model. In some embodiments, the machine learning model can comprise a plurality of machine learning models. In some embodiments, the machine learning model can comprise a neural network model. In some embodiments, the machine learning model can comprise a random forest model. In some embodiments, the machine learning model can comprise a manifold learning model. In some embodiments, the machine learning model can comprise a hyperparameter learning model. In some embodiments, the machine learning model can comprise an active learning model.

In some cases, a graph, graph model, and graphical model can refer to a method of conceptualizing or organizing information into a graphical representation comprising nodes and edges. In some embodiments, a graph can refer to the principle of conceptualizing or organizing data, wherein the data may be stored in a various and alternative forms such as linked lists, dictionaries, spreadsheets, arrays, in permanent storage, in transient storage, and so on, and is not limited to specific embodiments disclosed herein. In some embodiments, the machine learning model can comprise a graph model.

The machine learning model can comprise a neural network comprising various architectures, loss functions, optimization algorithms, priors, and various other neural network design choices. In some embodiments, the machine learning model can comprise a neural network. In some embodiments, the machine learning model can comprise an autoencoder. In some embodiments, the machine learning model can comprise a generative model. In some embodiments, the machine learning model can comprise a variational autoencoder. In some embodiments, the machine learning model can comprise a generative adversarial network. In some embodiments, the machine learning model can comprise a flow model. In some embodiments, the machine learning model can comprise an autoregressive model. In some embodiments, the machine learning model can comprise a neural network with one or more layers. In some embodiments, the machine learning model can comprise a neural network with one or more fully connected layers. In some embodiments, the machine learning model can comprise a neural network with one or more convolutional layers. In some embodiments, the machine learning model can comprise a neural network with one or more message-passing layers. In some embodiments, the machine learning model can comprise a neural network with a bottleneck layer.

In some embodiments, the machine learning model can comprise a neural network with residual blocks. In some embodiments, the machine learning model can comprise a neural network with attention. In some embodiments, the machine learning model can comprise a neural network with one or more non-linearities. In some embodiments, the machine learning model can comprise a neural network with one or more dropout layers. In some embodiments, the machine learning model can comprise a neural network with one or more batch normalization layers. In some embodiments, the machine learning model can comprise a regression loss function. In some embodiments, the machine learning model can comprise a logistic loss function. In some embodiments, the machine learning model can comprise a variational loss. In some embodiments, the machine learning model can comprise a prior. In some embodiments, the machine learning model can comprise a Gaussian prior. In some embodiments, the machine learning model can comprise a non-Gaussian prior. In some embodiments, the machine learning model can comprise an adversarial loss. In some embodiments, the machine learning model can comprise an autoencoding loss. In some embodiments, the machine learning model is trained with the Adam optimizer. In some embodiments, the machine learning model is trained with the stochastic gradient descent optimizer. In some embodiments, the model learning model hyperparameters are optimized with Gaussian Processes. In some embodiments, the machine learning model is trained with train/validation/test data splits. In some embodiments, the machine learning model is trained with k-fold data splits, with any positive integer for k.

The machine learning model can comprise a variety of manifold learning algorithms. In some embodiments, the machine learning model can comprise a manifold learning algorithm. In some embodiments, the manifold learning algorithm is principal component analysis. In some embodiments, the manifold learning algorithm is a uniform manifold approximation algorithm. In some embodiments, the manifold learning algorithm is an isomap algorithm. In some embodiments, the manifold learning algorithm is a locally linear embedding algorithm. In some embodiments, the manifold learning algorithm is a modified locally linear embedding algorithm. In some embodiments, the manifold learning algorithm is a Hessian eigenmapping algorithm. In some embodiments, the manifold learning algorithm is a spectral embedding algorithm. In some embodiments, the manifold learning algorithm is a local tangent space alignment algorithm. In some embodiments, the manifold learning algorithm is a multi-dimensional scaling algorithm. In some embodiments, the manifold learning algorithm is a t-distributed stochastic neighbor embedding algorithm (t-SNE). In some embodiments, the manifold learning algorithm is a Barnes-Hut t-SNE algorithm.

### EXAMPLES

### Example 1. Human Clinical Study of Safety, Efficacy, and Results of Treatment

### Methods and Study Design

A clinical study was performed to assess the safety, tolerability, and efficacy of long-term, daily use of gamma oscillation inducing non-invasive sensory stimulation therapy on cognition, functional ability, and biomarkers in a mild-to-moderate AD population via a prospective clinical study. The clinical study was a multi-center, randomized controlled trial evaluating daily gamma oscillation inducing sensory stimulation received at home for a 6-month treatment period. Subjects included in the study were adults 50 years and older with a clinical diagnosis of mild to moderate AD (MMSE: 14-26, inclusive), a reliable care partner, and successful tolerance and sensory induction of neural oscillations screening via EEG. Key exclusion criteria included profound hearing or visual impairment, use of memantine, major psychiatric illness, clinically relevant history of seizure, or contraindication to imaging studies.

Study Participants and Design. A total of 135 patients were assessed for eligibility to participate in the study. Patients were first given a screening EEG, and then split into groups. One group was a sham control group that was not given treatment; the other was a group that was subjected to 1 hour of therapy, which involved subjecting the subject each day to 40 Hz audio and visual stimulation. Of those assessed for eligibility, 76 were randomized between the active treatment and sham control. Forty-seven of the randomized patients were allocated to the active group and 29 were allocated to the sham group. Of the active group, two patients withdrew prior to therapy and three had no post-baseline efficacy and were not included in the modified intent to treat (mITT) population. In sham group, one patient received active treatment and was not in the sham population. Completers included 33 patients in the active group and 28 in the sham group, with 10 early discontinuations in the active group. Seven of those discontinuations were due to consent withdraw and 23 were attributed to adverse events, whereas in the sham group, only six withdrew consent and one discontinued as a result of adverse events.

The study employed various clinical outcome assessment scales to assess cognitive decline or dysfunction. These included the Neuropsychiatric Inventory (NPI), Clinical Dementia Rating-Sum of Boxes (CDR-sb), the Clinical Dementia Rating-Global Score (CDR global), the Mini-Mental State Exam (MMSE), the Alzheimer's Disease Assessment Scale-Cognitive Subscale-14 (ADAS-Cog14), and a variation of the Alzheimer's Disease Composite Score (ADCOMS) as optimized for patients with mild or moderate Alzheimer's Disease. NPI examines 12 sub-domains of behavioral functioning: delusions, hallucinations, agitation/aggression, dysphoria, anxiety, euphoria, apathy, disinhibition, irritability/lability, and aberrant motor activity, night-time behavioral disturbances, and appetite and eating abnormalities. The NPI can be used to screen for multiple types of dementia, and it involves giving the caregiver of a subject the questions and then, based on the answers, rating the frequency of the symptoms, their severity, and the distress the symptoms cause on a three, four, and five-point scale, respectively.

CDR global is calculated based on testing performed for six different cognitive and behavioral domains: memory, orientation, judgment, and problem solving, community affairs, home and hobbies performance, and personal care. To test these areas, an informant is given a set of questions about a subject's memory problem, judgment and problem-solving ability of the subject, community affairs of the subject, home life and hobbies of the subject, and personal questions related to the subject. The subject is given another set of questions that includes memory-related questions, orientation-related questions, and questions about judgment and problem-solving ability. The CDR global score is calculated based on the results of those questions, and it is measured using a scale of 0 to 3, with 0 representing no dementia, 0.5 indicating very mild dementia, 1 indicating mild dementia, 2 indicating moderate dementia/cognitive impairment, and 3 indicating severe dementia/cognitive impairment. CDR-sb is a clinical outcome assessment that looks at functional impact of cognitive impairment: memory, executive function, instrumental and basic activities of daily living and assesses them based on interviews with an informant and the patient. The CDR-sb score is based on assessment of items including memory, orientation, judgment, and problem solving, community affairs, home and hobbies, and personal care. The CDR-sb is scored from 0 to 18, with higher scores representing greater severity of cognitive and functional impairment.

The MMSE looks at 11 items to assess memory, language, praxis, and executive function based on a cognitive assessment of the patient. Items assessed include registration, recall, constructional praxis, attention and concentration, language, orientation time, and orientation place. The MMSE is scaled from 0 to 30, with higher scores representing lower severity of cognitive dysfunction. The ADAS-Cog14 assesses memory, language, praxis, and executive function. The score is based on a cognitive assessment of the patient and assesses fourteen items: spoken language, maze, comprehension spoken language, remembering word recognition test instructions, ideational praxis, commands, naming, word finding difficulty, constructional praxis, orientation, digit cancellation, word recognition, word recall, and delayed recall. A score is based on points allocated to each item, and the maximum total score is 90, with higher numbers indicating greater severity of cognitive dysfunction. The Alzheimer's Disease Composite Score (ADCOMS) considers items from all of the above-discussed scores: items from Alzheimer's Disease Assessment Scale-cognitive subscale items, MMSE items, and all of the CDR-sb items. ADCOMS combines portions of the ADAS-cog, Clinical Dementia Rating (CDR) scale, and MMSE that have been shown to change the most over time in people who do not have functional impairment yet. MADCOMS, which was used in the present example, optimizes the scale instead by combining items more significant for mild and moderate dementia.

The study design involved primary efficacy endpoints of MADCOMS, ADAS-cog14, and CDR-sb. Unlike ADCOMS, MADCOMS is optimized for patients with moderate or mild Alzheimer's Disease. These were optimized for AD-specific decline. A separate optimization was done for moderate and mild AD. Secondary efficacy endpoints consisted of ADCS-ADL, ADCOMs (adjusted), MMSE, CDR-global score and the Neuropsychiatric Inventory (NPI). Of the secondary endpoints, ADCS-ADL was measured monthly and MMSE was measured at the last time point.

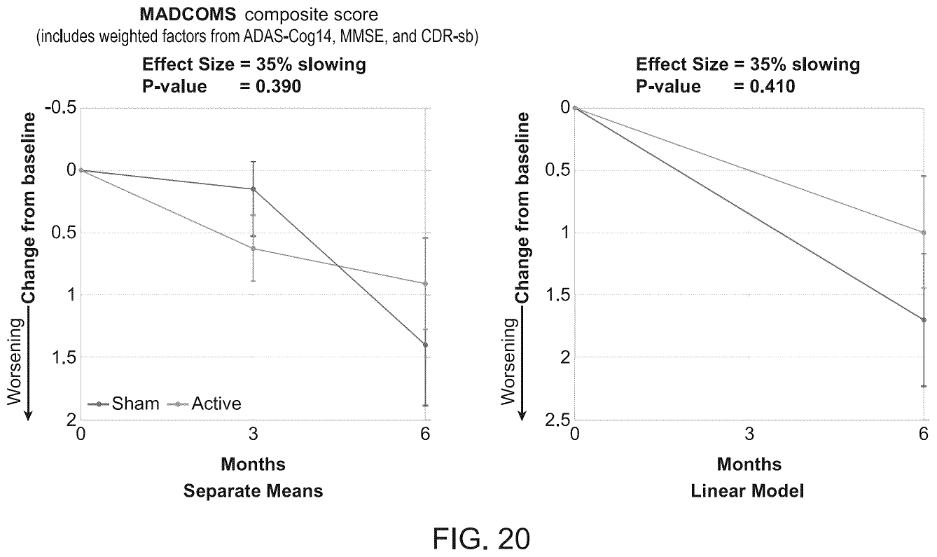

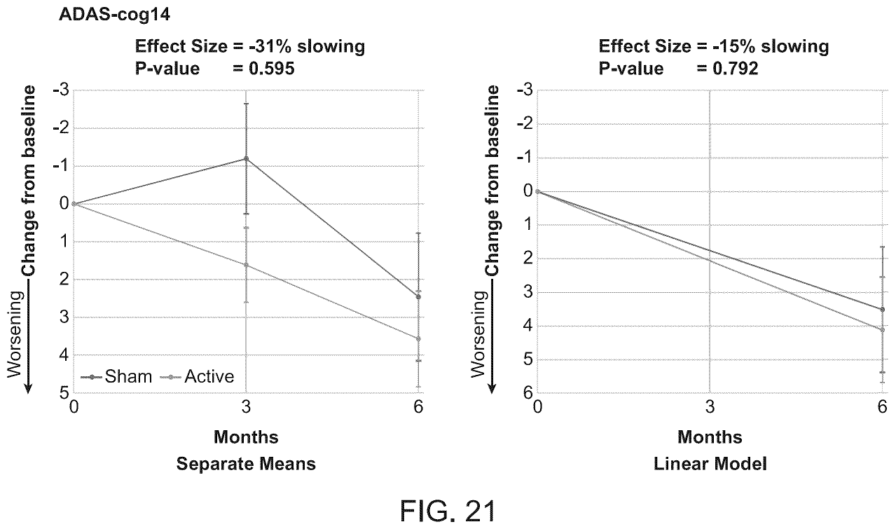

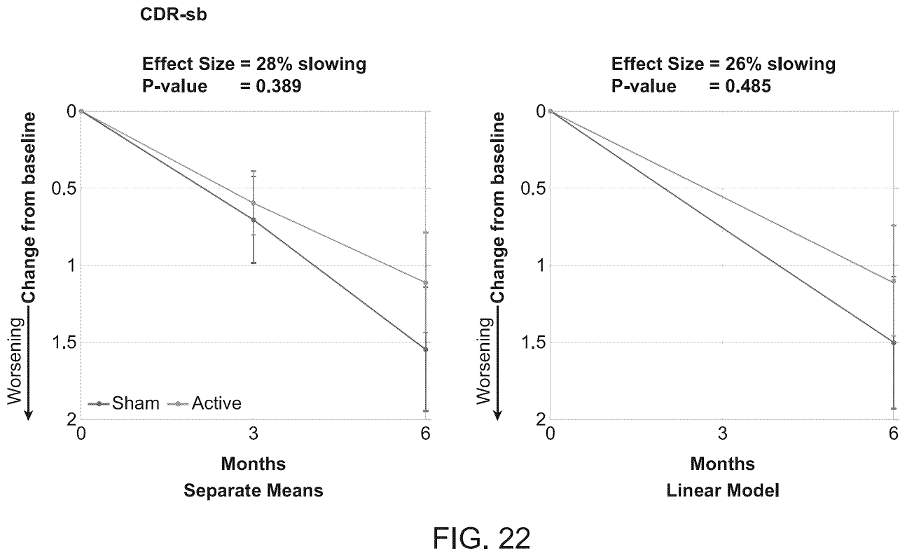

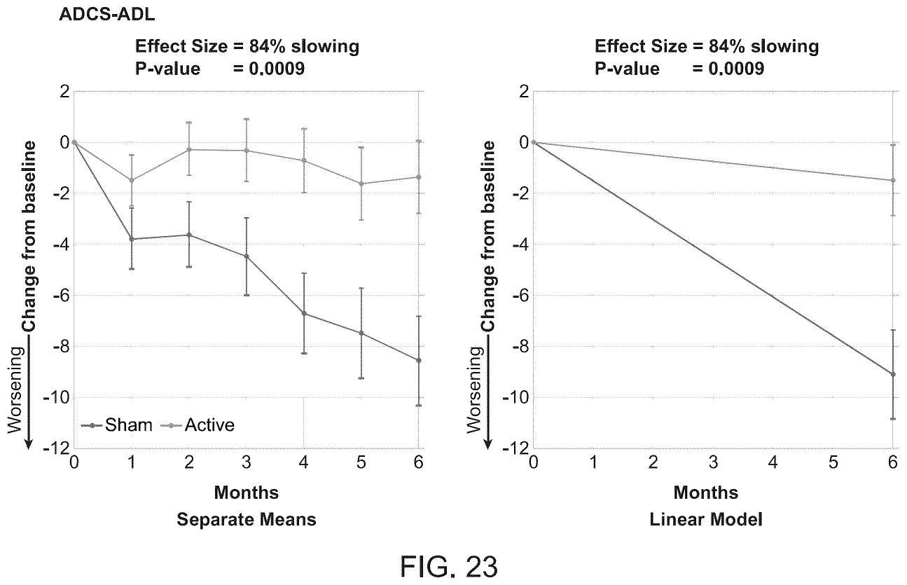

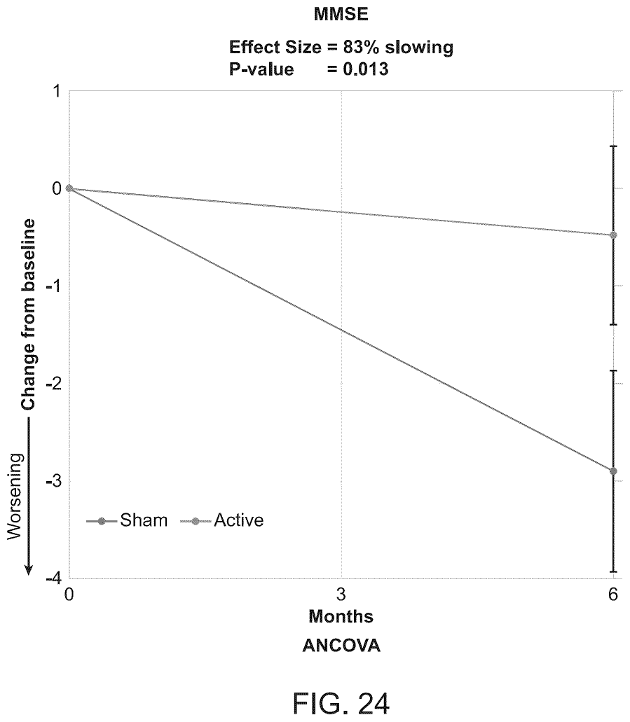

The efficacy endpoints were analyzed by applying a linear model of analysis and/or a separate means model of analysis. The linear model of analysis involved employing a linear fit model to determine a value at TO based on the difference from baseline in conditions at the end of the study. The separate means analysis employed estimates of mean values at each assess timepoint, which was either a monthly timepoint or at three and six months after treatment began, depending on the score that was being analyzed. In evaluating MADCOMS composite score, for example, the separate means analysis was applied using mean values that were estimated at three and six months. The linear model was applied by using the estimates of treatment difference at the end of the study and connecting a straight line to 0. Similar models were used for the other efficacy endpoints. FIGS. 20, 21, 22, 23, and 24 show the various linear and separate means models generated for these endpoints.

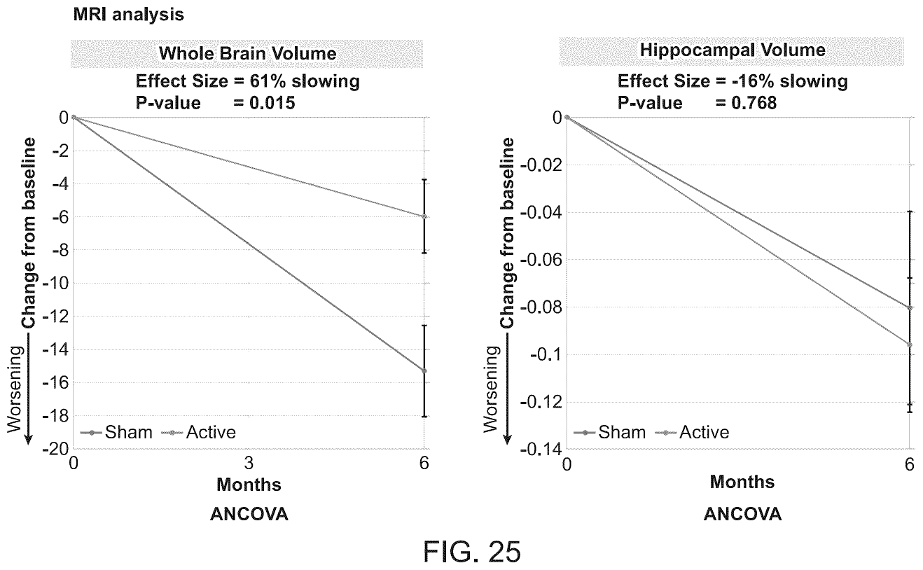

To assess biomarkers, researchers used vMRI, EEG, positron emission tomography (PET), actigraphy, and plasma biomarkers. The study employed structural MRIs, taken before any treatment began and at the end of the sixth months and assessed these for volume-base morphology. Volumetric changes for the hippocampus, lateral ventricles, whole cortex (cerebral cortical gray matter) and whole brain (cerebrum and cerebellum) were determined, and the rate of atrophy was compared for active and sham groups using a linear model, as demonstrated in FIG. 25. To analyze for safety and tolerability, researchers looked for adverse events and presence of amyloid related imaging abnormalities (ARIA) on MRI. Therapy adherence was also analyzed. Blinding effectiveness for subjects, care partners, and assessors were prospectively analyzed by assessing baseline and follow up ascertainment of whether the care partner, assessor, or patient thought the patient was on active or sham treatment.

### Analysis

For the MADCOMS composite scores, both means of analysis demonstrated 35% slowing in decline rate, indicating that the active group progressed less than the placebo arm over the six-month study. When a linear and means analysis were both employed, the sham group was slightly favored, but non-significantly. When these two separate means analyses were applied to the ADAS-cog14 data, both slightly favored the sham group, although not in a statistically significant manner. When CDR-sb results were analyzed, the mean-estimate model found a 28% slowing rate, whereas the linear extraction showed a 26% slowing rate, but the comparisons were not statistically significant.

Of the secondary endpoints, ADCS-ADL was measured monthly and MMSE was measured at the last time point. When analyzing ADCS-ADL values, the first analysis model employed used estimates for each month and showed 84% slowing over the 6-month time period. The linear fit model was again employed, and the same 84% slowing was found. When analyzing MMSE values, an 83% slowing was identified.

### Results

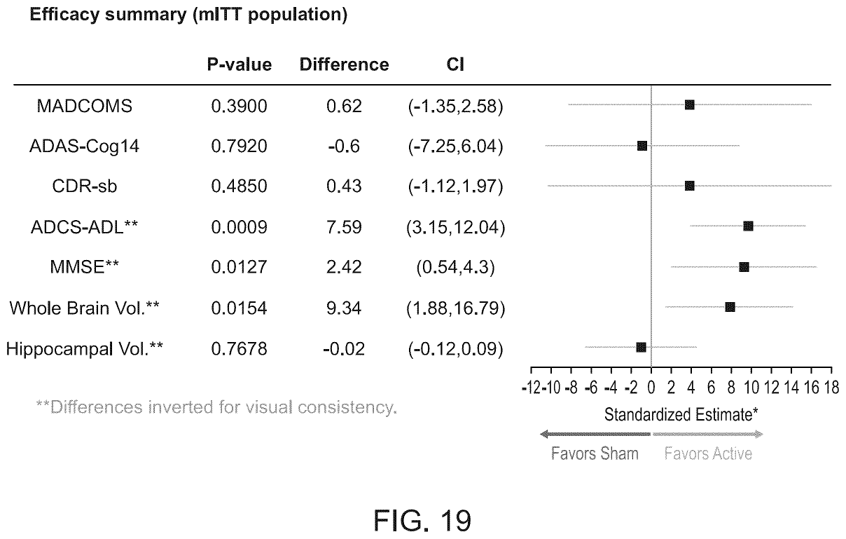

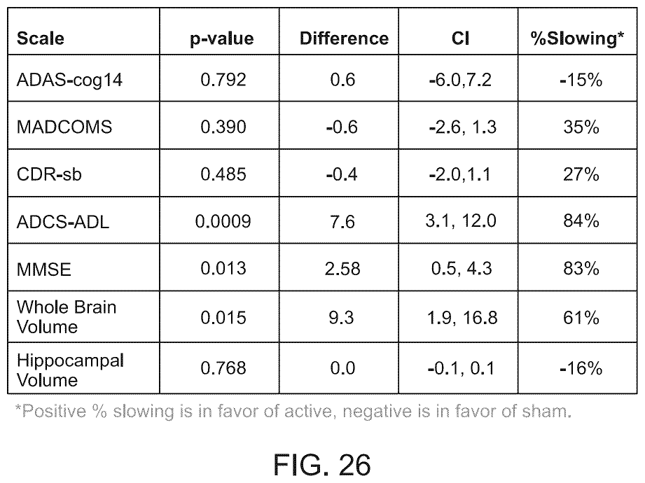

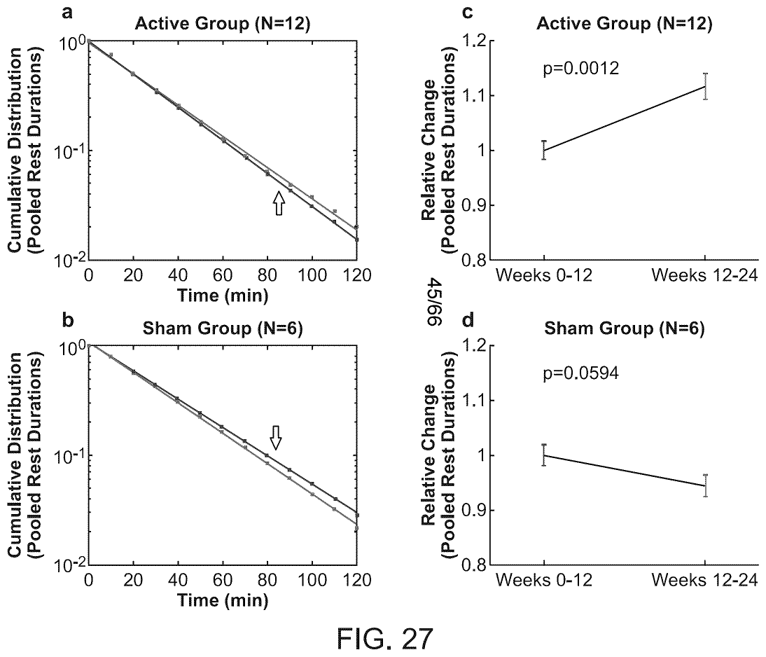

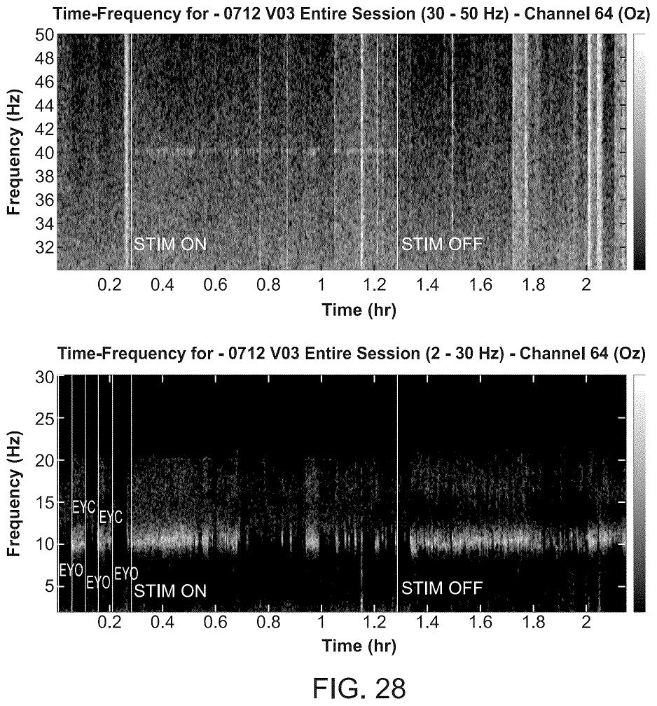

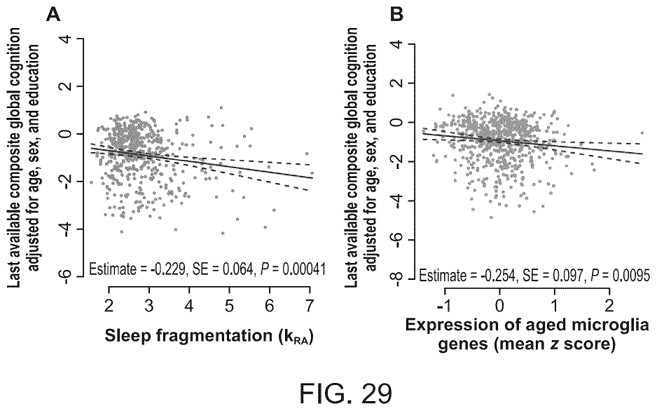

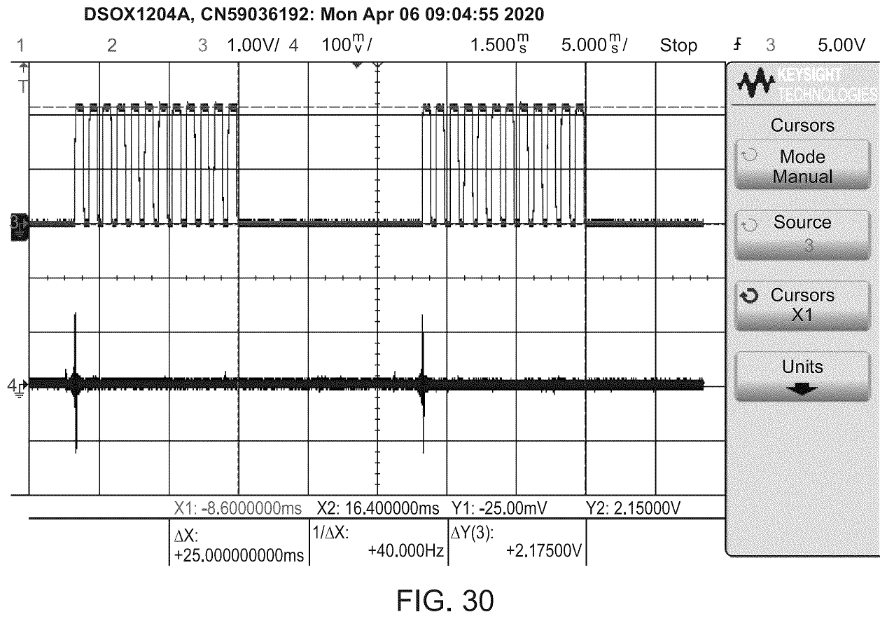

FIG. 19 and FIG. 26 summarize the efficacy findings of the study. Following informed consent and screening, a total of 76 subjects were randomized between the active treatment and sham control. The safety population for the study included 74 subjects who received at least one treatment, and the modified intent to treat (mITT) population included a total of 70 subjects, 53 of whom completed the 6-month study, which form the basis for analysis of outcome measures.

### Demographic and Baseline Characteristics

In terms of demographic and baseline characteristics of the mITT population, following randomization, the populations were balanced across gender, baseline MMSE, ApoE4 status, activities of daily living (ADL), and PET amyloid standardized uptake value ratio (SUVR) status; imbalances between the two groups were observed in age, ADAS-Cog11, and CDR-sb scores at baseline. Statistical models included covariates for age and MMSE at baseline.

### Safety and Tolerability

Non-invasive gamma oscillation inducing sensory stimulation was safe and well-tolerated in the mild and moderate AD subjects. The active group had a lower rate of treatment emergent adverse events (TEAE) than the sham group (67% vs 79%). Treatment related AEs (TRAEs) deemed “definitely”, “probably”, and “possibly”-related to the therapy were elevated in the active group versus the sham group (41% vs 32%). One treatment related SAE was noted in the active group for a patient hospitalized for wandering while their care partner was located; this subject discontinued the study subsequently. Of the randomized subjects, withdraw rates were similar between both groups (active 28%, sham 29%) including withdraw rates due to an adverse event (active 7%, sham 7%). TEAEs that occurred more often in the active group are tinnitus, delusions, broken bone. TEAEs that occurred more often in the sham group are upper respiratory infection, confusion, anxiety, and dizziness.

### Clinical Assessments

Over the treatment period of 6-months, subjects were evaluated in-clinic and via phone visits for cognitive, functional, and biomarker changes on multiple measures.

The primary efficacy endpoints demonstrated effects favoring the active group on the MADCOMS (35% slowing; n.s.) and CDR-sb (27%; n.s.) and favoring the Sham group on the ADAS-cog14 (−15% slowing; n.s.). MADCOMS initially leaned in favor of active group, but the results were not statistically different. ADAS-cog14 was slightly in favor of the sham group but not statistically different. CDR-sb was also in favor of the active group, but the difference was not significant, as shown by the p-values that ranged between 0.39 and 0.7920.

Selected secondary endpoints demonstrated significant effects favoring the treatment (active) group. The active group had significant benefit on functional ability as measured by the ADCS-ADL (p=0.0009), which represented an 84% slowing of decline and a treatment difference of 7.59 points over the six-month duration of the trial (FIG. 26). The active group demonstrated significant benefit on the MMSE (ANCOVA p=0.013), which represented an 83% slowing in the rate of decline versus the Sham group and a treatment difference of 2.42 points.

### Biomarker Changes—MRI

Structural MR imaging was analyzed for volume-base morphometry using an automated image processing pipeline (Biospective, Montreal, Canada). Volumetric changes of the hippocampus, lateral ventricles, whole cortex (cerebral cortical gray matter) and whole brain (cerebrum and cerebellum, no cerebrospinal fluid (CSF)) for each subject were determined; no manual corrections were performed. No significant benefit on hippocampal volume was determined. Statistically significant benefit favoring the active group (p=0.0154) on whole brain volume (WBV) was established, representing a 61% slowing compared to the Sham group progression. The treatment value for the active group was 9.34 cm³.

### Conclusions

Gamma oscillation inducing sensory stimulation was safe and well tolerated. Two of three primary efficacy outcomes (MADCOMS, CDR-sb) favored the active group but did not reach significance. Selected secondary endpoints demonstrated that active treatment with gamma oscillation inducing sensory stimulation therapy led to significant benefits in the ability to perform activities of daily living via the ADCS-ADL and cognition via the MMSE, representing important treatment and management objectives for AD patients. Quantitative MR analysis demonstrated slowing of brain atrophy as measured by whole brain volume in the active group. The combined clinical and biomarker findings suggest beneficial effects of gamma oscillation inducing sensory stimulation for AD subjects may be facilitated via differentiated pathways. These surprising results indicates that the gamma oscillation inducing sensory stimulation may be used to treat a range of diseases and disorders that cause or are caused by brain atrophy.

### Example 2. Human Clinical Study to Determine Efficacy of NSS Treatment for Sleep Abnormalities

### Methods

Study Participants and Design. Patients included in the present interim analysis were clinically diagnosed having mild to moderate AD and were under the care of their care neurologist. Inclusion criteria were age of 55 years or older, MMSE score 14-26 and participation of a caregiver, whereas exclusion criteria included profound hearing or visual impairment, seizure disorder, use of memantine, or implantable, non-MR compatible devices. Patients on therapy with an acetylcholine esterase inhibitor could enroll, but their dosing were maintained the same during the trial. Patients were randomized to receive either 40 Hz simultaneous auditory and visual sensory stimulation by a NSS (treatment group; n=14) or placebo treatment (sham group; n=8).

Neural Stimulation System (NSS). In the present study, the system used for the neural stimulation provided noninvasive sensory stimulation provided visual and audio stimulation to invoke gamma oscillations in a brain region, thereby improving sleep. Use of such a system is referred to herein as NSS therapy or NSS treatment. The system logs device usage and stimulation output settings for adherence monitoring. This information is uploaded to a secure cloud server for physician remote monitoring. The present experiment utilized a NSS that included a handheld controller, an eye-set for visual stimulation, and headphones for auditory stimulation that work together to deliver precisely timed, non-invasive stimulation to induce steady-state gamma brainwave activity. The visual stimulation generated by the NSS consisted of precisely timed flashes of visible light from light emitting diodes, and the auditory stimulation consisted of short duration clicks. The stimuli 141 occurred at a pulse repetition frequency of 40 Hz. The on-off periods of the visual stimulation were perceivable by the patient but not disruptive; an individual remained aware of their surroundings and could converse with a care partner during use of the system. The customized stimulation output was determined and verified by a physician based on both patient-reported comfort information and on the patient's quantitative electroencephalography (EEG) response to the stimulation. The NSS was then configured to the determined settings, and all subsequent use would be within this predefined operating range.

Monitoring Sleep Fragmentation and Arousals with Actigraphy and Signal Processing. Effects of the NSS therapy on sleep fragmentation were determined by continuous monitoring activity of AD patients with a wrist worn actigraphy watch (ActiGraph GT9X), and data was collected daily over a 6-month period. Collected data consisted of raw accelerometer readings in three orthogonal directions recorded at a 30 Hz sampling frequency.

Preprocessing the Data. Accelerometer data from three orthogonal dimensions are filtered with a Butterworth bandpass (0.3-3.5 Hz) filter. The magnitude of the bandpass filtered 3-d accelerometer vector was then down-sampled by a factor of 4. This process is done for all data collected from all patients over the six months period. Two representations of the data were made: (i) a binary representation and (ii) a smooth representation. For the binary representation, all data was pooled together and a histogram in the log scale was obtained. The resulting histogram had a bimodal distribution, one peak corresponding to higher changes in acceleration and hence high activity periods, and the second peak corresponding to lower changes in acceleration and hence rest periods. Taking the location of the minimum between the two peaks as a threshold, acceleration magnitudes higher than the threshold were represented by 1's and acceleration magnitudes smaller than the threshold were represented by 0's. For the smooth representation, a median filter with length of six hours was applied to the down-sampled data to get a smooth estimate of the activity levels.

Extracting Nighttime (Sleep Segment). Individual 24-hour data segments were extracted from 12:00 μm midday on a given day to the next day 12:00 μm midday. The data was labeled with the binary representation for an initial estimate of the active—1's and rest—0's periods during the given 24-hour window. This window consisted of three segments: daytime (segment prior to sleep), nighttime (sleep segment) and daytime (segment after sleep). We proposed that the nighttime segment would consist of more 0's than 1's and daytime segments would consist of more 1's than 0's. Therefore, an ideal nighttime model was defined which was built with a function that takes a value 0 within continuous period of duration “L” centered at a time “T” with a value 1 outside this region. Given an initial estimate of L and T, the difference between the ideal nighttime model and the binary representation of movement was computed using a quadratic cost function. In this cost function each mismatch, occurring when the binary value is 1 during nighttime or 0 during daytime, contributes 1, and each match, occurring when the binary value is 0 during nighttime and 1 during daytime, contributes 0. The initial estimate for T was taken to be the time point corresponding to the minimum of the smooth representation mentioned above. Initial estimate for L is set to eight hours. Cost function was minimized using unconstrained nonlinear optimization. This led to the best model estimate for L, the nighttime length, and T, the nighttime mid-point, and allowed us to locate the borders for the three segments (daytime, nighttime, daytime) from the 24-hour window. We then extracted the nighttime segment to evaluate the micro-changes within.

Identification of Rest and Active Durations During Nighttime and Relating Them to Sleep. Within the nighttime segments, periods with all 0's is attributable to lack of movement and periods with all 1's is attributable to movement. However, mapping these periods directly to sleep fragmentation faces the problem that the durations of these periods can range from milliseconds to hours in actigraphy data, whereas analysis of sleep is carried out by classifying non-overlapping epochs of 30 second duration into awake and asleep. To link our actigraphy analysis to the analysis timescales used in sleep studies, all segments of length N were taken and replaced the values in those segments by the median value over a window of 3N duration centered on the segment. While N=30 s was chosen, it was found that the results were not sensitive to this exact choice. After repeating this process for all short segments, consecutive time points in the nighttime segments corresponding to 0's were identified as rest durations and those corresponding to 1's were identified as active durations.

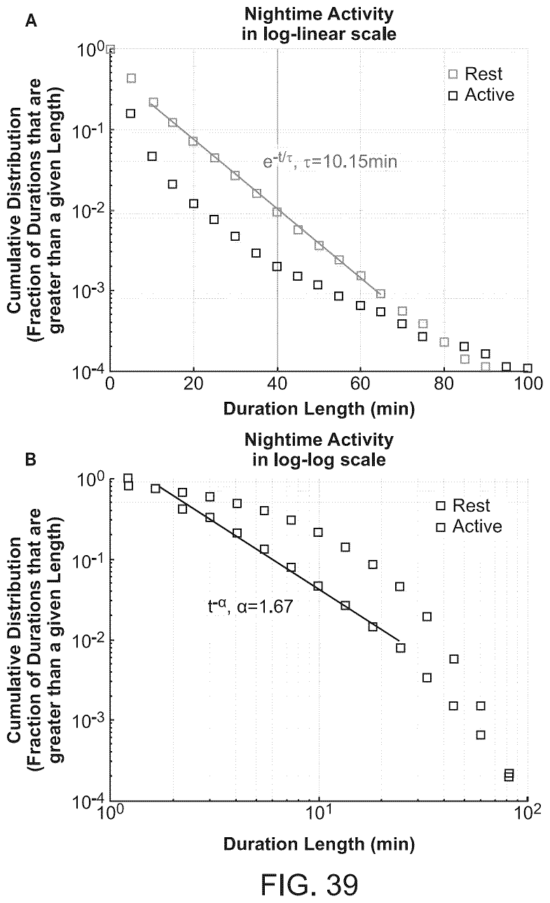

Determining the Distributions of Rest and Active Durations. Rest durations across all participants were pooled and the quantity, where p (w) is the probability density function of rest durations between w and w+dw, was examined. P (t) represents the fraction of rest durations that are greater than length t and is referred to as the cumulative distribution function. Similarly, the cumulative distribution of the active durations was also calculated, and distributions of both rest and active durations are displayed in FIG. 39.

Assessment of Functional Ability. Activities of daily living were also assessed at baseline and regular monthly intervals during the 24-week treatment period in the same study population of actigraphy recordings using the clinically established ADCS-ADL scale (Galasko, D., D. Bennett, M. Sano, C. Ernesto, R. Thomas, M. Grundman and S. Ferris (1997). “An inventory to assess activities of daily living for clinical trials in Alzheimer's disease. The Alzheimer's Disease Cooperative Study.” Alzheimer Dis Assoc Disord 11 Suppl 2: S33-39. The ADCS-ADL assesses the competence of AD patients in basic and instrumental activities of daily living. The assessments were by a caregiver in questionnaire format or administered by a healthcare professional as a structured interview with the caregiver. The six basic ADL items cover everyday activities, such as eating, personal grooming or dressing, also providing information on level of competence. The 16 instrumental ADL items ask the level of patient's engagement with basic instruments, such as a phone or kitchen appliances. ADCS-ADL has been a critical instrument to standardize assessment in AD clinical trials and is used widely as a functional outcome measure in disease modifying trials.

Assessment Cognitive Function. Subject cognitive function was assessed by the Mini-Mental State Exam (MMSE), which is a widely used instrument of cognitive function in AD patients, it tests patients' orientation, attention, memory, language, and visual-spatial skills.

Statistics. All statistical comparisons were done using Kolmogorov-Smirnov test.

### Results

This interim analysis reports results on 22 mild-to-moderate AD subjects who successfully completed the 6-month study. Demographic and clinical characteristics of all patients during the initial assessment are shown in TABLE 2.

TABLE 2
Demographic and Clinical Characteristics of
all Patients During the Initial Assessment

| | Treatment Group | | Sham Group | |
|---|---|---|---|---|
| Characteristic | (N = 14) | | (N = 8) | |
| Demographics | | | | |
| Age in years, mean ± sd | 66.5 ± 8.0 | | 73.5 ± 6.6 | |
| Gender, no (%) | | | | |
| Female | 10 | (71) | 5 | (63) |
| Male | 4 | (29) | 3 | (37) |
| Race and Ethnicity, no (%) | | | | |
| White | 14 | (100) | 8 | (100) |
| Hispanic or Latino | 1 | (7) | 0 | (0) |
| APOE-ε4 Allele Status, no (%) | | | | |
| 0 copies | 5 | (36) | 3 | (37.5) |
| 1 copy | 8 | (57) | 4 | (50) |
| 2 copies | 1 | (7) | 1 | (12.5) |
| Cognitive Assessment | | | | |

| | | |
|---|---|---|
| MMSE score†, mean ± sd | 19.9 ± 2.8 | 18.5 ± 2.7 |
| Functional Assessment | | |
| ADCS-ADL score‡, mean ± sd | 61.7 ± 9.2 | 65.0 ± 10.4 |

†Mini-Mental State Examination (MMSE) scores range between 0-30, higher scores indicating better cognitive performance.
‡Alzheimer’s Disease Cooperative Study - Activities of Daily Living (ADCS-ADL) scores range between 0-78, higher scores indicating better functioning.

### Data on Safety & Adherence

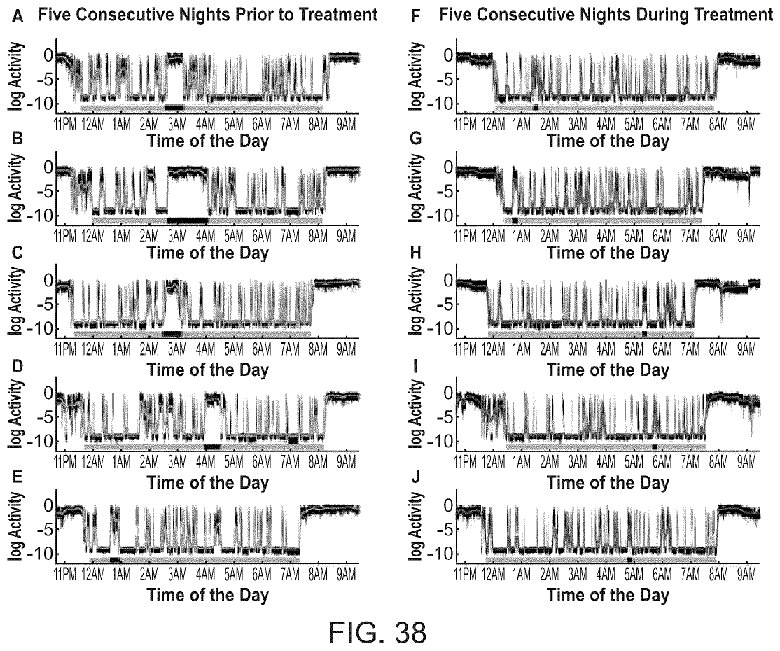

Sleep Evaluated by Continuous Actigraphy Recordings. Outcomes from the NSS treatment on sleep were revealed from continually recorded actigraphy data and constructing a nighttime sleep model, which allowed to assess the durations of rest and active periods during sleep. Results from this analysis of a single patient are shown in FIG. 38. FIG. 38 demonstrate nighttime active and rest periods; the level of continuous activity is determined and indicated by the black tracing. Furthermore, intervals were identified as sleep for each night (represented by horizontal light gray bars), and the longest movement periods are indicated by the dark gray bars. All rest and active durations identified by actigraphy data processing were pulled and analyzed from each participant as described in Methods section, and the results were compared to published data of rest and active periods obtained by polysomnography-based sleep analysis. As evidenced by straight-line fits on a log-linear scale, the rest durations follow an exponential distribution, e\hat{ }(−t/τ) with τ=10.15 min. In contrast, active durations follow power law distribution (straight-line fit on a log-log scale), t\hat{ }(−α) with α=1.67 (FIG. 39). As demonstrated by FIG. 39, the cumulative distributions for pooled, nighttime, rest (gray) and active (black) durations show exponential and power law distributions, respectively. The X axes of FIG. 39 show the nighttime durations. The Y axes show the cumulative distributions obtained from 14736 hours of data from 23 patients and the solid lines show the best straight-line fits. Such exponential and power law behaviors have been observed in sleep studies of healthy subjects (Lo, C. C., N. A. L. A., S. Havlin, P. C. Ivanov, T. Penzel, J. H. Peter and H. E. Stanley (2002). “Dynamics of Sleep-Wake Transitions During Sleep.” Europhys. Lett. 57 (5): 625-631; Lo, C. C., T. Chou, T. Penzel, T. E. Scammell, R. E. Strecker, H.-E. Stanley and P. C. Ivanov (2004). “Common scale-invariant patterns of sleep-wake transitions across mammalian species.” PNAS 101 (50): 17545-17548; Lo, C. C., R. P. Bartsch and P. C. Ivanov (2013). “Asymmetry and Basic Pathways in Sleep-Stage Transitions.” Europhys Lett 102 (1): 10008.). These authors analyzed nighttime sleep and awake states as obtained from polysomnographic recordings of healthy subjects and found that cumulative distribution of sleep state durations is characterized by an exponential distribution whereas those of awake state durations were characterized with a power law distribution. Thus, the exponential decay constant as τ=10.9 min for light sleep, τ=12.3 min for deep sleep, τ=9.9 min for REM sleep durations and the power law exponent as α=1.1 for awake durations were reported (Lo, Bartsch et al. 2013). It was found that the nighttime rest and active durations, estimated from actigraphy recordings of Alzheimer's disease patients show the same behavior as polysomnographic recordings of healthy subjects. Similarities in the form of the distributions between the results of the experiments described herein and previous work suggest that nighttime rest and active durations as assessed by actigraphy are analogous to sleep and awake states as assessed by polysomnography and that the effect of therapy on sleep may be indirectly assessed through its effect on active and rest durations.

Effects of NSS Treatment on Sleep Quality Determined by Continuous Actigraphy Recordings. Effects of NSS treatment on sleep were determined by comparing the distribution of the length of nighttime uninterrupted rest durations in the first and the second 12-week periods of the study (FIG. 40). Only subjects who wore the actigraphy device for at least six weeks during both the first and last 12-week period were used for assessing efficacy of NSS treatment on sleep (N=7 Treatment, N=6 Sham). To avoid subjects with more data dominating comparisons across periods, the first six weeks of available data closest to the study start and the last six weeks closest to the study end were considered for each subject. Actigraphy recordings from a single patient in the treatment group are shown in FIG. 38, displaying during 5 subsequent nights prior and during treatment period. The X-axis of FIG. 38 shows the time of day, and the Y-axis shows the activity level (in log scale). The black tracings represent the continuous activity levels, and the light gray horizontal bars represent the intervals identified as sleep in each night. The dark gray horizontal bars represent the longest movement periods within each night. The letters A through E correspond to five consecutive nights prior to treatment. The imposed curve shows a smooth (median filtered) activity level, with long movement intervals observed. Letters F through J correspond to five consecutive nights during treatment period. The imposed curve shows a smooth (median filtered) activity level. Compared to the pre-treatment period, patient showed fewer and shorter movement periods during treatment. In overall, nighttime active durations were significantly (p<0.03) reduced in the treatment group, whereas active durations were significantly (p<0.03) increased in patients of the sham group. Comparison of between treatment and sham groups were also done using normalized nighttime active durations. This normalization is done by dividing each active duration by the duration of the corresponding nighttime period. This measure eliminates potential variation in length of total sleep duration impacting numbers or durations of active periods. This analysis further confirmed opposite changes in nighttime active durations between treatment and sham groups. Changes in normalized active periods between the first and second 12-weeks period showed a significant (p<0.001) reduction in patients of the treatment group, in contrast to a significant increase (p<0.001) in patients of the sham group (FIG. 40). These findings demonstrate a reduction in nighttime active durations in response to NSS treatment, leading to reduction in sleep fragmentation and improvement in sleep quality, while the opposite can be assessed in the sham group.

Effects of NSS Treatment on Sleep Quality Determined by Continuous Actigraphy Recordings. MMSE changes were different in the treatment (n=13) and sham (n=8) groups. Initial assessment showed an MMSE value of 19.9±2.9, which did not change significantly during the duration of the treatment, showing an MMSE value of 19.3±3.4, measured at week 24. In contrast, the sham group showed the expected a significant decline in MMSE scored: initial score of 18.5±2.7 dropped to 16.8±5.7 (p<0.05).

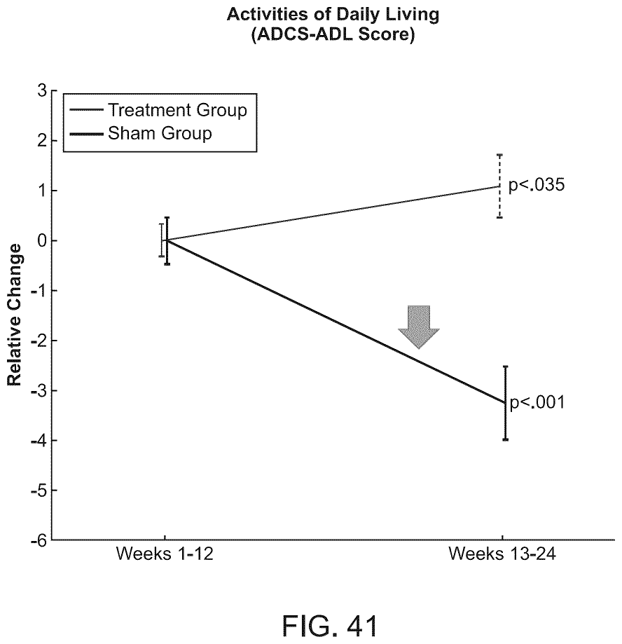

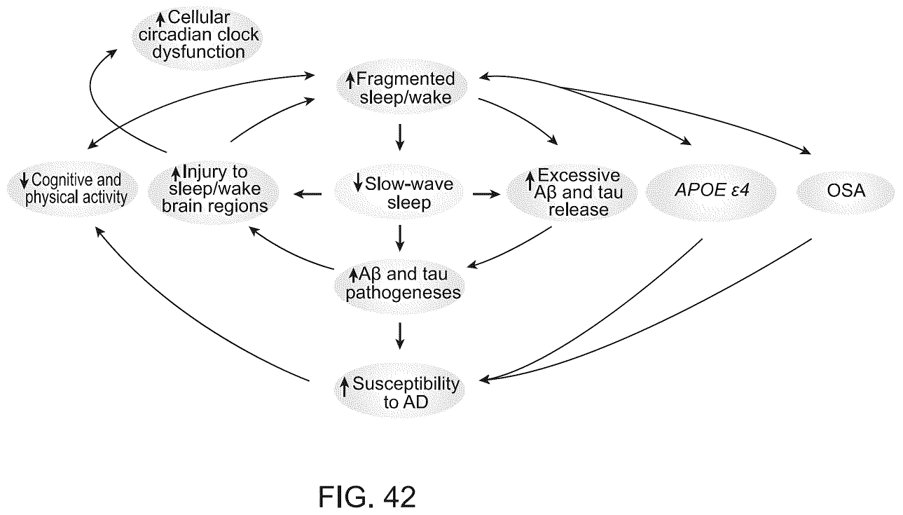

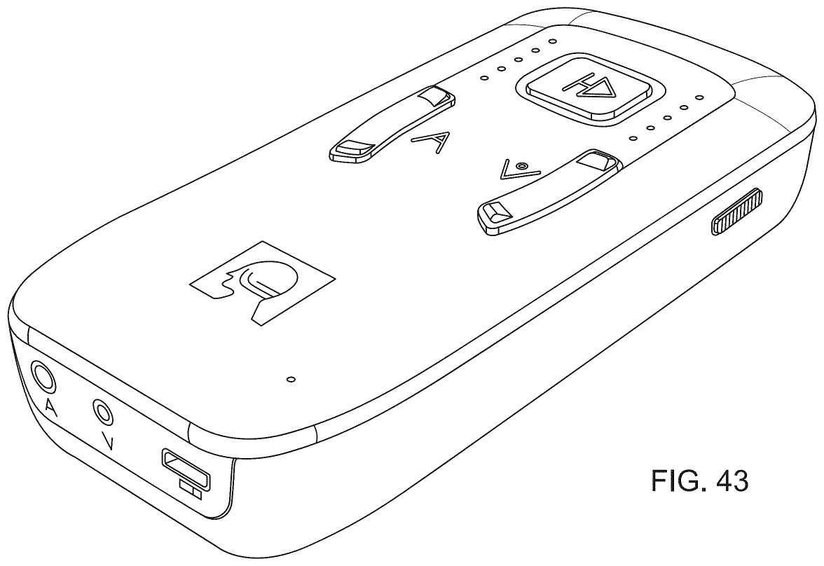

Maintenance of Functional Ability Assessed by ADCS-ADL. Effects of NSS treatment on patients' the ability to perform activities of daily living were assessed at baseline and regular monthly intervals during the 24-week treatment period using the clinically proven ADCS-ADL scale via structured interview with care partner. Average ADCS-ADL scores were calculated from the first 12-week and second 12-week periods in both treatment (n=14) and sham (n=8) groups (FIG. 41). The ADCS-ADL is a well-established instrument for testing function of mild to moderate AD patients, and numerous clinical trials have reported a significant decline in the ADCS-ADL scores in this patient population over a 6-month period (Loy, C., and L. Schneider (2006). “Galantamine for Alzheimer's disease and mild cognitive impairment.” Cochrane Database Syst Rev (1): CD001747; Peskind, E. R., S. G. Potkin, N. Pomara, B. R. Ott, S. M. Graham, J. T. Olin and S. McDonald (2006). “Memantine treatment in mild to moderate Alzheimer disease: a 24-week randomized, controlled trial.” Am J Geriatr Psychiatry 14 (8): 704-715). In our study, each patient in the sham group showed a decline in ADCS-ADL scores, resulting in this patient group significant (p<0.001), approximately 3 points decline over the trail period. In contrast, 9 out of 14 patients in the treatment group maintained or even showed improvement in their ADCS-ADL scores. Therefore, the average ADSC-ADL score in the treatment group significantly (p<0.035) increased during the treatment period. Accordingly, FIG. 41 demonstrates that changes in daytime activities showed a significant improvement in the treatment group and a significant decline in the sham group.

### DISCUSSION

This interim analysis of the Overture trial (NCT03556280) demonstrates a beneficial outcome of daily use of the NSS therapy over a six-month period in mild to moderate AD patients: NSS treatment resulted in improved sleep quality and maintained quality of daily living as compared to subjects in the control arm of the study.

Results, based on the collected actigraphy data over a 6-month period, demonstrate that NSS therapy can reduce sleep fragmentation, leading to significantly reduced active periods during night in mild to moderate AD patients. In contrast, patients in the sham group did not show improvement in sleep characteristics. Given the well-recognized architecture of human physiological sleep, consisting subsequent periods of different NREM stages starting from superficial to deep slow wave sleep and REM sleep period in a strictly subsequent order, it is obvious that sleep fragmentation can dramatically disrupts sleep architecture and consequently effectiveness of sleep. Sleep fragmentation, as a symptom of sleep disorders have multiple impact on human physiology, including dysfunction not only in the nervous system, but also overall health by impairing body metabolism or immune defense system. Nevertheless, decremental cognitive impacts of sleep abnormalities are particularly worrisome in MCI and AD patients. Therefore, application of NSS therapy offers novel intervention for in AD patients for improving sleep quality. Available clinical data revealed that this therapy is safe and can be applied daily in an extended period of time in patients. Considering that sleep disorders are contributing to impaired function and cognition, effective treatments for improving sleep quality potentially have multiple benefits in MCI and AD patients.

The clinical benefits of NSS therapy on sleep is particularly relevant, since pathomechanisms underlying sleep dysfunction in MCI and AD patients are not well understood, therefore developing specific sleep therapies are not feasible currently. AD-related pathological proteins, such as Aβ- and tau-oligomers are known to disrupt sleep, though their mode of action is unknown. From an early stage of the disease brainstem ascending neurons considered to play in role in sleep-wake regulation, including cholinergic, serotoninergic and norepinephrine neurons show profound degeneration (Smith, M. T., C. S. McCrae, J. Cheung, J. L. Martin, C. G. Harrod, J. L. Heald and K. A. Carden (2018). “Use of Actigraphy for the Evaluation of Sleep Disorders and Circadian Rhythm Sleep-Wake Disorders: An American Academy of Sleep Medicine Systematic Review, Meta-Analysis, and GRADE Assessment.” J Clin Sleep Med 14 (7): 1209-1230; Tiepolt, S., M. Patt, G. Aghakhanyan, P. M. Meyer, S. Hesse, H. Barthel and O. Sabri (2019). “Current radiotracers to image neurodegenerative diseases.” EJNMMI Radiopharm Chem 4 (1): 17; Kang, S. S., X. Liu, E. H. Ahn, J. Xiang, F. P. Manfredsson, X. Yang, H. R. Luo, L. C. Liles, D. Weinshenker and K. Ye (2020). “Norepinephrine metabolite DOPEGAL activates AEP and pathological Tau aggregation in locus coeruleus.” The Journal of Clinical Investigation 130 (1): 422-437). Similarly, suprachiasmatic nucleus-containing neurons playing the key role in regulating circadian rhythms also shows neurodegeneration early in the disease Van Erum, J., D. Van Dam and P. P. De Deyn (2018). “Sleep and Alzheimer's disease: A pivotal role for the suprachiasmatic nucleus.” Sleep Med Rev 40:17-27). There are only limited treatment options for sleep abnormalities in MCI and AD patients, and pharmacological treatments currently include antidepressant, antihistamines, anxiolytics, and sedative-hypnotic drugs such as benzodiazepines (Vitiello, M. V. and S. Borson (2001). “Sleep disturbances in patients with Alzheimer's disease: epidemiology, pathophysiology and treatment.” CNS Drugs 15 (10): 777-796; Deschenes, C. L. and S. M. McCurry (2009). “Current treatments for sleep disturbances in individuals with dementia.” Curr Psychiatry Rep 11 (1): 20-26; Ooms, S. and Y. E. Ju (2016). “Treatment of Sleep Disorders in Dementia.” Curr Treat Options Neurol 18 (9): 40). Some of the most frequently used anxiolytics/sedative-hypnotic drugs in the general clinical practice are GABA_{A} positive allosteric modulators, which are contraindicated in MCI and AD patients due to their negative effects on cognitive function, interference with motor behavior and addiction-forming profile. Recently, suvorexant, an orexin receptor antagonist has been approved as a sleep medication for AD patients having clinically diagnosed insomnia. The main effects of suvorexant are a prolonged total sleep time and delayed wake after sleep onset, without impacting sleep fragmentation or altering sleep architecture (Herring, W. J., P. Ceesay, E. Snyder, D. Bliwise, K. Budd, J. Hutzelmann, J. Stevens, C. Lines and D. Michelson (2020). “Polysomnographic assessment of suvorexant in patients with probable Alzheimer's disease dementia and insomnia: a randomized trial.” Alzheimers Dement 16 (3): 541-551). Non-pharmacological treatments include behavioral measures such as sleep hygiene education, exercise regimens, and reduction of noise during sleeping hours. Bright light therapy is one of the non-pharmacologic modalities that offers recommendations from the American Academy of Sleep Medicine for use in sleep disturbances due to circadian disorders. Clinical tests of light therapy in AD patients resulted in conflicting findings (Ouslander, J .G., Connell, B. R., Bliwise, D. L., Endeshaw, Y., Griffiths, P. and Schnelle, J. F. (2006). “A Nonpharmacological Intervention to Improve Sleep in Nursing Home Patients: Results of a Controlled Clinical Trial.” Journal of the American Geriatrics Society. 54:38-47; Deschenes et al., 2009), and currently no approved device or therapeutic intervention exists.

The current findings demonstrate a beneficial effect of NSS therapy in mild to moderate AD patients, prolonging nighttime undisturbed restful periods, indicating a reduced sleep fragmentation. There are no proved therapies for reducing sleep fragmentation which could improve sleep quality in MCI or AD patients, and frequently used sedative-hypnotic drugs are decremental on the physiological architecture of sleep. Having monthly interviews with patients and caregivers about everyday activities and sleep habits, there was not an indication that NSS treatment leads to daytime sleepiness or grogginess, which are typical side effects of most sleep medication, including the orexin receptor antagonist suvorexant. Furthermore, in the present trial clinically diagnosed sleep abnormality such as insomnia has not been a requirement, consequently beneficial effects of NSS treatment are not limited to AD patients suffering from clinically recognized sleep problems.

The present findings demonstrate that NSS treatment not only improves sleep quality but also helps to maintain functional ability reflected in activity of daily living in mild to moderate AD patients. Although some pharmacological treatments, such as the acetylcholine esterase inhibitor donepezil, delay decline in activity of daily living, currently there are no approved non-pharmacological therapies achieving this effect. Based on scientific and clinical observations demonstrating a close relationship between sleep quality and activity of daily living, it can be presumed that improving sleep quality in AD patients would provide multiple benefits: better sleep will enhance patients' daytime performance, including cognitive function, and reduce daytime sleepiness. In line with this hypothesis, patients on NSS treatment maintained functional activity as reflected by their unchanged ADSC-ADL score over the six-month treatment period. In contrast, ADSC scores of sham group patients dropped similarly to changes of placebo group patients in clinical trials (Doody, R. S., R. Raman, M. Farlow, T. Iwatsubo, B. Vellas, S. Joffe, K. Kieburtz, F. He, X. Sun, R. G. Thomas, P. S. Aisen, C. Alzheimer's Disease Cooperative Study Steering, E. Siemers, G. Sethuraman, R. Mohs and G. Semagacestat Study (2013). “A phase 3 trial of semagacestat for treatment of Alzheimer's disease.” N Engl J Med 369 (4): 341-350; Doody, R. S., R. G. Thomas, M. Farlow, T. Iwatsubo, B. Vellas, S. Joffe, K. Kieburtz, R. Raman, X. Sun, P. S. Aisen, E. Siemers, H. Liu-Seifert, R. Mohs, C. Alzheimer's Disease Cooperative Study Steering and G. Solanezumab Study (2014). “Phase 3 trials of solanezumab for mild-to-moderate Alzheimer's disease.” N Engl J Med 370 (4): 311-321). Even though the close relationship between sleep and daily activity is well documented, it is unknown at present whether improved sleep quality is the main factor contributing to the maintenance of ADSC-ADL scores in NSS treated patients, or improvement in sleep and continuation of functional ability are unrelated positive outcomes from the therapy.

Currently, the underlying mechanisms of improved sleep and maintained functional ability of AD patients in response to NSS treatment are not known. Preclinical studies indicate that gamma oscillation inducing 40 Hz sensory stimulation reverses Aß and tau pathologies leading to improved cognitive function in transgenic mice (Iaccarino, Singer et al. 2016; Adaikkan, C., S. J. Middleton, A. Marco, P. C. Pao, H. Mathys, D. N. Kim, F. Gao, J. Z. Young, H. J. Suk, E. S. Boyden, T. J. McHugh and L. H. Tsai (2019). “Gamma Entrainment Binds Higher-Order Brain Regions and Offers Neuroprotection.” Neuron 102 (5): 929-943 e928; Martorell, Paulson et al. 2019). Although human AD-related biomarker studies are in progress, it is unknown whether the same biochemical and neuroimmunology mechanisms are activated in AD patients as identified in mice. The bidirectional interaction between sleep and disease progression (Wang and Holtzman 2020) supports the notion that improved sleep in response to NSS treatment could also slow down disease progression.

### Conclusion

The present findings indicate that NSS treatment helps maintain everyday activity and quality of life of AD patients. Since measurements of both sleep fragmentation and ADCS-ADL were determined in the same patient cohort, the data suggest a positive treatment effect of maintaining ability to complete daily activities in patients having improved sleep quality. NSS treatment consists of a non-invasive sensory stimulation; with exceptional safety profile, its long-term, chronic application is feasible. Expanded and longer trials will uncover additional clinical benefits and potentially disease-modifying properties of NSS treatment.

### Example 3. Randomized Controlled Trial with Greater Amount of Participants

### Background

An additional randomized controlled trial was performed, with patients maintaining the same methods and inclusion criteria as the interim analysis of the trial disclosed herein, in EXAMPLE 2. This trial involved a greater number of participants than that which was subject to the interim analysis

### Methods

Patients with mild-to-moderate AD (MMSE 14-26, inclusive; n=74) were randomized to receive either 40 Hz noninvasive audio-visual stimulation or sham stimulation over a 6-month period. Functional abilities of patients were measured by Alzheimer's Disease Cooperative Study—Activities of Daily Living (ADCS-ADL) scale at baseline and every four weeks during the study and follow-up period. Sleep quality was assessed from nighttime activities of a subgroup of patients (n=7 in treatment, n=6 in sham groups) who were monitored continuously via a wrist worn actigraphy watch throughout the 6-month period.

### Results

The sham group contained 19 patients, and the treatment group contained 33 patients. Adjusted ADCS-ADL scores from beginning and the end of the trial were compared in patients who completed the trial. Over the 6-month period, patients in the sham group (n=19) showed the expected decline, a 5.40-point drop in ADCS-ADL scores, whereas patients in the treatment group (n=33) receiving therapy exhibited only a 0.57-point decline. Changes in ADCS-ADL scores were statistically significant between the sham and treatment groups (P<0.01). Nighttime active durations in the treatment group were significantly (p<0.03) reduced in the second 3 months compared to the first 3-months but such durations increased in the sham group. To evaluate the impact on active durations, normalization is done by dividing duration of each active period by the duration of the matching entire nighttime period. Analysis of normalized active durations by the corresponding nighttime period of each patient further confirmed opposite changes in nighttime active durations between treatment and sham groups (p<0.001), with the treatment group experiencing reduced nighttime active durations, and the sham group experiencing increased nighttime active durations.

### Conclusion

This trial confirmed that patients in gamma oscillation inducing non-invasive sensory stimulation therapy maintained their activities of daily living and showed an improved sleep quality over a 6-month treatment period; two outcome measures, functional ability and sleep quality known to be strongly linked in AD. Maintenance of functional ability represents an important treatment and management goal for AD patients, reducing formal and informal care, and delaying time to institutionalization.

### Example 4. Randomized Controlled Trial to Evaluate Impact on a Non-Patient Population

### Background

Participants will be recruited using social media advertisements and selected randomly. Criteria will simply include willingness and availability to participate in a six-month trial. Information will be collected on each individual to generate a profile associated with the individual.

### Methods

Participants will be recruited using social media advertisements and selected randomly. Criteria will simply include willingness and availability to participate in a six-month trial. Information will be collected on each individual to generate a profile associated with the individual.

Participants will be randomized into two groups initially, with a 1:1 ratio of treatment group to control group. Within the treatment group, subjects will remain blinded and receive a neural stimulation orchestration system device which outputs sensory stimulation at a 40 Hz frequency. Within the control group, subjects will remain blinded and receive a neural stimulation orchestration system device which outputs sensory stimulation at a random distribution of time around a mean of 35 Hz. Throughout the study, subjects will wear actigraphy watches. These watches will monitor any sleep fragmentation or disturbances experienced by a participant. Cognitive tests, or assessments, will be performed on each subject before neural stimulation orchestration devices are distributed to establish a baseline. These tests will be repeated on bimonthly basis, and the study will conclude after six months. Each assessment is of general cognitive functions, which pertain to both healthy individuals and individuals that have experienced or are at risk of experiencing cognitive deficits, including clinical patient populations. Such suitable tests include those that test any specific functions of a range of cognitions in cognitive or behavioral studies, including tests for perceptive abilities, reaction and other motor functions, visual acuity, long-term memory, working memory, short-term memory, logic, decision-making, and the like.

The following cognitive tests will be used: Visual Short-term memory; Spatial Working Memory; N-back; Stroop Task; Attention Blink; Task Switch; Trials A&B; Flanker Task; Visual Search Task; Perceptual Motor Speed; Basic Processing Speed; Digit Span. These tests are described as follows:

Visual Short-term memory (VSTM). In the visual short-term memory task, individuals are briefly presented with four color patches presented at the center of the screen and are asked to remember the colors. Following a short delay, a single color patch is shown, and the individual is asked whether the color was one of those presented or not. For example, on a given trial an individual can be briefly presented with color patches in blue, red, green, and yellow and asked to remember them. If they were then shown the color purple, they would respond no match because the color purple was not in the presented and remembered set. This test measures the ability to remember visual information in the short-term.

Spatial Working Memory (SPWM). In the spatial working memory task, one to three objects are briefly flashed on the screen and then disappear, and individuals are asked to remember the locations of each of the objects. After a brief delay, a single object appears on the screen and the participant responds to whether the object is in the same location as one of the objects being remembered. This task measures the ability to remember visuospatial information in the short-term.

N-back. In the N-back task individuals are presented with a continuous stream of letters at the center of the screen and are asked to respond whether the letter presented on the current trial matches the one presented on the previous trial. For example, an individual can see the letter W followed by the letter S, and then would be asked to respond to whether the W and S match identity. This test measures how well participants can hold and manipulate information in short-term memory. In another version of this task, individuals are presented with a continuous stream of letters at the center of the screen and are asked to respond whether the letter presented on the current trial matches the one presented two trials ago.

Stroop Task. In the Stroop task individuals are asked to name the color of a written word presented at the center of the screen as quickly as possible. The word can either be a color-word (e.g., the word red written in either green or red) or a non-color word (e.g., the word cat written in red). The ability to focus attention is assessed by seeing how much an incorrect color/word combination (e.g., the word red written in green) slows an individual's reaction time. This task provides a measure of how well an individual can control attention and executive function processes.

Attention Blink. In the Attentional Blink task an individual views a stream of letters presented rapidly at the center of the screen and is asked to search the stream for either one or two pre-defined target letters. On trials in which there are two targets, detecting the first target interferes with the ability of an individual to detect the second target, and the extent of this interference is used to assess attention function.

Task Switch. In the task-switch task, individuals see a digit (e.g., 1-10) at the center of the screen, and the digit appears on a color patch. Depending on the color of the patch, the individual responds to either the parity (e.g., high vs. low) of the number or whether the number is odd or even. Importantly, on each trial the color patch is either the same color as the previous trial, resulting in participants performing the same task from trial to trial, or a different color than the previous trial, resulting in a switch in the task. For example, on a given trial an observer can see the number two on a pink color patch. On this trial, the individual would perform the parity judgment task. On the following trial, if the color patch stays the same the individual would continue to perform the parity task. However, if the color patch changes color, this signals that the individual should switch and perform the odd/even task on this trial. This task measures the ability to rapidly switch tasks, a subset of executive function.

Trails A&B. In the Trails task, individuals are to connect dots in sequence as quickly as possible using their finger. In trails A, individuals are asked to connect dots 120 in sequence. In trails B, individuals are asked to connect many more dots or dots 110 and A-J in sequence, alternating between numbers and letters. This test measures how quickly individuals can search for and sequentially process information from the within a category (Trails A) or between categories (Trails B). The Trails test measures attention and executive function.

Flanker Task. In the flanker task, individuals are presented with a display containing several objects. One of the objects, the target, is always presented at the center of the screen, and participants are asked to identify which of two target types the item is. The target is flanked on both sides by distractor objects that are either identical to the target on a given trial or not. For example, participants can view a display containing multiple arrows. One arrow, the target, will be presented at the center of the screen and participants' task is to report whether the arrow is pointing to the left or to the right. This arrow is surrounded on both sides by arrows that are pointing in either the same or different directions. This task assesses how well individuals can focus attention on relevant and ignore irrelevant visual information, providing a measure of attention and executive function.

Visual Search Task. In the Visual Search Task, individuals are presented with an array of objects and are asked to find a target object as quickly as possible. For example, an individual can be told to search for a particular color box with a gap in the top or bottom and report the location (top or bottom) of the gap. This task assesses how quickly an individual can find and identify basic visual information, a subset of attention function.

Perceptual Motor Speed (PMS). In this task, individuals are presented with a schematic face and are asked to press a button as soon as possible in response to a happy face and withhold their response to a sad face. The ability to withhold a response to sad faces provides a measure of executive function, and the speed with which responses to happy faces are made provides a measure of processing speed.

Basic Processing Speed. In this task, individuals monitor a blank screen, and after a variable delay a small circle appears at the center of the screen. Participants are asked to press a button as quickly as possible when they see the circle appear. This task measures basic visual processing speed.

Digit Span. In this task observers see strings of two to eight numbers and are asked to remember their identities and their order. After the strings are removed from the screen, participants need to type as many of the numbers as they can remember. This test provides a measure of verbal short-term memory.

Participants will be given a list of activities to participate in. Each activity will involve a different type of cognitive processing. All study participants will be divided into six groups, with each group comprising an equal amount of control and treatment group members. Each participant will be instructed to reflect on their performance during each activity and record any observations in a journal. Participants will also be asked to record information about their sleep quality, mood, and energy levels in this journal.

The neural stimulation system will provide visual stimulation for one hour per use. One group will use the neurostimulation system for one hour prior to engaging in a selected group of activities, a second group will use the neurostimulation system during engagement in the selected group of activities, and a third group will use the neurostimulation system before, during, and after engaging in the selected group of activities. The fourth group will use the neurostimulation system both during and prior to engaging in the selected group of activities. The fifth group will use the system both prior to engaging in the activities and after engaging in the activities. The sixth group will use the system during and after engaging in the activities.

The impact of the stimulation on a particular group of activities will be measured by participants' self-assessment journals and the results of each cognitive test-based assessment. The impact will be compared for each of the six groups. Profile information obtained in the beginning of the study for each individual will be used to inform differences or discrepancies in response within each group.

The amount of time between use of the neural stimulation system and the start or end of an activity will be held constant within each group. Groups of activities will vary each month and will be rotated so that each participant, by the end of the trial, has engaged in the same activities as the others. Some activities will simulate a learning environment, with subjects being given a definite, supervised period to learn a particular subject and then tested on their ability to recall the learned material. Other activities will involve physical movement and coordination, such as an athletic activity, while some activities will require a participant to operate a vehicle.

Some activities will require little physical movement, such as rest or meditation. At the completion of the six months, each group will have participated in the same activities.

### Results

Based on the benefits of sensory induction of gamma neural oscillations seen in the studies involving patients with AD, such as slowing dementia, slowing brain atrophy, and improving sleep, it is predicted that the subjects in the treatment group will experience a slight improvement in cognitive capacity. Further, it is predicted that groups receiving 40 Hz neurostimulation during a particular activity will demonstrate the best improvement. Statistical analysis of these results can be used to inform the policy used by the machine learning algorithm in determining whether to adjust an output signal parameter that causes the stimulus-emitting component of the present invention to provide gamma-inducing sensory stimulus with certain characteristics (e.g., frequency, intensity) to a subject, thereby promoting gamma oscillations and assisting in predicting expected treatment outcome.

### Example 5. White Matter Atrophy and Myelination

### Objectives

The present study evaluated whether gamma oscillation inducing non-invasive sensory stimulation for a 6-month period could affect white matter atrophy and myelination in patients on AD spectrum.

### Methods and Materials

The neuroimaging data used in this study is collected in Cognito Therapeutics' Overture, a randomized, placebo-controlled feasibility study (NCT03556280) in patients (age of 50 years or older and Mini-Mental State Examination (MMSE) 14-26) on AD spectrum. In this study, participants in the active treatment arm received 1-hour daily, at-home, 40 Hz simultaneous auditory-visual sensory stimulation for a 6-month period while the placebo arm subjects received sham stimulation. Structural magnetic resonance imaging (MRI) data was acquired at baseline, month 3, and month 6 visits using 1.5 Tesla MRI. 38 participants (25 Treatment and 13 Placebo) who fulfilled the requirement of sufficient T1-weighted (T1w) image quality were included in the analysis. Volume assessments on multiple white matter structures were done using T1 MRI, and myelination assessments were done using T1w/T2w ratio. One treatment group and one placebo group participant were excluded from myelination analysis owing to T2w image quality. Patient characteristics at baseline are summarized in TABLE 3. Bayesian linear mixed effects modeling was used to assess the changes from baseline. Changes in white matter volume and myelination were compared between treatment group and placebo group participants after 6 months of treatment.

TABLE 3
Demographic and clinical characteristics of the treatment
and the placebo group participants at baseline.

| | Treatment | Placebo | [symbol] |
|---|---|---|---|
| | (n = 25) | (n = 13) | value |
| Age in years, mean ± SD | [symbol] 8.36 ± 7.69 | [symbol] 6.62 ± 9.97 | [symbol] .02 |
| Sex (Male/Female) | [symbol] (Male)/ | [symbol] (Male)/ | [symbol] .10 |
| | 18(Female) | 5(Female) | |
| MMSE score, mean ± SD | [symbol] 0.64 ± 3.15 | [symbol] 9.77 ± 3.27 | [symbol] .44 |
| ADCS-ADL scale, mean ± SD | [symbol] 4.88 ± 7.95 | [symbol] 6.23 ± 10.83 | [symbol] .70 |
| Number (%) of APOE ε4 positive | [symbol] 3(52.00%) | [symbol] (53.85%) | [symbol] |

Abbreviations:
MMSE, Mini-Mental State Exam;
ADCS-ADL, Alzheimer's Disease Cooperative Study - Activities of Daily Living;
APOE, apolipoprotein E [symbol]
indicates data missing or illegible when filed

Therapy Device. The device used in this study is a gamma oscillation inducing sensory stimulation device developed by Cognito Therapeutics, Inc. It consists of a handheld controller, an eye-set for visual stimulation and headphones for auditory stimulation. All the components work in synchrony to provide precisely timed non-invasive 40 Hz stimulation to evoke steady-state gamma brainwave activity. Prior to study, a physician determines the tolerable range of stimulus parameters for the participant. During the therapy, participants can also adjust the brightness of the visual stimulation and the volume of the auditory stimulation using push buttons on the controller. If assistance is needed, they can communicate with a care partner. The device captures usage information and adherence data. All the information is uploaded to a secured cloud server for remote monitoring.

MRI Data Acquisition. In an Overture feasibility study, structural magnetic resonance imaging (MRI) data were acquired at Baseline, month 3, and month 6 using 1.5 Tesla MRI scanner. The study adopted a ADNI1 comparable standardized MRI scan protocol. For T1w, it included 1.25×1.25 mm in-plane spatial resolution, 1.2 mm thickness, TR 2400 ms and TE 3.65 ms for Siemens Espree scanner, 0.94×0.94 mm in-plane spatial resolution, 1.2-mm thickness, TR ~3.9 ms and TE 1.35 ms in General Electric scanner Signa HDxt and 0.94×0.94 mm in-plane spatial resolution, 1.2-mm thickness, TR 9.5 ms and TE ~3.6 or 4 ms in Philips Ingenia scanner or Philips Achieva scanner. For T2w, it included 1×1 mm in-plane spatial resolution, 4 mm thickness, TR 3000 ms and TE 96 ms for Siemens and GE scanners and 1×1 mm in-plane spatial resolution, 4 mm thickness, TR 3000 ms and TE 92 ms for Philips scanner (Jack et al. 2008).

Image Analysis. The FreeSurfer pipeline is used to process and automatically parcellate T1 MRI into predefined cortical structures and segment the volume into predefined subcortical structures (Dale et al., 1999; Fischl et al., 2001; Fischl et al., 2008; Fischl et al., 2002; Fischl et al., 1999a; Fischl et al., 1999b; Ségonne et al., 2005; Desikan et al., 2006). Here, we focus on a total of 52 white matter structures to assess volumetric changes and evaluate myelin content.

Myelin Sensitive Imaging. A non-invasive myelin-sensitive imaging was employed by using T1w/T2w ratio to acquire a myelin-reflecting contrast (Glasser and Van Essen, 2011; Glasser et al., 2014, 2016). This process included co-registration of the T2w images to the T1w images using rigid transformation, inhomogeneity correction for both T1w and T2w images and linear calibration of image intensity using non-brain tissue masks to create T1w/T2w ratio images corresponding to myelin content (Ganzetti et al., 2014, 2015). T1w/T2w ratio was processed using MRTool (v. 1.4.3, https://www.nitrc.org/projects/mrtool/), the toolbox implemented in the SPM12 software (University College London, London, UK, http://www.fil.ion.ucl.ac.uk/spm).

Statistical Methods. Demographic and biomarker data of the treatment group participants and the placebo group participants were compared using two-sample T tests for numerical data or chi-square tests for categorical data. For efficacy analysis, a Bayesian linear mixed effects model was used to assess the changes in the volumetric data and myelination for each of the white matter structures. Fixed effects of the model include total intracranial volume, baseline MMSE score, baseline age, visit (as number of days from the start of the treatment), group, baseline MRI measures (volume for white matter atrophy assessment and sum of T1w/T2w ratio for myelination assessment), group-visit interaction and baseline MRI measures-visit interaction. Random effects of the model include subject and site information. The Kenward-Roger approximation of the degrees of freedom was used. For volumetric analysis, volume change (% change from baseline) and for myelination analysis, sum of T1w/T2w ratio change (% change from baseline) were assessed for each studied white matter structure. All statistical analyses were conducted using R (R version 4.1.1).

### Results

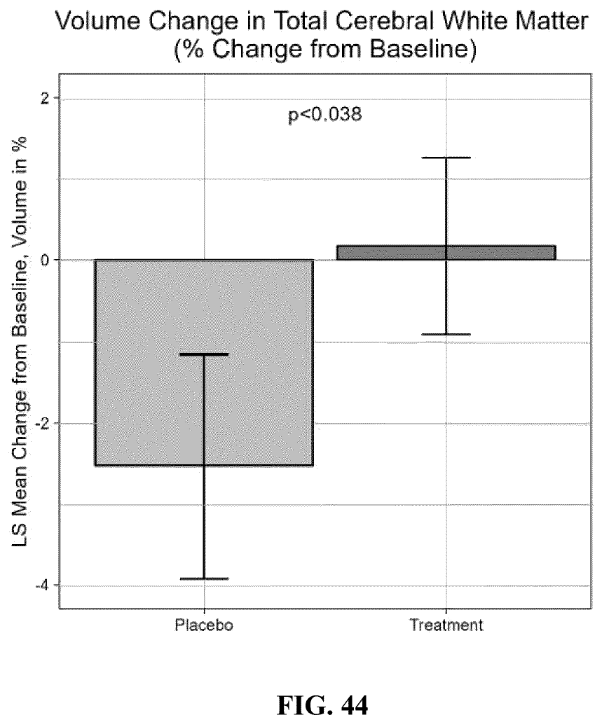

With respect to baseline levels, it was observed that the treatment group demonstrated a 0.17±1.08% increase and the placebo group demonstrated a −2.54±1.38% decrease in total cerebral white matter volume after a 6-month period. The difference between these two groups was statistically significant (p<0.038). See FIG. 44.

FIG. 44 provides white matter volume change from baseline (%) after gamma oscillation inducing 40 Hz sensory stimulation therapy for a 6-month period favors the treatment group. LS Mean volume changes for the total cerebral white matter show the significant difference (p<0.038) between the Treatment group participants (dark gray) and the Placebo group participants (light gray), favoring the Treatment group. Error bars indicate SE.

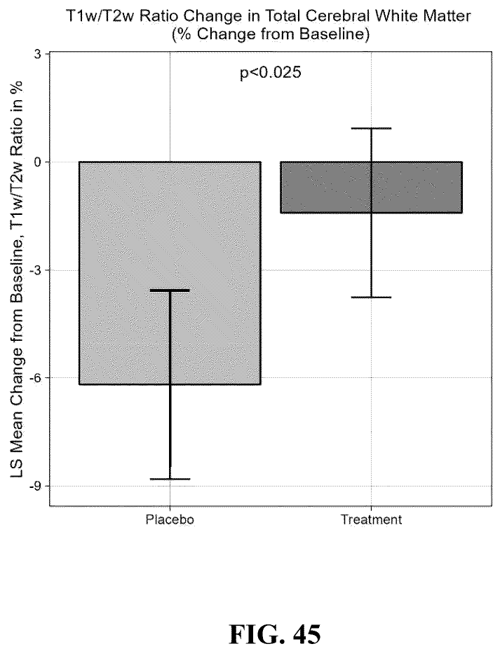

It was also observed that the treatment group demonstrated a −1.42±2.35% decrease and the placebo group demonstrated a −6.19±2.63% decrease in myelination as assessed by summing the ratio of T1 weighted (T1w) and T2 weighted (T2w) intensities across the MRI images. This difference was also statistically significant (p<0.025) between groups. See FIG. 45. FIG. 45 provides T1w/T2w ratio change in white matter (% change from baseline) after gamma oscillation inducting 40 Hz sensory stimulation therapy for a 6-month period. LS Mean sum of T1w/T2w ratio changes for the total cerebral white matter show the significant difference (p<0.025) between the Treatment group participants (dark gray) and the Placebo group participants (light gray), favoring the Treatment group. Error bars indicate SE.

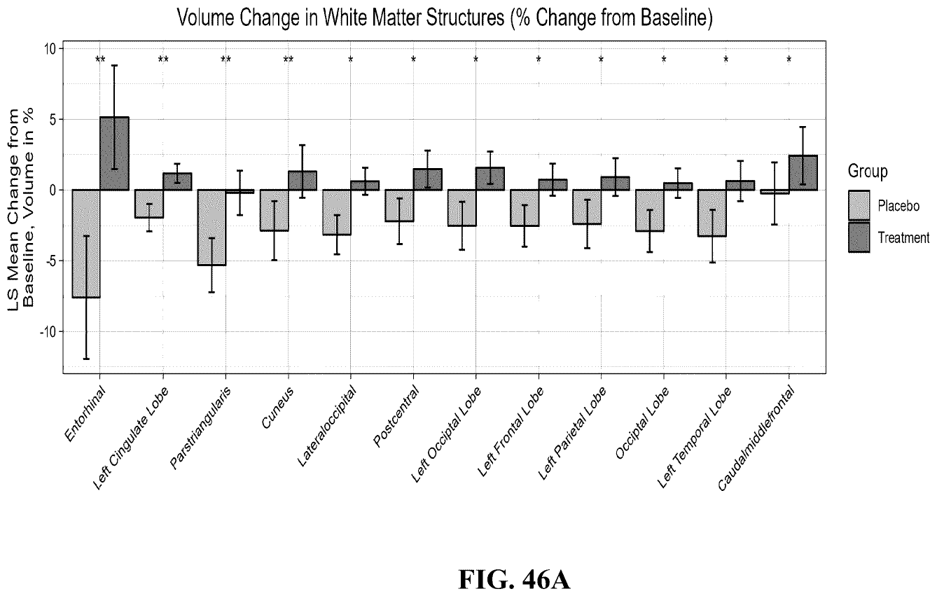

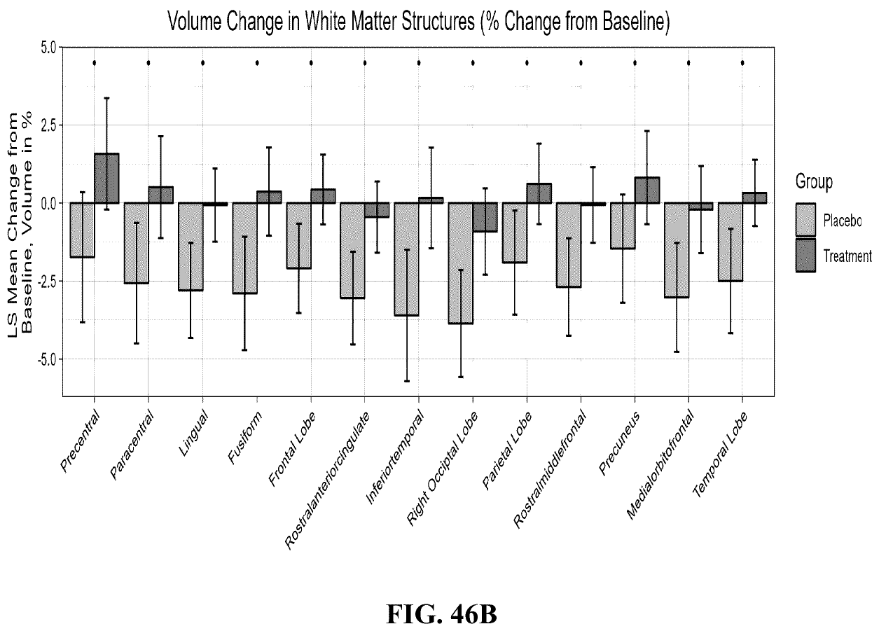

Next, the structures that respond to treatment the most, in volume and myelin-reflecting T1w/T2w ratio changes among 52 white matter structures, were examined. All statistically significant changes favored the treatment group. Compared to the placebo group, significant (p<0.05) attenuation in volume loss was identified in 12 of 52 structures: the entorhinal region, left cingulate lobe, pars triangularis region, cuneus region, lateral occipital region, postcentral region, left occipital lobe, left frontal lobe, left parietal lobe, occipital lobe, left temporal lobe, and caudal middle frontal region (sorted in ascending order by p value) for the treatment group after 6 months of treatment (FIG. 46A). Gamma oscillation inducing 40 Hz sensory stimulation therapy administered over a 6-month period most significantly reduced white matter atrophy in entorhinal region. The treatment group demonstrated a 5.14±3.66% (0.08±0.06 cm³) increase, while the placebo group demonstrated a −7.60±4.35% (−0.13±0.07 cm³) decrease in volume. The difference between these two groups was statistically significant (p<0.002). The treatment also trended in the direction of preventing volume loss (0.05≤p<0.1) in the precentral region, paracentral region, lingual region, fusiform region, frontal lobe, rostral anterior cingulate region, inferior temporal region, right occipital lobe, parietal lobe, rostral middle frontal, precuneus region, medial orbitofrontal region, and temporal lobe (sorted in ascending order by p value) (FIG. 46B).

FIG. 46A and FIG. 46B provide white matter structures volume change from baseline (%) after gamma oscillation inducing 40 Hz sensory stimulation therapy for a 6-month period. LS Mean volume changes for the white matter structures (FIG. 46A, sorted in ascending order by p value) show the significant difference (p<0.05) between the Treatment group participants (dark gray) and the Placebo group participants (light gray), favoring the treatment group. FIG. 46B (sorted in ascending order by p value) shows LS Mean volume changes for the white matter structures with the marginal difference (0.05≤p<0.1) between the Treatment group participants (dark gray) and the Placebo group participants (light gray), favoring the Treatment group. Error bars indicate SE. * p<0.05, ** for p<0.01, and · for 0.05<p<0.1.

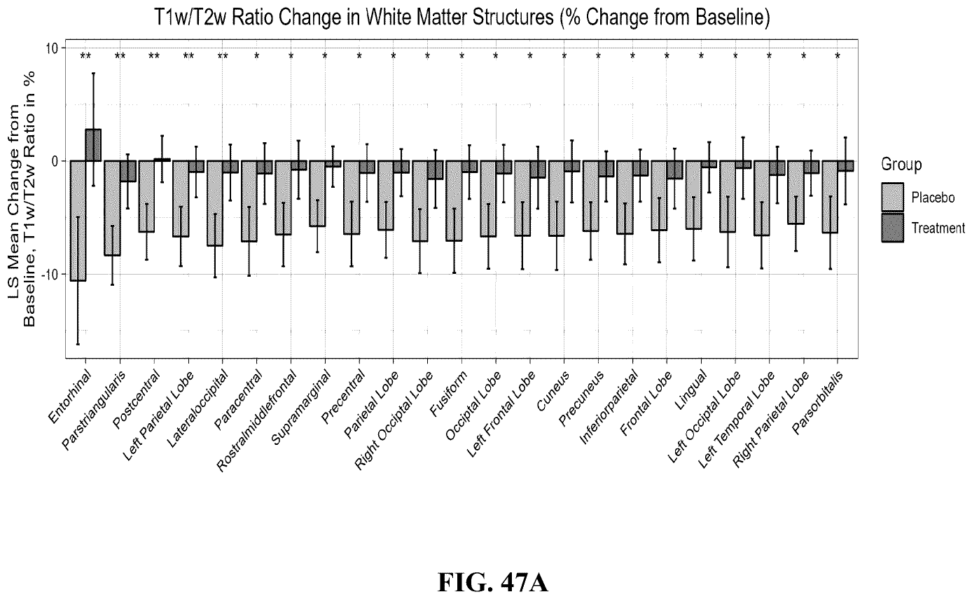

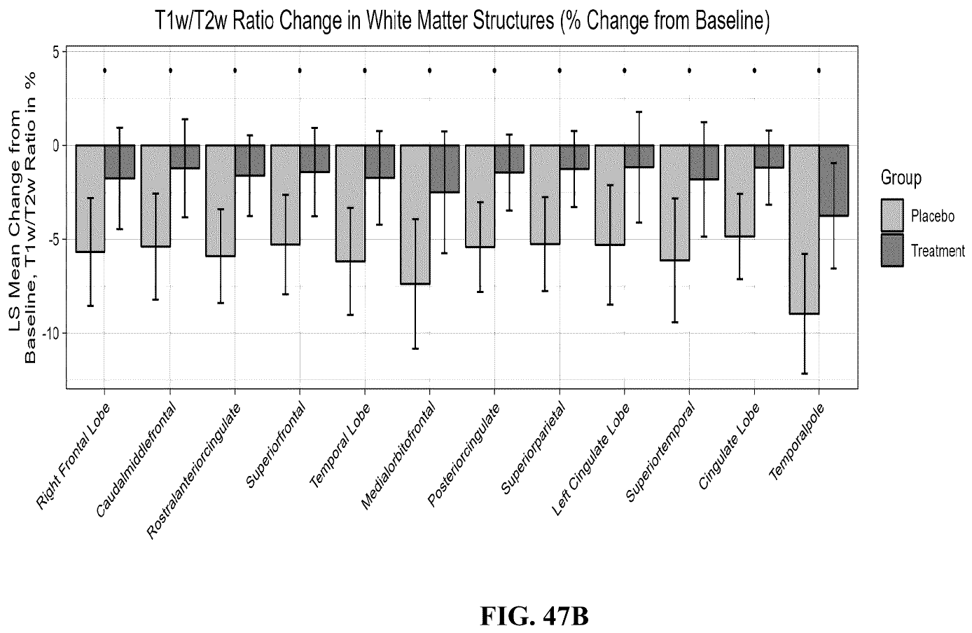

Compared to the placebo group, significantly less myelin damage (T1w/T2w ratio) was observed in entorhinal region, pars triangularis region, postcentral region, left parietal lobe, lateral occipital region, paracentral region, rostral middle frontal region, supramarginal region, precentral region, parietal lobe, right occipital lobe, fusiform region, occipital lobe, left frontal lobe, cuneus region, precuneus region, inferior parietal region, frontal lobe, lingual region, left occipital lobe, left temporal lobe, right parietal lobe and pars orbitalis region (FIG. 47A, white matter structures sorted in ascending order by p value), indicating significant differences (p<0.05) between the treatment group and the placebo group. Within the 52 studied white matter structures, the most significant myelin reflecting T1w/T2w ratio change was also in the entorhinal region. The treatment group participants exhibit a +2.78±4.97% increase from baseline on the sum of the T1w/T2w ratio while the placebo group participants exhibit a −10.59-5.63% decrease from baseline on the sum of the T1w/T2w ratio (p<0.003), suggesting that gamma oscillation inducing 40 Hz sensory stimulation therapy for a 6-month period may significantly protect myelin damage in this brain region. The treatment may also trend towards slowing down demyelination (0.05≤p<0.1) in right frontal lobe, caudal middle frontal region, rostral anterior cingulate region, superior frontal region, temporal lobe, medial orbitofrontal region, posterior cingulate region, superior parietal region, left cingulate lobe, superior temporal region, cingulate lobe, and temporal pole region (FIG. 47B, white matter structures sorted in ascending order by p value).

FIGS. 47A and 47B provide T1w/T2w ratio change in white matter structures (% change from baseline) after gamma oscillation inducing 40 Hz sensory stimulation therapy for a 6-month period. LS Mean sum of T1w/T2w ratio changes in the white matter structures (Panel A, sorted in ascending order by p value) shows the significant difference (p<0.05) between the Treatment group participants (dark gray) and the Placebo group participants (light gray), favoring the treatment group. Panel B (sorted in ascending order by p value) shows LS Mean sum of T1w/T2w ratio changes in the white matter structures with the marginal difference (0.05≤p<0.1) between the Treatment group participants (dark gray) and the Placebo group participants (light gray), favoring the Treatment group. Error bars indicate SE. * p<0.05, ** for p<0.01, and · for 0.05<p<0.1.

These results suggest that gamma oscillation inducing 40 Hz sensory stimulation therapy for a 6-month period may reduce white matter atrophy and that the changes are accompanied by significantly less demyelination in the treatment group compared to the placebo group.

### Conclusions

Administration of gamma oscillation inducing 40 Hz sensory stimulation for a 6-month period led to beneficial effects on total and regional white matter volume along with reduction in myelin damage. Among all white matter structures analyzed, the most significant changes were observed in the entorhinal region: The treatment group demonstrated a 5.14±3.66% increase, while the placebo group demonstrated a −7.60±4.35% decrease in volume. The difference between these two groups was statistically significant (p<0.002). The treatment group demonstrated a 2.78±4.97% increase and the placebo group demonstrated a −10.59±5.63% decrease in the myelin-reflecting T1w/T2w measurements. This difference was also statistically significant (p<0.003) between groups.

All white matter structures with statistically significant changes were in the treatment group and the most significant change was in the entorhinal region. Given its afferent connections to the hippocampus and the entorhinal cortex, and its relevance in AD pathology, reduction in white matter atrophy and myelin damage in the entorhinal region may play an important role in preventing disease progression.

### Example 6. Predicting Positive Outcome in Cognitive and Functional Abilities and Brain Atrophy in Subjects with Alzheimer's Disease: Coherent Activity Based on EEG Response to Gamma Oscillation Inducing Sensory Stimulation

Several resting-state EEG markers are identified as potential biomarkers for monitoring decline in integrity of neuronal network activities in AD. Changes in EEG patterns are considered for detecting early stage of AD and for differentiating AD from other neurodegenerative diseases.

This example describes a study conducted to evaluate neurophysiological response to gamma oscillation inducing sensory stimulation in AD patients as potential biomarkers for predicting clinical outcomes of cognitive and functional abilities and brain atrophy.

The study shows that patients in gamma oscillation inducing non-invasive sensory stimulation therapy maintain their activities of daily living and have improved sleep quality over a 6-month treatment period. Two outcome measures were evaluated: functional ability and sleep quality, which are strongly linked to AD. Maintenance of functional ability represents an important treatment and management goal for AD patients, which can reduce formal and informal care or delaying time to institutionalization when successful.

### Methods

The data presented here were from participants who participated in the Overture study (NCT03556280). Overture is a Phase I/II randomized, single-blind multi-center clinical trial. Participants in the treatment group of the trial used the GammaSense Stimulation System (Cognito Therapeutics, Inc., Cambridge, MA), a medical device that gives 40 Hz auditory and visual stimulation, for 1 hour every day during the 6-month trial. Participants in the sham group received sham stimulation. Prior to the start of the treatment period, baseline EEG recordings were acquired from all participants during 40 Hz auditory and visual stimulation. Here, it was investigated whether the baseline EEG responses during stimulation relate to the observed changes in patient outcomes during 6-month study period. First, it was identified the treatment and sham group participants who have baseline EEG recordings along with Mini-Mental State Examination (MMSE), Clinical Dementia Rating (CDR), Alzheimer's Disease Cooperative Study-Activities of Daily Living (ADCS-ADL) assessments and magnetic resonance imaging (MRI) volumetric data from lateral ventricles at baseline and after 6-month of stimulation (n=16). The participant scores were z-transformed, and a difference matrix was constructed, where each row represented a participant, and each column represented the observed change in a particular measure. The correlation between these z-transformed changes and the baseline EEG was examined. In addition, singular value decomposition was used to represent the changes in individual outcome variables (cognitive, functional, and structural) with a single unified measure, and the correlation between this measure and the EEG was also examined.

### Results

Dependence of the unified measure on the EEG coherence differed significantly between the two groups (p=0.0392; linear model, unified measure-EEG coherence interaction term). Within the treatment group, higher baseline EEG coherence corresponded to better response to therapy. Consistent with this observation, each of the individual outcome measures trended towards a positive correlation with the baseline EEG coherence.

In line with the predicted mechanism of gamma oscillation inducing sensory stimulation, higher degree of coherent activity in EEG response to sensory stimulation predicted positive outcome in clinical instruments assessing cognitive and functional abilities and brain atrophy.

### Conclusions

In the study, patients with high EEG coherence in response to gamma oscillation inducing sensory stimulation tended to exhibit better outcomes after 6-month treatment period. These results suggest that patients with higher EEG coherence at baseline may respond better to gamma oscillation inducing sensory stimulation. These findings raise the possibility that EEG response characteristics to sensory stimuli can be an important determinant for patient selection in clinical studies or specific treatments.

### Example 7. Randomized Controlled Trial of Gamma Oscillation Inducing Sensory Stimulation Treatment Demonstrates Maintained Cognitive and Functional Abilities and Reduced Brain Atrophy in Patients with Alzheimer's Disease

### Background

Recent experimental findings have shown that induction of synchronized 40 Hz gamma oscillation of neuronal networks by optogenetic or sensory (e.g., visual and/or auditory) stimulation effectively diminishes hallmarks of Alzheimer's disease (AD) pathology. Gamma oscillation inducing sensory stimulation reduces Aβ plaques, hyperphosphorylated tau, neurodegeneration, brain atrophy, and reverses synaptic loss and function, leading to improved learning abilities in transgenic mice carrying AD-related human pathological genes (Adaikkan & Tsai, 2020). These results initiated the development and validation of non-invasive, gamma oscillation inducing sensory stimulation as a potential therapeutic intervention for AD treatment.

### Objectives

A phase I/II randomized, controlled, double-blinded, US-based multi-center clinical trial (Overture trial; NCT03556280) was designed to evaluate feasibility, safety, tolerability, adherence, and efficacy of gamma oscillation inducing sensory stimulation, using Cognito Therapeutics medical device in subjects on the AD spectrum.

### Methods

Participants with AD (MMSE 14-26, inclusive) were randomized 2:1 to receive daily, one-hour, EEG-calibrated, 40 Hz noninvasive audio-visual stimulation or sham stimulation. At the start of the therapy, intensity of sensory stimulation was calibrated to each subject; following baseline EEG recordings, sensory evoked 40 Hz steady-state oscillation and cortical coherence were established at the tolerated intensities.

The randomized, controlled (RCT) phase of the trial lasted 6 months, during which therapy was self-administered at home with the help of a care partner. Patients, care partners, and assessment raters were blinded to group assignment. The RCT phase was followed by a 12 -month open label extension (OLE) during which all participants received active treatment. Safety was evaluated by MRI, a suicidality scale, and physical and neurological exams at baseline, 12-, and 24-weeks, and by monthly assessments of adverse events (AEs) during the trial. Tolerability was measured by device use data, a daily diary, and user experience interviews. Symptomatic changes were assessed daily via a diary, monthly via Alzheimer's Disease Cooperative Study-Activities of Daily Living (ADCS-ADL), Quality of Life, and care partner burden scales, quarterly via the Alzheimer's Disease Assessment Scale cognitive subscale (ADAS-Cog14), Neuropsychiatric Inventory, Clinical Dementia Rating, and bi-annually via Clock Drawing Test, and the Mini-Mental State Examination (MMSE). Plasma biomarkers, EEG recordings and brain volumetric changes (assessed by MRI), including whole brain, lateral ventricle, occipital and temporal lobe volumes and temporal lobe composite cortical thickness were assessed at baseline and after 3 and 6-months of treatment. APOE status was characterized at baseline. Actigraphy devices were worn continuously throughout the trial to assess daytime and nighttime activity.

### Results

A total of 135 subjects were screened, 74 (55%) were randomized, and 53 completed (72%) the trial. The rate of AEs during the trial were roughly equivalent between groups (active: 2.5/subject, sham: 2.9/subject). There were no unexpected serious treatment emergent adverse events. Review of MRI data demonstrated absence of ARIA in all subjects. High adherence rates (over 90%) were observed in both sham and treatment subjects. Participants easily adopted and adhered to daily self-administered therapy, with 80% of participants completed the RTC phase chose to continue into the OLE.

Among clinical instruments assessing cognitive and functional abilities, ADCS-ADL and MMSE scores demonstrated the most effective outcomes of the therapy. Over the 6-month treatment period, changes in ADCS-ADL scores were statistically significant between the sham and treatment groups, indicating a 78% slowing in functional decline by treatment (P<0.0003). Similarly, the treatment group demonstrated a statistically significant 83% (p<0.013) reduction in cognitive decline, as shown by changes in MMSE scores, compared to sham group. Other independent cognitive tests demonstrated a diminished cognitive decline in the treatment group compared to the sham group, although differences were not statistically significant. Nighttime active durations in the treatment group were significantly (p<0.03) reduced in the second 3 months compared to the first 3-months and the opposite change was observed in the sham group. Quantitative MRI analysis revealed that whole brain volume loss in the treatment group was 0.6%, whereas in the sham group this value was 1.5%, (comparable to the historic value of 1.12%), demonstrating a significant, 65% reduction in brain atrophy (p<0.01) by gamma oscillation inducing sensory stimulation over a 6-month period in this patient population. In line with the predicted mechanism of gamma oscillation inducing sensory stimulation, higher degree of coherent activity in EEG response to sensory stimulation predicted positive outcome in clinical instruments assessing cognitive and functional abilities and brain atrophy.

### Conclusion

Long-term, daily, self-administered, home-use of gamma oscillation inducing sensory stimulation is both safe and well tolerated in AD subjects. Patients given gamma oscillation inducing non-invasive sensory stimulation therapy maintained cognitive and functional abilities and showed improved sleep quality. In addition to ameliorating clinical symptoms, gamma oscillation inducing sensory stimulation reduces brain atrophy, indicating potential disease-modifying effects in AD.

### Example 8. Gamma Therapy Adjusted Based on Subject Response

A subject receives audio and visual stimulation having a frequency within the gamma range. Sensory induction of gamma neural oscillations in one or more brain regions is monitored via an EEG. The frequency and/or intensity of audio and/or visual stimulation is adjusted until an improvement in sensory induction of gamma neural oscillations is detected. A reduction or slowing of neurodegeneration, and an improvement in symptoms thereof, is expected.

### Example 9. Machine Learning Algorithm for Identifying Biomarkers

A machine learning algorithm is trained to identify a statistical relationship between (i) a first dataset of response measurements to gamma oscillation inducing non-invasive sensory from a patient population, and (ii) a second dataset of clinical measurements from the patient population, which is then used to determine biomarkers associated with a distinct clinical outcome (e.g., slowing of neurodegeneration). The algorithm is used to provide gamma oscillation inducing non-invasive sensory stimulation to a subject, the parameters (e.g., frequency, duration, and/or intensity) of which are adjusted to provide a therapeutic benefit to the subject.

### Example 10. Coherent Activity in EEG Response to Sensory Stimulation Predicts Positive Outcome in Cognitive and Functional Abilities, and Brain Atrophy in Subjects with Alzheimer's Disease

### Objectives

The present study evaluated whether neurophysiological responses to gamma oscillation inducing non-invasive sensory stimulation for a 6-month period could predict clinical outcome in subjects on the Alzheimer's Disease (AD) spectrum.

### Methods

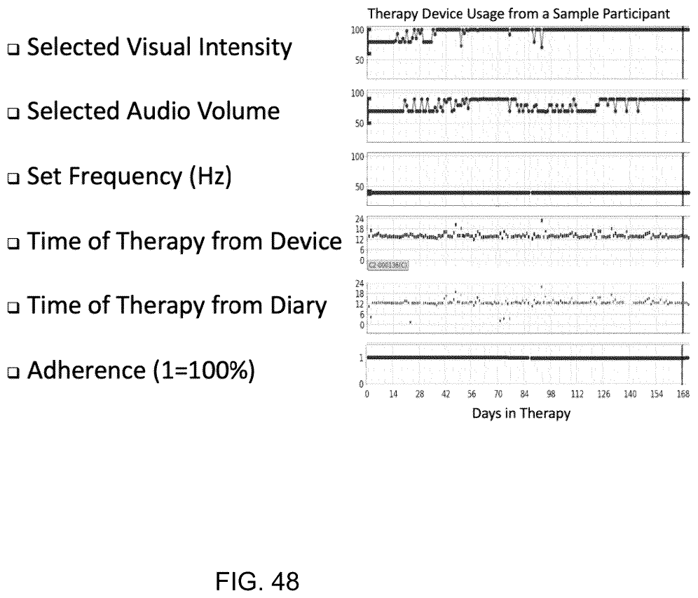

The data presented were from the Overture clinical trial (NCT03556280). In this trial, participants in the control group received sham stimulation and participants in the treatment group received 40 Hz auditory and visual stimulation, for daily, 1 hour treatment at home throughout the 6-month trial (FIG. 48). FIG. 48 provides an example of a participant's usage of a 40 Hz auditory and visual stimulation device throughout a 6-month period. The participant selected different visual and audio settings (first and second rows from the top) while the frequency of the device was set to 40 Hz (third row from the top). The time of the day the device was used was recorded by the device (fourth row from the top). Independently, the participant entered the therapy times into a diary (fifth row from the top). This participant shows close to 100% adherence (bottom row).

Participants were studied (n=16; Treatment: Placebo=10:6; Table 1) who had baseline EEG recordings during non-invasive sensory stimulation along with baseline and month 6 cognitive (assessed by Clinical Dementia Rating, CDR; Alzheimer's Disease Assessment Scale cognitive subscale, ADAS-Cog; Mini-Mental State Examination, MMSE; Neuropsychiatric Inventory Questionnaire Severity, NPIQ Severity), functional (Alzheimer's Disease Cooperative Study Activities of Daily Living, ADCS-ADL), and neural (from lateral ventricles vMRI-LV as percentage of total intracranial volume; MRI temporal cortex thickness; PET Composite amyloid standardized uptake value ratio, SUVR; PET Occipital amyloid SUVR) evaluations.

### Results

Changes in z-transformed clinical evaluations, relative to baseline, were examined as a function of baseline EEG coherence. In the treatment arm, a positive correlation between the baseline coherence and improvements in multiple domains was observed. To represent changes in these different domains as a single unified measure, a singular value decomposition was used, and the degree of correlation with the changes in the unified measure and baseline EEG coherence was studied.

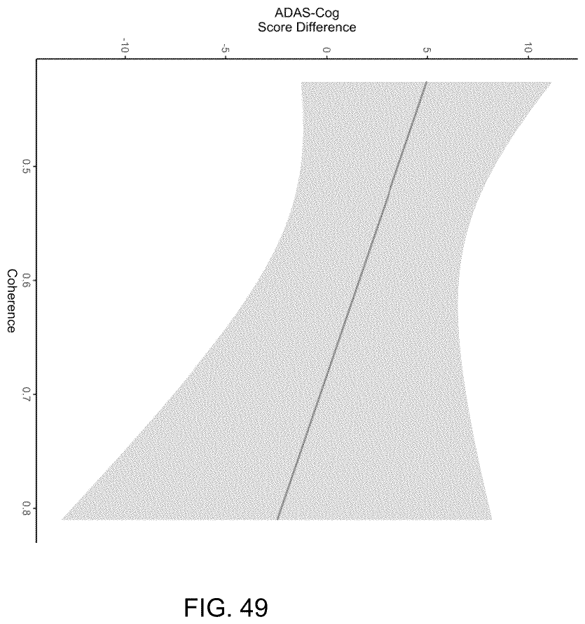

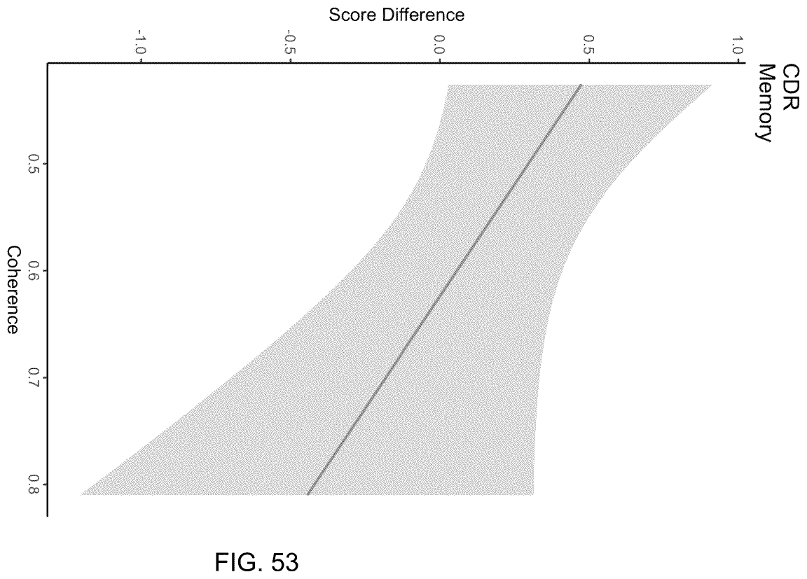

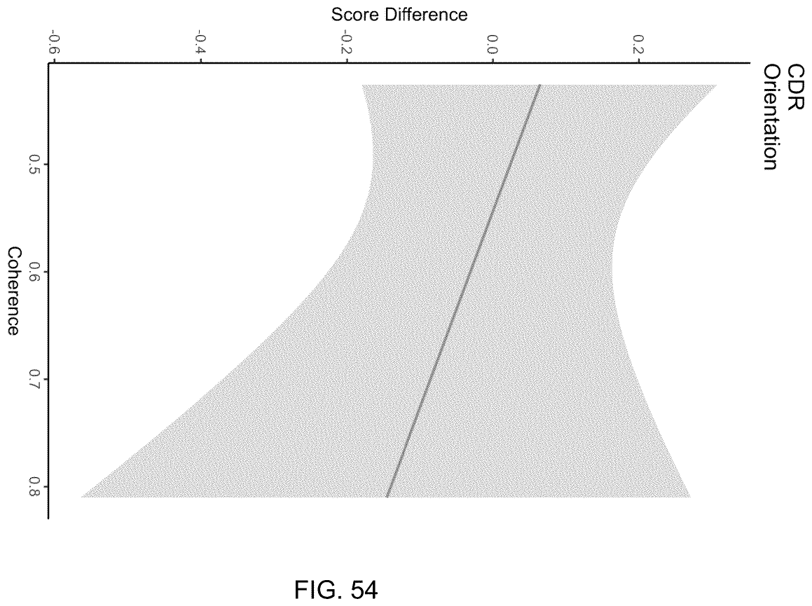

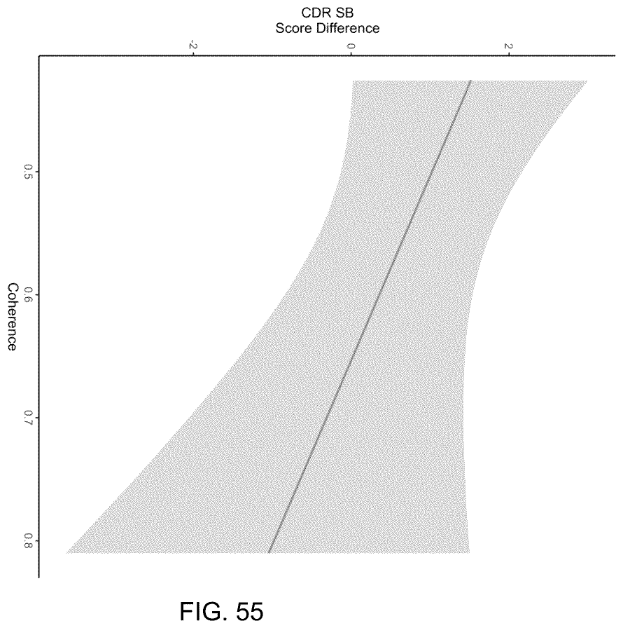

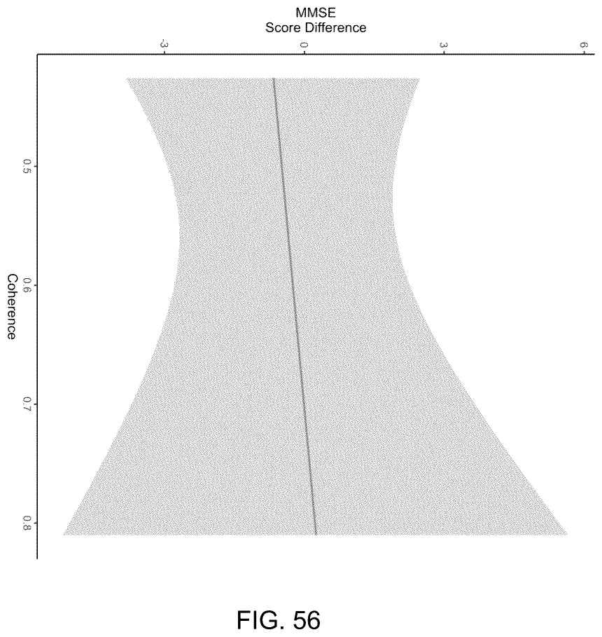

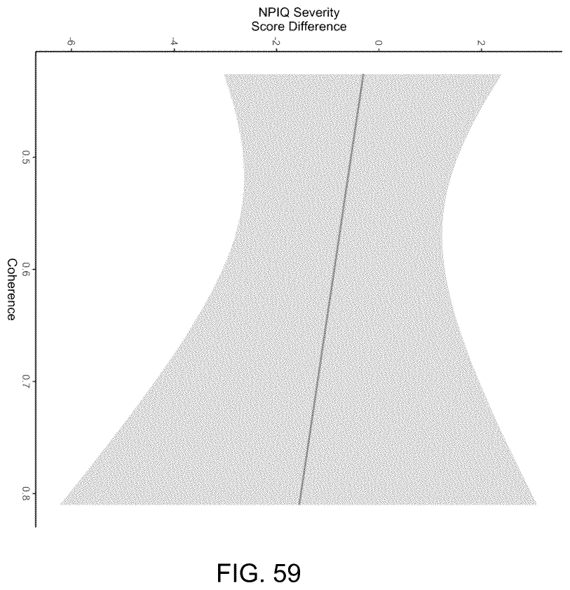

A positive correlation between the baseline coherence and improvements in cognition was observed. Cognition was assessed by: Alzheimer's Disease Assessment Scale cognitive subscale (ADAS-Cog FIG. 49); Clinical Dementia Rating, Memory (CDR-Memory, FIG. 53); Clinical Dementia Rating, Orientation (CDR-Orientation, FIG. 54); Clinical Dementia Rating scale Sum of Boxes (CDR-SB, FIG. 55); Mini-Mental State Examination (MMSE, FIG. 56); and Neuropsychiatric Inventory Questionnaire Severity (NPIQ Severity, FIG. 59).

FIG. 49 shows changes in the ADAS-Cog score as a function of baseline coherence after six months of active treatment using a 40 Hz auditory and visual stimulation device. As shown, an overall negative correlation between baseline coherence and ADAS-Cog score is observed. FIG. 53 provides changes in the Clinical Dementia Rating (CDR) scale, Memory score as a function of baseline coherence after six months of active treatment using a 40 Hz auditory and visual stimulation device. An overall negative correlation between baseline coherence and CDR, Memory score is observed. FIG. 54 provides changes in the Clinical Dementia Rating (CDR) scale, Orientation score as a function of baseline coherence after six months of active treatment using a 40 Hz auditory and visual stimulation device. As shown in FIG. 54, an overall negative correlation between baseline coherence and CDR, Orientation score is observed. FIG. 55 provides changes in the Clinical Dementia Rating scale, Sum of Boxes (CDR SB) score as a function of baseline coherence after six months of active treatment using a 40 Hz auditory and visual stimulation device. As shown in FIG. 55, overall negative correlation between baseline coherence and CDR SB score is observed. FIG. 56 provides changes in MMSE score as a function of baseline coherence after six months of active treatment using a 40 Hz auditory and visual stimulation device. As shown, an overall positive correlation between baseline coherence and MMSE score is observed. FIG. 59 shows changes in the NPIQ Severity score as a function of baseline coherence after six months of active treatment using a 40 Hz auditory and visual stimulation device. As shown in FIG. 59, an overall negative correlation between baseline coherence and NPIQ Severity score is observed.

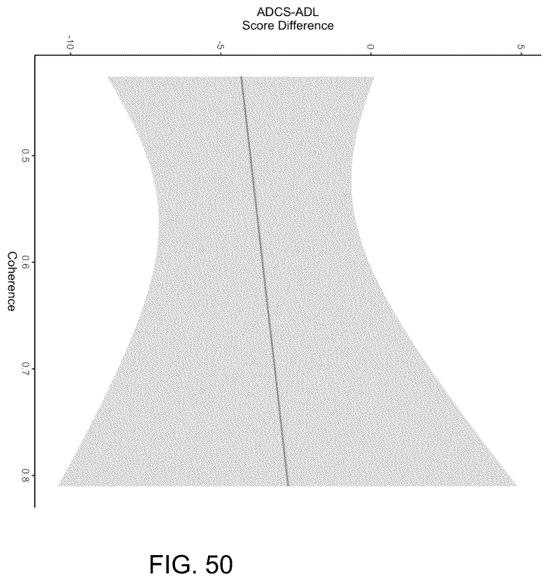

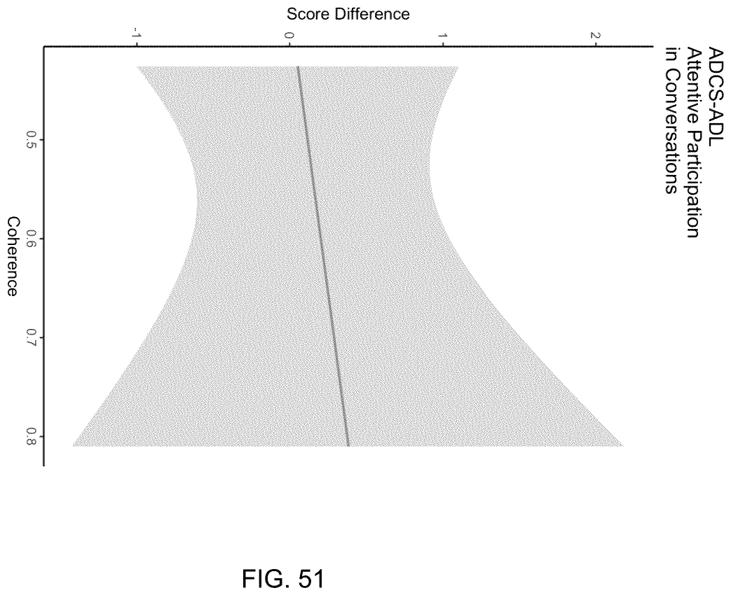

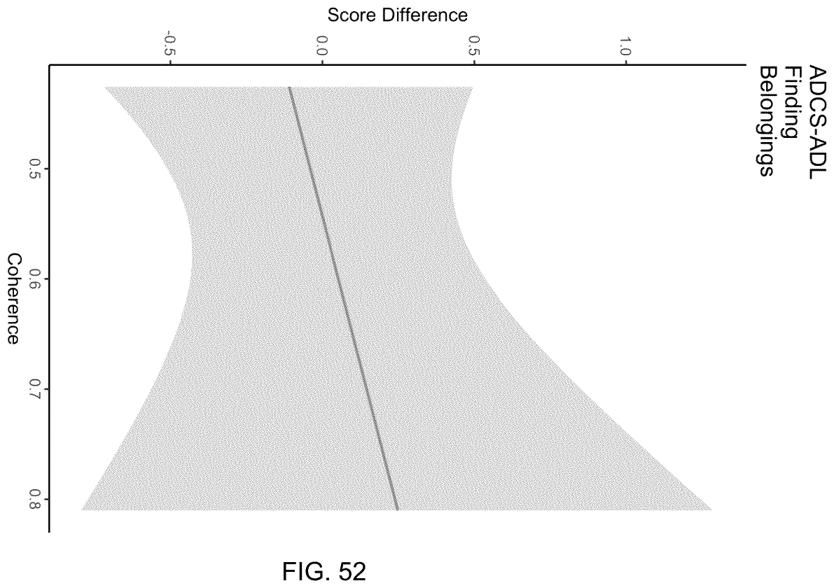

A positive correlation between the baseline coherence and improvements in function was also observed. Function was assessed by: Alzheimer's Disease Cooperative Study Activities of Daily Living (ADCS-ADL, FIG. 50); ADCS-ADL, Attentive Participation in Conversations (FIG. 51); and ADCS-ADL, Finding Belongings (FIG. 52). FIG. 50 ADCS-ADL score as a function of baseline coherence after six months of active treatment using a 40 Hz auditory and visual stimulation device. An overall positive correlation between baseline coherence and ADCS-ADL score is observed. FIG. 51 provides changes in the ADCS-ADL, Attentive Participation in Conversations score as a function of baseline coherence after six months of active treatment using a 40 Hz auditory and visual stimulation device. As shown, an overall positive correlation between baseline coherence and ADCS-ADL, Attentive Participation in Conversations score is observed. FIG. 52 provides changes in the ADCS-ADL, Finding Belongings score as a function of baseline coherence after six months of active treatment using a 40 Hz auditory and visual stimulation device. As shown, an overall positive correlation between baseline coherence and ADCS-ADL, Finding Belongings score is observed.

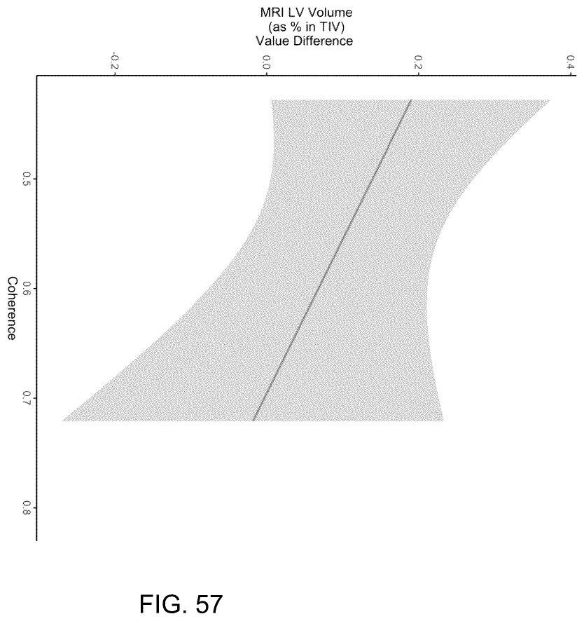

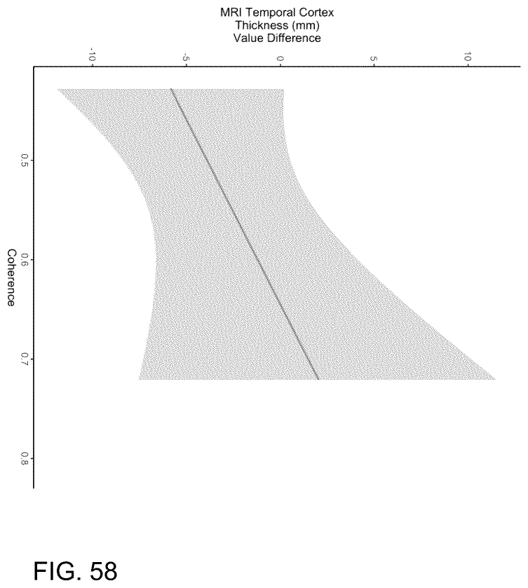

A positive correlation between the baseline coherence and improvements in nervous system structure (e.g., brain atrophy) was observed. Nervous system structure was assessed by: magnetic resonance imaging (MRI) lateral ventricle volume as a percentage of total intracranial volume (vMRI-LV as % in TIV, FIG. 57); and MRI temporal cortex thickness (mm, FIG. 58). FIG. 57 shows changes in the magnetic resonance imaging (MRI) lateral ventricle volume as a percentage of total intracranial volume (vMRI-LV as % in TIV) as a function of baseline coherence after six months of active treatment using a 40 Hz auditory and visual stimulation device. As shown in FIG. 57, an overall negative correlation between baseline coherence and lateral ventricle (LV) volume is observed. FIG. 58 shows changes in the MRI temporal cortex thickness (mm) as a function of baseline coherence after six months of active treatment using a 40 Hz auditory and visual stimulation device. As shown in FIG. 58, an overall positive correlation between baseline coherence and temporal thickness is observed.

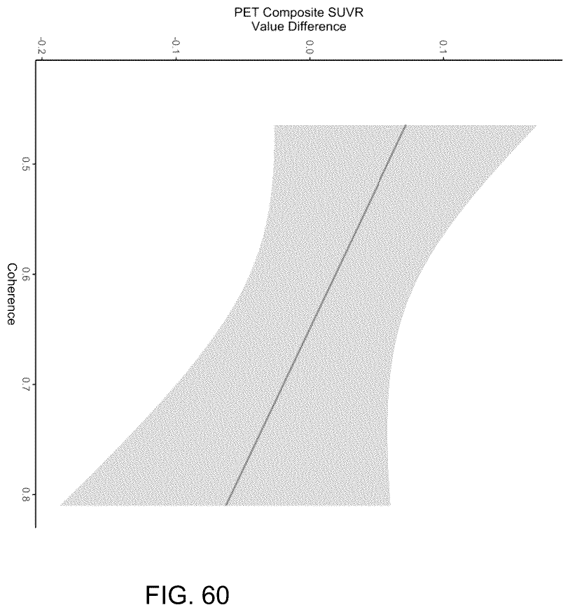

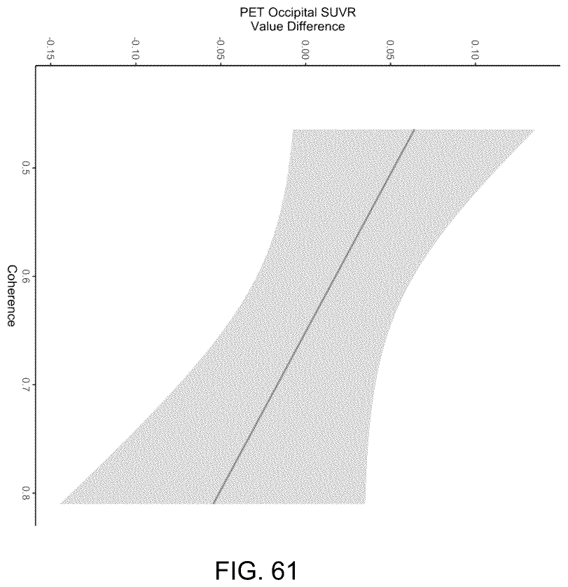

A positive correlation between the baseline coherence and improvements in biomarkers (e.g., amyloid load) was observed. Biomarkers were assessed by: positron emission tomography (PET) Composite amyloid standardized uptake value ratio (SUVR, FIG. 60); and PET Occipital amyloid SUVR (FIG. 61). FIG. 60 shows changes in the positron emission tomography (PET) Composite amyloid standardized uptake value ratio (SUVR) as a function of baseline coherence after six months of active treatment using a 40 Hz auditory and visual stimulation device. An overall negative correlation between baseline coherence and PET Composite SUVR is observed. FIG. 61 shows changes in the PET Occipital amyloid SUVR as a function of baseline coherence after six months of active treatment using a 40 Hz auditory and visual stimulation device. An overall negative correlation between baseline coherence and PET Occipital SUVR is observed.

### CONCLUSIONS

These results suggest that patients with higher EEG coherence in response to sensory stimulation respond better to gamma oscillation inducing 40 Hz sensory stimulation therapy. EEG response characteristics to sensory stimuli can be a determinant for patient selection in clinical studies.

What is claimed is:

1. A method of predicting a therapeutic benefit to a subject from a non-invasive sensory stimulus, the method comprising:
(a) delivering a gamma oscillation inducing non-invasive sensory stimulus to the subject, wherein the gamma oscillation inducing non-invasive sensory stimulus comprises an auditory stimulus and a visual stimulus;
(b) measuring a response from the subject; and
(c) predicting, using a statistical algorithm, a machine learning algorithm, or a combination thereof, the therapeutic benefit to the subject, wherein the predicting is based at least partially on the response measured in (b), and wherein the therapeutic benefit comprises a change in a rate of neurodegeneration, a change in a rate of aging, a change in cognition, and a change in physical function of the subject, or any combination thereof;
(d) responsive to the predicting, providing an additional oscillation inducing non-invasive sensory stimulus to the subject.

2. (canceled)

3. The method of claim 1, wherein the gamma oscillation inducing non-invasive sensory stimulus comprises a frequency component from about 20 Hz to about 60 Hz.

4. (canceled)

5. The method of claim 1, wherein the gamma oscillation inducing non-invasive sensory stimulus comprises a frequency component from about 30 Hz to about 50 Hz.

6.-9. (canceled)

10. The method of claim 1, wherein the gamma oscillation inducing non-invasive sensory stimulus is a visual stimulus, an auditory stimulus, a kinesthetic stimulus, or any combination thereof.

11. The method of claim 1, wherein the subject is diagnosed with a neurodegenerative disorder associated with cognitive decline, or is determined to be at a risk of developing the neurodegenerative disorder associated with the cognitive decline.

12. The method of claim 11, wherein the neurodegenerative disorder comprises supranuclear palsy (PSP), or transmissible spongiform encephalopathy.

13. (canceled)

14. The method of claim 11, wherein the neurodegenerative disorder is Alzheimer's disease, Creutzfeldt-Jakob disease (CJD), variant CJD, Gerstmann-Sträussler Scheinker Syndrome, Fatal Familial Insomnia, Kuru, or any combination thereof.

15. The method of claim 14, wherein the subject is diagnosed with or is at a risk of developing behavioral and psychological symptoms of dementia (BPSD).

16. The method of claim 15, wherein the subject is diagnosed with or at risk of developing a mood disorder, depression, bipolar disorder, anxiety, addiction, neurosis, anorexia, bulimia, apathy, agitation, dementia, mild cognitive impairment, subjective cognitive decline, Lewy body dementia, Parkinson's disease, sleep fragmentation, schizophrenia, or any combination thereof.

17.-19. (canceled)

20. The method of claim 1, wherein the response is measured using an electroencephalogram (EEG).

21. The method of claim 1, wherein the predicting is based, at least in part, on a measure of a change in EEG coherence compared to a baseline EEG coherence of the subject.

22. The method of claim 1, wherein the predicting is based, at least in part, on a measure of a change in EEG power compared to a baseline EEG power of the subject.

23. The method of claim 1, wherein the predicting is based, at least in part, on a change in power in an alpha, delta, theta, or beta frequency band, or combinations thereof.

24. The method of claim 1, wherein the measuring the response comprises using a Mini-Mental State Examination (MMSE), Alzheimer's Disease Assessment Scale (ADAS-Cog), Clinical Dementia Rating (CDR), Alzheimer's Disease Cooperative Study—Activities of Daily Living (ADCS-ADL), Neuropsychiatric Inventory (NPI), positron emission tomography (PET), or magnetic resonance imaging (MRI) volumetric data assessment.

25.-29. (canceled)

30. The method of claim 1, wherein the gamma oscillation inducing non-invasive sensory stimulus is continuously administered delivered for a predetermined duration of time.

31. The method of claim 30, wherein the predetermined duration of time is from 10 minutes to 2 hours.

32. The method of claim 1, wherein the gamma oscillation inducing non-invasive sensory stimulus is delivered for a plurality of discrete times.

33. The method of claim 32, wherein the plurality of discrete times occur at least once a day, at least once every two days, at least once a week, at least once every two weeks, at least once a month, or at least once every other month.

34. The method of claim 33, wherein the plurality of discrete times span over at least two days, at least a week, at least two weeks, at least a month, at least three months, at least six months, at least a year, at least two years, or at least five years.

35.-38. (canceled)

39. The method of claim 1, wherein the machine learning algorithm comprises a neural network, a deep learning algorithm, an ensemble, a regularization, a rule system, a regression, a Bayesian analysis, a decision tree, a dimensionality reduction, an instance-based algorithm, or a clustering algorithm.

40. The method of claim 1, wherein the predicting comprises using a separate means analysis.

41. The method of claim 1, further comprising using biometric data of the subject to predict the patient therapeutic benefit of the subject.

42. The method of claim 41, wherein the biometric comprises heart rate, blood pressure, breathing rate, body temperature, an electrical signal from any region of the subject's body, or sleep data.

43. The method of claim 42, wherein the sleep data comprises data on sleep fragmentation.

44.-175. (canceled)

176. The method of claim 1, wherein the gamma oscillation inducing non-invasive sensory stimulus comprises a plurality of pulses comprising a pulse rate interval, wherein the pulse rate interval is between 0.02 seconds and 0.033 seconds.

177. The method of claim 1, wherein the response is measured using an infrared optical sensor.

178. The method of claim 1, wherein the change in the rate of neurodegeneration, or the change in the rate of aging, comprises a reduction of neurodegeneration, a slowing of neurodegeneration, a reduction or prevention of brain atrophy, or a slowing of brain atrophy.

179. The method of claim 1, wherein the change in cognition or the change in physical function of the subject comprises a slowing of cognitive decline and a slowing of functional decline, an improvement in cognition, or an improvement in physical function.

180. The method of claim 1, wherein the additional oscillation inducing non-invasive sensory stimulus is a different stimulus than the gamma oscillation inducing non-invasive sensory stimulus.

181. The method of claim 180, wherein the different stimulus than the oscillation inducing non-invasive sensory stimulus is an alpha oscillation inducing stimulus, a beta oscillation inducing stimulus, a theta oscillation inducing stimulus, a delta oscillation inducing stimulus, or a slow wave oscillation stimulus.

182. The method of claim 1, wherein the additional oscillation inducing non-invasive sensory stimulus is a same stimulus as the gamma oscillation inducing non-invasive sensory stimulus.
